(19) United States
(12) Patent Application Publication
(10) Pub. No.: US 2022/0048895 A1
(43) Pub. Date: Feb. 17, 2022

(54) SUBSTITUTED BENZAZEPINE COMPOUNDS, CONJUGATES, AND USES THEREOF

(71) Applicant: SILVERBACK THERAPEUTICS, INC., Seattle, WA (US)

(72) Inventors: Craig Alan COBURN, Seattle, WA (US); Sean Wesley SMITH, Seattle, WA (US)

(21) Appl. No.: 17/275,528

(22) PCT Filed: Sep. 12, 2019
(86) PCT No.: PCT/US2019/050900

§ 371 Date: Mar. 11, 2021

Related U.S. Application Data
(60) Provisional application No. 62/730,492, filed on Sep. 12, 2018.

(51) Int. Cl. C07D 403/10; A61K 47/68; A61K 47/65

(52) U.S. Cl. CPC C07D 403/10; A61K 47/65; A61K 47/6851; A61K 47/6803

(57) ABSTRACT

Benzazepine compounds and salts thereof, conjugates and pharmaceutical compositions for use in the treatment of disease, such as cancer, are disclosed herein. The disclosed benzazepine compounds and salts thereof are useful, among other things, in treating of cancer and activating an immune response. Additionally, benzazepine compounds or salts thereof attached to an antibody construct to form an antibody conjugate are described herein.

### RELATED APPLICATION INFORMATION

This application claims the benefit of U.S. Provisional Application No. 62/730,492 filed Sep. 12, 2018, the content of which is incorporated herein by reference in its entirety.

### BACKGROUND OF THE INVENTION

One of the leading causes of death in the United States is cancer. The conventional methods of cancer treatment, like chemotherapy, surgery, or radiation therapy, tend to be either highly toxic or nonspecific to a cancer, or both, resulting in limited efficacy and harmful side effects. However, the immune system has the potential to be a powerful, specific tool in fighting cancers. In many cases tumors can specifically express genes whose products are required for inducing or maintaining the malignant state. These proteins may serve as antigen markers for the development and establishment of more specific anti-cancer immune response. The boosting of this specific immune response has the potential to be a powerful anti-cancer treatment that can be more effective than conventional methods of cancer treatment and can have fewer side effects.

### SUMMARY OF THE INVENTION

The disclosure provides compounds and conjugates for use as anti-cancer agents. In certain embodiments, compounds or conjugates of the disclosure stimulate an immune response for treating cancer.

In some aspects, the present disclosure provides a compound represented by the structure of Formula (IA):

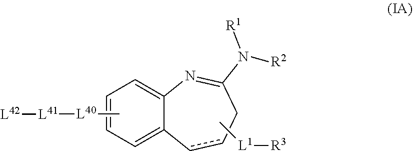

or a pharmaceutically acceptable salt thereof, wherein:
[symbol] represents an optional double bond;
L⁴⁰ is selected from C₃₋₁₂ carbocyclene and 3- to 12-membered heterocyclene, wherein the C₃₋₁₂ carbocyclene and the 3- to 12-membered heterocyclene are optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆alkynyl;
L¹ and L⁴¹ are independently selected from a bond, C₁-C₂ alkylene optionally substituted with one or more R³¹, —O—, —S—, —N(R¹⁰)—, —C(O)—, —C(O)O—, —OC(O)—, —C(O)N(R¹⁰)—, —N(R¹⁰)C(O)—, —C(NR¹⁰)—, —P(O)(OR¹⁰)O—, —O(R¹⁰O)(O)P—, —OS(O)—, —S(O)O—, —S(O)—, —OS(O)₂—, —S(O)₂O—, —N(R¹⁰)S(O)₂—, —S(O)₂N(R¹⁰)—, —N(R¹⁰)S(O)—, and —S(O)N(R¹⁰)—;
L⁴² is selected from: 3- to 8-membered saturated heterocycle substituted with a substituent selected from R³⁰, and the 3- to 8-membered saturated heterocycle is optionally substituted with one or more additional substituents selected from R³¹; and optionally substituted C₃₋₁₂ carbocycle, optionally substituted 3- to 12-membered unsaturated heterocycle, optionally substituted heteroaryl, and optionally substituted 8-14 membered bicyclic heterocycle each of which is optionally substituted with one or more substituents independently selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R¹ and R² are independently selected from hydrogen; and C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), and —CN;
R³ is selected from: —OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle, and C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R¹⁰ is independently selected at each occurrence from:
hydrogen; and
C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R¹¹ is independently selected at each occurrence from C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R³⁰ is selected from:
halogen, —OR¹¹, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R³¹ is selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each is which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl; and
wherein any substitutable carbon on the benzazepine core is optionally substituted by a substituent selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —P(O)(OR¹⁰)₂, —OP(O)(OR¹⁰)₂, —CN, C₁₋₁₀ alkyl, C₂₋₁₀ alkenyl, and C₂₋₁₀ alkynyl, or two substituents on a single carbon atom or two adjacent carbons combine to form a 3- to 7-membered carbocycle.

In some embodiments, the compound of Formula (IA) is represented by Formula (IB):

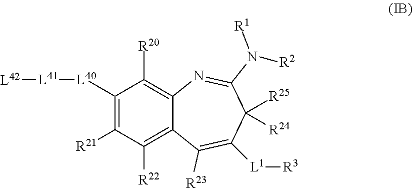

or a pharmaceutically acceptable salt thereof, wherein:
R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and
R²⁴ and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some aspects, the present disclosure provides a compound represented by the structure of Formula (IIIA):

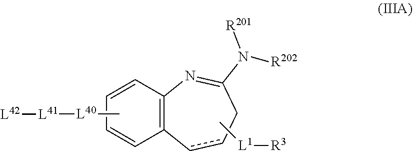

or a pharmaceutically acceptable salt thereof, wherein:
[symbol] represents an optional double bond;
L⁴⁰ is selected from C₃₋₁₂ carbocyclene and 3- to 12-membered heterocyclene, wherein the C₃₋₁₂ carbocyclene and the 3- to 12-membered heterocyclene are optionally substituted with one or more substituents independently selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
L¹ and L⁴¹ are independently selected from a bond, C₁-C₂ alkylene optionally substituted with one or more R³¹, —O—, —S—, —N(R¹⁰)—, —C(O)—, —C(O)O—, —OC(O)—, —C(O)N(R¹⁰)—, —N(R¹⁰)C(O)—, —C(NR¹⁰)—, —P(O)(OR¹⁰)O—, —O(R¹⁰O)(O)P—, —OS(O)—, —S(O)O—, —S(O)—, —OS(O)₂—, —S(O)₂O—, —N(R¹⁰)S(O)₂—, —S(O)₂N(R¹⁰)—, —N(R¹⁰)S(O)—, and —S(O)N(R¹⁰)—;
L⁴² is selected from: 3- to 8-membered saturated heterocycle substituted with a substituent selected from R³⁰, and optionally substituted with one or more additional substituents selected from R³¹; optionally substituted C₃₋₁₂ carbocycle, optionally substituted 3- to 12-membered unsaturated heterocycle, optionally substituted heteroaryl, and optionally substituted 8-14 membered bicyclic heterocycle each of which is optionally substituted with one or more substituents independently selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R²⁰¹ is hydrogen;
R²⁰² is an amine masking group;
R³ is selected from:
—OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R¹⁰ is independently selected at each occurrence from:
hydrogen; and
C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R¹¹ is independently selected at each occurrence from C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R³⁰ is selected from:
halogen, —OR¹¹, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; and C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R³¹ is selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each is which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl; and

wherein any substitutable carbon on the benzazepine core is optionally substituted by a substituent selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —P(O)(OR¹⁰)₂, —OP(O)(OR¹⁰)₂, —CN, C₁₋₁₀ alkyl, C₂₋₁₀ alkenyl, and C₂₋₁₀ alkynyl, or two substituents on a single carbon atom or two adjacent carbons combine to form a 3- to 7-membered carbocycle.

In some embodiments, the compound of Formula (IIIA) is represented by Formula (IIIB):

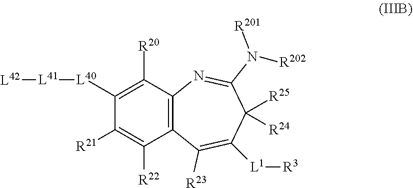

or a pharmaceutically acceptable salt thereof, wherein:
R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and
R²⁴ and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some embodiments for a compound or salt of Formula (IA), (IB), (IIIA), or (IIIB), R²⁰, R²¹, R²², and R²³ are independently selected from hydrogen, halogen, —OH, —NO₂, —CN, and C₁₋₁₀ alkyl. In some embodiments, R²⁰, R²¹, R²², and R²³ are each hydrogen. R²⁴ and R²⁵ may be independently selected from hydrogen, halogen, —OH, —NO₂, —CN, and C₁₋₁₀ alkyl, or R²⁴ and R²⁵ taken together form an optionally substituted saturated C₃₋₇ carbocycle. In some embodiments, R²⁴ and R²⁵ are each hydrogen. In other embodiments, R²⁴ and R²⁵ taken together form an optionally substituted saturated C₃₋₅ carbocycle.

In some embodiments for a compound or salt of Formula (IA) or (IB), R¹ is hydrogen. In some embodiments, R² is hydrogen.

In some embodiments for a compound or salt of Formula (IIIA) or (IIIB), R²⁰² is an enzymatically-cleavable group. R²⁰² may be represented by the formula:

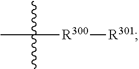

wherein:
R³⁰¹ is selected from an amino acid, a peptide, —O—(C₁-C₆ alkyl) and —C₁-C₆ alkyl, wherein alkyl of —O—(C₁-C₆ alkyl) and —C₁-C₆ alkyl is optionally substituted by one or more substituents independently selected from halogen, —OR¹⁰, —SR¹⁰, —N(R¹⁰)₂, —C(O)R¹⁰, —C(O)N(R¹⁰)₂, —NO₂, —CN, C₃₋₁₃ carbocycle, and 3- to 12-membered heterocycle; and
R³⁰⁰ is C(═O), wherein when R³⁰¹ is selected from an amino acid or peptide R³⁰⁰ is the C-terminus of the amino acid or peptide. In some embodiments, R³⁰¹ is a peptide selected from a dipeptide, tripeptide and tetrapeptide.

In some embodiments for a compound or salt of Formula (IA), (IB), (IIIA), or (IIIB), L¹ is selected from —C(O)—, and —C(O)NR¹⁰—. L¹ may be —C(O)—. L¹ may be —C(O)NR¹⁰—. In certain embodiments, R¹⁰ of —C(O)NR¹⁰— is selected from hydrogen and C₁₋₆ alkyl. For example, L¹ is —C(O)NH—.

In some embodiments for a compound or salt of Formula (IA), (IB), (IIIA), or (IIIB), R³ is selected from: —OR¹⁰, and —N(R¹⁰)₂; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl. R³ may be —N(R¹⁰)₂. In certain embodiments, R¹⁰ of —N(R¹⁰)₂ is independently selected at each occurrence from optionally substituted C₁₋₆ alkyl. For example, R¹⁰ of —N(R¹⁰)₂ may be independently selected at each occurrence from methyl, ethyl, propyl, and butyl, any one of which is optionally substituted. In some embodiments,

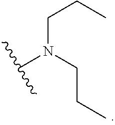 [SMILES: *N(CCC)CCC]

In some embodiments for a compound or salt of Formula (IA), (IB), (IIIA), or (IIIB), L⁴⁰ is an optionally substituted C₃₋₁₂ carbocyclene. L⁴⁰ may be an optionally substituted C₃₋₈ carbocyclene. L⁴⁰ may be an optionally substituted C₅₋₆ carbocyclene. L⁴⁰ may be an optionally substituted arylene. In certain embodiments, L⁴⁰ is an optionally substituted arylene wherein substituents are independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl. For example, L⁴⁰ may be an optionally substituted phenylene. In some embodiments, L⁴⁰ is an optionally substituted 3- to 12-membered heterocyclene. L⁴⁰ may be an optionally substituted 3- to 8-membered heterocyclene. L⁴⁰ may be an optionally substituted 5- to 6-membered heterocyclene. L⁴⁰ may be an optionally substituted heteroarylene. In certain embodiments, L⁴⁰ is an optionally substituted heteroarylene substituted with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl. L⁴⁰ may be an optionally substituted 5- or 6-membered heteroarylene. L⁴⁰ may be an optionally substituted 6-membered heteroaryl ene. For example, L⁴⁰ may be an optionally substituted pyridinylene.

In some embodiments for a compound or salt of Formula (IA), (IB), (IIIA), or (IIIB), L⁴¹ is selected from —N(R¹⁰)—, —C(O)N(R¹⁰)—, and —C(O)—. L⁴¹ may be —C(O)—. In some embodiments, L⁴² is selected from optionally substituted C₃₋₁₂ carbocycle, optionally substituted 3- to 12-membered unsaturated heterocycle, optionally substituted heteroaryl, and optionally substituted 8-14 membered bicyclic heterocycle. L⁴² may be an optionally substituted 8- to 14-membered bicyclic heterocycle. L⁴² may be an optionally substituted 8- to 12-membered bicyclic heterocycle. In certain embodiments, L⁴² is an optionally substituted 8- to 12-membered bicyclic heterocycle with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆alkynyl. L⁴² may be an optionally substituted 8- to 12-membered bicyclic heterocycle with one or more substituents independently selected from —OR¹⁰, —N(R¹⁰)₂, and ═O. In some embodiments, L⁴² is a 3- to 8-membered saturated heterocycle substituted with a substituent selected from R³⁰, and optionally substituted with one or more substituents selected from R³¹. L⁴² may be a 5- to 6-membered saturated heterocycle substituted with a substituent selected from R³⁰, and optionally substituted with one or more substituents selected from R³¹.

In some embodiments for a compound or salt of Formula (IA), (IB), (IIIA), or (IIIB), R³⁰ is selected from halogen, —OR¹¹, —SR¹⁰, —C(O)N(R¹⁰)₂, —N(R¹⁰)₂, —C(O)OR¹⁰, —NO₂, and —CN; C₁₋₁₀ alkyl, C₂₋₁₀ alkenyl, and C₂₋₁₀ alkynyl, each of which is independently optionally substituted at each occurrence with one or more substituents; and C₃₋₁₂ carbocycle and 3- to 12-membered heterocycle, each of which is independently optionally substituted with one or more substituents. R³⁰ may be selected from —OR¹¹; C₁₋₁₀ alkyl, C₂₋₁₀ alkenyl, and C₂₋₁₀ alkynyl, each of which is independently optionally substituted at each occurrence with one or more substituents; and C₃₋₁₂ carbocycle and 3- to 12-membered heterocycle, each of which is optionally substituted with one or more substituents.

In some embodiments for a compound or salt of Formula (IA), (IB), (IIIA), or (IIIB), R³¹ is selected from halogen, —OR¹⁰, —SR¹⁰, —C(O)N(R¹⁰)₂, —N(R¹⁰)₂, —C(O)OR¹⁰, —NO₂, and —CN; C₁₋₁₀ alkyl, C₂₋₁₀ alkenyl, and C₂₋₁₀ alkynyl, each of which is optionally substituted with one or more independently selected substituents; and C₃₋₁₂ carbocycle and 3- to 12-membered heterocycle, each of which is optionally substituted with one or more independently selected substituents. R³¹ may be selected from —OR¹⁰; C₁₋₁₀ alkyl, C₂₋₁₀ alkenyl, and C₂₋₁₀ alkynyl, each of which is optionally substituted with one or more independently selected substituents; and C₃₋₁₂ carbocycle and 3- to 12-membered heterocycle, wherein each of which is optionally substituted with one or more independently selected substituents.

In some embodiments for a compound or salt of Formula (IA), (IB), (IIIA), or (IIIB), L⁴² is pyrrolidine substituted with a substituent selected from R³⁰, and optionally substituted with one or more substituents selected from R³¹. In some embodiments, L⁴² is piperidine substituted with a substituent selected from R³⁰, and optionally substituted with one or more substituents selected from R³¹.

In some embodiments for a compound or salt of Formula (IA), the compound is selected from:

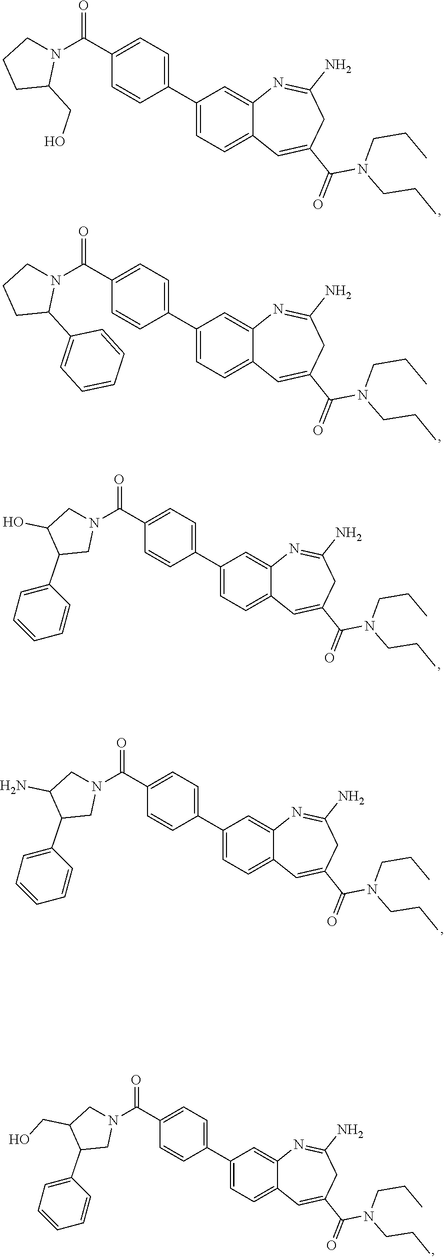 [SMILES: CCCN(CCC)C(=O)C1=Cc2ccc(-c3ccc(C(=O)N4CCCC4CO)cc3)cc2N=C(N)C1 ; CCCN(CCC)C(=O)C1=Cc2ccc(-c3ccc(C(=O)N4CCCC4c4ccccc4)cc3)cc2N=C(N)C1 ; CCCN(CCC)C(=O)C1=Cc2ccc(-c3ccc(C(=O)N4CC(O)C(c5ccccc5)C4)cc3)cc2N=C(N)C1 ; CCCN(CCC)C(=O)C1=Cc2ccc(-c3ccc(C(=O)N4CC(N)C(c5ccccc5)C4)cc3)cc2N=C(N)C1 ; CCCN(CCC)C(=O)C1=Cc2ccc(-c3ccc(C(=O)N4CC(CO)C(c5ccccc5)C4)cc3)cc2N=C(N)C1]

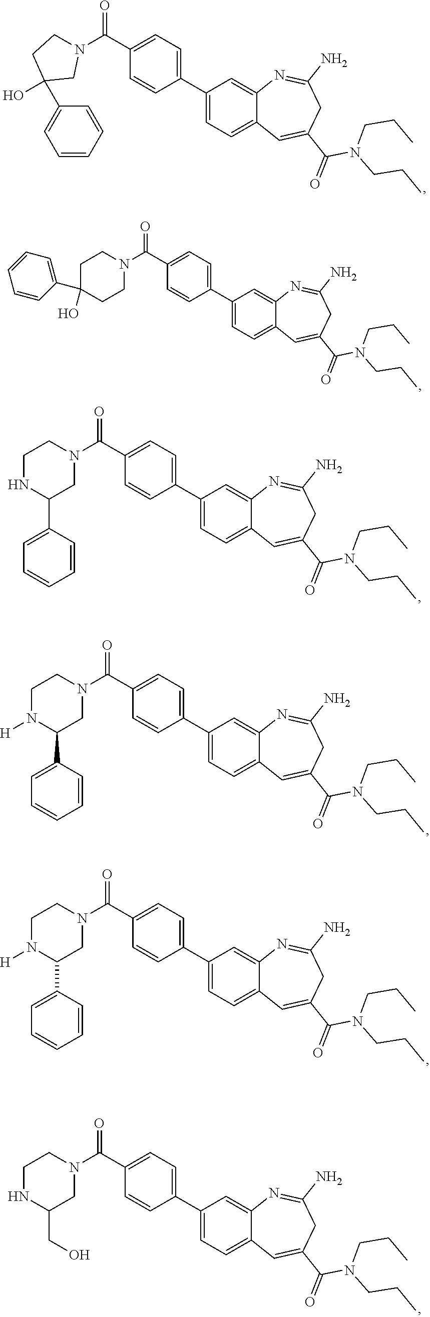 [SMILES: CCCN(CCC)C(=O)C1=Cc2ccc(-c3ccc(C(=O)N4CCC(O)(c5ccccc5)C4)cc3)cc2N=C(N)C1 ; CCCN(CCC)C(=O)C1=Cc2ccc(-c3ccc(C(=O)N4CCC(O)(c5ccccc5)CC4)cc3)cc2N=C(N)C1 ; CCCN(CCC)C(=O)C1=Cc2ccc(-c3ccc(C(=O)N4CCNC(c5ccccc5)C4)cc3)cc2N=C(N)C1 ; CCCN(CCC)C(=O)C1=Cc2ccc(-c3ccc(C(=O)N4CCN[C@H](c5ccccc5)C4)cc3)cc2N=C(N)C1 ; CCCN(CCC)C(=O)C1=Cc2ccc(-c3ccc(C(=O)N4CCN[C@@H](c5ccccc5)C4)cc3)cc2N=C(N)C1 ; CCCN(CCC)C(=O)C1=Cc2ccc(-c3ccc(C(=O)N4CCNC(CO)C4)cc3)cc2N=C(N)C1]

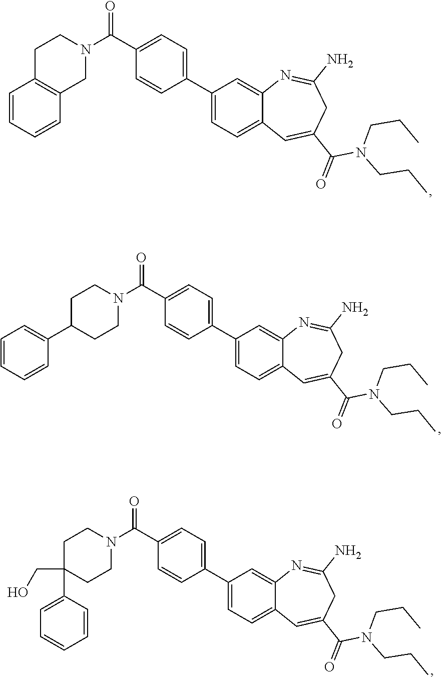 [SMILES: CCCN(CCC)C(=O)C1=Cc2ccc(-c3ccc(C(=O)N4CCc5ccccc5C4)cc3)cc2N=C(N)C1 ; CCCN(CCC)C(=O)C1=Cc2ccc(-c3ccc(C(=O)N4CCC(c5ccccc5)CC4)cc3)cc2N=C(N)C1 ; CCCN(CCC)C(=O)C1=Cc2ccc(-c3ccc(C(=O)N4CCC(CO)(c5ccccc5)CC4)cc3)cc2N=C(N)C1]

and a salt of any one thereof.

In some aspects, the present disclosure provides a compound represented by the structure of Formula (IIA):

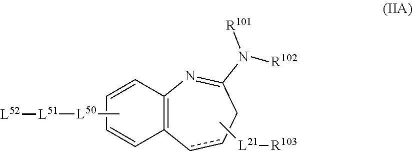

or a pharmaceutically acceptable salt thereof, wherein:
[symbol] represents an optional double bond;
L⁵⁰ is selected from C₃₋₁₂ carbocyclene and 3- to 12-membered heterocyclene, wherein the C₃₋₁₂ carbocyclene and the 3- to 12-membered heterocyclene are optionally substituted with one or more substituents independently selected at each occurrence from:
halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
L²¹ and L⁵¹ are independently selected from a bond, C₁-C₂ alkylene optionally substituted with one or more R³¹⁰, —O—, —S—, —N(R¹⁰⁰)—, —C(O)—, —C(O)O—, —OC(O)—, —C(O)N(R¹⁰⁰)—, —N(R¹⁰⁰)C(O)—, —C(NR¹⁰⁰)—, —P(O)(OR¹⁰⁰)O—, —O(R¹⁰⁰O)(O)P—, —OS(O)—, —S(O)O—, —S(O)—, —OS(O)₂—, —S(O)₂O—, —N(R¹⁰⁰)S(O)₂—, —S(O)₂N(R¹⁰⁰)—, —N(R¹⁰⁰)S(O)—, and —S(O)N(R¹⁰⁰)—;
L⁵² is selected from optionally substituted C₃₋₁₂ carbocycle, optionally substituted 3- to 12-membered unsaturated heterocycle, optionally substituted heteroaryl, optionally substituted 8-14 membered bicyclic heterocycle, and optionally substituted 3- to 8-membered saturated heterocycle, each of which is optionally substituted with one or more substituents independently selected from:
halogen, -L², —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
R¹⁰¹ and R¹⁰² are independently selected from L², and hydrogen; and C₁₋₁₀ alkyl, C₂₋₁₀ alkenyl, and C₂₋₁₀ alkynyl, each of which is optionally substituted with one or more substituents independently selected from L², halogen, —OR¹⁰⁰, —SR¹⁰⁰, —C(O)N(R¹⁰⁰)₂, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), and —CN;
R¹⁰³ is selected from:
-L², —OR¹⁰⁰, —N(R¹⁰⁰)₂, —C(O)N(R¹⁰⁰)₂, —C(O)R¹⁰⁰, —C(O)OR¹⁰⁰, —S(O)R¹⁰⁰, and —S(O)₂R¹⁰⁰; and
C₁₋₁₀ alkyl, C₂₋₁₀ alkenyl, and C₂₋₁₀ alkynyl, each of which is optionally substituted with one or more substituents independently selected from L², halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from L², halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
R¹⁰⁰ is independently selected at each occurrence from L² and hydrogen; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R³¹⁰ is selected from:
halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰,
—OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
L² is a linker wherein at least one of R¹⁰¹, R¹⁰², R¹⁰³, and R¹⁰⁰ is L² or at least one substituent on R¹⁰¹, R¹⁰², R¹⁰³, L⁵², L²¹ and L⁵¹ is -L²; and
wherein any substitutable carbon on the benzazepine core is optionally substituted by a substituent selected from halogen, —OR¹⁰⁰, —SR¹⁰⁰, —C(O)N(R¹⁰⁰)₂, —N(R¹⁰⁰)₂, —S(O)R¹⁰⁰, —S(O)₂R¹⁰⁰,
   —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₁₀ alkyl, C₂₋₁₀ alkenyl, and C₂₋₁₀ alkynyl, or two substituents on a single carbon atom or two adjacent carbons combine to form a 3- to 7-membered carbocycle.

In some embodiments, the compound of Formula (IIA) is represented by Formula (IIB):

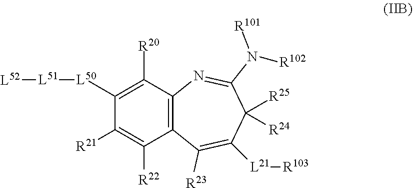

or a pharmaceutically acceptable salt thereof, wherein:
R²⁰, R²¹, R²², and R²³ are independently selected from hydrogen, halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl; and
R²⁴, and R²⁵ are independently selected from hydrogen, halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some aspects, the present disclosure provides a compound represented by the structure of Formula (IVA):

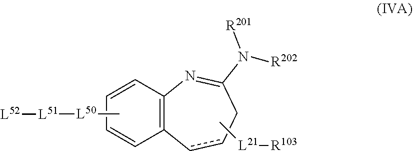

or a pharmaceutically acceptable salt thereof, wherein:
[symbol] represents an optional double bond;
L⁵⁰ is selected from C₃₋₁₂ carbocyclene and 3- to 12-membered heterocyclene, wherein the C₃₋₁₂ carbocyclene and the 3- to 12-membered heterocyclene are optionally substituted with one or more substituents independently selected at each occurrence from:
halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
L²¹ and L⁵¹ are independently selected from a bond, C₁-C₂ alkylene optionally substituted with one or more R³¹⁰, —O—, —S—, —N(R¹⁰⁰)—, —C(O)—, —C(O)O—, —OC(O)—, —C(O)N(R¹⁰⁰)—, —N(R¹⁰⁰)C(O)—, —C(NR¹⁰⁰)—, —P(O)(OR¹⁰⁰)O—, —O(R¹⁰⁰O)(O)P—, —OS(O)—, —S(O)O—, —S(O)—, —OS(O)₂—, —S(O)₂O—, —N(R¹⁰⁰)S(O)₂—, —S(O)₂N(R¹⁰⁰)—, —N(R¹⁰⁰)S(O)—, and —S(O)N(R¹⁰⁰)—;
L⁵² is selected from optionally substituted C₃₋₁₂ carbocycle, optionally substituted 3- to 12-membered unsaturated heterocycle, optionally substituted heteroaryl, optionally substituted 8-14 membered bicyclic heterocycle, and optionally substituted 3- to 8-membered saturated heterocycle, each of which is optionally substituted with one or more substituents independently selected from:
halogen, -L², —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
R²⁰¹ is hydrogen;
R²⁰² is an amine masking group;
R¹⁰³ is selected from:
-L², —OR¹⁰⁰, —N(R¹⁰⁰)₂, —C(O)N(R¹⁰⁰)₂, —C(O)R¹⁰⁰, —C(O)OR¹⁰⁰, —S(O)R¹⁰⁰, and —S(O)₂R¹⁰⁰; and
C₁₋₁₀ alkyl, C₂₋₁₀ alkenyl, and C₂₋₁₀ alkynyl, each of which is optionally substituted with one or more substituents independently selected from L², halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from L², halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
R¹⁰⁰ is independently selected at each occurrence from L² and hydrogen; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R³¹⁰ is selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
L² is a linker, wherein at least one of R²⁰¹, R²⁰², R¹⁰³, and R¹⁰⁰ is L² or at least one substituent on R²⁰¹, R²⁰², R¹⁰³, L⁵², L²¹ and L⁵¹ is -L²; and
wherein any substitutable carbon on the benzazepine core is optionally substituted by a substituent selected from halogen, —OR¹⁰⁰, —SR¹⁰⁰, —C(O)N(R¹⁰⁰)₂, —N(R¹⁰⁰)₂, —S(O)R¹⁰⁰, —S(O)₂R¹⁰⁰,
   —C(O)R¹⁰⁰, —C(O)OR¹⁰⁰, —OC(O)R¹⁰⁰, —NO₂, ═O, ═S,
   ═N(R¹⁰⁰), —P(O)(OR¹⁰⁰)₂, —OP(O)(OR¹⁰⁰)₂, —CN, C₁₋₁₀ alkyl, C₂₋₁₀ alkenyl, and C₂₋₁₀ alkynyl, or two substituents on a single carbon atom or two adjacent carbons combine to form a 3- to 7-membered carbocycle.

In some embodiments, the compound of Formula (IVA) is represented by Formula (IVB):

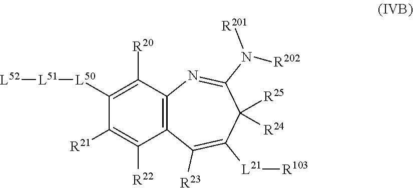

or a pharmaceutically acceptable salt thereof, wherein:
R²⁰, R²¹, R²², and R²³ are independently selected from hydrogen, halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl; and
R²⁴, and R²⁵ are independently selected from hydrogen, halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some embodiments for a compound or salt of Formula (IIA) or (IIB), R¹⁰¹ is -L². In some embodiments, R¹⁰² is -L².

In some embodiments for a compound or salt of Formula (IVA) or (IVB), R²⁰² is an enzymatically-cleavable group. R²⁰² is represented by the formula:

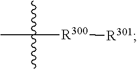

wherein:
R³⁰¹ is selected from an amino acid, a peptide, —O—(C₁-C₆ alkyl) and —C₁-C₆ alkyl, wherein alkyl of —O—(C₁-C₆ alkyl) and —C₁-C₆ alkyl is optionally substituted by one or more substituents independently selected from halogen, —OR¹⁰, —SR¹⁰, —N(R¹⁰)₂, —C(O)R¹⁰, —C(O)N(R¹⁰)₂, —NO₂, —CN, C₃₋₁₃ carbocycle, and 3- to 12-membered heterocycle; and
R³⁰⁰ is C(═O), wherein when R³⁰¹ is selected from an amino acid or peptide R³⁰⁰ is the C-terminus of the amino acid or peptide. In some embodiments, R³⁰¹ is a peptide selected from a dipeptide, tripeptide and tetrapeptide.

In some embodiments for a compound or salt of Formula (IIA), (IIB), (IVA), or (IVB), L²¹ is —C(O)—. In some embodiments, L²¹ is —C(O)NR¹⁰⁰—. R¹⁰⁰ of —C(O)NR¹⁰⁰— may be selected from hydrogen, C₁₋₆ alkyl, and -L². In some embodiments, L²¹ is —C(O)NH—.

In some embodiments for a compound or salt of Formula (IIA), (IIB), (IVA), or (IVB), R¹⁰³ is selected from -L², —OR¹⁰⁰, and —N(R¹⁰⁰)₂; and C₁₋₁₀ alkyl, C₂₋₁₀ alkenyl, C₂₋₁₀ alkynyl, C₃₋₁₂ carbocycle, 3- to 12-membered heterocycle, aryl, and heteroaryl, each of which is independently optionally substituted at each occurrence with one or more substituents selected from -L², halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl. In certain embodiments, R¹⁰⁰ of —N(R¹⁰⁰)₂ is selected from -L² and hydrogen, and wherein no more than one R¹⁰⁰ of —N(R¹⁰⁰)₂ is -L².

In some embodiments for a compound or salt of Formula (IIA), (IIB), (IVA), or (IVB), L⁵⁰ is an optionally substituted arylene wherein substituents are independently selected from halogen, —OR¹⁰⁰, —SR¹⁰⁰, —N(R¹⁰⁰)₂, —C(O)R¹⁰⁰, —C(O)OR¹⁰⁰, —OC(O)R¹⁰⁰, —NO₂, ═O, ═S, —CN, C₁₋₆ alkyl, C₂₋₆ alkenyl, and C₂₋₆ alkynyl. For example, L⁵⁰ may be an optionally substituted phenylene.

In some embodiments for a compound or salt of Formula (IIA), (IIB), (IVA), or (IVB), L⁵¹ is —C(O)N(R¹⁰⁰)—. R¹⁰⁰ of —C(O)N(R¹⁰⁰)— may be selected from hydrogen, C₁₋₆ alkyl, and -L². For example, L⁵¹ may be —C(O)NH—.

In some embodiments for a compound or salt of Formula (IIA), (IIB), (IVA), or (IVB), L⁵² is an optionally substituted 8- to 14-membered bicyclic heterocycle. L⁵² may be an optionally substituted 8- to 12-membered bicyclic heterocycle with one or more substituents independently selected from —OR¹⁰⁰, —N(R¹⁰⁰)₂, and ═O. In some embodiments, L⁵² is a 3- to 8-membered saturated heterocycle optionally substituted with one or more substituents selected from R³¹⁰. R³¹⁰ may be selected from L² and —OR¹⁰⁰; C₁₋₁₀ alkyl, C₂₋₁₀ alkenyl, and C₂₋₁₀ alkynyl, each of which is optionally substituted with one or more independently selected substituents; and C₃₋₁₂ carbocycle and 3- to 12-membered heterocycle each of which is optionally substituted with one or more independently selected substituents. In certain embodiments, L⁵² is pyrrolidine optionally substituted with one or more substituents selected from R³¹⁰. In certain embodiments, L⁵² is piperidine optionally substituted with one or more substituents selected from R³¹⁰.

In some embodiments for a compound or salt of Formula (IIA), (IIB), (IVA), or (IVB), L² is a cleavable linker or a noncleavable linker. L² may be a cleavable linker that is cleavable by a lysosomal enzyme.

In some embodiments for a compound or salt of Formula (IIA), (IIB), (IVA), or (IVB), L² is represented by the formula:

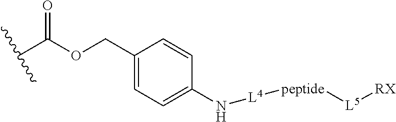

wherein:

L⁴ represents the C-terminus of the peptide and L⁵ is selected from a bond, alkylene and heteroalkylene, wherein L⁵ is optionally substituted with one or more groups independently selected from R³⁰, and RX is a reactive moiety; and

R³⁰ is independently selected at each occurrence from halogen, —OH, —CN, —O-alkyl, —SH, ═O, ═S, —NH₂, and —NO₂; and C₁-C₁₀ alkyl, C₂-C₁₀ alkenyl, and C₂-C₁₀ alkynyl, each of which is independently optionally substituted at each occurrence with one or more substituents selected from halogen, —OH, —CN, —O-alkyl, —SH, ═O, ═S, —NH₂, and —NO₂.

In some embodiments for a compound or salt of Formula (IIA), (IIB), (IVA), or (IVB), RX comprises a leaving group. RX may be a maleimide or an alpha-halo carbonyl. In some embodiments, the peptide of L² comprises Val-Cit or Val-Ala.

In some embodiments for a compound or salt of Formula (IIA), (IIB), (IVA), or (IVB), L² is represented by the formula:

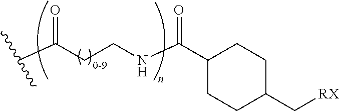

wherein:
RX comprises a reactive moiety; and
n is 0-9.

In some embodiments for a compound or salt of Formula (IIA), (IIB), (IVA), or (IVB), RX comprises a leaving group. RX may be a maleimide or an alpha-halo carbonyl.

In some embodiments for a compound or salt of Formula (IIA), (IIB), (IVA), or (IVB), L² is further covalently bound to a residue of an antibody construct to form a conjugate, the antibody construct comprising an antigen binding domain and an Fc domain.

In some aspects, the present disclosure provides a conjugate represented by the formula:

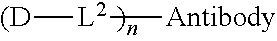

wherein:
Antibody is an antibody construct, the antibody construct comprising an antigen binding domain and an Fc domain;
n is 1 to 20;
D is the compound or salt disclosed herein; and
L² is a linker moiety attached to a residue of the antibody construct and to D.

In some embodiments, n is selected from 1 to 8. In certain embodiments, n is selected from 2 to 5. In certain embodiments, n is 2 or 4.

In some embodiments, -L² is represented by the formula:

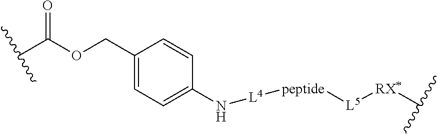

wherein:
L⁴ represents the C-terminus of the peptide and L⁵ is selected from a bond, alkylene and heteroalkylene, wherein L⁵ is optionally substituted with one or more groups independently selected from R³⁰; RX* is a bond, a succinimide moiety, or a hydrolyzed succinimide moiety bound to the residue of the antibody construct, wherein [symbol] on RX* represents the point of attachment to the residue of the antibody construct; and
R³⁰ is independently selected at each occurrence from halogen, —OH, —CN, —O-alkyl, —SH, ═O, ═S, —NH₂, and —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂.

In some embodiments, RX* is a succinamide moiety, hydrolyzed succinamide moiety or a mixture thereof and is bound to a cysteine residue of an antibody construct.

In some embodiments, -L² is represented by the formula:

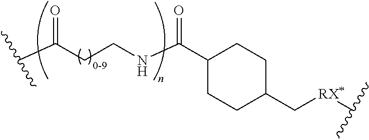

wherein:
RX* is a bond, a succinimide moiety, or a hydrolyzed succinimide moiety bound to the residue of the antibody construct, wherein [symbol] on RX* represents the point of attachment to the residue of the antibody construct; and
n is 0-9.

In some embodiments, the antigen binding domain specifically binds to an antigen selected from the group consisting of HER2, TROP2 and MUC16. In some embodiments, the Fc domain is an Fc null.

In some aspects, the present disclosure provides a pharmaceutical composition, comprising a conjugate described herein, and a pharmaceutically acceptable excipient. The average Drug-to-Antibody Ratio (DAR) may be from 1 to 8.

In some aspects, the present disclosure provides a method for the treatment of cancer, comprising administering an effective amount of the compound or salt described herein to a subject in need thereof.

In some aspects, the present disclosure provides a method for the treatment of cancer, comprising administering an effective amount of the conjugate described herein or the pharmaceutical composition described herein to a subject in need thereof.

In some aspects, the present disclosure provides a method of killing tumor cells in vivo, comprising contacting a tumor cell population with the conjugate described herein or the pharmaceutical composition described herein.

In some aspects, the present disclosure provides a method for treatment, comprising administering to a subject the conjugate described herein or the pharmaceutical composition described herein.

In some aspects, the present disclosure provides a method for the treatment of cancer, comprising administering to a subject in need thereof the conjugate described herein or the pharmaceutical composition described herein. In some embodiments, the cancer is breast cancer, gastric cancer or lung cancer.

In some aspects, the present disclosure provides a compound or salt described herein for use in a method of treatment of a subject's body by therapy.

In some aspects, the present disclosure provides a compound or salt described herein for use in a method of treating cancer.

In some aspects, the present disclosure provides a conjugate described herein or the pharmaceutical composition described herein for use in a method of treatment of a subject's body by therapy.

In some aspects, the present disclosure provides a conjugate described herein or the pharmaceutical composition described herein for use in a method of treating cancer.

In some aspects, the present disclosure provides a method of preparing an antibody conjugate of the formula:

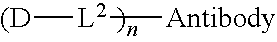

wherein:
Antibody is an antibody construct;
n is selected from 1 to 20; and
D-L² is selected from a compound or salt described herein,

comprising contacting D-L² with an antibody construct to form the antibody conjugate.

In some aspects, the present disclosure provides a method of preparing an antibody conjugate of the formula:

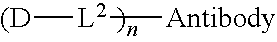

wherein:
Antibody is an antibody construct;
n is selected from 1 to 20;
L² is a linker; and
D is selected from a compound or salt disclosed herein,

comprising contacting L² with the antibody construct to form L²-antibody and contacting L²-antibody with D to form the antibody conjugate.

In some embodiments, the antibody construct comprises an antigen binding domain that specifically binds to an antigen selected from the group consisting of HER2, TROP2 and MUC16. In some embodiments, the methods of the present disclosure further comprise purifying the antibody conjugate.

In some aspects, the present disclosure provides a compound or salt thereof selected from compounds 1.1-1.11.

In some embodiments for a compound or salt of Formula (IIA) or (IIB), one of R¹⁰¹, R¹⁰², R¹⁰³, and R¹⁰⁰ is L² or one substituent on R¹⁰¹, R¹⁰², R¹⁰³, L⁵², L²¹ and L⁵¹ is -L².

In some embodiments for a compound or salt of Formula (IVA) or (IVB), one of R²⁰¹, R²⁰², R¹⁰³, and R¹⁰⁰ is L² or one substituent on R²⁰¹, R²⁰², R¹⁰³, L⁵², L²¹ and L⁵¹ is -L².

In some embodiments, L² is covalently bound to a nitrogen atom or oxygen atom. In some embodiments, L² is covalently bound to a nitrogen atom. In some embodiments, L² comprises 15 or more consecutive atoms.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

While preferred embodiments of the present invention have been shown and described herein, it will be evident to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present disclosure provides compounds, conjugates and pharmaceutical compositions for use in the treatment or prevention of disease. In certain embodiments, the compounds of the disclosure are TLR8 modulators. In certain embodiments, the compounds are TLR8 agonists. Toll-like receptors (TLRs) are a family of membrane-spanning receptors that are expressed on cells of the immune system like dendritic cells, macrophages, monocytes, T cells, B cells, NK cells and mast cells but also on a variety of non-immune cells such as endothelial cells, epithelial cells and even tumor cells. TLRs can have many isoforms, including TLR4, TLR7 and TLR8.

In certain aspects, compounds or conjugates of the disclosure are administered in a form suitable to attenuate or eliminate immune-modulatory activity until the compound or conjugate reaches a desired target and the active site amine is unmasked. While not wishing to be bound by a mechanistic theory, the modification of compounds to attenuate or eliminate immune-modulatory activity may prevent undesired off-target immune-stimulatory activity, e.g., immune-stimulation in healthy tissue.

In certain embodiments, a compound such as a TLR8 agonist is modified with a removable masking group, such that the TLR8 agonist has limited activity or is inactive until it reaches an environment where the masking group is removed to reveal the active compound. For example, the TLR8 agonist is covalently modified at an amine involved in binding to the active site of a TLR8 receptor such that the compound is unable to bind the active site of the receptor in its modified form. In such an example, the masking group may be removed under physiological conditions, e.g., enzymatic or acidic conditions, specific to the intended site of delivery, e.g., intracellular or extracellular adjacent to target cells. In certain embodiments, the amine masking group inhibits binding of the amine group of the compound with residues of a TLR8 receptor. The amine masking group may be removable under physiological conditions within a cell but remains covalently bound to the amine outside of a cell. Masking groups that may be used to inhibit or attenuate binding of an amine group of a compound with residues of a TLR8 receptor include, for example, peptides and carbamates.

TLR8 receptors are localized to the endolysosomal/phagosomal compartment and predominantly found to be expressed by cells of the myeloid lineage. TLR ligation leads to activation of NF-κB and IRF-dependent pathways with the specific activation sequence and response with respect to the specific TLR and cell type. While TLR7 is mainly expressed in all dendritic cells subtypes (DC and here highly in pDC, plasmacytoid DC) and can be induced in B cells upon IFNα stimulation, TLR8 expression is rather restricted to monocytes, macrophages and myeloid DC. TLR8 signaling via MyD88 can be activated by bacterial single stranded RNA, small molecule agonists and microRNAs. The activation of TLR8 results in the production of various pro-inflammatory cytokines such as IL-6, IL-12 and TNF-α as well as enhanced expression of co-stimulatory molecules, such as CD80, CD86, and chemokine receptors. In addition, TLR8 activation can induce type I interferon (IFNβ) in primary human monocytes.

Several agonists targeting activation of different TLRs can be used in various immunotherapies, including vaccine adjuvants and in cancer immunotherapies. TLR agonists can range from simple molecules to complex macromolecules. Likewise, the sizes of TLR agonists can range from small to large. TLR agonists can be synthetic or biosynthetic agonists. TLR agonists can also be Pathogen-Associated Molecular Pattern molecules (PAMPs).

The compounds of the present disclosure may be useful for the treatment and prevention, e.g., vaccination of cancer, autoimmune diseases, inflammation, sepsis, allergy, asthma, graft rejection, graft-versus-host disease, immunodeficiencies, and infectious diseases.

In certain embodiments, the compounds have utility in the treatment of cancer either as single agents or in combination therapy. In certain embodiments, the compounds have utility as single agent immunomodulators, vaccine adjuvants and in combination with conventional cancer therapies. In certain embodiments, the compounds described herein are incorporated into an antibody conjugate that can be utilized to enhance immune responses. In certain embodiments, the disclosure provides antibody construct-benzazepine compounds conjugates.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

As used herein, an “amine masking group” refers to any moiety covalently bound to the nitrogen of an amine, e.g., primary amine, which attenuates the interaction or activity, or blocks the amine from interacting with a TLR8 receptor, and that is removable from the amine in vivo. Exemplary amine masking groups include enzymatically-cleavable promoieties such as amino acids or peptides.

As used herein, “sequence identity”, “% identity” and the like refer to the identity of a DNA, RNA, nucleotide, amino acid, or protein sequence to another DNA, RNA, nucleotide, amino acid, or protein sequence, respectively, according to context. Sequence identity can be expressed in terms of a percentage of sequence identity of a first sequence to a second sequence. Percent (%) sequence identity with respect to a reference DNA sequence is the percentage of DNA nucleotides in a candidate sequence that are identical with the DNA nucleotides in the reference DNA sequence after aligning the sequences and introducing gaps, as necessary. Percent (%) sequence identity with respect to a reference amino acid sequence is the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity.

As used herein, the term “antibody” refers to an immunoglobulin molecule that specifically binds to, or is immunologically reactive toward, a specific antigen. Antibody can include, for example, polyclonal, monoclonal, genetically engineered, and antigen binding fragments thereof. An antibody can be, for example, murine, chimeric, humanized, heteroconjugate, bispecific, a diabody, a triabody, or a tetrabody. The antigen binding fragment can include, for example, Fab′, F(ab′)₂, Fab, Fv, rIgG, and scFv.

As used herein, an “antigen binding domain” refers to a region on a molecule that binds to an antigen. An antigen binding domain of the disclosure may be a domain that can specifically bind to an antigen. An antigen binding domain can be an antigen-binding portion of an antibody or an antibody fragment. An antigen binding domain can be one or more fragments of an antibody that can retain the ability to specifically bind to an antigen. An antigen binding domain can be an antigen binding fragment. An antigen binding domain can recognize a single antigen. An antigen binding domain can recognize, for example, two, three, four, five, six, seven, eight, nine, ten, or more antigens.

As used herein, an “antibody construct” refers to a molecule, e.g., a protein, peptide, antibody or portion thereof, that contains an antigen binding domain and an Fc domain. An antibody construct can recognize, for example, multiple antigens.

“Conjugate”, as used herein, refers to an antibody construct that is covalently linked, either directly or through a linker, to a compound or compound-linker described herein, e.g., a benzazepine compound or salt thereof.

As used herein, an “Fc domain” can be an Fc domain from an antibody or from a non-antibody that can bind to an Fc receptor.

As used herein, an “Fc null” refers to an Fc domain that exhibits weak to no binding to any of the Fcgamma receptors. In some embodiments, an Fc null domain or region exhibits a reduction in binding affinity (e.g., increase in Kd) to Fc gamma receptors of at least 1000-fold.

As used herein, “recognize” with regard to antibody interactions refers to the specific association or specific binding between an antigen binding domain of an antibody or portion thereof and an antigen.

As used herein, “specifically binds” with regard to an antigen binding domain interaction with an antigen refers to the specific binding between the antigen binding domain and the antigen, as compared with the interaction of the antigen binding domain with a different antigen (i.e., non-specific binding). In some embodiments, an antigen binding domain that recognizes or specifically binds to an antigen has a dissociation constant (KD) of <<100 nM, <10 nM, <1 nM, <0.1 nM, <0.01 nM, or <0.001 nM (e.g. 10⁻⁸ M or less, e.g. from 10⁻⁸ M to 10⁻¹³ M, e.g., from 10⁻⁹ M to 10⁻¹³ M).

As used herein, a “target binding domain” refers to a construct that contains an antigen binding domain from an antibody or from a non-antibody that can bind to the antigen.

As used herein, a “tumor antigen” is an antigenic substance associated with a tumor or cancer cell and can trigger an immune response in a host.

The phrase “targeting moiety” refers to a structure that has a selective affinity for a target molecule relative to other non-target molecules. The targeting moiety binds to a target molecule. A targeting moiety may include, for example, an antibody, a peptide, a ligand, a receptor, or a binding portion thereof. The target biological molecule may be a biological receptor or other structure of a cell such as a tumor antigen.

As used herein, the abbreviations for the natural L-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gin); glycine (G, Gly); histidine (H, His); isoleucine (I, lie); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid. In some aspects, X can be asparagine (N), glutamine (Q), histidine (H), lysine (K), or arginine (R).

The terms “salt” or “pharmaceutically acceptable salt” refer to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “C_{x-y}” when used in conjunction with a chemical moiety, such as alkyl, alkenyl, or alkynyl is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond respectively. The term —C_{x-y}alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene-refers to a substituted or unsubstituted alkynylene chain with from x to y carbons in the alkynylene chain. For example, —C₂₋₆alkynylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” refers to a divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene chain is optionally substituted by one or more substituents such as those substituents described herein.

“Alkenylene” refers to a divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₁ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene). Unless stated otherwise specifically in the specification, an alkenylene chain is optionally substituted by one or more substituents such as those substituents described herein.

“Alkynylene” refers to a divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene). Unless stated otherwise specifically in the specification, an alkynylene chain is optionally substituted by one or more substituents such as those substituents described herein.

“Heteroalkylene” refers to a divalent hydrocarbon chain including at least one heteroatom in the chain, containing no unsaturation, and preferably having from one to twelve carbon atoms and from one to 6 heteroatoms, e.g., —O—, —NH—, —S—. The heteroalkylene chain is attached to the rest of the molecule through a single bond and to the radical group through a single bond. The points of attachment of the heteroalkylene chain to the rest of the molecule and to the radical group are through the terminal atoms of the chain. In other embodiments, a heteroalkylene comprises one to five carbon atoms and from one to three heteroatoms. In other embodiments, a heteroalkylene comprises one to four carbon atoms and from one to three heteroatoms. In other embodiments, a heteroalkylene comprises one to three carbon atoms and from one to two heteroatoms. In other embodiments, a heteroalkylene comprises one to two carbon atoms and from one to two heteroatoms. In other embodiments, a heteroalkylene comprises one carbon atom and from one to two heteroatoms. In other embodiments, a heteroalkylene comprises five to eight carbon atoms and from one to four heteroatoms. In other embodiments, a heteroalkylene comprises two to five carbon atoms and from one to three heteroatoms. In other embodiments, a heteroalkylene comprises three to five carbon atoms and from one to three heteroatoms. Unless stated otherwise specifically in the specification, a heteroalkylene chain is optionally substituted by one or more substituents such as those substituents described herein.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, and 6-6 fused ring systems. Exemplary carbocycles include cyclopentyl, cyclohexyl, cyclohexenyl, adamantyl, phenyl, indanyl, and naphthyl. The term “unsaturated carbocycle” refers to carbocycles with at least one degree of unsaturation and excluding aromatic carbocycles. Examples of unsaturated carbocycles include cyclohexadiene, cyclohexene, and cyclopentene.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, and 6-6 fused ring systems. The term “unsaturated heterocycle” refers to heterocycles with at least one degree of unsaturation and excluding aromatic heterocycles. Examples of unsaturated heterocycles include dihydropyrrole, dihydrofuran, oxazoline, pyrazoline, and dihydropyridine.

The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also include polycyclic ring systems having two or more cyclic rings in which two or more carbons are common to two adjoining rings wherein at least one of the rings is heteroaromatic, e.g., the other cyclic rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazine (═NNH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a}) C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine

(═NNH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═NNH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants, unless specified otherwise.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

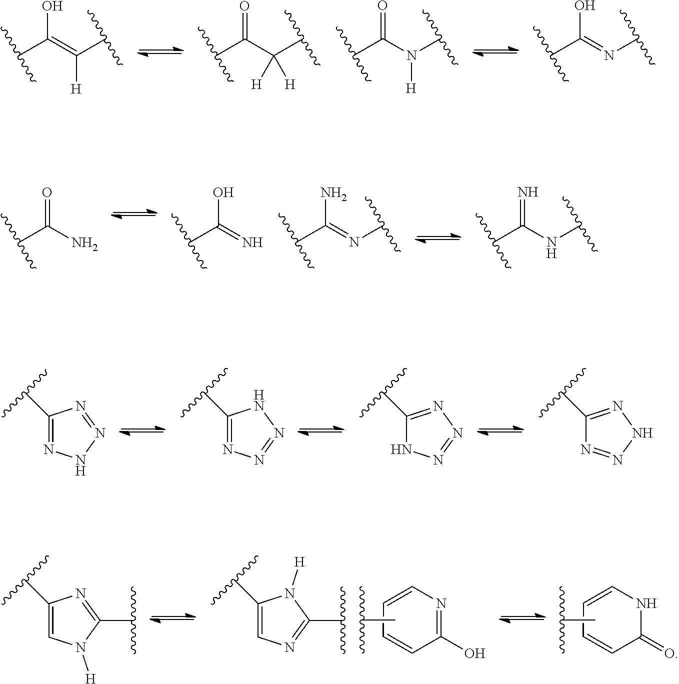

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made, for example, by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and/or ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds can be synthesized, for example, using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

### Antibody Construct

Disclosed herein are targeting moieties and antibody constructs that may be used together with compounds of the disclosure. In certain embodiments, compounds of the disclosure are conjugated either directly or through a linker group to an antibody construct or a targeting moiety forming conjugates. In certain embodiments, antibody conjugates are represented by the following formula:

A [symbol] L-D)ₙ,

wherein A is an antibody construct, Lisa linker, D is a benzazepine compound or salt thereof as described herein and n is from 1 to 20. In certain embodiments, n is from 1 to 10, such as from 1 to 9, such as from 1 to 8, such as from 2 to 8, such as from 1 to 6, such as from 3 to 5 or such as about 2. In certain embodiments, n is 4.

In some aspects, the present disclosure provides a conjugate represented by the formula:

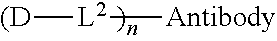

wherein:
Antibody is an antibody construct, the antibody construct comprising an antigen binding domain and an Fc domain;
n is 1 to 20;
D is the compound or salt disclosed herein; and
L² is a linker moiety attached to a residue of the antibody construct and to D.

In some embodiments, n is selected from 1 to 8. In certain embodiments, n is selected from 2 to 5. In certain embodiments, n is 2 or 4.

In some embodiments, -L² is represented by the formula:

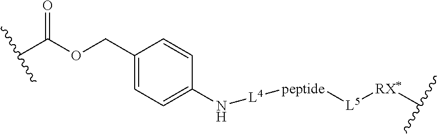

wherein:
L⁴ represents the C-terminus of the peptide and L⁵ is selected from a bond, alkylene and heteroalkylene, wherein L⁵ is optionally substituted with one or more groups independently selected from R³⁰; RX* is a bond, a succinimide moiety, or a hydrolyzed succinimide moiety bound to the residue of the antibody construct, wherein [symbol] on RX* represents the point of attachment to the residue of the antibody construct; and
R³⁰ is independently selected at each occurrence from halogen, —OH, —CN, —O-alkyl, —SH, ═O, ═S, —NH₂, and —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂.

In some embodiments, RX* is a succinamide moiety, hydrolyzed succinamide moiety or a mixture thereof and is bound to a cysteine residue of an antibody construct.

In some embodiments, -L² is represented by the formula:

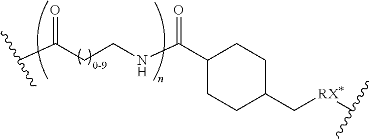

wherein:
RX* is a bond, a succinimide moiety, or a hydrolyzed succinimide moiety bound to the residue of the antibody construct, wherein [symbol] on RX* represents the point of attachment to the residue of the antibody construct; and
n is 0-9.

In some embodiments, the antigen binding domain specifically binds to an antigen selected from the group consisting of HER2, TROP2 and MUC16. In some embodiments, the Fc domain is an Fc null.

An antibody construct may contain, for example, two, three, four, five, six, seven, eight, nine, ten, or more antigen binding domains. An antibody construct may contain two antigen binding domains in which each antigen binding domain can recognize the same antigen. An antibody construct may contain two antigen binding domains in which each antigen binding domain can recognize different antigens. An antigen binding domain may be in a scaffold, in which a scaffold is a supporting framework for the antigen binding domain. An antigen binding domain may be in a non-antibody scaffold. An antigen binding domain may be in an antibody scaffold. An antibody construct may comprise an antigen binding domain in a scaffold. The antibody construct may comprise an Fc fusion protein. In some embodiments, the antibody construct is an Fc fusion protein. An antigen binding domain may specifically bind to a tumor antigen. An antigen binding domain may specifically bind to an antigen that is at least 80%, at least 90%, at least 95%, at least 99%, or 100% identical to a tumor antigen. An antigen binding domain may specifically bind to an antigen on an antigen presenting cell (APC). An antigen binding domain may specifically bind to an antigen that is at least 80%, at least 90%, at least 95%, at least 99%, or 100% identical to an antigen on an antigen presenting cell (APC).

An antigen binding domain of an antibody may comprise one or more light chain (LC) CDRs and one or more heavy chain (HC) CDRs. For example, an antigen binding domain of an antibody may comprise one or more of the following: a light chain complementary determining region 1 (LCDR1), a light chain complementary determining region 2 (LCDR2), or a light chain complementary determining region 3 (LCDR3). For another example, an antigen binding domain may comprise one or more of the following: a heavy chain complementary determining region 1 (HCDR1), a heavy chain complementary determining region 2 (HCDR2), or a heavy chain complementary determining region 3 (HCDR3). As an additional example, an antibody binding domain of an antibody may comprise one or more of the following: LCDR1, LCDR2, LCDR3, HCDR1, HCDR2, and HCDR3. An antigen binding domain of an antibody may comprise all six of the following: LCDR1, LCDR2, LCDR3, HCDR1, HCDR2, and HCDR3.

In some embodiments, the antigen binding domain of an antibody construct may be selected from any domain that specifically binds the antigen including, but not limited to, a monoclonal antibody, a polyclonal antibody, a recombinant antibody, or a functional fragment thereof, for example, a heavy chain variable domain (V_{H}) and a light chain variable domain (V_{L}), or a DARPin, an affimer, an avimer, a knottin, a monobody, an affinity clamp, an ectodomain, a receptor ectodomain, a receptor, a cytokine, a ligand, an immunocytokine, a T cell receptor, a bicyclic peptide, a fynomer, or a recombinant T cell receptor. In some embodiments, the antigen binding domain is of an antibody construct may be selected from any domain that specifically binds the antigen including, but not limited to, a monoclonal antibody, a polyclonal antibody, a recombinant antibody, or a functional fragment thereof, for example, a heavy chain variable domain (V_{H}) and a light chain variable domain (V_{L}), or a DARPin, an affimer, an avimer, a knottin, a monobody, a bicyclic peptide, or a fynomer.

The antigen binding domain of an antibody construct may be at least 80% identical to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), or a DARPin, an affimer, an avimer, a knottin, a monobody, an affinity clamp, an ectodomain, a receptor ectodomain, a receptor, a cytokine, a ligand, an immunocytokine, a T cell receptor, a bicyclic peptide, a fynomer, or a recombinant T cell receptor.

An antigen binding domain can specifically bind to a tumor antigen, such as for example, a tumor antigen such as CD5, CD19, CD20, CD25, CD37, CD30, CD33, CD45, CAMPATH-1, BCMA, CS-1, PD-L1, B7-H3, B7-DC, HLD-DR, carcinoembryonic antigen (CEA), TAG-72, EpCAM, MUC1, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, fibroblast activation protein (FAP), tenascin, a metalloproteinase, endosialin, vascular endothelial growth factor, avB3, WT1, LMP2, HPV E6, HPV E7, EGFRvIII (de2-7 EGFR), Her-2/neu, MAGE A3, p53 nonmutant, NY-ESO-1, MelanA/MART1, Ras mutant, gp100, p53 mutant, PR1, bcr-abl, tyronsinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL 1, MUC16, MAGE A4, MAGE C2, GAGE, EGFR, CMET, HER3, MUC1, MUC15, CA6, NAPI2B, TROP2, CLDN18.2, RON, LY6E, FRA, DLL3, PTK7, LIV1, ROR1, or Fos-related antigen 1.

In certain embodiments, an antigen binding domain specifically binds to a tumor antigen, such as those selected from CD5, CD25, CD37, CD33, CD45, BCMA, CS-1, PD-L1, B7-H3, B7-DC (PD-L2), HLD-DR, carcinoembryonic antigen (CEA), TAG-72, EpCAM, MUC1, folate-binding protein (FOLR1), A33, G250 (carbonic anhydrase IX), prostate-specific membrane antigen (PSMA), GD2, GD3, GM2, Ley, CA-125, CA19-9 (MUC1 sLe(a)), epidermal growth factor, HER2, IL-2 receptor, EGFRvIII (de2-7 EGFR), fibroblast activation protein (FAP), a tenascin, a metalloproteinase, endosialin, avB3, LMP2, EphA2, PAP, AFP, ALK, polysialic acid, TRP-2, fucosyl GM1, mesothelin (MSLN), PSCA, sLe(a), GM3, BORIS, Tn, TF, GloboH, STn, CSPG4, AKAP-4, SSX2, Legumain, Tie 2, Tim 3, VEGFR2, PDGFR-B, ROR2, TRAIL 1, MUC16, EGFR, CMET, HER3, MUC1, MUC15, CA6, NAPI2B, TROP2, CLDN18.2, RON, LY6E, FRAlpha, DLL3, PTK7, LIV1, ROR1, CLDN6, GPC3, ADAM 12, LRRC15, CDH6, TMEFF2, TMEM238, GPNMB, ALPPL2, UPK1B, UPK2, LAMP-1, LY6K, EphB2, STEAP, ENPP3, CDH3, Nectin4, LYPD3, EFNA4, GPA33, SLITRK6 or HAVCR1.

In certain embodiments, an antigen binding domain specifically binds to a carbohydrate antigen, such as GD2, GD3, GM2, Ley, polysialic acid, fucosyl GM1, GM3, Tn, STn, sLe(animal), or GloboH.

In certain embodiments, an antibody construct comprises an Fc region or an Fc domain, in which the Fc domain may be the part of the Fc region that interacts with one or more Fc receptors. The Fc domain of an antibody construct may interact with Fc-receptors (FcRs) found on immune cells. The Fc domain may also mediate the interaction between effector molecules and cells, which can lead to activation of the immune system. The Fc region may be derived from IgG, IgA, or IgD antibody isotypes, and may comprise two identical protein fragments, which are derived from the second and third constant domains of the antibody's heavy chains. In an Fc region derived from an IgG antibody isotype, the Fc region comprises a highly-conserved N-glycosylation site, which may be essential for FcR-mediated downstream effects. The Fc region may be derived from IgM or IgE antibody isotypes, in which the Fc region may comprise three heavy chain constant domains.

An Fc domain may interact with different types of FcRs. The different types of FcRs may include, for example, FcγRI, FcγRIIA, FcγRIIB, FcγRIIIA, FcγRIIIB, FcαRI, FcμR, FcεRI, FcεRII, and FcRn. FcRs are located on the membrane of certain immune cells including, for example, B lymphocytes, natural killer cells, macrophages, neutrophils, follicular dendritic cells, eosinophils, basophils, platelets, and mast cells. Once the FcR is engaged by the Fc domain, the FcR may initiate functions including, for example, clearance of an antigen-antibody complex via receptor-mediated endocytosis, antibody-dependent cell-mediated cytotoxicity (ADCC), antibody dependent cell-mediated phagocytosis (ADCP), and ligand-triggered transmission of signals across the plasma membrane that can result in alterations in secretion, exocytosis, and cellular metabolism. FcRs may deliver signals when FcRs are aggregated by antibodies and multivalent antigens at the cell surface. The aggregation of FcRs with immunoreceptor tyrosine-based activation motifs (ITAMs) may sequentially activate SRC family tyrosine kinases and SYK family tyrosine kinases. An ITAM comprises a twice-repeated YxxL sequence flanking seven variable residues. The SRC and SYK kinases may connect the transduced signals with common activation pathways.

In some embodiments, an Fc domain or region can exhibit reduced binding affinity to one or more Fc receptors. In some embodiments, an Fc domain or region can exhibit reduced binding affinity to one or more Fcgamma receptors. In some embodiments, an Fc domain or region can exhibit reduced binding affinity to FcRn receptors. In some embodiments, an Fc domain or region can exhibit reduced binding affinity to Fcgamma and FcRn receptors. In some embodiments, an Fc domain is an Fc null domain or region. As used herein, an “Fc null” refers to an Fc domain that exhibits weak to no binding to any of the Fcgamma receptors. In some embodiments, an Fc null domain or region exhibits a reduction in binding affinity (e.g., increase in Kd) to Fc gamma receptors of at least 1000-fold.

The Fc domain may have one or more, two or more, three or more, or four or more amino acid substitutions that decrease binding of the Fc domain to an Fc receptor. In certain embodiments, an Fc domain has decreased binding affinity for one or more of FcγRI (CD64), FcγRIIA (CD32), FcγRIIIA (CD16a), FcγRIIIB (CD16b), or any combination thereof. In order to decrease binding affinity of an Fc domain or region to an Fc receptor, the Fc domain or region may comprise one or more amino acid substitutions that reduces the binding affinity of the Fc domain or region to an Fc receptor.

In certain embodiments, the one or more substitutions comprise any one or more of IgG1 heavy chain mutations corresponding to E233P, L234V, L234A, L235A, L235E, ΔG236, G237A, E318A, K320A, K322A, A327G, A330S, or P331S according to the EU index of Rabat numbering.

In some embodiments, the Fc domain or region can comprise a sequence of an IgG isoform that has been modified from the wild-type IgG sequence. In some embodiments, the Fc domain or region can comprise a sequence of the IgG1 isoform that has been modified from the wild-type IgG1 sequence. In some embodiments, the modification comprises substitution of one or more amino acids that reduce binding affinity of an IgG Fc domain or region to all Fcγ receptors. A modification can be substitution of E233, L234 and L235, such as E233P/L234V/L235A or E233P/L234V/L235A/ΔG236, according to the EU index of Kabat. A modification can be a substitution of P238, such as P238A, according to the EU index of Kabat. A modification can be a substitution of D265, such as D265A, according to the EU index of Kabat. A modification can be a substitution of N297, such as N297A, according to the EU index of Kabat. A modification can be a substitution of A327, such as A327Q, according to the EU index of Kabat. A modification can be a substitution of P329, such as P239A, according to the EU index of Kabat.

In some embodiments, an IgG Fc domain or region comprises at least one amino acid substitution that reduces its binding affinity to FcγR1, as compared to a wild-type or reference IgG Fc domain. A modification can comprise a substitution at F241, such as F241A, according to the EU index of Kabat. A modification can comprise a substitution at F243, such as F243A, according to the EU index of Kabat. A modification can comprise a substitution at V264, such as V264A, according to the EU index of Kabat. A modification can comprise a substitution at D265, such as D265A according to the EU index of Kabat.

In some embodiments, an IgG Fc domain or region comprises at least one amino acid substitution that increases its binding affinity to FcγR1, as compared to a wild-type or reference IgG Fc domain. A modification can comprise a substitution at A327 and P329, such as A327Q/P329A, according to the EU index of Kabat.

In some embodiments, the modification comprises substitution of one or more amino acids that reduce binding affinity of an IgG Fc domain or region to FcγRII and FcγRIIIA receptors. A modification can be a substitution of D270, such as D270A, according to the EU index of Kabat. A modification can be a substitution of Q295, such as Q295A, according to the EU index of Kabat. A modification can be a substitution of A327, such as A237S, according to the EU index of Kabat.

In some embodiments, the modification comprises substitution of one or more amino acids that increases binding affinity of an IgG Fc domain or region to FcγRII and FcγRIIIA receptors. A modification can be a substitution of T256, such as T256A, according to the EU index of Kabat. A modification can be a substitution of K290, such as K290A, according to the EU index of Kabat.

In some embodiments, the modification comprises substitution of one or more amino acids that increases binding affinity of an IgG Fc domain or region to FcγRII receptor. A modification can be a substitution of R255, such as R255A, according to the EU index of Kabat. A modification can be a substitution of E258, such as E258A, according to the EU index of Kabat. A modification can be a substitution of S267, such as S267A, according to the EU index of Kabat. A modification can be a substitution of E272, such as E272A, according to the EU index of Kabat. A modification can be a substitution of N276, such as N276A, according to the EU index of Kabat. A modification can be a substitution of D280, such as D280A, according to the EU index of Kabat. A modification can be a substitution of H285, such as H285A, according to the EU index of Kabat. A modification can be a substitution of N286, such as N286A, according to the EU index of Kabat. A modification can be a substitution of T307, such as T307A, according to the EU index of Kabat. A modification can be a substitution of L309, such as L309A, according to the EU index of Kabat. A modification can be a substitution of N315, such as N315A, according to the EU index of Kabat. A modification can be a substitution of K326, such as K326A, according to the EU index of Kabat. A modification can be a substitution of P331, such as P331A, according to the EU index of Kabat. A modification can be a substitution of S337, such as S337A, according to the EU index of Kabat. A modification can be a substitution of A378, such as A378A, according to the EU index of Kabat. A modification can be a substitution of E430, such as E430, according to the EU index of Kabat.

In some embodiments, the modification comprises substitution of one or more amino acids that increases binding affinity of an IgG Fc domain or region to FcγRII receptor and reduces the binding affinity to FcγRIIIA receptor. A modification can be a substitution of H268, such as H268A, according to the EU index of Kabat. A modification can be a substitution of R301, such as R301A, according to the EU index of Kabat. A modification can be a substitution of K322, such as K322A, according to the EU index of Kabat.

In some embodiments, the modification comprises substitution of one or more amino acids that decreases binding affinity of an IgG Fc domain or region to FcγRII receptor but does not affect the binding affinity to FcγRIIIA receptor. A modification can be a substitution of R292, such as R292A, according to the EU index of Kabat. A modification can be a substitution of K414, such as K414A, according to the EU index of Kabat.

In some embodiments, the modification comprises substitution of one or more amino acids that decreases binding affinity of an IgG Fc domain or region to FcγRII receptor and increases the binding affinity to FcγRIIIA receptor. A modification can be a substitution of S298, such as S298A, according to the EU index of Kabat. A modification can be substitution of S239, I332 and A330, such as S239D/I332E/A330L. A modification can be substitution of S239 and I332, such as S239D/I332E.

In some embodiments, the modification comprises substitution of one or more amino acids that decreases binding affinity of an IgG Fc domain or region to FcγRIIIA receptor. A modification can be substitution of F241 and F243, such as F241S/F243S or F241I/F243I, according to the EU index of Kabat.

In some embodiments, the modification comprises substitution of one or more amino acids that decreases binding affinity of an IgG Fc domain or region to FcγRIIIA receptor and does not affect the binding affinity to FcγRII receptor. A modification can be a substitution of S239, such as S239A, according to the EU index of Kabat. A modification can be a substitution of E269, such as E269A, according to the EU index of Kabat. A modification can be a substitution of E293, such as E293A, according to the EU index of Kabat. A modification can be a substitution of Y296, such as Y296F, according to the EU index of Kabat. A modification can be a substitution of V303, such as V303A, according to the EU index of Kabat. A modification can be a substitution of A327, such as A327G, according to the EU index of Kabat. A modification can be a substitution of K338, such as K338A, according to the EU index of Kabat. A modification can be a substitution of D376, such as D376A, according to the EU index of Kabat.

In some embodiments, the modification comprises substitution of one or more amino acids that increases binding affinity of an IgG Fc domain or region to FcγRIIIA receptor and does not affect the binding affinity to FcγRII receptor. A modification can be a substitution of E333, such as E333A, according to the EU index of Kabat. A modification can be a substitution of K334, such as K334A, according to the EU index of Kabat. A modification can be a substitution of A339, such as A339T, according to the EU index of Kabat. A modification can be substitution of S239 and I332, such as S239D/I332E.

In some embodiments, the modification comprises substitution of one or more amino acids that increases binding affinity of an IgG Fc domain or region to FcγRIIIA receptor. A modification can be substitution of L235, F243, R292, Y300 and P396, such as L235V/F243L/R292P/Y300L/P396L (IgG1VLPLL) according to the EU index of Kabat. A modification can be substitution of S298, E333 and K334, such as S298A/E333A/K334A, according to the EU index of Kabat. A modification can be substitution of K246, such as K246F, according to the EU index of Kabat.

Other substitutions in an IgG Fc domain that affect its interaction with one or more Fcγ receptors are disclosed in U.S. Pat. Nos. 7,317,091 and 8,969,526 (the disclosures of which are incorporated by reference herein).

In some embodiments, an IgG Fc domain or region comprises at least one amino acid substitution that reduces the binding affinity to FcRn, as compared to a wild-type or reference IgG Fc domain. A modification can comprise a substitution at H435, such as H435A according to the EU index of Kabat. A modification can comprise a substitution at I253, such as I253A according to the EU index of Kabat. A modification can comprise a substitution at H310, such as H310A according to the EU index of Kabat. A modification can comprise substitutions at I253, H310 and H435, such as I253A/H310A/H435A according to the EU index of Kabat.

A modification can comprise a substitution of one amino acid residue that increases the binding affinity of an IgG Fc domain for FcRn, relative to a wildtype or reference IgG Fc domain. A modification can comprise a substitution at V308, such as V308P according to the EU index of Kabat. A modification can comprise a substitution at M428, such as M428L according to the EU index of Kabat. A modification can comprise a substitution at N434, such as N434A according to the EU index of Kabat or N434H according to the EU index of Kabat. A modification can comprise substitutions at T250 and M428, such as T250Q and M428L according to the EU index of Kabat. A modification can comprise substitutions at M428 and N434, such as M428L and N434S, N434A or N434H according to the EU index of Kabat. A modification can comprise substitutions at M252, S254 and T256, such as M252Y/S254T/T256E according to the EU index of Kabat. A modification can be a substitution of one or more amino acids selected from P257L, P257N, P257I, V279E, V279Q, V279Y, A281S, E283F, V284E, L306Y, T307V, V308F, Q311V, D376V, and N434H. Other substitutions in an IgG Fc domain that affect its interaction with FcRn are disclosed in U.S. Pat. No. 9,803,023 (the disclosure of which is incorporated by reference herein).

An antibody construct may be an antibody. An antibody may consist of two identical light protein chains and two identical heavy protein chains, all held together covalently by disulfide linkages. The N-terminal regions of the light and heavy chains together form the antigen recognition site of an antibody. Structurally, various functions of an antibody may be confined to discrete protein domains (i.e., regions). The sites that can recognize and can bind to antigen may consist of three complementarities determining regions (CDRs) that lie within the variable heavy chain region and variable light chain region at the N-terminal end of the heavy chain and the light chain. The constant domains may provide the general framework of the antibody and may not be involved directly in binding the antibody to an antigen, but may be involved in various effector functions, such as participation of the antibody in antibody-dependent cellular cytotoxicity, and may bind to one or more Fc receptors. The constant domains may form an Fc region. The constant domains may form an Fc domain. The domains of natural light and heavy chains may have the same general structures, and each chain may comprise four framework regions, whose sequences can be somewhat conserved, connected by three CDRs. The four framework regions may largely adopt a β-sheet conformation and the CDRs can form loops connecting, and in some aspects forming part of, the β-sheet structure. The CDRs in each chain may be held in close proximity by the framework regions and, with the CDRs from the other chain, contribute to the formation of the antigen binding site.

An antibody construct may comprise a light chain of an amino acid sequence having at least one, two, three, four, five, six, seven, eight, nine or ten modifications and in certain embodiments, not more than 40, 35, 30, 25, 20, 15 or 10 modifications of the amino acid sequence relative to the natural or original amino acid sequence. An antibody construct may comprise a heavy chain of an amino acid sequence having at least one, two, three, four, five, six, seven, eight, nine or ten modifications and in certain embodiments, not more than 40, 35, 30, 25, 20, 15 or 10 modifications of the amino acid sequence relative to the natural or original amino acid sequence.

An antibody of an antibody construct may be an antibody of any type, which may be assigned to different classes of immunoglobins, e.g., IgA, IgD, IgE, IgG, and IgM. Some classes are further divided into isotypes, e.g., IgG1, IgG2, IgG3, IgG4, IgA1, and IgA2. An antibody may further comprise a light chain and a heavy chain, often more than one chain of each. The heavy-chain constant regions (Fc) that corresponds to the different classes of immunoglobulins may be α, δ, ε, γ, and μ, respectively. The light chains may be one of either kappa (κ) or lambda (λ), based on the amino acid sequences of the constant domains. The Fc region typically contains multiple Fc domains. An Fc receptor may bind an Fc domain. Antibody constructs may also include any fragment or recombinant forms thereof, including but not limited to, single chain variable fragments (scFvs), or or other antibody fragment.

An antibody construct may comprise an antibody fragment. An antibody fragment may include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; and (iii) a Fv fragment consisting of the V_{L} and V_{H} domains of a single arm of an antibody. Although the two domains of the Fv fragment, V_{L} and V_{H}, may be coded for by separate genes, they may be linked by a synthetic linker to be made as a single protein chain in which the V_{L} and V_{H} regions pair to form monovalent molecules.

F(ab′)₂ and Fab′ moieties may be produced recombinantly. The Fab fragment may also contain the constant domain of the light chain and the first constant domain (C_{H1}) of the heavy chain. Fab′ fragments may differ from Fab fragments by the addition of a few residues at the carboxyl terminus of the heavy chain C_{H1} domain including one or more cysteine(s) from the antibody hinge region.

An Fv may be the minimum antibody fragment which contains a complete antigen-recognition and antigen-binding site. This region may consist of a dimer of one heavy chain and one light chain variable domain in tight, non-covalent association. In this configuration, the three CDRs of each variable domain may interact to define an antigen-binding site on the surface of the V_{H}-V_{L} dimer. A single variable domain (or half of an Fv comprising only three CDRs specific for an antigen) may recognize and bind antigen, although the binding can be at a lower affinity than the affinity of the entire binding site.

An antibody may include an Fc region comprising an Fc domain. The Fc domain of an antibody may interact with FcRs found on immune cells. The Fc domain may also mediate the interaction between effector molecules and cells, which may lead to activation of the immune system. In the IgG, IgA, and IgD antibody isotypes, the Fc region may comprise two identical protein fragments, which can be derived from the second and third constant domains of the antibody's heavy chains. In the IgM and IgE antibody isotypes, the Fc regions may comprise three heavy chain constant domains. In the IgG antibody isotype, the Fc regions may comprise a highly-conserved N-glycosylation site, which may be important for FcR-mediated downstream effects.

An antibody used herein may be chimeric or “humanized.” Chimeric or humanized forms of non-human (e.g., murine) antibodies can be chimeric immunoglobulins, immunoglobulin chains or fragments thereof (such as Fv, Fab, Fab′, F(ab′)₂ or other target-binding subdomains of antibodies), which may contain minimal sequences derived from non-human immunoglobulin. In general, the humanized antibody may comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody may also comprise at least a portion of an immunoglobulin constant region (Fc), typically that of a human immunoglobulin consensus sequence.

An antibody may be a human antibody. As used herein, “human antibodies” can include antibodies having, for example, the amino acid sequence of a human immunoglobulin and may include antibodies isolated from human immunoglobulin libraries or from animals transgenic for one or more human immunoglobulins that do not express endogenous immunoglobulins. Human antibodies may be produced using transgenic mice which are incapable of expressing functional endogenous immunoglobulins, but which may express human immunoglobulin genes. Completely human antibodies that recognize a selected epitope may be generated using guided selection. In this approach, a selected non-human monoclonal antibody, e.g., a mouse antibody, may be used to guide the selection of a completely human antibody recognizing the same epitope.

An antibody may be a bispecific antibody or a dual variable domain antibody (DVD). Bispecific and DVD antibodies may be monoclonal, often human or humanized, antibodies that can have binding specificities for at least two different antigens.

An antibody may be a derivatized antibody. For example, derivatized antibodies may be modified by glycosylation, acetylation, pegylation, phosphorylation, amidation, derivatization by known protecting/blocking groups, proteolytic cleavage, or linkage to a cellular ligand or other protein.

An antibody may have a sequence that has been modified to alter at least one constant region-mediated biological effector function relative to the corresponding wild type sequence. For example, in some embodiments, the antibody can be modified to reduce at least one constant region-mediated biological effector function relative to an unmodified antibody, e.g., reduced binding to the Fc receptor (FcR). FcR binding may be reduced by, for example, mutating the immunoglobulin constant region segment of the antibody at particular regions necessary for FcR interactions.

An antibody Fc domain may be modified to acquire or improve at least one constant region-mediated biological effector function relative to an unmodified antibody or Fc domain, e.g., to enhance FcγR interactions. For example, an antibody with a constant region that binds FcγRIIA, FcγRIIB and/or FcγRIIIA with greater affinity than the corresponding wild type constant region may be produced as known in the art. An Fc domain that binds FcγRIIA, FcγRIIB and/or FcγRIIIA with greater affinity than the corresponding wild type Fc domain may be produced as known in the art.

An antibody construct may comprise an antibody with modifications of at least one amino acid residue. Modifications may be substitutions, additions, deletions, or the like. An antibody modification can be an insertion of an unnatural amino acid.

In certain embodiments, the antigen binding domain specifically binds to HER2, TROP2 or MUC16. In certain embodiments, the antigen binding domain specifically binds to HER2 or TROP2.

In certain embodiments, the antibody construct comprises a human antibody or a humanized antibody or an antigen binding portion thereof, e.g., a human or humanized CD40, a human or humanized HER2 or a human or humanized TROP2 antibody. In certain embodiments, the antibody construct comprises a TROP2 antibody, e.g., sacituzumab, or an antigen binding portion thereof. In certain embodiments, the antibody construct comprises the heavy and light chain variable region sequences of sacituzumab (SEQ ID NOs:3 and 4, respectively). In certain embodiments, the antibody construct comprises LC CDR1, LC CDR2 and LC CDR3 of the light chain variable region of sacituzumab (SEQ ID NO:4), and HC CDR1, HC CDR2 and HC CDR3 of the heavy chain variable region of sacituzumab (SEQ ID NO:3), as determined by the Rabat index. In certain embodiments, the antibody construct comprises LC CDR1, LC CDR2 and LC CDR3 of the light chain variable region of sacituzumab (SEQ ID NO:4), and HC CDR1, HC CDR2 and HC CDR3 of the heavy chain variable region of sacituzumab (SEQ ID NO:3), as determined by IMGT (ImMunoGeneTics). In certain embodiments, the antibody construct comprises a HER2 antibody, e.g., pertuzumab, trastuzumab, or an antigen binding portion thereof. In certain embodiments, the antibody construct comprises the heavy and light chain variable region sequences of pertuzumab (SEQ ID NOs: 1 and 2, respectively). In certain embodiments, the antibody construct comprises LC CDR1, LC CDR2 and LC CDR3 of the light chain variable region of pertuzumab (SEQ ID NO:2), and HC CDR1, HC CDR2 and HC CDR3 of the heavy chain variable region of pertuzumab (SEQ ID NO: 1), as determined by the Rabat index. In certain embodiments, the antibody construct comprises LC CDR1, LC CDR2 and LC CDR3 of the light chain variable region of pertuzumab (SEQ ID NO:2), and HC CDR1, HC CDR2 and HC CDR3 of the heavy chain variable region of pertuzumab (SEQ ID NO:1), as determined by IMGT. In certain embodiments, the antibody construct comprises a humanized antibody or an antigen binding fragment thereof comprising LC CDR1, LC CDR2 and LC CDR3 of the light chain variable region of pertuzumab (SEQ ID NO:2), and HC CDR1, HC CDR2 and HC CDR3 of the heavy chain variable region of pertuzumab (SEQ ID NO: 1), as determined by the Rabat index. In certain embodiments, the antibody construct comprises a humanized antibody or an antigen binding fragment thereof comprising LC CDR1, LC CDR2 and LC CDR3 of the light chain variable region of pertuzumab (SEQ ID NO:2), and HC CDR1, HC CDR2 and HC CDR3 of the heavy chain variable region of pertuzumab (SEQ ID NO: 1), as determined by IMGT. In certain embodiments, the antibody construct comprises the heavy and light chain variable region sequences of trastuzumab (SEQ ID NOs:7 and 8, respectively). In certain embodiments, the antibody construct comprises LC CDR1, LC CDR2 and LC CDR3 of the light chain variable region of trastuzumab (SEQ ID NO: 8), and HC CDR1, HC CDR2 and HC CDR3 of the heavy chain variable region of trastuzumab (SEQ ID NO:7), as determined by the Rabat index. In certain embodiments, the antibody construct comprises LC CDR1, LC CDR2 and LC CDR3 of the light chain variable region of trastuzumab (SEQ ID NO:8), and HC CDR1, HC CDR2 and HC CDR3 of the heavy chain variable region of trastuzumab (SEQ ID NO:7), as determined by IMGT. In certain embodiments, the antibody construct comprises a humanized antibody or an antigen binding fragment thereof comprising LC CDR1, LC CDR2 and LC CDR3 of the light chain variable region of trastuzumab (SEQ ID NO: 8), and HC CDR1, HC CDR2 and HC CDR3 of the heavy chain variable region of trastuzumab (SEQ ID NO:7), as determined by the Rabat index. In certain embodiments, the antibody construct comprises a humanized antibody or an antigen binding fragment thereof comprising LC CDR1, LC CDR2 and LC CDR3 of the light chain variable region of trastuzumab (SEQ ID NO: 8), and HC CDR1, HC CDR2 and HC CDR3 of the heavy chain variable region of trastuzumab (SEQ ID NO:7), as determined by IMGT. In certain embodiments, the antibody construct comprises a CD40 antibody or an antigen binding portion thereof.

In certain embodiments, the antibody construct comprises a Liv-1 antibody, e.g., ladiratuzumab, huLiv1-14 (WO 2012078688), Liv1-1.7A4 (US 2011/0117013), huLiv1-22 (WO 2012078688) or an antigen binding portion thereof. In certain embodiments, the antibody construct comprises the heavy and light chain variable region sequences of ladiratuzumab (SEQ ID NOs:5 and 6, respectively). In certain embodiments, the antibody construct comprises LC CDR1, LC CDR2 and LC CDR3 of the light chain variable region of ladiratuzumab (SEQ ID NO:6), and HC CDR1, HC CDR2 and HC CDR3 of the heavy chain variable region of ladiratuzumab (SEQ ID NO:5), as determined by Rabat index. In certain embodiments, the antibody construct comprises LC CDR1, LC CDR2 and LC CDR3 of the light chain variable region of ladiratuzumab (SEQ ID NO:6), and HC CDR1, HC CDR2 and HC CDR3 of the heavy chain variable region of ladiratuzumab (SEQ ID NO:5), as determined by IMGT. In certain embodiments, the antibody construct comprises a humanized antibody or an antigen binding fragment thereof comprising LC CDR1, LC CDR2 and LC CDR3 of the light chain variable region of ladiratuzumab (SEQ ID NO:6), and HC CDR1, HC CDR2 and HC CDR3 of the heavy chain variable region of ladiratuzumab (SEQ ID NO:5), as determined by Rabat index. In certain embodiments, the antibody construct comprises a humanized antibody or an antigen binding fragment thereof comprising LC CDR1, LC CDR2 and LC CDR3 of the light chain variable region of ladiratuzumab (SEQ ID NO:6), and HC CDR1, HC CDR2 and HC CDR3 of the heavy chain variable region of ladiratuzumab (SEQ ID NO:5), as determined by IMGT. In certain embodiments, the antibody construct comprises the heavy and light chain variable region sequences of huLiv1-14 (SEQ ID NOs:17 and 18, respectively). In certain embodiments, the antibody construct comprises LC CDR1, LC CDR2 and LC CDR3 of the light chain variable region of huLiv1-14 (SEQ ID NO: 18), and HC CDR1, HC CDR2 and HC CDR3 of the heavy chain variable region of huLiv1-14 (SEQ ID NO: 17), as determined by Rabat index. In certain embodiments, the antibody construct comprises LC CDR1, LC CDR2 and LC CDR3 of the light chain variable region of huLiv1-14 (SEQ ID NO: 18), and HC CDR1, HC CDR2 and HC CDR3 of the heavy chain variable region of huLiv1-14 (SEQ ID NO: 17), as determined by IMGT. In certain embodiments, the antibody construct comprises the heavy and light chain variable region sequences of Liv1-1.7A4 (SEQ ID NOs:19 and 20, respectively). In certain embodiments, the antibody construct comprises LC CDR1, LC CDR2 and LC CDR3 of the light chain variable region of Liv1-1.7A4 (SEQ ID NO:20), and HC CDR1, HC CDR2 and HC CDR3 of the heavy chain variable region of Liv1-1.7A4 (SEQ ID NO: 19), as determined by Rabat index. In certain embodiments, the antibody construct comprises a humanized antibody or antigen binding fragment thereof comprising LC CDR1, LC CDR2 and LC CDR3 of the light chain variable region of Liv1-1.7A4 (SEQ ID NO:20), and HC CDR1, HC CDR2 and HC CDR3 of the heavy chain variable region of Liv1-1.7A4 (SEQ ID NO:19), as determined by Kabat index. In certain embodiments, the antibody construct comprises LC CDR1, LC CDR2 and LC CDR3 of the light chain variable region of Liv1-1.7A4 (SEQ ID NO:20), and HC CDR1, HC CDR2 and HC CDR3 of the heavy chain variable region of Liv1-1.7A4 (SEQ ID NO: 19), as determined by IMGT. In certain embodiments, the antibody construct comprises a humanized antibody or antigen binding fragment thereof comprising LC CDR1, LC CDR2 and LC CDR3 of the light chain variable region of Liv1-1.7A4 (SEQ ID NO:20), and HC CDR1, HC CDR2 and HC CDR3 of the heavy chain variable region of Liv1-1.7A4 (SEQ ID NO:19), as determined by IMGT. In certain embodiments, the antibody construct comprises the heavy and light chain variable region sequences of huLiv1-22 (SEQ ID NOs:21 and 22, respectively). In certain embodiments, the antibody construct comprises LC CDR1, LC CDR2 and LC CDR3 of the light chain variable region of huLiv1-22 (SEQ ID NO:22), and HC CDR1, HC CDR2 and HC CDR3 of the heavy chain variable region of huLiv1-22 (SEQ ID NO:21), as determined by Kabat index. In certain embodiments, the antibody construct comprises LC CDR1, LC CDR2 and LC CDR3 of the light chain variable region of huLiv1-22 (SEQ ID NO:22), and HC CDR1, HC CDR2 and HC CDR3 of the heavy chain variable region of huLiv1-22 (SEQ ID NO:21), as determined by IMGT. In certain embodiments, the antibody construct comprises a humanized antibody or antigen binding fragment thereof comprising LC CDR1, LC CDR2 and LC CDR3 of the light chain variable region of huLiv1-22 (SEQ ID NO:22), and HC CDR1, HC CDR2 and HC CDR3 of the heavy chain variable region of huLiv1-22 (SEQ ID NO:21), as determined by Kabat index. In certain embodiments, the antibody construct comprises a humanized antibody or antigen binding fragment thereof comprising LC CDR1, LC CDR2 and LC CDR3 of the light chain variable region of huLiv1-22 (SEQ ID NO:22), and HC CDR1, HC CDR2 and HC CDR3 of the heavy chain variable region of huLiv1-22 (SEQ ID NO:21), as determined by IMGT. comprises a humanized antibody or antigen binding fragment thereof comprising

In certain embodiments, the antibody construct comprises a MUC16 antibody, e.g., sofituzumab, 4H11 (US2013/0171152), 4H5 (US2013/0171152) or an antigen binding portion thereof. In certain embodiments, the antibody construct comprises the heavy and light chain variable region sequences of sofituzumab (SEQ ID NOs:23 and 24, respectively). In certain embodiments, the antibody construct comprises LC CDR1, LC CDR2 and LC CDR3 of the light chain variable region of sofituzumab (SEQ ID NO:24), and HC CDR1, HC CDR2 and HC CDR3 of the heavy chain variable region of sofituzumab (SEQ ID NO:23), as determined by Kabat index. In certain embodiments, the antibody construct comprises LC CDR1, LC CDR2 and LC CDR3 of the light chain variable region of sofituzumab (SEQ ID NO:24), and HC CDR1, HC CDR2 and HC CDR3 of the heavy chain variable region of sofituzumab (SEQ ID NO:23), as determined by IMGT. In certain embodiments, the antibody construct comprises a humanized antibody or an antigen binding fragment thereof comprising LC CDR1, LC CDR2 and LC CDR3 of the light chain variable region of sofituzumab (SEQ ID NO:24), and HC CDR1, HC CDR2 and HC CDR3 of the heavy chain variable region of sofituzumab (SEQ ID NO:23), as determined by Rabat index. In certain embodiments, the antibody construct comprises a humanized antibody or an antigen binding fragment thereof comprising LC CDR1, LC CDR2 and LC CDR3 of the light chain variable region of sofituzumab (SEQ ID NO:24), and HC CDR1, HC CDR2 and HC CDR3 of the heavy chain variable region of sofituzumab (SEQ ID NO:23), as determined by IMGT. In certain embodiments, the antibody construct comprises a humanized antibody or an antigen binding fragment thereof comprising LC CDR1, LC CDR2 and LC CDR3 of the light chain variable region of sofituzumab (SEQ ID NO:24), and HC CDR1, HC CDR2 and HC CDR3 of the heavy chain variable region of sofituzumab (SEQ ID NO:23), as determined by Rabat index. In certain embodiments, the antibody construct comprises a humanized antibody or an antigen binding fragment thereof comprising LC CDR1, LC CDR2 and LC CDR3 of the light chain variable region of sofituzumab (SEQ ID NO:24), and HC CDR1, HC CDR2 and HC CDR3 of the heavy chain variable region of sofituzumab (SEQ ID NO:23), as determined by IMGT. In certain embodiments, the antibody construct comprises the heavy and light chain variable region sequences of antibody 4H11 (SEQ ID NOs:13 and 14, respectively). In certain embodiments, the antibody construct comprises LC CDR1, LC CDR2 and LC CDR3 of the light chain variable region of antibody 4H11 (SEQ ID NO: 14), and HC CDR1, HC CDR2 and HC CDR3 of the heavy chain variable region of antibody 4H11 (SEQ ID NO: 13), as determined by Rabat index. In certain embodiments, the antibody construct comprises a humanized antibody or an antigen binding fragment thereof comprising LC CDR1, LC CDR2 and LC CDR3 of the light chain variable region of antibody 4H11 (SEQ ID NO: 14), and HC CDR1, HC CDR2 and HC CDR3 of the heavy chain variable region of antibody 4H11 (SEQ ID NO: 13), as determined by Rabat index. In certain embodiments, the antibody construct comprises LC CDR1, LC CDR2 and LC CDR3 of the light chain variable region of antibody 4H11 (SEQ ID NO: 14), and HC CDR1, HC CDR2 and HC CDR3 of the heavy chain variable region of 4H11 (SEQ ID NO: 13), as determined by IMGT. In certain embodiments, the antibody construct comprises a humanized antibody or antigen binding fragment thereof comprising LC CDR1, LC CDR2 and LC CDR3 of the light chain variable region of antibody 4H11 (SEQ ID NO: 14), and HC CDR1, HC CDR2 and HC CDR3 of the heavy chain variable region of 4H11 (SEQ ID NO: 13), as determined by IMGT. In certain embodiments, the antibody construct comprises the heavy and light chain variable region sequences of antibody 4A5 (SEQ ID NOs: 15 and 16, respectively). In certain embodiments, the antibody construct comprises LC CDR1, LC CDR2 and LC CDR3 of the light chain variable region of antibody 4A5 (SEQ ID NO: 16), and HC CDR1, HC CDR2 and HC CDR3 of the heavy chain variable region of 4A5 (SEQ ID NO: 15), as determined by Rabat index. In certain embodiments, the antibody construct comprises a humanized antibody or an antigen binding fragment thereof comprising LC CDR1, LC CDR2 and LC CDR3 of the light chain variable region of antibody 4A5 (SEQ ID NO: 16), and HC CDR1, HC CDR2 and HC CDR3 of the heavy chain variable region of antibody 4A5 (SEQ ID NO: 15), as determined by Rabat index. In certain embodiments, the antibody construct comprises LC CDR1, LC CDR2 and LC CDR3 of the light chain variable region of antibody 4A5 (SEQ ID NO: 16), and HC CDR1, HC CDR2 and HC CDR3 of the heavy chain variable region of antibody 4A5 (SEQ ID NO: 15), as determined by IMGT. In certain embodiments, the antibody construct comprises a humanized antibody or antigen binding fragment thereof comprising LC CDR1, LC CDR2 and LC CDR3 of the light chain variable region of 4A5 (SEQ ID NO: 16), and HC CDR1, HC CDR2 and HC CDR3 of the heavy chain variable region of 4A5 (SEQ ID NO: 15), as determined by IMGT.

In certain embodiments, the antibody construct comprises a PD-L1 antibody, e.g., atezolizumab, MDX-1105 (WO 2007/005874) or an antigen binding portion thereof. In certain embodiments, the antibody construct comprises the heavy and light chain variable region sequences of atezolizumab (SEQ ID NOs: 11 and 12, respectively). In certain embodiments, the antibody construct comprises LC CDR1, LC CDR2 and LC CDR3 of the light chain variable region of atezolizumab (SEQ ID NO: 12), and HC CDR1, HC CDR2 and HC CDR3 of the heavy chain variable region of atezolizumab (SEQ ID NO: 11), as determined by Rabat index. In certain embodiments, the antibody construct comprises LC CDR1, LC CDR2 and LC CDR3 of the light chain variable region of atezolizumab (SEQ ID NO: 12), and HC CDR1, HC CDR2 and HC CDR3 of the heavy chain variable region of atezolizumab (SEQ ID NO: 11), as determined by IMGT. In certain embodiments, the antibody construct comprises a humanized antibody or an antigen binding fragment thereof comprising LC CDR1, LC CDR2 and LC CDR3 of the light chain variable region of atezolizumab (SEQ ID NO: 12), and HC CDR1, HC CDR2 and HC CDR3 of the heavy chain variable region of atezolizumab (SEQ ID NO: 11), as determined by Rabat index. In certain embodiments, the antibody construct comprises a humanized antibody or an antigen binding fragment thereof comprising LC CDR1, LC CDR2 and LC CDR3 of the light chain variable region of atezolizumab (SEQ ID NO: 12), and HC CDR1, HC CDR2 and HC CDR3 of the heavy chain variable region of atezolizumab (SEQ ID NO: 11), as determined by IMGT. In certain embodiments, the antibody construct comprises the heavy and light chain variable region sequences of MDX-1105 (SEQ ID NOs:9 and 10). In certain embodiments, the antibody construct comprises LC CDR1, LC CDR2 and LC CDR3 of the light chain variable region of MDX-1105 (SEQ ID NO: 10), and HC CDR1, HC CDR2 and HC CDR3 of the heavy chain variable region of MDX-1105 (SEQ ID NO:9), as determined by Rabat index. In certain embodiments, the antibody construct comprises a humanized antibody or antigen binding fragment thereof comprising LC CDR1, LC CDR2 and LC CDR3 of the light chain variable region of MDX-1105 (SEQ ID NO: 10), and HC CDR1, HC CDR2 and HC CDR3 of the heavy chain variable region of MDX-1105 (SEQ ID NO:9), as determined by Rabat index. In certain embodiments, the antibody construct comprises LC CDR1, LC CDR2 and LC CDR3 of the light chain variable region of MDX-1105 (SEQ ID NO: 10), and HC CDR1, HC CDR2 and HC CDR3 of the heavy chain variable region of MDX-1105 (SEQ ID NO:9), as determined by IMGT. In certain embodiments, the antibody construct comprises a humanized antibody or antigen binding fragment thereof comprising LC CDR1, LC CDR2 and LC CDR3 of the light chain variable region of MDX-1105 (SEQ ID NO: 10), and HC CDR1, HC CDR2 and HC CDR3 of the heavy chain variable region of MDX-1105 (SEQ ID NO:9), as determined by IMGT.

The exemplary antibody construct V_{H} sequences and V_{L} sequences are illustrated in Table A below.

TABLE A
Exemplary Antibody Construct V_{H} sequences and V_{L} sequences

| | | SEQ ID | Sequence |
|---|---|---|---|
| Antibody | Region | NO: | |
| Pertuzumab | V_{H} | 1 | EVQLVESGGGLVQPGGSLRLSCAASGFTFTDYTMDW |
| | | | VRQAPGKGLEWVADVNPNSGGSIYNQRFKGRFTLSV |
| | | | DRSKNTLYLQMNSLRAEDTAVYYCARNLGPSFYFDY |
| | | | WGQGTLVTVSS |
| | V_{L} | 2 | DIQMTQSPSSLSASVGDRVTITCKASQDVSIGVAWYQ |
| | | | QKPGKAPKLLIYSASYRYTGVPSRFSGSGSGTDFTLTIS |
| | | | SLQPEDFATYYCQQYYIYPYTFGQGTKVEIK |
| Sacituzumab | V_{H} | 3 | QVQLQQSGSELKKPGASVKVSCKASGYTFTNYGMNW |
| | | | VKQAPGQGLKWMGWINTYTGEPTYTDDFKGRFAFSL |
| | | | DTSVSTAYLQISSLKADDTAVYFCARGGFGSSYWYFD |
| | | | VWGQGSLVTVSS |
| | V_{L} | 4 | DIQLTQSPSSLSASVGDRVSITCKASQDVSIAVAWYQQ |
| | | | KPGKAPKLLIYSASYRYTGVPDRFSGSGSGTDFTLTISS |
| | | | LQPEDFAVYYCQQHYITPLTFGAGTKVEIK |
| Ladiratuzumab | V_{H} | 5 | QVQLVQSGAEVKKPGASVKVSCKASGLTIEDYYMHW |
| | | | VRQAPGQGLEWMGWIDPENGDTEYGPKFQGRVTMT |
| | | | RDTSINTAYMELSRLRSDDTAVYYCAVHNAHYGTWF |
| | | | AYWGQGTLVTVSS |
| | V_{L} | 6 | DVVMTQSPLSLPVTLGQPASISCRSSQSLLHSSGNTYL |
| | | | EYFQQRPGQSPRPLIYKISTRFSGVPDRFSGSGSGTDFT |
| | | | LKISRVEAEDVGVYYCFQGSHVPYTFGGGTKVEIK |
| Trastuzumab | V_{H} | 7 | EVQLVESGGGLVQPGGSLRLSCAASGFNIKDTYIHWV |
| | | | RQAPGKGLEWVARIYPTNGYTRYADSVKGRFTISADT |
| | | | SKNTAYLQMNSLRAEDTAVYYCSRWGGDGFYAMDY |
| | | | WGQGTLVTVSS |
| | V_{L} | 8 | DIQMTQSPSSLSASVGDRVTITCRASQDVNTAVAWYQ |
| | | | QKPGKAPKLLIYSASFLYSGVPSRFSGSRSGTDFTLTIS |
| | | | SLQPEDFATYYCQQHYTTPPTFGQGTKVEIK |
| MDX-1105 | V_{H} | 9 | QVQLVQSGAEVKKPGSSVKVSCKTSGDTFSTYAISWV |
| | | | RQAPGQGLEWMGGIIPIFGKAHYAQKFQGRVTITADE |
| | | | STSTAYMELSSLRSEDTAVYFCARKFHFVSGSPFGMD |
| | | | VWGQGTTVTVSS |
| | V_{L} | 10 | EIVLTQSPATLSLSPGERATLSCRASQSVSSYLAWYQQ |
| | | | KPGQAPRLLIYDASNRATGIPARFSGSGSGTDFTLTISS |
| | | | LEPEDFAVYYCQQRSNWPTFGQGTKVEIK |
| Atezolizumab | V_{H} | 11 | EVQLVESGGGLVQPGGSLRLSCAASGFTFSDSWIHWV |
| | | | RQAPGKGLEWVAWISPYGGSTYYADSVKGRFTISADT |
| | | | SKNTAYLQMNSLRAEDTAVYYCARRHWPGGFDYWG |
| | | | QGTLVTVSS |
| | V_{L} | 12 | DIQMTQSPSSLSASVGDRVTITCRASQDVSTAVAWYQ |
| | | | QKPGKAPKLLIYSASFLYSGVPSRFSGSGSGTDFTLTIS |
| | | | SLQPEDFATYYCQQYLYHPATFGQGTKVEIK |
| 4H11 | V_{H} | 13 | EVKLQESGGGFVKPGGSLKVSCAASGFTFSSYAMSWV |
| | | | RLSPEMRLEWVATISSAGGYIFYSDSVQGRFTISRDNA |
| | | | KNTLHLQMGSLRSGDTAMYYCARQGFGNYGDYYAM |
| | | | DYWGQGTTVTVSS |
| | V_{L} | 14 | DIELTQSPSSLAVSAGEKVTMSCKSSQSLLNSRTRKNQ |
| | | | LAWYQQKPGQSPELLIYWASTRQSGVPDRFTGSGSGT |
| | | | DFTLTISSVQAEDLAVYYCQQSYNLLTFGPGTKLEVK |
| 4A5 | V_{H} | 15 | EVKLEESGGGFVKPGGSLKISCAASGFTFRNYAMSWV |
| | | | RLSPEMRLEWVATISSAGGYIFYSDSVQGRFTISRDNA |
| | | | KNTLHLQMGSLRSGDTAMYYCARQGFGNYGDYYAM |
| | | | DYWGQGTTVTVSS |
| | V_{L} | 16 | DIELTQSPSSLAVSAGEKVTMSCKSSQSLLNSRTRKNQ |
| | | | LAWYQQKTGQSPELLIYWASTRQSGVPDRFTGSGSGT |
| | | | DFTLTISSVQAEDLAVYYCQQSYNLLTFGPGTKLEIK |
| huLiv1-14 | V_{H} | 17 | QVQLVQSGAEVKKPGASVKVSCKASGYTIEDYYMHW |
| | | | VRQAPGQGLEWMGWIDPENGDTEYAPTFQGRVTMTR |
| | | | DTSINTAYMELSRLRSDDTAVYYCARHDAHYGTWFA |
| | | | YWGQGTLVTVSS |
| | V_{L} | 18 | DVVMTQSPLSLPVTLGQPASISCRSSQSIIRNDGNTYLE |
| | | | WYQQRPGQSPRRLIYRVSNRFSGVPDRFSGSGSGTDFT |
| | | | LKISRVEAEDVGVYYCFQGSHVPYTFGGGTKVEIK |
| Liv1-1.7A4 | V_{H} | 19 | EIQLQQSGPELMKPGASVKISCKASTYSFTRYFMHWV |
| | | | KQSHGESLEWIGYIDPFNGGTGYNQKFKGKATLTVDK |
| | | | SSSTAYMHLSSLTSEDSAVYYCVTYGSDYFDYWGQG |
| | | | TTLTVSS |
| | V_{L} | 20 | DIVMTQPQKFMSTSVGDRVSVTCKASQNVETDVVWY |
| | | | QQKPGQPPKALIYSASYRHSGVPDRFTGSGSGTNFTLT |
| | | | ISTVQSEDLAEYFCQQYNNYPFTFGSGTKLEIIR |
| huLiv1-22 | V_{H} | 21 | QVQLVQSGAEVKKPGASVKVSCKASGLTIEDYYMHW |
| | | | VRQAPGQGLEWMGWIDPENGDTEYGPKFQGRVTMT |
| | | | RDTSINTAYMELSRLRSDDTAVYYCAVHNAHYGTWF |
| | | | AYWGQGTLVTVSS |
| | V_{L} | 22 | DVVMTQSPLSLPVTLGQPASISCRSSQSLLHSSGNTYL |
| | | | EWYQQRPGQSPRPLIYKISTRFSGVPDRFSGSGSGTDFT |
| | | | LKISRVEAEDVGVYYCFQGSHVPYTFGGGTKVEIK |
| Sofituzumab | V_{H} | 23 | EVQLVESGGGLVQPGGSLRLSCAASGYSITNDYAWN |
| | | | WVRQAPGKGLEWVGYISYSGYTTYNPSLKSRFTISRD |
| | | | TSKNTLYLQMNSLRAEDTAVYYCARWTSGLDYWGQ |
| | | | GTLVTVSS |
| | V_{L} | 24 | DIQMTQSPSSLSASVGDRVTITCKASDLIHNWLAWYQ |
| | | | QKPGKAPKLLIYGATSLETGVPSRFSGSGSGTDFTLTIS |
| | | | SLQPEDFATYYCQQYWTTPFTFGQGTKVEIK |

### Target Binding Domain

An antibody construct may further comprise a target binding domain. A target binding domain may comprise a domain that specifically binds to a target. A target may be an antigen. A target binding domain may comprise an antigen binding domain. A target binding domain may be an antigen-binding portion of an antibody or an antibody fragment. A target binding domain may be one or more fragments of an antibody that can retain the ability to specifically bind to an antigen. A target binding domain may be any antigen binding fragment. A target binding domain may be in a scaffold, in which a scaffold is a supporting framework for the antigen binding domain. A target binding domain may comprise an antigen binding domain in a scaffold.

A target binding domain may comprise an antigen binding domain such as a portion of an antibody comprising the antigen recognition portion, i.e., an antigenic determining variable region of an antibody sufficient to confer recognition and binding of the antigen recognition portion to a target, such as an antigen, i.e., the epitope. A target binding domain may comprise an antigen binding domain of an antibody. A target binding domain may comprise an antigen binding domain of an antibody fragment, such as an Fv or an scFv. An Fv is the minimum antibody fragment which contains a complete antigen-recognition and antigen-binding site. This region may consist of a dimer of one heavy chain and one light chain variable domain in tight, non-covalent association. In this configuration, the three hypervariable regions (CDRs) of each variable domain may interact to define an antigen-binding site on the surface of the V_{H}-V_{L} dimer. A single variable domain (or half of an Fv comprising only three hypervariable regions (CDRs) specific for an antigen) can recognize and bind antigen, although at a lower affinity than the entire binding site.

A target binding domain may be at least 80% identical to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), a single chain variable fragment (scFv), or a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor. In some embodiments, a target binding domain may be at least 80% identical to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), or a single chain variable fragment (scFv).

A target binding domain may be an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), a single chain variable fragment (scFv), or a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor. In some embodiments, a target binding domain may be an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), or a single chain variable fragment (scFv). In some embodiments, a target binding domain may be an antibody or a antigen binding fragment thereof. In some embodiments, a target binding domain is other than an antibody or an antigen binding fragment thereof, such as a protein, polypeptide or peptide, optionally comprising non-natural amino acids.

In some embodiments, a target binding domain a polypeptide, such as a bicyclic peptide (e.g., a Bicycle®), as described in Published International Application No. WO2014/140342, WO2013/050615, WO2013/050616, and WO2013/050617 (the disclosures of which are incorporated by reference herein).

A target binding domain may be attached to an antibody construct. For example, an antibody construct may be fused with a target binding domain to create an antibody construct with a target binding domain fusion. The antibody construct including the target binding domain may be the result of the nucleic acid sequence of the target binding domain being expressed in frame with the nucleic acid sequence of the antibody construct. The antibody construct-target binding domain fusion may be the result of an in-frame genetic nucleotide sequence encoding the antibody construct with the target binding domain. As another example, a target binding domain may be linked to an antibody construct. A target binding domain may be linked to an antibody construct by chemical conjugation. A target binding domain may be attached to a terminus of an Fc region. A target binding domain may be attached to a terminus of an Fc region. A target binding domain may be attached to a terminus of an antibody construct. A target binding domain may be attached to a terminus of an antibody. A target binding domain may be attached to a light chain of an antibody. A target binding domain may be attached to a terminus of a light chain of an antibody. A target binding domain may be attached to a heavy chain of an antibody. A target binding domain may be attached to terminus of a heavy chain of an antibody. The terminus may be a C-terminus. An antibody construct may be attached to 1, 2, 3, and/or 4 target binding domains. The target binding domain may direct the antibody construct to, for example, a particular cell or cell type. A target binding domain of an antibody construct may be selected in order to recognize an antigen, e.g., an antigen expressed on an immune cell. An antigen can be a peptide or fragment thereof. An antigen may be expressed on an antigen-presenting cell. An antigen may be expressed on a dendritic cell, a macrophage, or a B cell. As another example, an antigen may be a tumor antigen. The tumor antigen may be any tumor antigen described herein. When multiple target binding domains are attached to an antibody construct, the target binding domains may bind to the same antigen. When multiple target binding domains are attached to an antibody construct, the target binding domains may bind different antigens.

### Attachment of Linkers to Antibody Construct

The antibody conjugates may comprise a linker, e.g., a cleavable or noncleavable linker. A linker forms a linkage between different parts of a conjugate, e.g., between an antibody construct and a benzazepine compound of the disclosure. In certain embodiments, an antibody conjugate comprises multiple linkers. In certain embodiments, wherein an antibody conjugate comprises multiple linkers, the linkers may be the same linkers or different linkers. Linkers of the conjugates and methods described herein may not affect the binding of active portions of a conjugate (e.g., active portions include antigen binding domains, Fc domains, target binding domains, antibodies, benzazepine compounds or salts, or the like) to a target, which can be a cognate binding partner such as an antigen. In some embodiments, linkers of the conjugates and methods described herein may selectivey affect the binding of active portions of a conjugate (e.g, Fc domains, antibodies, benzazepine compounds or salts, or the like), such an an interaction with an Fc receptor.

A linker is covalently bound to an antibody construct by a bond between the antibody construct and the linker. A linker may be covalently bound to an anti-tumor antigen antibody construct by a bond between the anti-APC antigen antibody construct and the linker. A linker may be covalently bound to an anti-APC antigen-antibody construct at an attachment site by a bond between the anti-tumor antigen antibody construct and the linker. A linker may be covalently bound to an anti-immune cell antigen antibody by a bond between the anti-immune cell antigen antibody and the linker. For example, a linker may be covalently bound to a terminus of an amino acid sequence of an antibody construct or could be covalently bound to a side chain or side chain modification to the antibody construct, such as the side chain of a lysine, serine, threonine, cysteine, tyrosine, aspartic acid, a non-natural amino acid residue, glutamine or glutamic acid residue. A linker may be covalently bound to a terminus of an amino acid sequence of an Fc region of an antibody construct, or may be covalently bound to a side chain or side chain modification of an Fc region of an antibody construct, such as the side chain of a lysine, serine, threonine, cysteine, tyrosine, aspartic acid, a non-natural amino acid residue, glutamine or glutamic acid residue. A linker may be covalently bound to a terminus of an amino acid sequence of an Fc domain of an antibody construct, or may be covalently bound to a side chain or side chain modification of an Fc domain of an antibody construct, such as the side chain of a lysine, serine, threonine, cysteine, tyrosine, aspartic acid, a non-natural amino acid residue, glutamine or glutamic acid residue.

A linker may be covalently bound to an antibody construct at a hinge cysteine. A linker may be covalently bound to an antibody construct at interchain cysteine. A linker may be covalently bound to an antibody construct at a light chain constant domain lysine. A linker may be covalently bound to an antibody construct at an engineered cysteine in the light chain. A linker may be covalently bound to an antibody construct at an interchain cysteine in the light chain. A linker may be covalently bound to an antibody construct at a glutamine in the light chain. A linker may be covalently bound to an antibody construct at an engineered light chain glutamine. A linker may be covalently bound to an antibody construct at an unnatural amino acid engineered into the light chain. A linker may be covalently bound to an antibody construct at an unnatural amino acid engineered into the heavy chain. A linker may be covalently bound to an antibody construct at an Fc region lysine. A linker may be covalently bound to an antibody construct at an Fc domain lysine. A linker may be covalently bound to an antibody construct at an Fc region cysteine. A linker may be covalently bound to an antibody construct at an Fc domain cysteine. A linker may be covalently bound to an antibody construct at an Fc region interchain cysteine. A linker may be covalently bound to an antibody construct at an Fc domain interchain cysteine. A linker may be covalently bound to an antibody construct at an Fc region glutamine. A linker may be covalently bound to an antibody construct at an Fc domain glutamine. A linker may be covalently bound to an antibody construct at an unnatural amino acid engineered into the Fc region. A linker may be covalently bound to an antibody construct at an unnatural amino acid engineered into the Fc domain. A linker may be covalently bound to an antibody construct at an unnatural amino acid engineered into the heavy chain. Amino acids can be engineered into an amino acid sequence of an antibody construct, for example, a linker of a conjugate. Engineered amino acids may be added to a sequence of existing amino acids. Engineered amino acids may be substituted for one or more existing amino acids of a sequence of amino acids.

A linker may be conjugated to an antibody construct via a sulfhydryl group. A linker may be conjugated to an antibody construct via a primary amine. A linker may be a link created between an unnatural amino acid on an antibody construct reacting with oxime bond that was formed by modifying a ketone group with an alkoxyamine on a benzazepine compound or salt thereof.

In some embodiments, when one or more linkers are covalently bound to an antibody construct, an Fc domain of the antibody construct can bind to Fc receptors. In certain embodiments, an antibody construct bound to a linker or an antibody construct bound to a linker bound to a benzazepine compound or salt thereof, retains the ability of the Fc domain of the antibody to bind to one or more Fc receptors. In some embodiments, when one or more linkers are bound to an antibody construct at an attachment site(s), an Fc domain of the antibody construct can not bind to one or more Fc receptors. In certain embodiments, for an antibody construct bound to a linker or an antibody construct bound to a linker bound to a benzazepine compound, the Fc domain of the antibody contruct can not bind to one or more Fc receptors. In certain embodiments, when a linker is connected to an antibody construct at an attachment site(s), the antigen binding domain of an antibody construct bound to a linker or an antibody construct bound to a linker bound to a benzazepine compound or salt thereof can bind its antigen. In certain embodiments, when a linker is connected to an antibody construct at an attachment site(s), a target binding domain of an antibody construct bound to a linker or an antibody construct bound to a linker bound to a benzazepine compound or salt thereof can bind its antigen.

In certain embodiment, a linker or linker bound to a benzazepine compound or salt thereof disclosed herein is not be attached to an amino acid residue of an Fc domain disclosed herein selected from: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335 336, 396, or 428, wherein numbering of amino acid residues in the Fc domain or Fc region is according to the EU index as in Kabat.

In certain embodiment, a linker or linker bound to a benzazepine compound or salt thereof disclosed herein is attached to an amino acid residue of an Fc domain selected from: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335 336, 396, or 428, wherein numbering of amino acid residues in the Fc domain or region is according to the EU index as in Kabat.

In some aspects, the present disclosure provides a method of preparing an antibody conjugate of the formula:

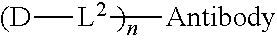

wherein:
Antibody is an antibody construct;
n is selected from 1 to 20; and
D-L² is selected from a compound or salt described herein,

comprising contacting D-L² with an antibody construct to form the antibody conjugate.

In some aspects, the present disclosure provides a method of preparing an antibody conjugate of the formula:

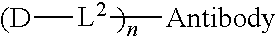

wherein:
Antibody is an antibody construct;
n is selected from 1 to 20;
L² is a linker; and
D is selected from a compound or salt disclosed herein,

comprising contacting L² with the antibody construct to form L²-antibody and contacting L²-antibody with D to form the antibody conjugate.

In some embodiments, the antibody construct comprises an antigen binding domain that specifically binds to an antigen selected from the group consisting of HER2, TROP2 and MUC16. In some embodiments, the methods of the present disclosure further comprise purifying the antibody conjugate.

### Lysine-Based Bioconjugation

An antibody construct can be conjugated to a linker via lysine-based bioconjugation. An antibody construct can be exchanged into an appropriate buffer, for example, phosphate, borate, PBS, histidine, Tris-Acetate at a concentration of about 2 mg/mL to about 10 mg/mL. An appropriate number of equivalents of a construct of a benzazepine compound or salt described herein and a linker, linker-payload, as described herein, can be added as a solution with stirring. Dependent on the physical properties of the linker-payload, a co-solvent can be introduced prior to the addition of the linker-payload to facilitate solubility. The reaction can be stirred at room temperature for 2 hours to about 12 hours depending on the observed reactivity. The progression of the reaction can be monitored by LC-MS. Once the reaction is deemed complete, the remaining linker-payloads can be removed by applicable methods and the antibody conjugate can be exchanged into the desired formulation buffer. Lysine-linked conjugates can be synthesized starting with antibody (mAb) and linker-payload, e.g., 10 equivalents, following Scheme A below (Conjugate=antibody conjugate). Monomer content and drug-antibody construct ratios (molar ratios) can be determined by methods described herein.

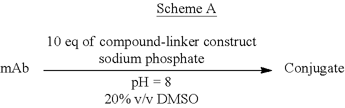

### Cysteine-Based Bioconjugation

An antibody construct can be conjugated to a linker via cysteine-based bioconjugation. An antibody construct can be exchanged into an appropriate buffer, for example, phosphate, borate, PBS, histidine, Tris-Acetate at a concentration of about 2 mg/mL to about 10 mg/mL with an appropriate number of equivalents of a reducing agent, for example, dithiothreitol or tris(2-carboxyethyl)phosphine. The resultant solution can be stirred for an appropriate amount of time and temperature to effect the desired reduction. A construct of a benzazepine compound or salt disclosed herein and a linker, can be added as a solution with stirring. Dependent on the physical properties of the linker-payload, a co-solvent can be introduced prior to the addition of the linker-payload to facilitate solubility. The reaction can be stirred at room temperature for about 1 hour to about 12 hours depending on the observed reactivity. The progression of the reaction can be monitored by liquid chromatography-mass spectrometry (LC-MS). Once the reaction is deemed complete, the remaining free linker-payload can be removed by applicable methods and the antibody conjugate can be exchanged into the desired formulation buffer. Such cysteine-based conjugates can be synthesized starting with antibody (mAb) and linker-payload, e.g., 7 equivalents, using the conditions described in Scheme B below (Conjugate=antibody conjugate). Monomer content and drug-antibody ratios can be determined by methods described herein.

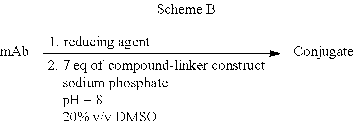

### Benzazepine Compounds and Salts

In some aspects, the present disclosure provides a compound represented by the structure of Formula (IA):

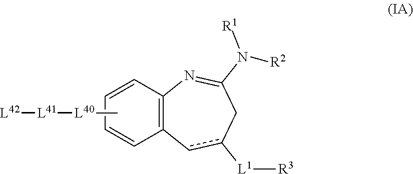

or a pharmaceutically acceptable salt thereof, wherein:
[symbol] represents an optional double bond;
L⁴⁰ is selected from C₃₋₁₂ carbocyclene and 3- to 12-membered heterocyclene, wherein the C₃₋₁₂ carbocyclene and the 3- to 12-membered heterocyclene are optionally substituted with one or more substituents independently selected from:
halogen, —OR¹⁰, —SR¹⁰,
—C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
L¹ and L⁴¹ are independently selected from a bond, C₁-C₂ alkylene optionally substituted with one or more R³¹, —O—, —S—, —N(R¹⁰)—, —C(O)—, —C(O)O—, —OC(O)—, —C(O)N(R¹⁰)—, —N(R¹⁰)C(O)—, —C(NR¹⁰)—, —P(O)(OR¹⁰), —O(R¹⁰O)(O)P—, —OS(O)—, —S(O)O—, —S(O), —OS(O)₂—, —S(O)₂O—, —N(R¹⁰)S(O)₂—, —S(O)₂N(R¹⁰)—, .N(R¹⁰)S(O)—, and —S(O)N(R¹⁰)—;
L⁴² is selected from: 3- to 8-membered saturated heterocycle substituted with a substituent selected from R³⁰, and the 3- to 8-membered saturated heterocycle is optionally substituted with one or more additional substituents selected from R³¹; and optionally substituted C₃₋₁₂ carbocycle, optionally substituted 3- to 12-membered unsaturated heterocycle, optionally substituted heteroaryl, and optionally substituted 8-14 membered bicyclic heterocycle each of which is optionally substituted with one or more substituents independently selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R¹ and R² are independently selected from hydrogen; and C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), and —CN;
R³ is selected from:
—OR¹⁰, —N(R¹⁰)₂, —C(O)N(R¹⁰)₂, —C(O)R¹⁰, —C(O)OR¹⁰, —S(O)R¹⁰, and —S(O)₂R¹⁰; and
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R¹⁰ is independently selected at each occurrence from:
hydrogen, —NH₂; and
C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R¹¹ is independently selected at each occurrence from C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R³⁰ is selected from:
halogen, —OR¹¹, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle, and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R³¹ is selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle, and 3- to 12-membered heterocycle, wherein each C₃₋₁₂ carbocycle, and 3- to 12-membered heterocycle in R³¹ is independently optionally substituted with one or more substituents selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl; and
wherein any substitutable carbon on the benzazepine core is optionally substituted by a substituent selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —P(O)(OR¹⁰)₂, —OP(O)(OR¹⁰)₂, —CN, C₁₋₁₀ alkyl, C₂₋₁₀ alkenyl, and C₂₋₁₀ alkynyl, or two substituents on a single carbon atom or two adjacent carbons combine to form a 3- to 7-membered carbocycle.

In some embodiments for a compound or salt of Formula (IA), L¹ can be attached at C2, C3, C4 or C5 of the benzazepine core, wherein the numbering of the benzazepine is as follows:

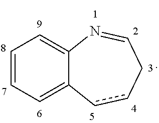

In certain embodiments, for a compound or salt of Formula (IA), L¹ is attached to the benzazepine core at C4. In certain embodiments for a compound or salt of Formula (IA), [symbol] represents a double bond and L¹ is attached to the benzazepine core at C4.

In some embodiments for a compound or salt of Formula (IA), L⁴⁰ can be attached at C6, C7, C8 or C9. In certain embodiments, for a compound or salt of Formula (IA), L⁴⁰ is attached to the benzazepine core at C8. In certain embodiments for a compound or salt of Formula (IA), [symbol] represents a double bond, L¹ is attached to the benzazepine core at C4 and L⁴⁰ is attached to the benzazepine core at C8.

In some embodiments for a compound or salt of Formula (IA), the substitutable carbon on the benzazepine core is selected from C2, C3, C4, C5, C6, C7, C8, and C9. The benzazepine core for a compound or salt of Formula (IA), can be optionally substituted by a substituent selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —P(O)(OR¹⁰)₂, —OP(O)(OR¹⁰)₂, —CN, C₁₋₁₀ alkyl, C₂₋₁₀ alkenyl, and C₂₋₁₀ alkynyl, or two substituents on a single carbon atom combine to form a 3- to 7-membered carbocycle. In some embodiments for a compound or salt of Formula (IA), a moiety at any one of C2, C3, C4, C5, C6, C7, C8, and C9 of the benzazepine core is independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl.

In some embodiments, the compound of Formula (IA) is represented by Formula (IB):

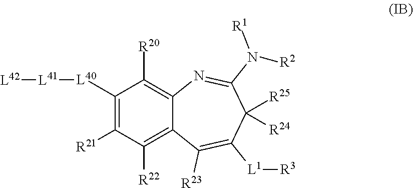

or a pharmaceutically acceptable salt thereof, wherein:
R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and
R²⁴ and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some embodiments, the compound of Formula (IA) is represented by Formula (IC):

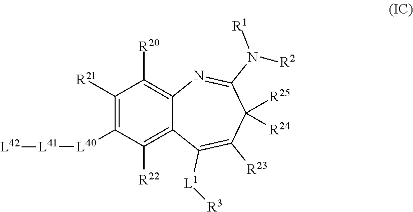

or a pharmaceutically acceptable salt thereof.

In some aspects, the present disclosure provides a compound represented by the structure of Formula (IIIA):

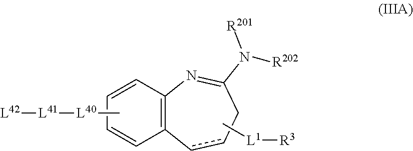

or a pharmaceutically acceptable salt thereof, wherein:
[symbol] represents an optional double bond;
L⁴⁰ is selected from C₃₋₁₂ carbocyclene and 3- to 12-membered heterocyclene, wherein the C₃₋₁₂ carbocyclene and the 3- to 12-membered heterocyclene are optionally substituted with one or more substituents independently selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
L¹ and L⁴¹ are independently selected from a bond, C₁-C₂ alkylene optionally substituted with one or more R³¹, —O—, —S—, —N(R¹⁰)—, —C(O)—, —C(O)O—, —OC(O)—, —C(O)N(R¹⁰)—, —N(R¹⁰)C(O)—, —C(NR¹⁰)—, —P(O)(OR¹⁰)O—, —O(R¹⁰O)(O)P—, —OS(O)—, —S(O)O—, —S(O)—, —OS(O)₂—, —S(O)₂O—, —N(R¹⁰)S(O)₂—, —S(O)₂N(R¹⁰)—, —N(R¹⁰)S(O)—, and —S(O)N(R¹⁰)—;
L⁴² is selected from: 3- to 8-membered saturated heterocycle substituted with a substituent selected from R³⁰, and optionally substituted with one or more additional substituents selected from R³¹; optionally substituted C₃₋₁₂ carbocycle, optionally substituted 3- to 12-membered unsaturated heterocycle, optionally substituted heteroaryl, and optionally substituted 8-14 membered bicyclic heterocycle each of which is optionally substituted with one or more substituents independently selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R²⁰¹ is hydrogen;
R²⁰² is an amine masking group;
R³ is selected from:
—OR¹⁰, —N(R¹⁰)₂, —C(O)N(R¹⁰)₂, —C(O)R¹⁰, —C(O)OR¹⁰, —S(O)R¹⁰, and —S(O)₂R¹⁰;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R¹⁰ is independently selected at each occurrence from:
hydrogen; and
C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R¹¹ is independently selected at each occurrence from C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R³⁰ is selected from:
halogen, —OR¹¹, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R³¹ is selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each is which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl; and

wherein any substitutable carbon on the benzazepine core is optionally substituted by a substituent selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —P(O)(OR¹⁰)₂, —OP(O)(OR¹⁰)₂, —CN, C₁₋₁₀ alkyl, C₂₋₁₀ alkenyl, and C₂₋₁₀ alkynyl, or two substituents on a single carbon atom or two adjacent carbons combine to form a 3- to 7-membered carbocycle.

In some embodiments, the compound of Formula (IIIA) is represented by Formula (IIIB):

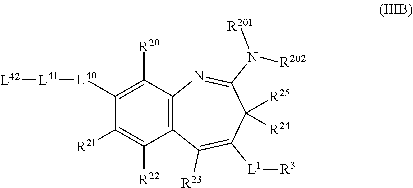

or a pharmaceutically acceptable salt thereof, wherein:
R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and

R²⁴ and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some embodiments, the compound of Formula (IIIA) is represented by Formula (IIIC):

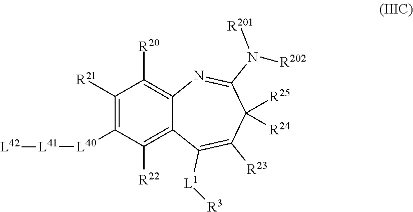

or a pharmaceutically acceptable salt thereof.

In some embodiments for a compound or salt of Formula (IA), (IB) or (IC), R¹ and R² are independently selected from hydrogen; C₁₋₁₀ alkyl, C₂₋₁₀ alkenyl, and C₂₋₁₀ alkynyl, each of which is independently optionally substituted at each occurrence with one or more substituents selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), and —CN. In certain embodiments, R¹ and R² are independently selected from hydrogen and optionally substituted C₁₋₅ alkyl. In an exemplary embodiment, R¹ is hydrogen. In an exemplary embodiment, R² is hydrogen. In an embodiment, R¹ and R² are both hydrogen.

In some embodiments for a compound or salt of Formula (IA), (IB), (IC), (IIIA), (IIIB), or (IIIC), L¹ is selected from —C(O)—, and —C(O)NR¹⁰—. In certain embodiments, L¹ is —C(O)—. In certain embodiments, L¹ is —C(O)NR¹⁰—. R¹⁰ of —C(O)NR¹⁰— may be selected from hydrogen and C₁₋₆ alkyl. For example, L¹ may be —C(O)NH—.

In some embodiments for a compound or salt of Formula (IA), (IB), (IC), (IIIA), (IIIB), or (IIIC), R³ is selected from: —OR¹⁰, and —N(R¹⁰)₂; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl. In certain embodiments, R³ is —N(R¹⁰)₂. In some embodiments, R¹⁰ of —N(R¹⁰)₂ is independently selected at each occurrence from optionally substituted C₁₋₆ alkyl. R¹⁰ of —N(R¹⁰)₂ may be independently selected at each occurrence from methyl, ethyl, propyl, and butyl, any one of which is optionally substituted. In certain embodiments, at least one R³ is optionally substituted propyl. For example, R³ may be

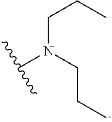 [SMILES: *N(CCC)CCC]

In some embodiments for a compound or salt of Formula (IA), (IB), (IC), (IIIA), (IIIB), or (IIIC), L⁴⁰ is selected from C₃₋₁₂ carbocyclene and 3- to 12-membered heterocyclene, each of which is optionally substituted. In certain embodiments, L⁴⁰ is an optionally substituted C₃₋₁₂ carbocyclene. L⁴⁰ may be an optionally substituted C₃₋₈ carbocyclene, such as an optionally substituted C₅₋₆ carbocyclene. For example, L⁴⁰ may be an optionally substituted arylene. In certain embodiments, L⁴⁰ is an optionally substituted arylene wherein substituents are independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl. In an exemplary embodiment, L⁴⁰ is an optionally substituted phenylene. L⁴⁰ may be

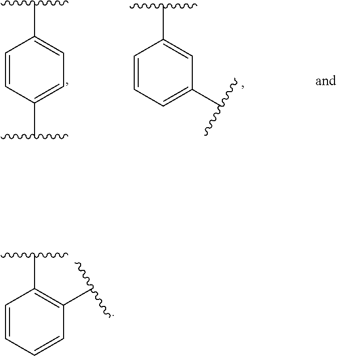 [SMILES: *c1ccc(*)cc1 ; *c1cccc(*)c1 ; *c1ccccc1*]

In some embodiments for a compound or salt of Formula (IA), (IB), (IC), (IIIA), (IIIB), or (IIIC), L⁴⁰ is an optionally substituted 3- to 12-membered heterocyclene. L⁴⁰ may be an optionally substituted 3- to 8-membered heterocyclene, such as an optionally substituted 5- to 6-membered heterocyclene. In certain embodiments, L⁴⁰ is an optionally substituted heteroarylene. In some embodiments, L⁴⁰ is an optionally substituted heteroarylene substituted with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl. L⁴⁰ may be an optionally substituted 5- or 6-membered heteroarylene. For example, L⁴⁰ may be selected from:

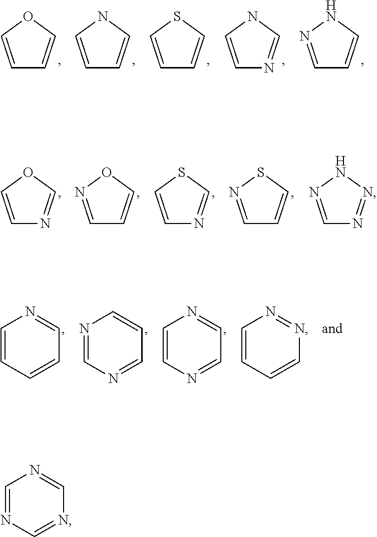

any one of which is optionally substituted. In some embodiments, L⁴⁰ is selected from an optionally substituted 6-membered heteroarylene, such as an optionally substituted pyridinylene. For example, L⁴⁰ may be

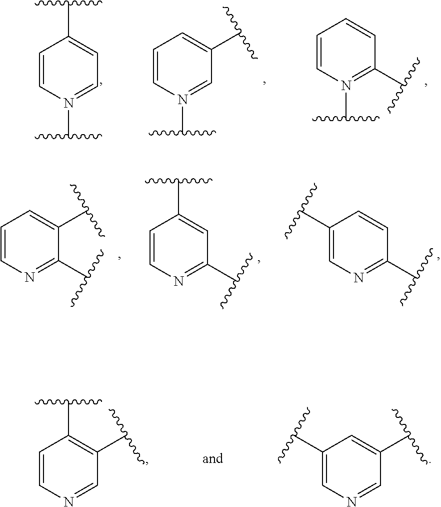

In some embodiments for a compound or salt of Formula (IA), (IB), (IC), (IIIA), (IIIB), or (IIIC), L⁴¹ is selected from —N(R¹⁰)—, —C(O)N(R¹⁰)—, and —C(O)—. In certain embodiments, L⁴¹ is —N(R¹⁰)—, in which R¹⁰ may be selected from hydrogen and C₁₋₆ alkyl. In certain embodiments, L⁴¹ is —C(O)N(R¹⁰)—, in which R¹⁰ may be selected from hydrogen and C₁₋₆ alkyl. In an exemplary embodiment, L⁴¹ is —C(O)—.

In some embodiments for a compound or salt of Formula (IA), (IB), (IC), (IIIA), (IIIB), or (IIIC), L⁴² is selected from optionally substituted C₃₋₁₂ carbocycle, optionally substituted 3- to 12-membered unsaturated heterocycle, optionally substituted heteroaryl, and optionally substituted 8-14 membered bicyclic heterocycle.

In certain embodiments for a compound or salt of Formula (IA), (IB), (IC), (IIIA), (IIIB), or (IIIC), L⁴² is an optionally substituted C₃₋₁₂ carbocycle. In an embodiment, L⁴² is an optionally substituted C₃₋₈ carbocycle. In an embodiment, L⁴² is an optionally substituted C₃₋₆ carbocycle.

In certain embodiments for a compound or salt of Formula (IA), (IB), (IC), (IIIA), (IIIB), or (IIIC), L⁴² is an optionally substituted 3- to 12-membered unsaturated heterocycle. L⁴² may be an optionally substituted 3- to 8-membered unsaturated heterocycle. In an embodiment, L⁴² is an optionally substituted 5- to 6-membered heterocyclene.

In certain embodiments for a compound or salt of Formula (IA), (IB), (IC), (IIIA), (IIIB), or (IIIC), L⁴² is an optionally substituted heteroaryl. In certain embodiments, L⁴² is an optionally substituted heteroaryl substituted with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl. In some embodiments, L⁴² is selected from an optionally substituted 5- or 6-membered heteroaryl. For example, L⁴² may be selected from:

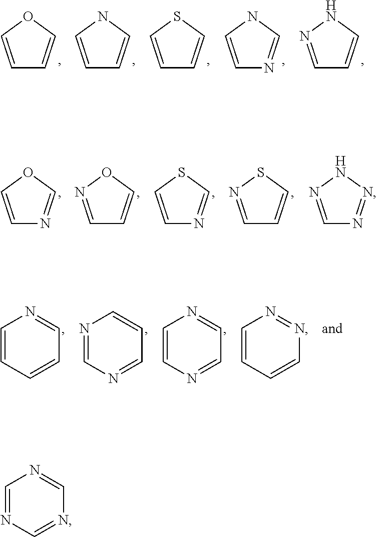

any one of which is optionally substituted. In some embodiments, L⁴² is an optionally substituted 6-membered heteroaryl, such as pyridine.

In some embodiments for a compound or salt of Formula (IA), (IB), (IC), (IIIA), (IIIB), or (IIIC), L⁴² is an optionally substituted 8-14 membered bicyclic heterocycle, optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl. In certain embodiments, L⁴² is an optionally substituted 8- to 12-membered bicyclic heterocycle. In certain embodiments, L⁴² is an optionally substituted 8- to 12-membered bicyclic heterocycle with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl. In an exemplary embodiment, L⁴² is an optionally substituted 8- to 12-membered bicyclic heterocycle with one or more substituents independently selected from —OR¹⁰, —N(R¹⁰)₂, —C(O)OR¹⁰, ═O, and C₁₋₆ alkyl, such as tetrahydroquinoline and cyclopentapyridine. For example, L⁴² may be selected from

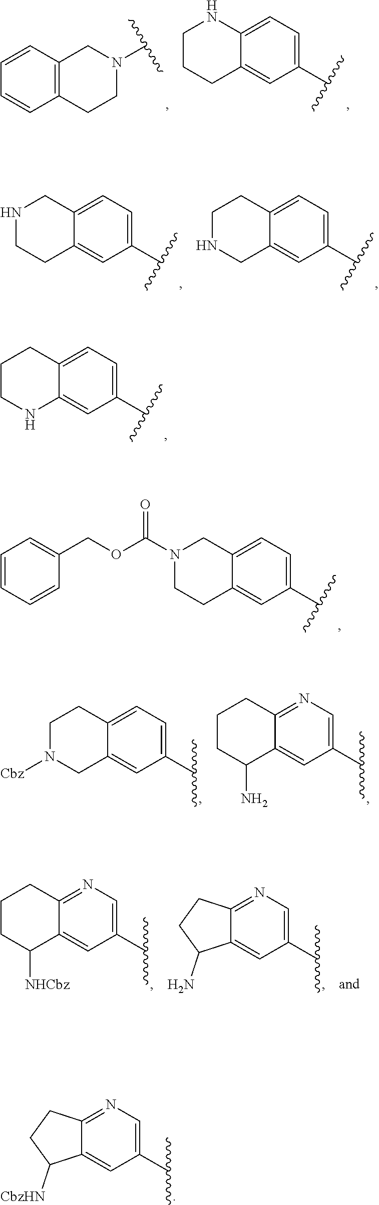

In some embodiments for a compound or salt of Formula (IA), (IB), (IC), (IIIA), (IIIB), or (IIIC), L⁴² is a 3- to 8-membered saturated heterocycle, such as a 5- to 6-membered saturated heterocycle, substituted with a substituent selected from R³⁰, and optionally substituted with one or more substituents selected from R³¹. In some embodiments, R³⁰ is selected from halogen, —OR¹¹, —SR¹⁰, —C(O)N(R¹⁰)₂, —N(R¹⁰)₂, —C(O)OR¹⁰, —NO₂, and —CN; and C₁₋₁₀ alkyl, C₂₋₁₀ alkenyl, and C₂₋₁₀ alkynyl, each of which is independently optionally substituted at each occurrence with one or more substituents (as set forth in the definition of R³⁰); and C₃₋₁₂ carbocycle, and 3- to 12-membered heterocycle, each of which is independently optionally substituted with one or more substituents (as set forth in the definition of R³⁰). R³⁰ may be selected from —OR¹¹; C₁₋₁₀ alkyl, C₂₋₁₀ alkenyl, and C₂₋₁₀ alkynyl, each of which is independently optionally substituted at each occurrence with one or more substituents (as set forth in the definition of R³⁰); and C₃₋₁₂ carbocycle, and 3- to 12-membered heterocycle, each of which is optionally substituted with one or more substituents (as set forth in the definition of R³⁰). In some embodiments, R³¹ is selected from halogen, —OR¹⁰, —SR¹⁰, —C(O)N(R¹⁰)₂, —N(R¹⁰)₂, —C(O)OR¹⁰, —NO₂, and —CN; C₁₋₁₀ alkyl, C₂₋₁₀ alkenyl, and C₂₋₁₀ alkynyl, each of which is optionally substituted with one or more independently selected substituents (as set forth in the definition of R³¹); and C₃₋₁₂ carbocycle, and 3- to 12-membered heterocycle, each of which is optionally substituted with one or more independently selected substituents (as set forth in the definition of R³¹). R³¹ may be selected from —OR¹⁰; C₁₋₁₀ alkyl, C₂₋₁₀ alkenyl, and C₂₋₁₀ alkynyl, each of which is optionally substituted with one or more independently selected substituents (as set forth in the definition of R³¹); and C₃₋₁₂ carbocycle and 3- to 12-membered heterocycle, wherein each of which is optionally substituted with one or more independently selected substituents (as set forth in the definition of R³¹). The 5- to 6-membered saturated heterocycle may be pyrrolidine, piperidine, morpholine, or pyrazolidine. In an exemplary embodiment, L⁴² is pyrrolidine substituted with a substituent selected from R³⁰, and optionally substituted with one or more substituents selected from R³¹. In an exemplary embodiment, L⁴² is piperidine substituted with a substituent selected from R³⁰, and optionally substituted with one or more substituents selected from R³¹.

Any combination of the groups described above for the various variables is contemplated herein.

Throughout the specification, groups and substituents thereof can be chosen to provide stable moieties and compounds.

In some other embodiments, exemplary compounds may include, but are not limited to, a compound or salt of any one of the following compounds:

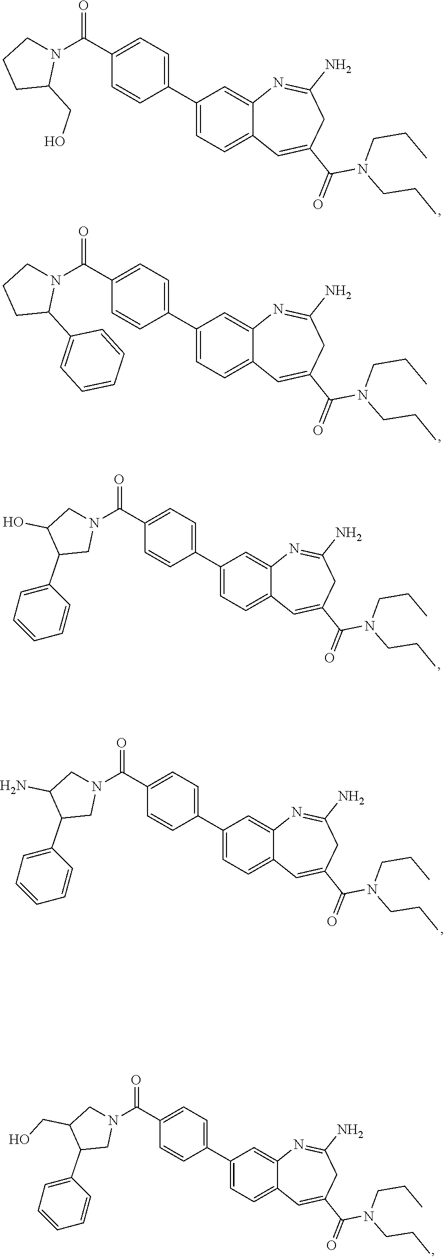 [SMILES: CCCN(CCC)C(=O)C1=Cc2ccc(-c3ccc(C(=O)N4CCCC4CO)cc3)cc2N=C(N)C1 ; CCCN(CCC)C(=O)C1=Cc2ccc(-c3ccc(C(=O)N4CCCC4c4ccccc4)cc3)cc2N=C(N)C1 ; CCCN(CCC)C(=O)C1=Cc2ccc(-c3ccc(C(=O)N4CC(O)C(c5ccccc5)C4)cc3)cc2N=C(N)C1 ; CCCN(CCC)C(=O)C1=Cc2ccc(-c3ccc(C(=O)N4CC(N)C(c5ccccc5)C4)cc3)cc2N=C(N)C1 ; CCCN(CCC)C(=O)C1=Cc2ccc(-c3ccc(C(=O)N4CC(CO)C(c5ccccc5)C4)cc3)cc2N=C(N)C1]

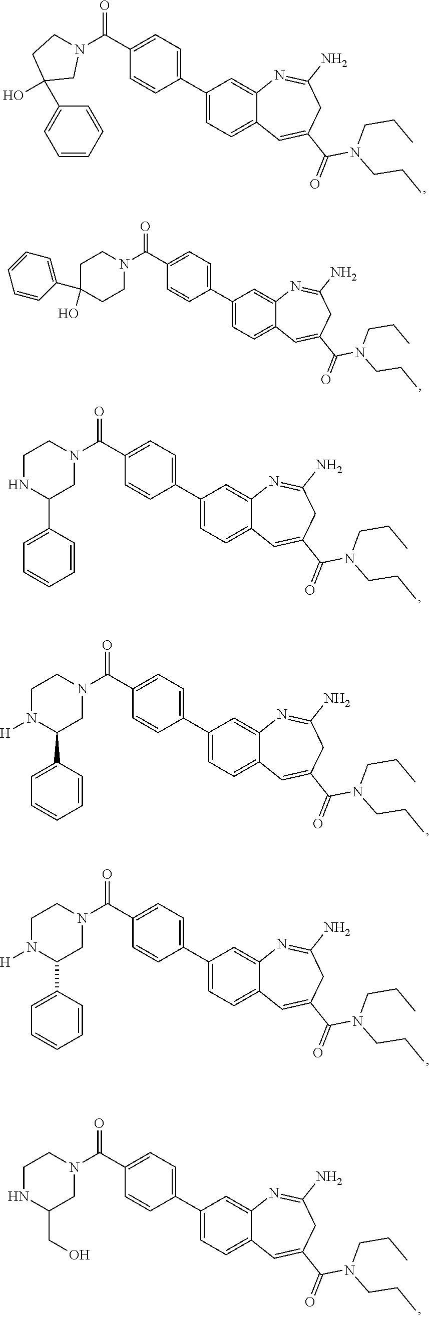 [SMILES: CCCN(CCC)C(=O)C1=Cc2ccc(-c3ccc(C(=O)N4CCC(O)(c5ccccc5)C4)cc3)cc2N=C(N)C1 ; CCCN(CCC)C(=O)C1=Cc2ccc(-c3ccc(C(=O)N4CCC(O)(c5ccccc5)CC4)cc3)cc2N=C(N)C1 ; CCCN(CCC)C(=O)C1=Cc2ccc(-c3ccc(C(=O)N4CCNC(c5ccccc5)C4)cc3)cc2N=C(N)C1 ; CCCN(CCC)C(=O)C1=Cc2ccc(-c3ccc(C(=O)N4CCN[C@H](c5ccccc5)C4)cc3)cc2N=C(N)C1 ; CCCN(CCC)C(=O)C1=Cc2ccc(-c3ccc(C(=O)N4CCN[C@@H](c5ccccc5)C4)cc3)cc2N=C(N)C1 ; CCCN(CCC)C(=O)C1=Cc2ccc(-c3ccc(C(=O)N4CCNC(CO)C4)cc3)cc2N=C(N)C1]

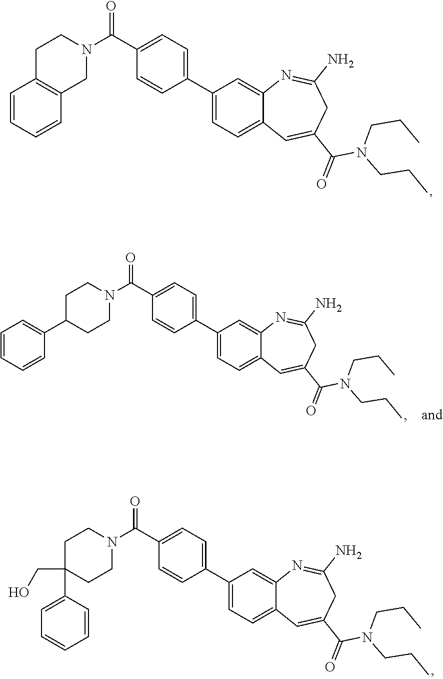 [SMILES: CCCN(CCC)C(=O)C1=Cc2ccc(-c3ccc(C(=O)N4CCc5ccccc5C4)cc3)cc2N=C(N)C1 ; CCCN(CCC)C(=O)C1=Cc2ccc(-c3ccc(C(=O)N4CCC(c5ccccc5)CC4)cc3)cc2N=C(N)C1 ; CCCN(CCC)C(=O)C1=Cc2ccc(-c3ccc(C(=O)N4CCC(CO)(c5ccccc5)CC4)cc3)cc2N=C(N)C1]

In some embodiments, a compound or salt of Formula (IA), (IB), (IC), (IIIA), (IIIB), or (IIIC) is covalently bound to a linker. The linker may be covently bound to any position, valence permitting, on a compound or salt of Formula (IA), (IB), (IC), (IIIA), (IIIB), or (IIIC). The linker may comprise a reactive moiety, e.g., an electrophile, that can react to form a covalent bond with a moiety of an antibody, e.g, an attachment site such as a cysteine side chain or interchain cysteine. In some embodiments, a compound or salt of Formula (IA), (IB), (IC), (IIIA), (IIIB), or (IIIC) may be covalently bound throughout the linker to an antibody.

In some aspects, the present disclosure provides a compound represented by the structure of Formula (IIA):

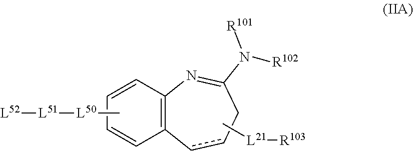

or a pharmaceutically acceptable salt thereof, wherein:
[symbol] represents an optional double bond;
L⁵⁰ is selected from C₃₋₁₂ carbocyclene and 3- to 12-membered heterocyclene, wherein the C₃₋₁₂ carbocyclene and the 3- to 12-membered heterocyclene are optionally substituted with one or more substituents independently selected at each occurrence from:
halogen, —OR¹⁰⁰, —SR¹⁰⁰,
—C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
L²¹ and L⁵¹ are independently selected from a bond, C₁-C₂ alkylene optionally substituted with one or more R³¹⁰, —O—, —S—, —N(R¹⁰⁰)—, —C(O)—, —C(O)O—, —OC(O)—, —C(O)N(R¹⁰⁰)—, —N(R¹⁰⁰)C(O)—, —C(NR¹⁰⁰)—, —P(O)(OR¹⁰⁰)O—, —O(R¹⁰⁰O)(O)P—, —OS(O)—, —S(O)O—, —S(O)—, —OS(O)₂—, —S(O)₂O—, —N(R¹⁰⁰)S(O)₂—, —S(O)₂N(R¹⁰⁰)—, —N(R¹⁰⁰)S(O)—, and —S(O)N(R¹⁰⁰)—;
L⁵² is selected from optionally substituted C₃₋₁₂ carbocycle, optionally substituted 3- to 12-membered unsaturated heterocycle, optionally substituted heteroaryl, and optionally substituted 8-14 membered bicyclic heterocycle; and optionally substituted 3- to 8-membered saturated heterocycle, each of which is optionally substituted with one or more substituents independently selected from:
halogen, -L², —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
R¹⁰¹ and R¹⁰² are independently selected from L², and hydrogen; and C₁₋₁₀ alkyl, C₂₋₁₀ alkenyl, and C₂₋₁₀ alkynyl, each of which is optionally substituted with one or more substituents independently selected from L², halogen, —OR¹⁰⁰, —SR¹⁰⁰, —C(O)N(R¹⁰⁰)₂, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), and —CN;
R¹⁰³ is selected from:
-L², —OR¹⁰⁰, —N(R¹⁰⁰)₂, —C(O)N(R¹⁰⁰)₂, —C(O)R¹⁰⁰, —C(O)OR¹⁰⁰, —S(O)R¹⁰⁰, and —S(O)₂R¹⁰⁰;
C₁₋₁₀ alkyl, C₂₋₁₀ alkenyl, and C₂₋₁₀ alkynyl, each of which is optionally substituted with one or more substituents independently selected from L², halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle and 3- to 12-membered heterocycle; and
C₃₋₁₂ carbocycle and 3- to 12-membered heterocycle, each of which is optionally substituted with one or more substituents independently selected from L², halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
R¹⁰⁰ is independently selected at each occurrence from L² and hydrogen; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R³¹⁰ is selected from:
halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
L² is a linker, wherein at least one of R¹⁰¹, R¹⁰², R¹⁰³, and R¹⁰⁰ is L² or at least one substituent on R¹⁰¹, R¹⁰², R¹⁰³, L⁵², L²¹ and L⁵¹ is -L²; and
wherein any substitutable carbon on the benzazepine core is optionally substituted by a substituent selected from halogen, —OR¹⁰⁰, —SR¹⁰⁰, C(O)N(R¹⁰⁰)₂, —N(R¹⁰⁰)₂, —S(O)R¹⁰⁰, —S(O)₂R¹⁰⁰,
   —C(O)R¹⁰⁰, —C(O)OR¹⁰⁰, —OC(O)R¹⁰⁰, —NO₂, ═O, ═S,
   ═N(R¹⁰⁰), —P(O)(OR¹⁰⁰)₂, —OP(O)(OR¹⁰⁰)₂, —CN, C₁₋₁₀ alkyl, C₂₋₁₀ alkenyl, and C₂₋₁₀ alkynyl, or two substituents on a single carbon atom or two adjacent carbons combine to form a 3- to 7-membered carbocycle.

In some embodiments for a compound or salt of Formula (IIA), L²¹ can be attached at C2, C3, C4 or C5 of the benzazepine core, wherein the numbering of the benzazepine is as follows:

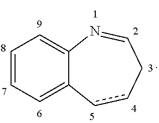

In certain embodiments, for a compound or salt of Formula (IIA), L²¹ is attached to the benzazepine core at C4. In certain embodiments for a compound or salt of Formula (IIA), [symbol] represents a double bond and L²¹ is attached to the benzazepine core at C4.

In some embodiments for a compound or salt of Formula (IIA), L⁵⁰ can be attached at C6, C7, C8 or C9. In certain embodiments, for a compound or salt of Formula (IIA), L⁵⁰ is attached to the benzazepine core at C8. In certain embodiments for a compound or salt of Formula (IIA), [symbol] represents a double bond, L is attached to the benzazepine core at C4 and L is attached to the benzazepine core at C8.

In some embodiments for a compound or salt of Formula (IIA), the substitutable carbon on the benzazepine core is selected from C2, C3, C4, C5, C6, C7, C8, and C9. The benzazepine core for a compound or salt of Formula (IIA), can be optionally substituted by a substituent selected from halogen, —OR¹⁰⁰, —SR¹⁰⁰, —C(O)N(R¹⁰⁰)₂, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₁₀ alkyl, C₂₋₁₀ alkenyl, and C₂₋₁₀ alkynyl, or two substituents on a single carbon atom combine to form a 3- to 7-membered carbocycle. In some embodiments for a compound or salt of Formula (IIA), a moiety at any one of C2, C3, C4, C5, C6, C7, C8, and C9 of the benzazepine core is independently selected from hydrogen, halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl.

In some embodiments, the compound of Formula (IIA) is represented by Formula (IIB):

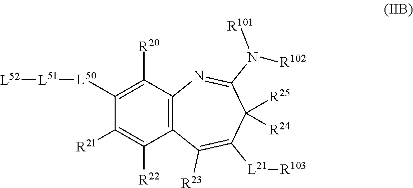

or a pharmaceutically acceptable salt thereof, wherein:
R²⁰, R²¹, R²², and R²³ are independently selected from hydrogen, halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl; and
R²⁴, and R²⁵ are independently selected from hydrogen, halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some embodiments, the compound of Formula (IIA) is represented by Formula (IIC):

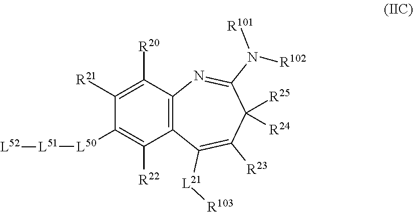

or a pharmaceutically acceptable salt thereof.

In some aspects, the present disclosure provides a compound represented by the structure of Formula (IVA):

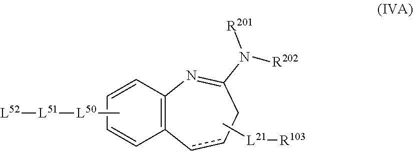

or a pharmaceutically acceptable salt thereof, wherein:
[symbol] represents an optional double bond;
L⁵⁰ is selected from C₃₋₁₂ carbocyclene and 3- to 12-membered heterocyclene, wherein the C₃₋₁₂ carbocyclene and the 3- to 12-membered heterocyclene are optionally substituted with one or more substituents independently selected at each occurrence from:
halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
L²¹ and L⁵¹ are independently selected from a bond, C₁-C₂ alkylene optionally substituted with one or more R³¹⁰, —O—, —S—, —N(R¹⁰⁰)—, —C(O)—, —C(O)O—, —OC(O)—, —C(O)N(R¹⁰⁰)—, —N(R¹⁰⁰)C(O)—, —C(NR¹⁰⁰)—, —P(O)(OR¹⁰⁰)O—, —O(R¹⁰⁰O)(O)P—, —OS(O)—, —S(O)O—, —S(O)—, —OS(O)₂—, —S(O)₂O—, —N(R¹⁰⁰)S(O)₂—, —S(O)₂N(R¹⁰⁰)—, —N(R¹⁰⁰)S(O)—, and —S(O)N(R¹⁰⁰)—;
L⁵² is selected from optionally substituted C₃₋₁₂ carbocycle, optionally substituted 3- to 12-membered unsaturated heterocycle, optionally substituted heteroaryl, optionally substituted 8-14 membered bicyclic heterocycle, and optionally substituted 3- to 8-membered saturated heterocycle, each of which is optionally substituted with one or more substituents independently selected from:
halogen, -L², —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
R²⁰¹ is hydrogen;
R²⁰² is an amine masking group;
R¹⁰³ is selected from:
-L², —OR¹⁰⁰, —N(R¹⁰⁰)₂, —C(O)N(R¹⁰⁰)₂, —C(O)R¹⁰⁰, —C(O)OR¹⁰⁰, —S(O)R¹⁰⁰, and —S(O)₂R¹⁰⁰; and
C₁₋₁₀ alkyl, C₂₋₁₀ alkenyl, and C₂₋₁₀ alkynyl, each of which is optionally substituted with one or more substituents independently selected from L², halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from L², halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
R¹⁰⁰ is independently selected at each occurrence from L² and hydrogen; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R³¹⁰ is selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
L² is a linker wherein at least one of R²⁰¹, R²⁰², R¹⁰³, and R¹⁰⁰ is L² or at least one substituent on R²⁰¹, R²⁰², R¹⁰³, L⁵², L²¹ and L⁵¹ is -L²; and
wherein any substitutable carbon on the benzazepine core is optionally substituted by a substituent selected from halogen, —OR¹⁰⁰, —SR¹⁰⁰, —C(O)N(R¹⁰⁰)₂, —N(R¹⁰⁰)₂, —S(O)R¹⁰⁰, —S(O)₂R¹⁰⁰,
   —C(O)R¹⁰⁰, —C(O)OR¹⁰⁰, —OC(O)R¹⁰⁰, —NO₂, ═O, ═S,
   ═N(R¹⁰⁰), —P(O)(OR¹⁰⁰)₂, —OP(O)(OR¹⁰⁰)₂, —CN, C₁₋₁₀ alkyl, C₂₋₁₀ alkenyl, and C₂₋₁₀ alkynyl, or two substituents on a single carbon atom or two adjacent carbons combine to form a 3- to 7-membered carbocycle.

In some embodiments, the compound of Formula (IVA) is represented by Formula (IVB):

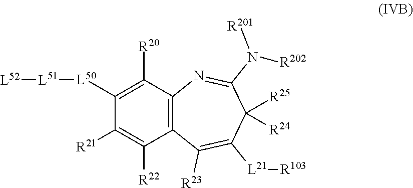

or a pharmaceutically acceptable salt thereof, wherein:
R²⁰, R²¹, R²², and R²³ are independently selected from hydrogen, halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl; and
R²⁴, and R²⁵ are independently selected from hydrogen, halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some embodiments, the compound of Formula (IVA) is represented by Formula (IVC):

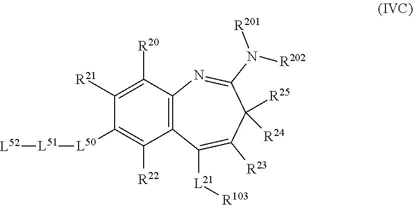

or a pharmaceutically acceptable salt thereof, wherein:
R²⁰, R²¹, R²², and R²³ are independently selected from hydrogen, halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl; and
R²⁴, and R²⁵ are independently selected from hydrogen, halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

In some embodiments for a compound or salt of Formula (IA), (IB), (IC), (IIA), (IIB), (IIC), (IIIA), (IIIB), (IIIC), (IVA), (IVB) and (IVC), R²⁰, R²¹, R²², and R²³ are independently selected from hydrogen, halogen, —OH, —NO₂, —CN, and C₁₋₁₀ alkyl. In certain embodiments, R²⁰, R²¹, R²², and R²³ are each hydrogen.

In some embodiments for a compound or salt of Formula (IA), (IB), (IC), (IIA), (IIB), (IIC), (IIIA), (IIIB), (IIIC), (IVA), (IVB) and (IVC), R²⁴ and R²⁵ are independently selected from hydrogen, halogen, —OH, —NO₂, —CN, and C₁₋₁₀ alkyl, or R²⁴ and R²⁵ taken together form an optionally substituted saturated C₃₋₇ carbocycle. In certain embodiments, R²⁴ and R²⁵ are each hydrogen. In other embodiments, R²⁴ and R²⁵ taken together form an optionally substituted saturated C₃₋₅ carbocycle.

In some embodiments, for a compound of any one of Formulas (IIIA), (IIIB), (IIIC), (IVA), (IVB) and (IVC), R²⁰² is an amine masking group selected from an acid-labile promoiety or an enzymatically-labile promoiety. In certain embodiments, R²⁰² is selected from a group having a bond to an amine that is selectively cleaved under intracellular conditions.

In certain embodiments, R²⁰² together with the nitrogen to which it is attached forms a carbamate or an amide. In certain embodiments, R²⁰² is represented by the formula:

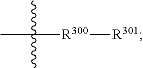

wherein:
R³⁰¹ is selected from an amino acid, a peptide, —O—(C₁-C₆ alkyl) and —C₁-C₆ alkyl, wherein alkyl of —O—(C₁-C₆ alkyl) and —C₁-C₆ alkyl is optionally substituted by one or more substituents independently selected from halogen, —OR¹⁰, —SR¹⁰, —N(R¹⁰)₂, —C(O)R¹⁰, —C(O)N(R¹⁰)₂, —NO₂, —CN, C₃₋₁₃ carbocycle, and 3- to 12-membered heterocycle and R¹⁰ is as previously defined; and
R³⁰⁰ is C(═O), wherein when R³⁰¹ is selected from an amino acid or peptide R³⁰⁰ is the C-terminus of the amino acid or peptide.

In certain embodiments, R³⁰¹ is selected from —O—(C₁-C₄ alkyl) and —C₁-C₄ alkyl, wherein alkyl of —O—(C₁-C₄ alkyl) and —C₁-C₄ alkyl is optionally substituted by one or more substituents independently selected from halogen, —OR¹⁰, —SR¹⁰, —N(R¹⁰)₂, —C(O)R¹⁰, —C(O)N(R¹⁰)₂, —NO₂, —CN, C₃₋₁₃ carbocycle, and 3- to 12-membered heterocycle. In certain embodiments, R²⁰² is selected from 9-fluorenylmethylcarbonyl-, tert-butoxy carbonyl-, benzyloxycarbonyl-, acetyl-, and trifluoroacetyl-.

In certain embodiments, the amino acid of R³⁰¹ is selected from any natural or non-natural amino acid. The amino acid may be selected from arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan. In certain embodiments, the amino acid is an L-amino acid.

In certain embodiments, the peptide of R³⁰¹ includes amino acids each independently selected from any natural or non-natural amino acid. The first amino acid (including R³⁰⁰) may each be independently selected from arginine, histidine, lysine, aspartic acid, glutamic acid, serine, threonine, asparagine, glutamine, cysteine, selenocysteine, glycine, alanine, valine, isoleucine, leucine, methionine, phenylalanine, tyrosine, and tryptophan. In certain embodiments, the amino acids are each independently L-amino acids or D-amino acids. In certain embodiments, the peptide is a dipeptide, tripeptide or tetrapeptide. In certain embodiments, each amino acid of a dipeptide, tripeptide or tetrapeptide, is independently selected from a D- and L-amino acid. In certain embodiments, the amino acid immediately attached to the amine is an L-amino acid, e.g., R³⁰¹ is represented by the formula: -aa1-aa2, or -aa1-aa2-aa3, where aa1 is an L-amino acid and aa2 and aa3 are independently selected from D- and L-amino acids. In certain embodiments, the first amino acid (including R³⁰⁰) is an L-amino acid selected from arginine, histidine, lysine, aspartic acid, glutamic acid, serine, threonine, asparagine, glutamine, cysteine, selenocysteine, glycine, alanine, valine, isoleucine, leucine, methionine, phenylalanine, tyrosine, and tryptophan and the remaining amino acids are D or L amino acids selected from arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan.

In certain embodiments, an amine masking group is selected from those removable groups described in Protective Groups in Organic Synthesis (T. W. Green, P. G. M. Wuts, Wiley-Intersience, N Y, 1999).

In some embodiments for a compound or salt of Formula (IIA), (IIB), (IIC), (IVA), (IVB), or (IVC), L²¹ is —C(O)—. In certain embodiments, L²¹ is —C(O)NR¹⁰⁰—. R¹⁰⁰ of —C(O)NR¹⁰⁰— may be selected from hydrogen, C₁₋₆ alkyl, and -L². For example, L²¹ may be —C(O)NH—. In an embodiment, L²¹ is —C(O)N(L²)-.

In some embodiments for a compound or salt of Formula (IIA), (IIB), (IIC), (IVA), (IVB), or (IVC), R¹⁰³ is selected from: -L², —OR¹⁰⁰, and —N(R¹⁰⁰)₂; and C₁₋₁₀ alkyl, C₂₋₁₀ alkenyl, C₂₋₁₀ alkynyl, C₃₋₁₂ carbocycle, 3- to 12-membered heterocycle, aryl, and heteroaryl, each of which is independently optionally substituted at each occurrence with one or more substituents selected from -L², halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl. In certain embodiments, R¹⁰³ is —N(R¹⁰⁰)₂ and R¹⁰⁰ of —N(R¹⁰⁰)₂ is selected from -L² and hydrogen, and wherein at least one R¹⁰⁰ of —N(R¹⁰⁰)₂ is -L².

In some embodiments for a compound or salt of Formula (IIA), (IIB), (IIC), (IVA), (IVB), or (IVC), L⁵⁰ is an optionally substituted arylene wherein substituents are independently selected from halogen, —OR¹⁰⁰, —SR¹⁰⁰, —N(R¹⁰⁰)₂, —C(O)R¹⁰⁰, —C(O)OR¹⁰⁰, —OC(O)R¹⁰⁰, —NO₂, ═O, ═S, —CN, C₁₋₆ alkyl, C₂₋₆ alkenyl, and C₂₋₆ alkynyl. In an exemplary embodiment, L⁵⁰ is an optionally substituted phenylene. L⁵⁰ may be

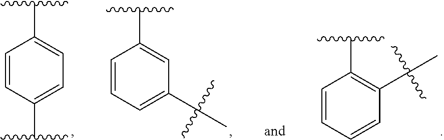 [SMILES: *c1ccc(*)cc1 ; *c1cccc(*)c1 ; *c1ccccc1*]

In some embodiments for a compound or salt of Formula (IIA), (IIB), (IIC), (IVA), (IVB), or (IVC), L⁵¹ is —C(O)N(R¹⁰⁰)—. R¹⁰⁰ of —C(O)N(R¹⁰⁰)— may be selected from hydrogen, C₁₋₆ alkyl, and -L². In certain embodiments, L⁵¹ is —C(O)NH—. In certain embodiments, L⁵¹ is —C(O)NL²-.

In some embodiments for a compound or salt of Formula (IIA), (IIB), (IIC), (IVA), (IVB), or (IVC), L⁵² is an optionally substituted 8- to 14-membered bicyclic heterocycle. In some embodiments, L⁵² is an optionally substituted 8- to 12-membered bicyclic heterocycle with one or more substituents independently selected from L², —OR¹⁰⁰, —N(R¹⁰⁰)₂, and ═O. In an embodiment, L⁵² is a 8- to 12-membered bicyclic heterocycle with at least one L².

In some embodiments for a compound or salt of Formula (IIA), (IIB), (IIC), (IVA), (IVB), or (IVC), L⁵² is a 3- to 8-membered saturated heterocycle optionally substituted with one or more substituents selected from R³¹⁰. In some embodiments, R³¹⁰ is selected from L² and —OR¹⁰⁰; C₁₋₁₀ alkyl, C₂₋₁₀ alkenyl, and C₂₋₁₀ alkynyl, each of which is optionally substituted with one or more independently selected substituents (as set forth in the definition of R³¹⁰); and C₃₋₁₂ carbocycle, and 3- to 12-membered heterocycle each of which is optionally substituted with one or more independently selected substituents (as set forth in the definition of R³¹⁰). In an embodiment, the 3- to 8-membered saturated heterocycle substituted with at least one L². In an exemplary embodiment, L⁵² is pyrrolidine or piperidine optionally substituted with one or more substituents selected from R³¹⁰.

In some aspects, the present disclosure provides a compound or salt thereof selected from compounds 1.1-1.11.

In some embodiments for a compound or salt of Formula (IIA) or (IIB), one of R¹⁰¹, R¹⁰², R¹⁰³, and R¹⁰⁰ is L² or one substituent on R¹⁰¹, R¹⁰², R¹⁰³, L⁵², L²¹ and L⁵¹ is -L².

In some embodiments for a compound or salt of Formula (IVA) or (IVB), one of R²⁰¹, R²⁰², R¹⁰³, and R¹⁰⁰ is L² or one substituent on R²⁰¹, R²⁰², R¹⁰³, L⁵², L²¹ and L⁵¹ is -L².

In some embodiments, L² is covalently bound to a nitrogen atom or oxygen atom. In some embodiments, L² is covalently bound to a nitrogen atom. In some embodiments, L² comprises 15 or more consecutive atoms.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods, conjugates and pharmaceutical compositions include the use of amorphous forms as well as crystalline forms (also known as polymorphs). The compounds described herein may be in the form of pharmaceutically acceptable salts. In certain embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds of any one of Formulas IA, IB, IIA, and IIB may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds, wherein the prodrug is metabolized in vivo to produce a compound of any one of Formulas (IA), (IB), (IC), (IIA), (IIB), and (IIC) or conjugates including any of these, as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In certain embodiments, the prodrug may be converted, e.g., enzymatically or chemically, to the parent compound under the conditions within a cell. In certain embodiments, the parent compound comprises an acidic moiety, e.g., resulting from the hydrolysis of the prodrug, which may be charged under the conditions within the cell. In particular embodiments, the prodrug is converted to the parent compound once it has passed through the cell membrane into a cell. In certain embodiments, the parent compound has diminished cell membrane permeability properties relative to the prodrug, such as decreased lipophilicity and increased hydrophilicity.

In particular embodiments, the parent compound with the acidic moiety is retained within a cell for a longer duration than the same compound without the acidic moiety.

The parent compound, with an acidic moiety, may be retained within the cell, i.e., drug residence, for 10% or longer, such as 15% or longer, such as 20% or longer, such as 25% or longer, such as 30% or longer, such as 35% or longer, such as 40% or longer, such as 45% or longer, such as 50% or longer, such as 55% or longer, such as 60% or longer, such as 65% or longer, such as 70% or longer, such as 75% or longer, such as 80% or longer, such as 85% or longer, or even 90% or longer relative to the same compound without an acidic moiety.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Linkers

The compounds and salts described herein may be covalently bound to a linker, e.g., a peptide linker. In certain embodiments, the linker is also covalently bound to an antibody construct, such as an antibody, and referred to as an antibody conjugate or a conjugate. A conjugate can comprise multiple linkers. These linkers can be the same linkers or different linkers. Linkers of the conjugates described herein may not affect the binding of active portions of a conjugate, e.g., the antigen binding domains, Fc domains, target binding domains, antibodies, benzazepine compounds or salts thereof, or the like, to antigen, which can be a cognate binding partner such as an antigen. Linkers of the conjugates may selectively affect the binding of active portions of a conjugate, e.g., Fc domain or Fc region, benzazepine compounds or salts thereof, or the like, to an Fc domain or Fc region or the cognate binding partner of the benzazepine compound or salt thereof.

A linker can be short, flexible, rigid, cleavable, non-cleavable, hydrophilic, or hydrophobic. A linker can contain segments that have different characteristics, such as segments of flexibility or segments of rigidity. The linker can be chemically stable to extracellular environments, for example, chemically stable in the blood stream, or may include linkages that are not stable. The linker can include linkages that are designed to cleave and/or immolate or otherwise breakdown specifically or non-specifically inside cells. A cleavable linker can be sensitive to enzymes. A cleavable linker can be cleaved by enzymes, such as proteases. A cleavable linker can be a valine-citrullinepeptide containing linker or a valine-alaninepeptide containing linker. A valine-citrulline peptidecontaining or valine-alanine peptide containing linker can contain a pentafluorophenyl group. A valine-citrulline peptide containing or valine-alanine peptide containing linker can contain a succinimide group. A valine-citrulline peptide-containing or valine-alanine peptide containing linker can contain a maleimide group. A valine-citrulline peptide containing or valine-alanine peptide containing linker can contain a para-aminobenzoic acid (PABA) group. A valine-citrulline peptide containing or valine-alanine peptide containing linker can contain a PABA group and a pentafluorophenyl group. A valine-citrulline peptide containing or valine-alanine peptide containing linker can contain a PABA group and a succinimide group. A valine-citrulline peptide containing or valine-alanine-containing linker can contain a PABA group and a maleimide group.

A non-cleavable linker can be protease insensitive. A non-cleavable linker can be maleimidocaproyl linker. A maleimidocaproyl linker can comprise N-maleimidomethylcyclohexane-1-carboxylate. A maleimidocaproyl linker can contain a succinimide group. A maleimidocaproyl linker can contain a maleimide group. A maleimidocaproyl linker can contain pentafluorophenyl group. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonyl) linker.

A linker can contain segments of alkylene, alkenylene, alkynylene, polyether, polyester, polyamide, polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LXPTG recognition motif (SEQ ID NO: 25) to an N-terminal GGG motif to regenerate a native amide bond. The linker created can therefore link a moiety attached to the LXPTG recognition motif (SEQ ID NO: 25) with a moiety attached to the N-terminal GGG motif.

In the conjugates described herein, a compound or salt described herein is linked to the antibody construct by way of linkers. The linker linking the compound or salt to the antibody construct of a conjugate may be short, long, hydrophobic, hydrophilic, flexible or rigid, or may be composed of segments that each independently have one or more of the above-mentioned properties such that the linker may include segments having different properties. The linkers may be polyvalent such that they covalently link more than one compound or salt to a single site on the antibody construct, or monovalent such that covalently they link a single compound or salt to a single site on the antibody.

As will be appreciated by skilled artisans, the linkers may link a compound or salt described herein to the antibody construct (e.g., an antibody) by a covalent linkage(s) between the linker and the antibody construct and compound. As used herein, the expression “linker” is intended to include (i) unconjugated forms of the linker that include a functional group capable of covalently linking the linker to a benzazepine compound or salt thereof and a functional group capable of covalently linking the linker to an antibody; (ii) partially conjugated forms of the linker that include a functional group capable of covalently linking the linker to an antibody construct and that is covalently linked to a compound or salt described herein, or vice versa; and (iii) fully conjugated forms of the linker that is covalently linked to both a compound or salt described herein and an antibody construct. One embodiment pertains to a conjugate formed by contacting an antibody construct that binds a cell surface receptor or tumor associated antigen expressed on a tumor cell with a compound or compound-linker under conditions in which the compound or compound-linker covalently links to the antibody construct. One embodiment pertains to a method of making a conjugate formed by contacting a compound or compound-linker under conditions in which the compound or compound-linker covalently links to the antibody. One embodiment pertains to a method of stimulating immune activity in a cell that expresses a target antigen, comprising contacting the cell with an antibody conjugate that is capable of binding to the cell, under conditions in which the conjugate binds to the cell.

In some embodiments, L² is a cleavable linker or a noncleavable linker. L² may be a cleavable linker that is cleavable by a lysosomal enzyme.

In some embodiments, L² is represented by the formula:

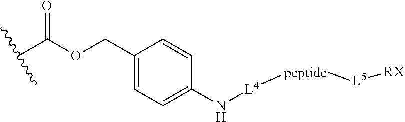

wherein:
L⁴ represents the C-terminus of the peptide and L⁵ is selected from a bond, alkylene and heteroalkylene, wherein L⁵ is optionally substituted with one or more groups independently selected from R³⁰, and RX is a reactive moiety; and
R³⁰ is independently selected at each occurrence from halogen, —OH, —CN, —O-alkyl, —SH, ═O, ═S, —NH₂, and —NO₂; and C₁-C₁₀ alkyl, C₂-C₁₀ alkenyl, and C₂-C₁₀ alkynyl, each of which is independently optionally substituted at each occurrence with one or more substituents selected from halogen, —OH, —CN, —O-alkyl, —SH, ═O, ═S, —NH₂, and —NO₂.

In some embodiments, RX comprises a leaving group. RX may be a maleimide or an alpha-halo carbonyl. In some embodiments, the peptide of L² comprises Val-Cit or Val-Ala.

In some embodiments, L² is represented by the formula:

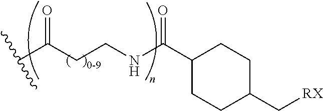

wherein:
RX comprises a reactive moiety; and
n is 0-9.

In some embodiments, RX comprises a leaving group. RX may be a maleimide or an alpha-halo carbonyl.

In some embodiments, L² is further covalently bound to a residue of an antibody construct to form a conjugate, the antibody construct comprising an antigen binding domain and an Fc domain.

Exemplary polyvalent linkers that may be used to link many benzazepine compounds or salts thereof to an antibody construct, such as an antibody, are described. For example, Fleximer® linker technology has the potential to enable high-DAR conjugate with good physicochemical properties. As shown below, the Fleximer® linker technology is based on incorporating drug molecules into a solubilizing poly-acetal backbone via a sequence of ester bonds. The methodology renders highly-loaded conjugates (DAR up to 20) whilst maintaining good physicochemical properties. This methodology could be utilized with benzazepine compounds or salts thereof as shown in the Scheme below.

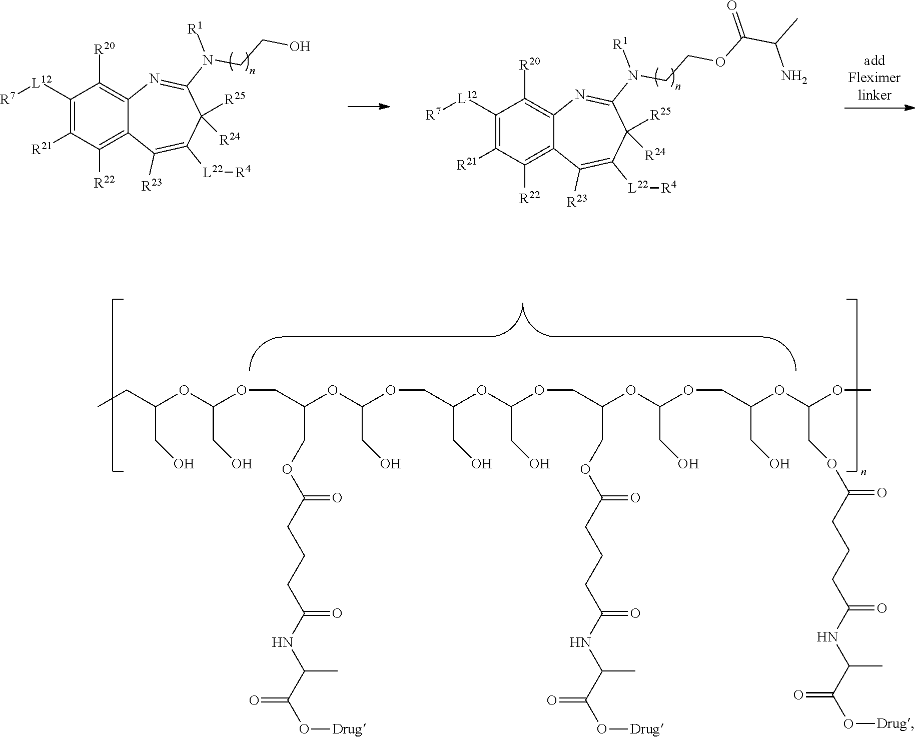

wherein L²² refers to L¹ and R⁷-L¹² refers to L⁴²-L⁴¹-L⁴⁰.

To utilize the Fleximer® linker technology depicted in the scheme above, an aliphatic alcohol can be present or introduced into the benzazepine compound or salt thereof. The alcohol moiety is then conjugated to an alanine moiety, which is then synthetically incorporated into the Fleximer® linker. Liposomal processing of the conjugate in vitro releases the parent alcohol-containing drug.

By way of example and not limitation, some cleavable and noncleavable linkers that may be included in the conjugates are described below.

Cleavable linkers can be cleavable in vitro and in vivo. Cleavable linkers can include chemically or enzymatically unstable or degradable linkages. Cleavable linkers can rely on processing inside the cell to liberate a benzazepine compound or salt thereof, such as reduction in the cytoplasm, exposure to acidic conditions in the lysosome, or cleavage by specific proteases or other enzymes within the cell. Cleavable linkers can incorporate one or more chemical bonds that are either chemically or enzymatically cleavable while the remainder of the linker can be non-cleavable.

A linker can contain a chemically labile group such as hydrazone and/or disulfide groups. Linkers comprising chemically labile groups can exploit differential properties between the plasma and some cytoplasmic compartments. The intracellular conditions that can facilitate benzazepine compound or salt thereof release for hydrazone containing linkers can be the acidic environment of endosomes and lysosomes, while the disulfide containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release the benzazepine compound or salt thereof once the antibody construct benzazepine compound conjugate is internalized into mildly acidic endosomal (pH 5.0-6.5) and lysosomal (pH 4.5-5.0) compartments of the cell. This pH dependent release mechanism can be associated with nonspecific release of the drug (e.g., benzazepine compound or salt thereof). To increase the stability of the hydrazone group of the linker, the linker can be varied by chemical modification, e.g., substitution, allowing tuning to achieve more efficient release in the lysosome with a minimized loss in circulation.

Hydrazone-containing linkers can contain additional cleavage sites, such as additional acid-labile cleavage sites and/or enzymatically labile cleavage sites. Antibody construct benzazepine compound conjugates including exemplary hydrazone-containing linkers can include, for example, the following structures:

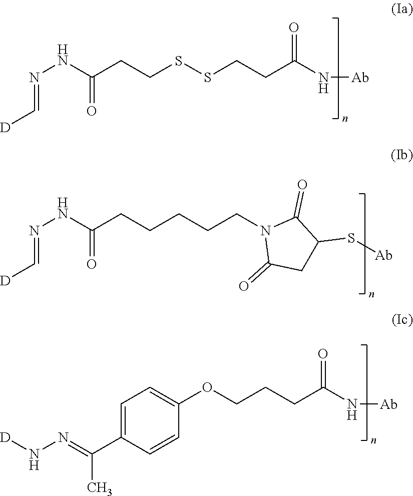

wherein D is a compound or salt described herein, and Ab is an antibody construct, respectively, and n represents the number of—compounds bound to linkers (LP) bound to the antibody construct. In certain linkers, such as linker (Ia), the linker can comprise two cleavable groups—a disulfide and a hydrazone moiety. For such linkers, effective release of the unmodified free benzazepine compound or salt thereof can require acidic pH or disulfide reduction and acidic pH. Linkers such as (Ib) and (Ic) can be effective with a single hydrazone cleavage site.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release the benzazepine compound or salt thereof upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing the benzazepine compound or salt thereof in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Antibody construct benzazepine compound conjugates including exemplary disulfide-containing linkers can include the following structures:

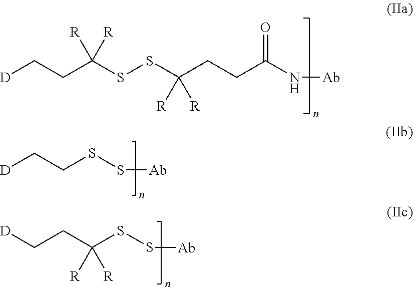

wherein D is a benzazepine compound or salt described herein, and Ab is an antibody construct, respectively, n represents the number of compounds bound to linkers (LP) bound to the antibody construct and R is independently selected at each occurrence from hydrogen or alkyl, for example. Increasing steric hindrance adjacent to the disulfide bond can increase the stability of the linker. Structures such as (IIa) and (IIc) can show increased in vivo stability when one or more R groups is selected from a lower alkyl such as methyl.

Another type of linker that can be used is a linker that is specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers.

Peptide bonds can have good serum stability, as lysosomal proteolytic enzymes can have very low activity in blood due to endogenous inhibitors and the unfavorably high pH value of blood compared to lysosomes. Release of a benzazepine compound or salt thereof from an antibody construct can occur due to the action of lysosomal proteases, e.g., cathepsin and plasmin. These proteases can be present at elevated levels in certain tumor tissues. The linker can be cleavable by a lysosomal enzyme. The lysosomal enzyme can be, for example, cathepsin B, β-glucuronidase, or β-galactosidase.

The cleavable peptide can be selected from tetrapeptides such as Gly-Phe-Leu-Gly, Ala-Leu-Ala-Leu or dipeptides such as Val-Cit, Val-Ala, and Phe-Lys. Dipeptides can have lower hydrophobicity compared to longer peptides.

A variety of dipeptide-based cleavable linkers can be used in the antibody construct-benzazepine compound conjugates described herein.

Enzymatically cleavable linkers can include a self-immolative spacer to spatially separate the benzazepine compound or salt thereof from the site of enzymatic cleavage. The direct attachment of a benzazepine compound or salt thereof to a peptide linker can result in proteolytic release of an amino acid adduct of the benzazepine compound or salt thereof, thereby impairing its activity. The use of a self-immolative spacer can allow for the elimination of the fully active, chemically unmodified benzazepine compound or salt thereof upon amide bond hydrolysis.

One self-immolative spacer can be a bifunctional para-aminobenzyl alcohol group, which can link to the peptide through the amino group, forming an amide bond, while amine containing benzazepine compounds or salts thereof can be attached through carbamate functionalities to the benzylic hydroxyl group of the linker (to give a p-amidobenzylcarbarnate, PABC). The resulting pro-benzazepine compound can be activated upon protease-mediated cleavage, leading to a 1,6-elimination reaction releasing the unmodified benzazepine compound or salt thereof, carbon dioxide, and remnants of the linker group. The following scheme depicts the fragmentation of p-amidobenzyl carbamate and release of the benzazepine compound or salt thereof:

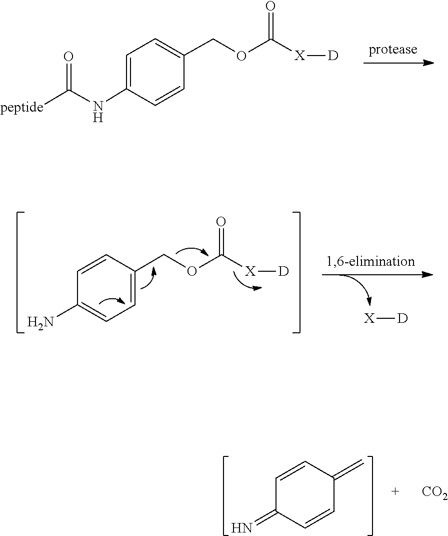

wherein X-D represents the unmodified benzazepine compound or salt thereof and the carbonyl group adjacent peptide is part of the peptide. Heterocyclic variants of this self-immolative group have also been described.

The enzymatically cleavable linker can be a ß-glucuronic acid-based linker. Facile release of the benzazepine compound or salt thereof can be realized through cleavage of the ß-glucuronide glycosidic bond by the lysosomal enzyme ß-glucuronidase. This enzyme can be abundantly present within lysosomes and can be overexpressed in some tumor types, while the enzyme activity outside cells can be low. ß-Glucuronic acid-based linkers can be used to circumvent the tendency of an antibody construct benzazepine compound conjugate to undergo aggregation due to the hydrophilic nature of ß-glucuronides. In certain embodiments, ß-glucuronic acid-based linkers can link an antibody construct to a hydrophobic benzazepine compound. The following scheme depicts the release of a benzazepine compound or salt thereof (D) from an antibody construct (Ab) benzazepine compound conjugate containing a ß-glucuronic acid-based linker:

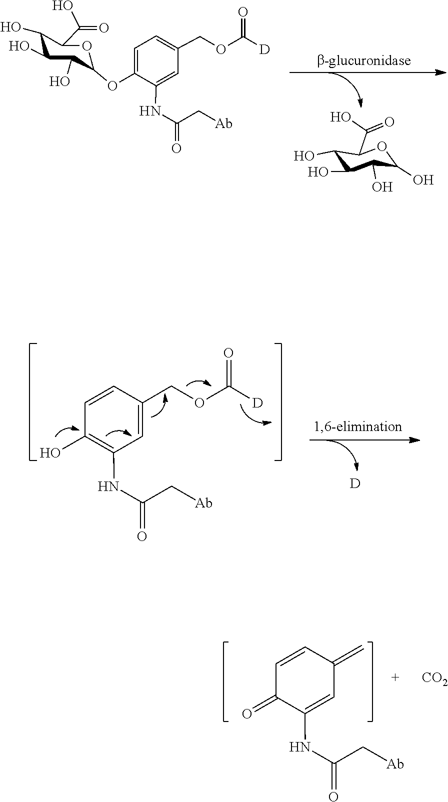

A variety of cleavable β-glucuronic acid-based linkers useful for linking drugs such as auristatins, camptothecin and doxorubicin analogues, CBI minor-groove binders, and psymberin to antibodies have been described. These β-glucuronic acid-based linkers may be used in the conjugates described herein. In certain embodiments, the enzymatically cleavable linker is a β-galactoside-based linker. β-Galactoside is present abundantly within lysosomes, while the enzyme activity outside cells is low.

Additionally, benzazepine compounds or salts thereof containing a phenol group can be covalently bonded to a linker through the phenolic oxygen. One such linker relies on a methodology in which a diamino-ethane “Space Link” is used in conjunction with traditional “PABO”-based self-immolative groups to deliver phenols. Other methods of attaching linkers to hydroxyl groups of compounds are disclosed in WO 2015/095755.

Cleavable linkers can include non-cleavable portions or segments, and/or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more cleavable groups such as a disulfide, a hydrazone or a dipeptide.

Other degradable linkages that can be included in linkers can include ester linkages formed by the reaction of PEG carboxylic acids or activated PEG carboxylic acids with alcohol groups on a benzazepine compound or salt thereof, wherein such ester groups can hydrolyze under physiological conditions to release the benzazepine compound or salt thereof. Hydrolytically degradable linkages can include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

A linker can contain an enzymatically cleavable peptide moiety, for example, a linker comprising structural formula (IIIa), (IIIb), (IIIc), or (IIId):

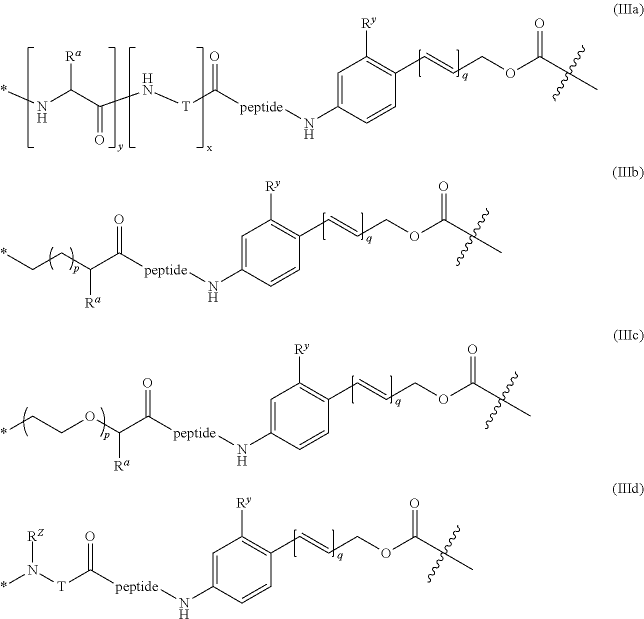

or a salt thereof, wherein: peptide represents a peptide (illustrated N→C, wherein peptide includes the amino and carboxy “termini”) cleavable by a lysosomal enzyme; T represents a polymer comprising one or more ethylene glycol units or an alkylene chain, or combinations thereof; R^{a} is selected from hydrogen, alkyl, sulfonate and methyl sulfonate; R^{y} is hydrogen or C₁₋₄ alkyl-(O)ᵣ—(C₁₋₄ alkylene)ₛ-G¹ or C₁₋₄ alkyl-(N)—[(C₁₋₄ alkylene)-G¹]₂; R^{z} is C₁₋₄ alkyl-(O)ᵣ—(C₁₋₄ alkylene)ₛ-G²; G¹ is SO₃H, CO₂H, PEG 4-32, or sugar moiety; G² is SO₃H, CO₂H, or PEG 4-32 moiety; r is 0 or 1; s is 0 or 1; p is an integer ranging from 0 to 5; q is 0 or 1; x is 0 or 1; y is 0 or 1; [symbol] represents the point of attachment of the linker to a compound or salt described herein; and * represents the point of attachment to the remainder of the linker.

In certain embodiments, the peptide can be selected from a tripeptide or a dipeptide. In particular embodiments, the dipeptide can be selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Exemplary embodiments of linkers according to structural formula (IIIa) that can be included in the conjugates described herein can include the linkers illustrated below (as illustrated, the linkers include a group suitable for covalently linking the linker to an antibody construct):

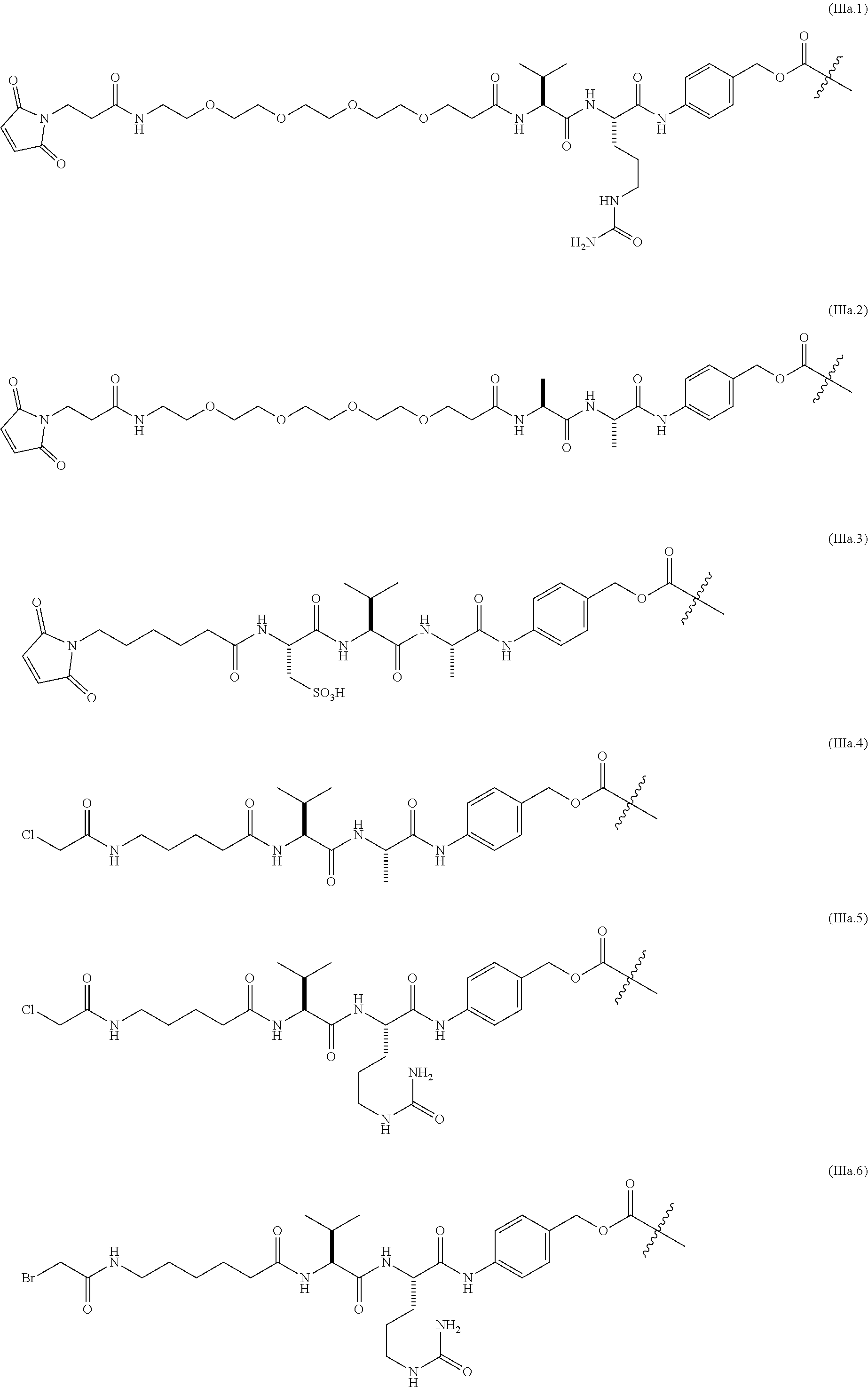

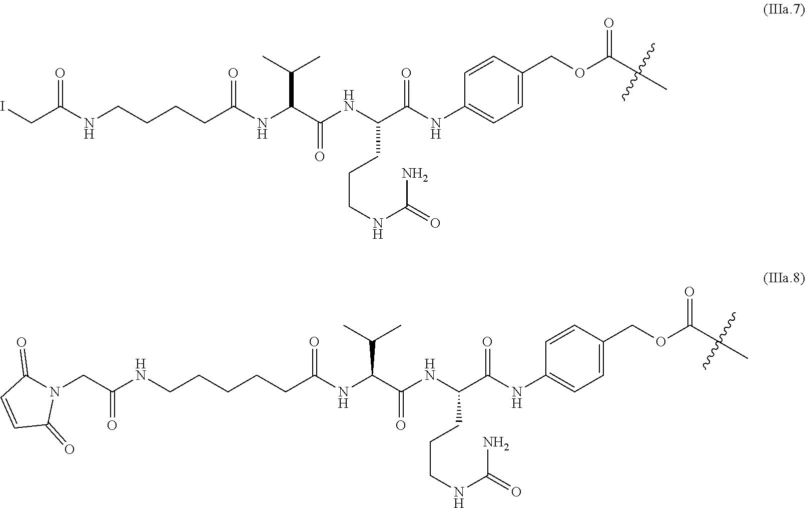 [SMILES: *C(=O)OCc1ccc(NC(=O)[C@H](CCCNC(N)=O)NC(=O)[C@@H](NC(=O)CCCCNC(=O)CI)C(C)C)cc1 ; *C(=O)OCc1ccc(NC(=O)[C@H](CCCNC(N)=O)NC(=O)[C@@H](NC(=O)CCCCCNC(=O)CN2C(=O)C=CC2=O)C(C)C)cc1]

Exemplary embodiments of linkers according to structural formula (IIIb), (IIIc), or (IIId) that can be included in the conjugates can include the linkers illustrated below (as illustrated, the linkers can include a group suitable for covalently linking the linker to an antibody construct):

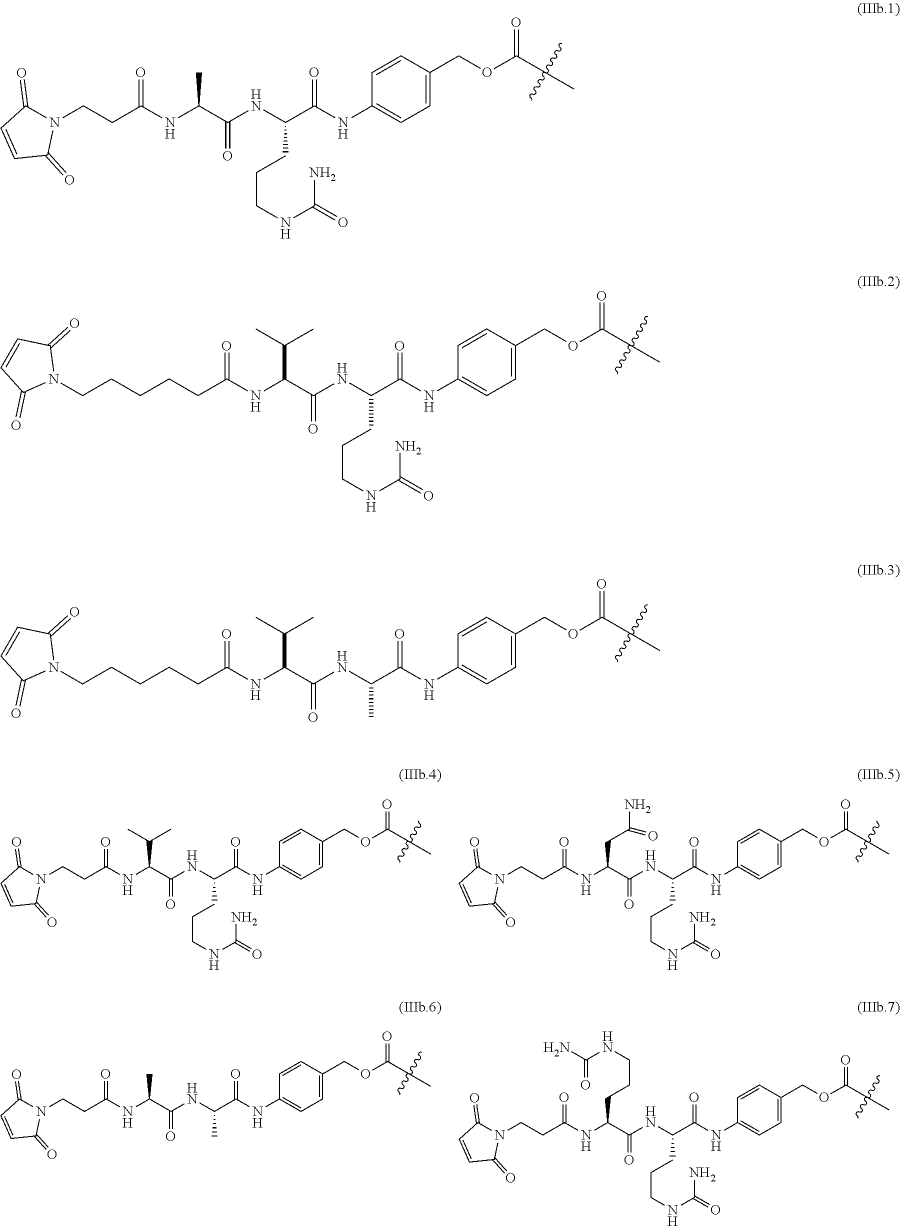

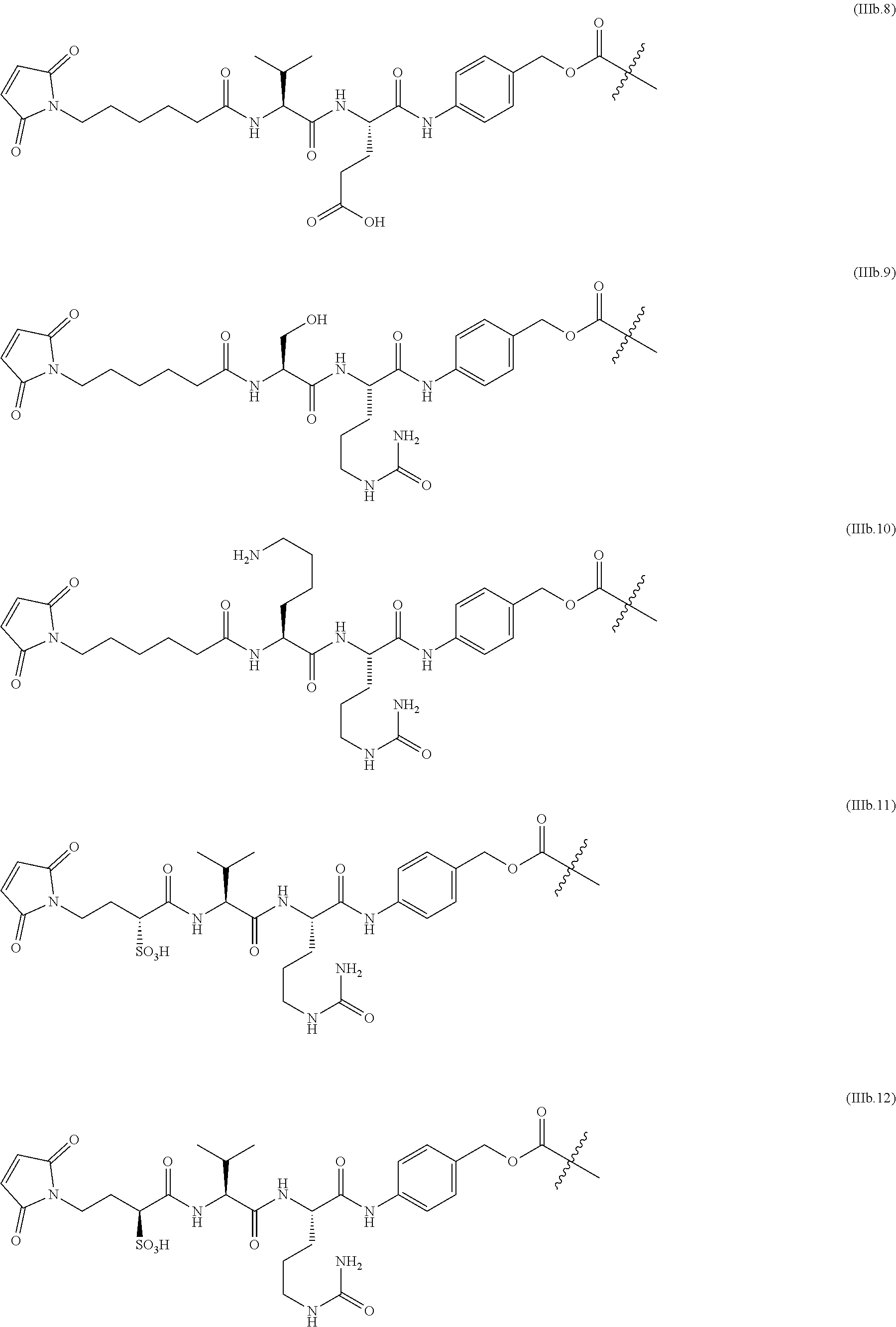 [SMILES: *C(=O)OCc1ccc(NC(=O)[C@H](CCC(=O)O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1 ; *C(=O)OCc1ccc(NC(=O)[C@H](CCCNC(N)=O)NC(=O)[C@@H](NC(=O)[C@@H](CCN2C(=O)C=CC2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1]

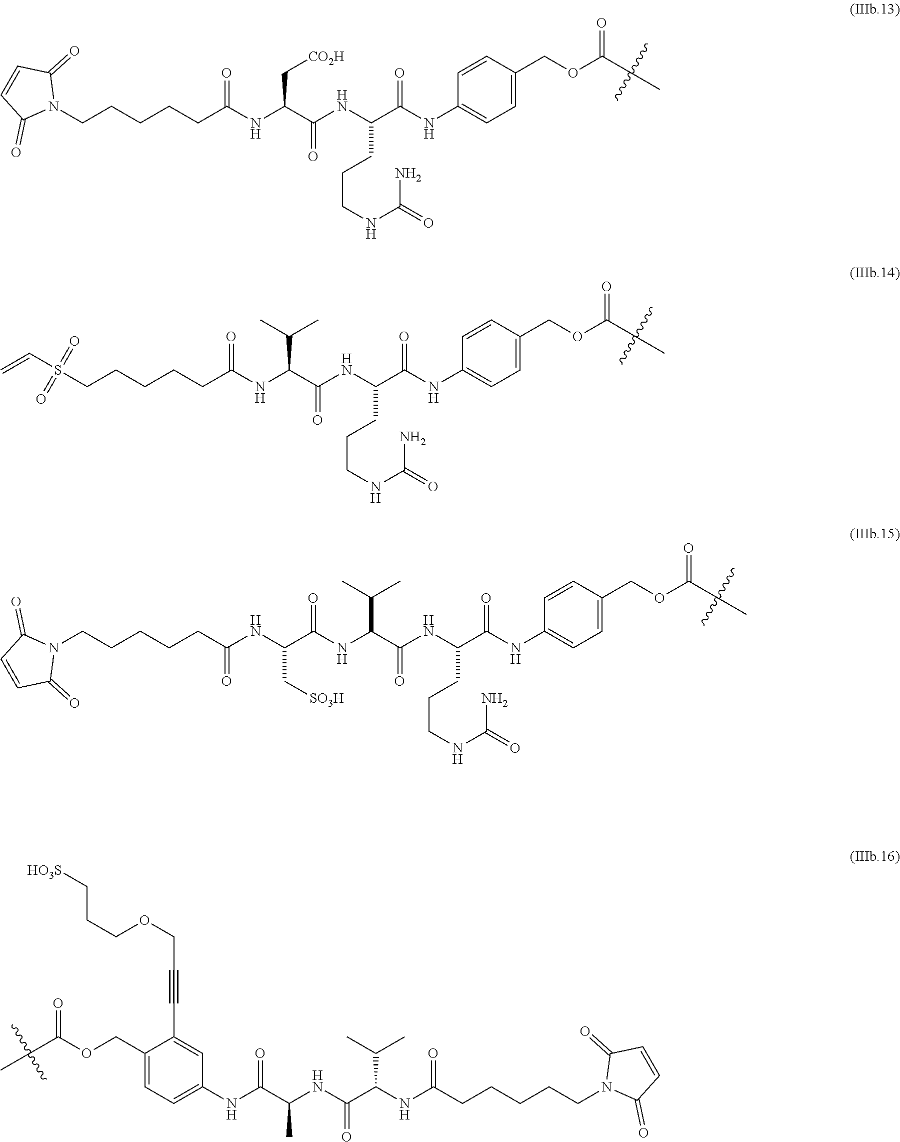 [SMILES: *C(=O)OCc1ccc(NC(=O)[C@H](CCCNC(N)=O)NC(=O)[C@H](CC(=O)O)NC(=O)CCCCCN2C(=O)C=CC2=O)cc1 ; *C(=O)OCc1ccc(NC(=O)[C@H](CCCNC(N)=O)NC(=O)[C@@H](NC(=O)CCCCCS(=O)(=O)C=C)C(C)C)cc1 ; *C(=O)OCc1ccc(NC(=O)[C@H](CCCNC(N)=O)NC(=O)[C@@H](NC(=O)[C@H](CS(=O)(=O)O)NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1C#CCOCCCS(=O)(=O)O]

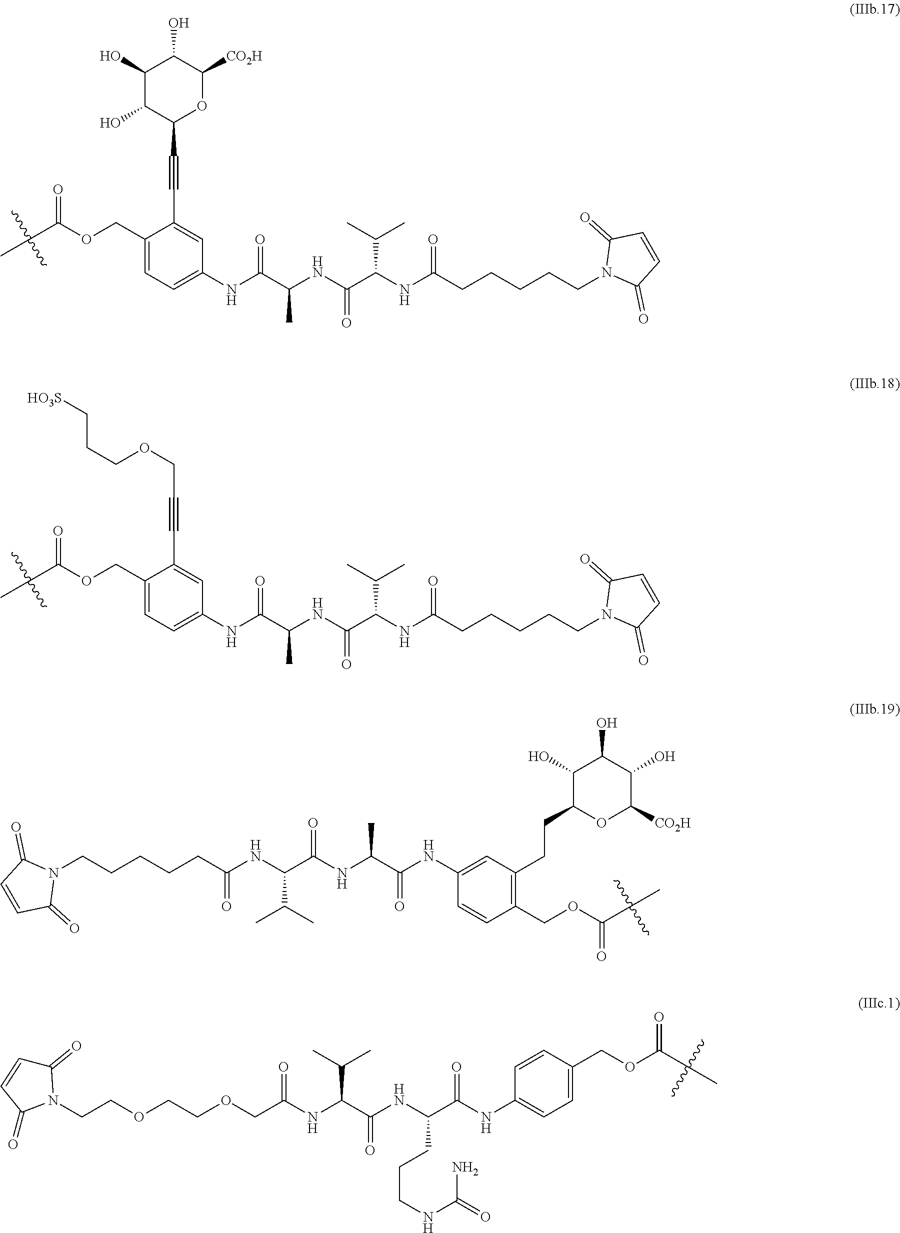 [SMILES: *C(=O)OCc1ccc(NC(=O)[C@H](C)NC(=O)[C@@H](NC(=O)CCCCCN2C(=O)C=CC2=O)C(C)C)cc1C#C[C@@H]1O[C@H](C(=O)O)[C@@H](O)[C@H](O)[C@H]1O ; *C(=O)OCc1ccc(NC(=O)[C@H](C)NC(=O)[C@@H](NC(=O)CCCCCN2C(=O)C=CC2=O)C(C)C)cc1C#CCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](CCCNC(N)=O)NC(=O)[C@@H](NC(=O)COCCOCCN2C(=O)C=CC2=O)C(C)C)cc1]

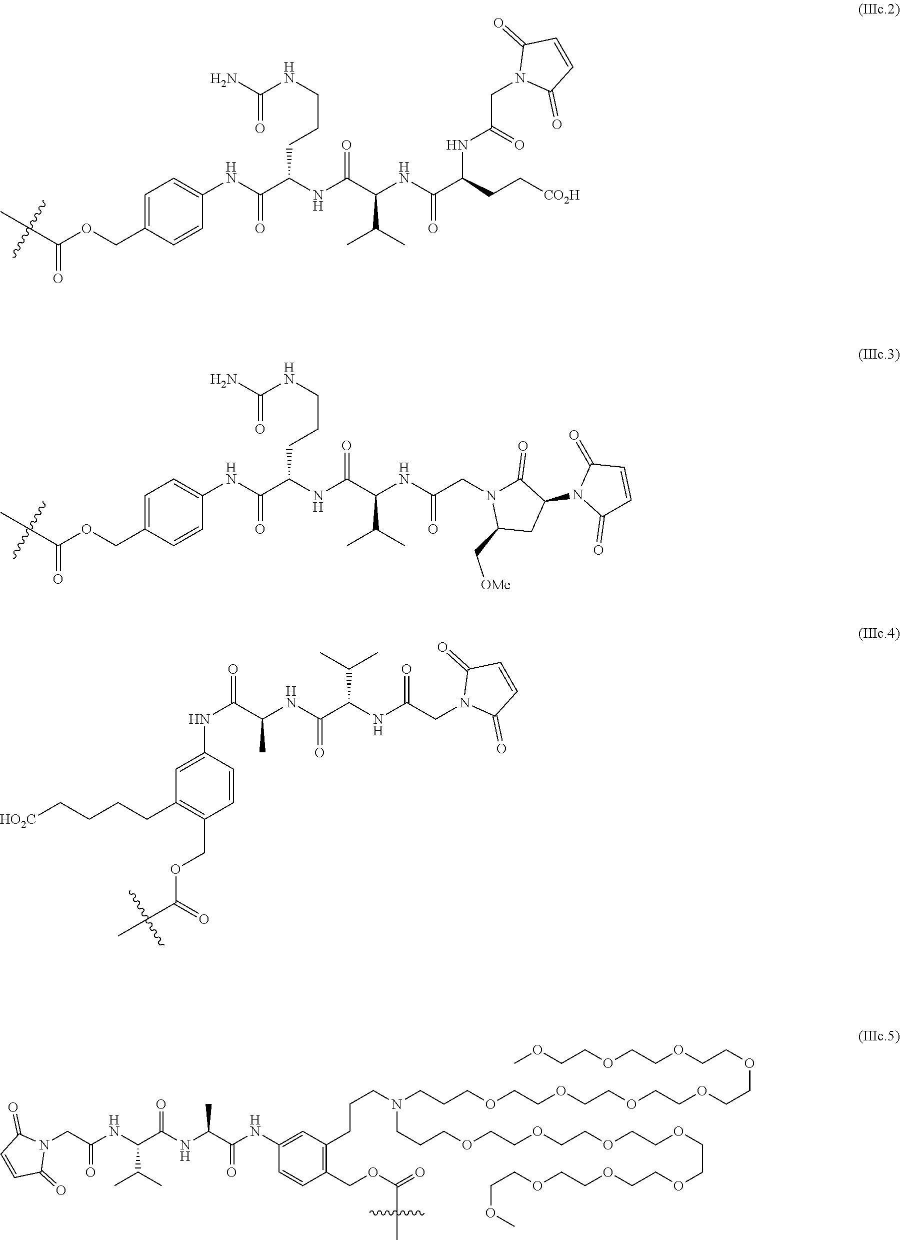 [SMILES: *C(=O)OCc1ccc(NC(=O)[C@H](CCCNC(N)=O)NC(=O)[C@@H](NC(=O)[C@H](CCC(=O)O)NC(=O)CN2C(=O)C=CC2=O)C(C)C)cc1 ; *C(=O)OCc1ccc(NC(=O)[C@H](CCCNC(N)=O)NC(=O)[C@@H](NC(=O)CN2C(=O)[C@@H](N3C(=O)C=CC3=O)C[C@H]2COC)C(C)C)cc1 ; *C(=O)OCc1ccc(NC(=O)[C@H](C)NC(=O)[C@@H](NC(=O)CN2C(=O)C=CC2=O)C(C)C)cc1CCCCC(=O)O ; *C(=O)OCc1ccc(NC(=O)[C@H](C)NC(=O)[C@@H](NC(=O)CN2C(=O)C=CC2=O)C(C)C)cc1CCCN(CCCOCCOCCOCCOCCOCCOCCOCCOC)CCCOCCOCCOCCOCCOCCOCCOCCOC]

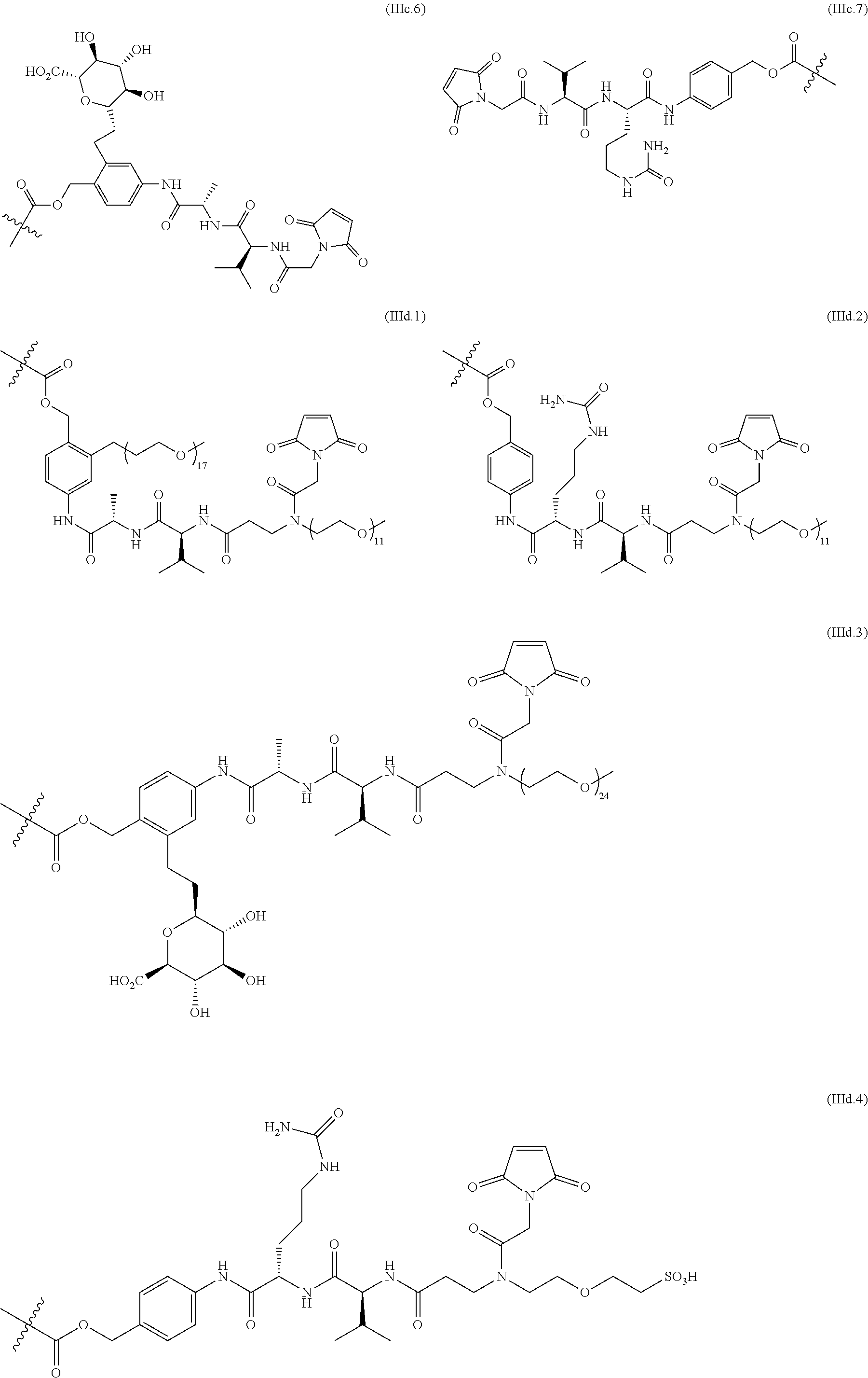

The linker can contain an enzymatically cleavable sugar moiety, for example, a linker comprising structural formula (IVa), (IVb), (IVc), (IVd), or (IVe):

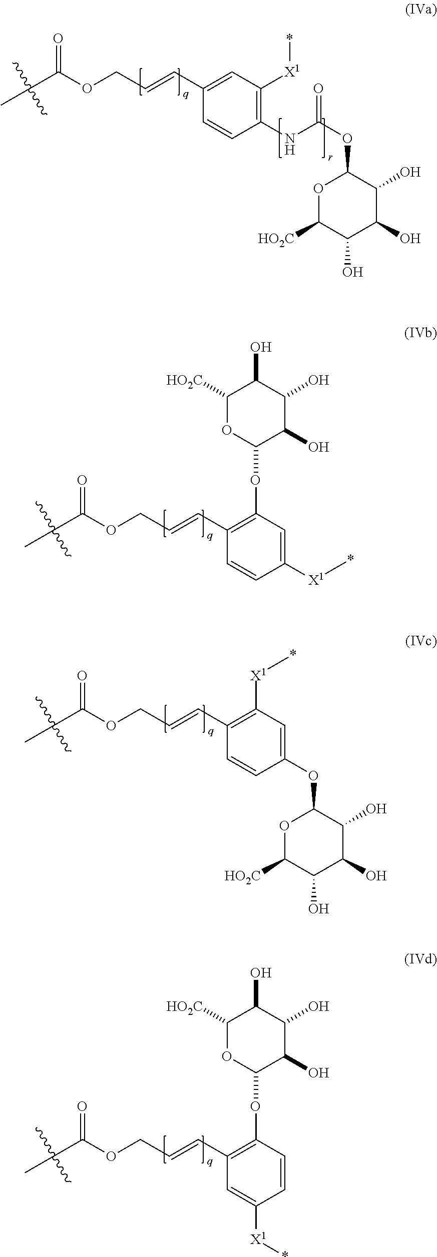

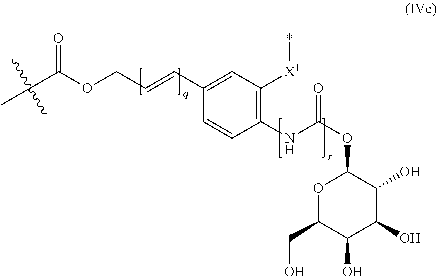

or a salt thereof, wherein: q is 0 or 1; r is 0 or 1; X¹ is CH₂, O or NH; [symbol] represents the point of attachment of the linker to the compound or salt of any one of Formulas (IA), (IB) and (IC); and * represents the point of attachment to the remainder of the linker.

Exemplary embodiments of linkers according to structural formula (IVa) that may be included in the antibody construct benzazepine compound conjugates described herein can include the linkers illustrated below (as illustrated, the linkers include a group suitable for covalently linking the linker to an antibody construct):

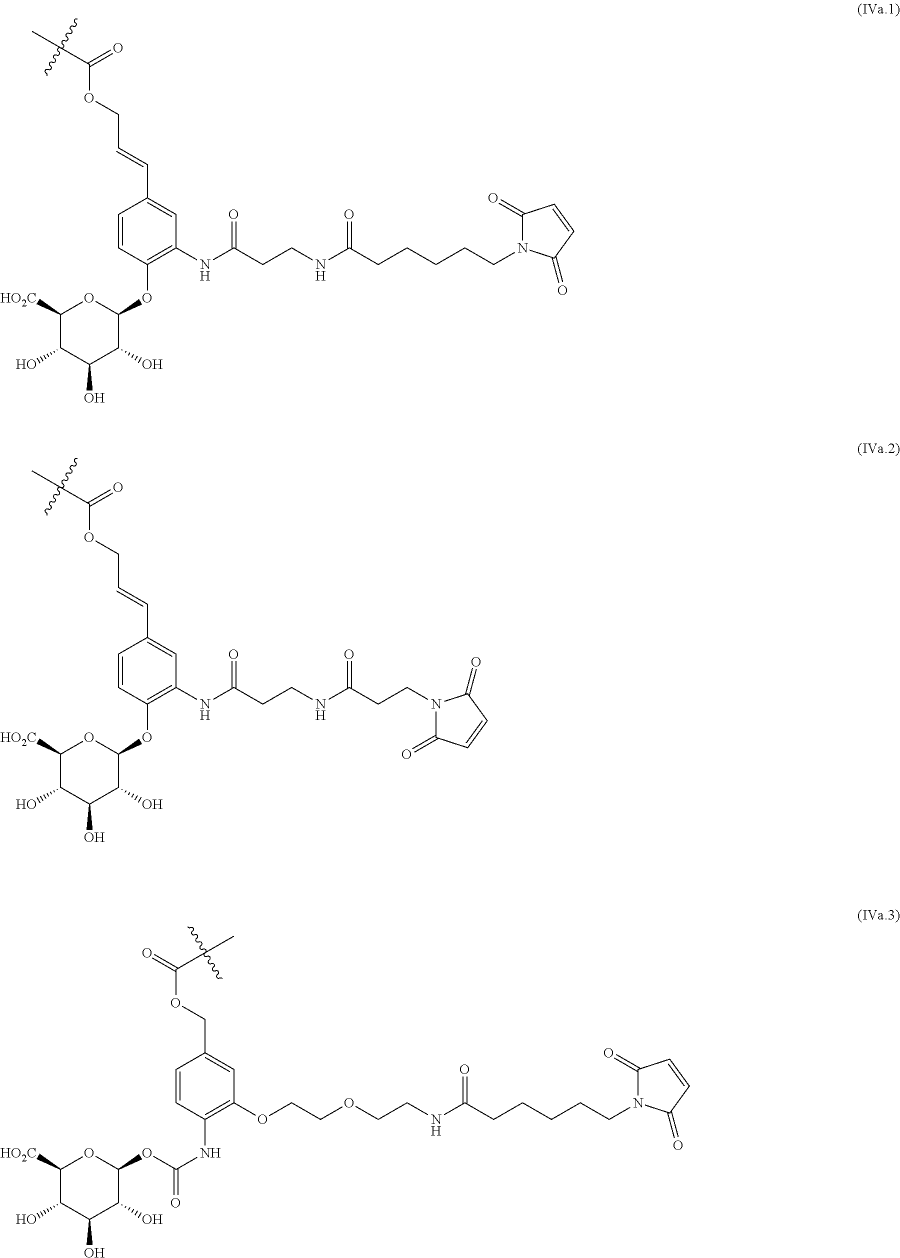 [SMILES: *C(=O)OC/C=C/c1ccc(O[C@@H]2O[C@H](C(=O)O)[C@@H](O)[C@H](O)[C@H]2O)c(NC(=O)CCNC(=O)CCCCCN2C(=O)C=CC2=O)c1 ; *C(=O)OC/C=C/c1ccc(O[C@@H]2O[C@H](C(=O)O)[C@@H](O)[C@H](O)[C@H]2O)c(NC(=O)CCNC(=O)CCN2C(=O)C=CC2=O)c1 ; *C(=O)OCc1ccc(NC(=O)O[C@@H]2O[C@H](C(=O)O)[C@@H](O)[C@H](O)[C@H]2O)c(OCCOCCNC(=O)CCCCCN2C(=O)C=CC2=O)c1]

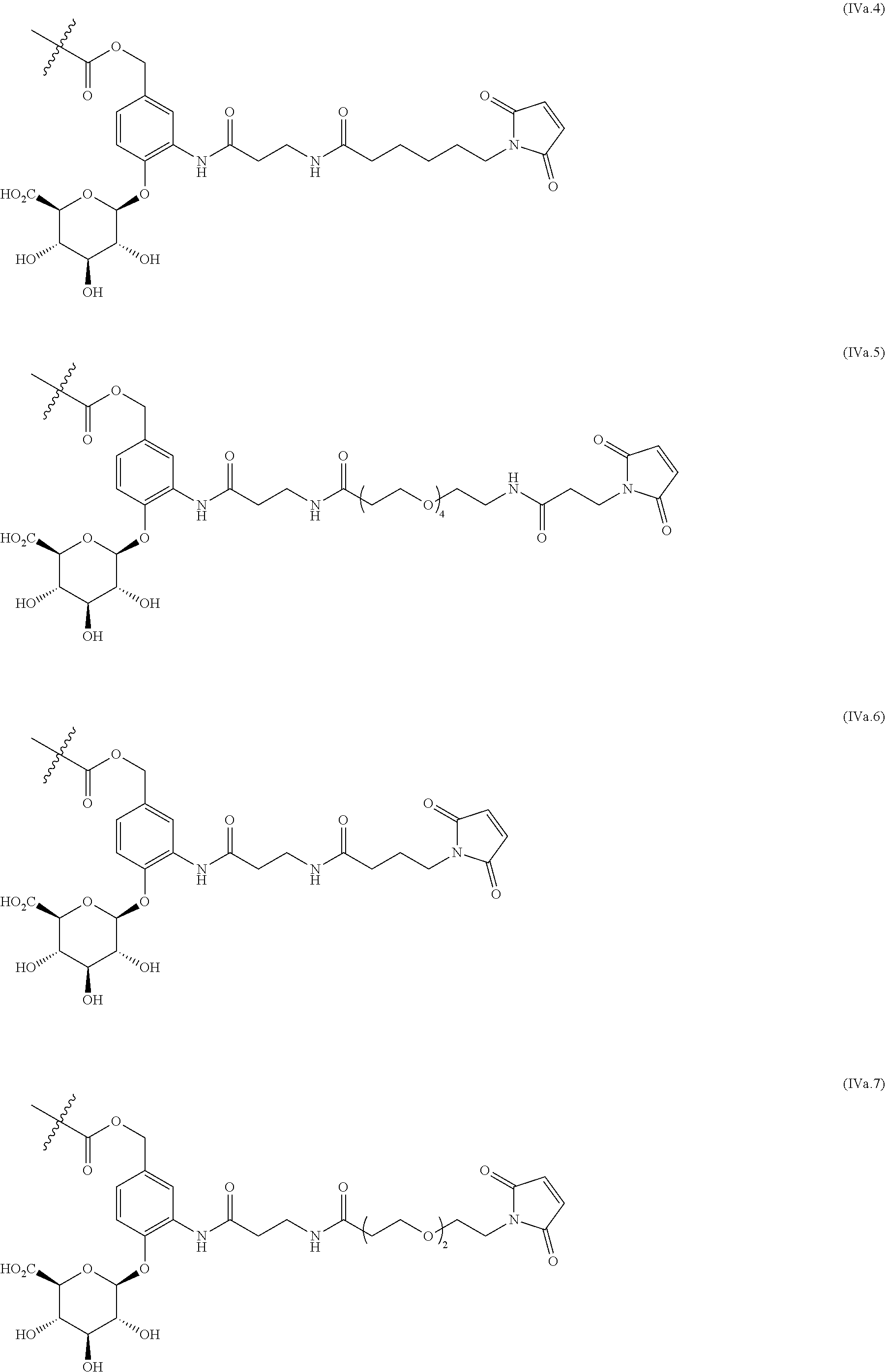

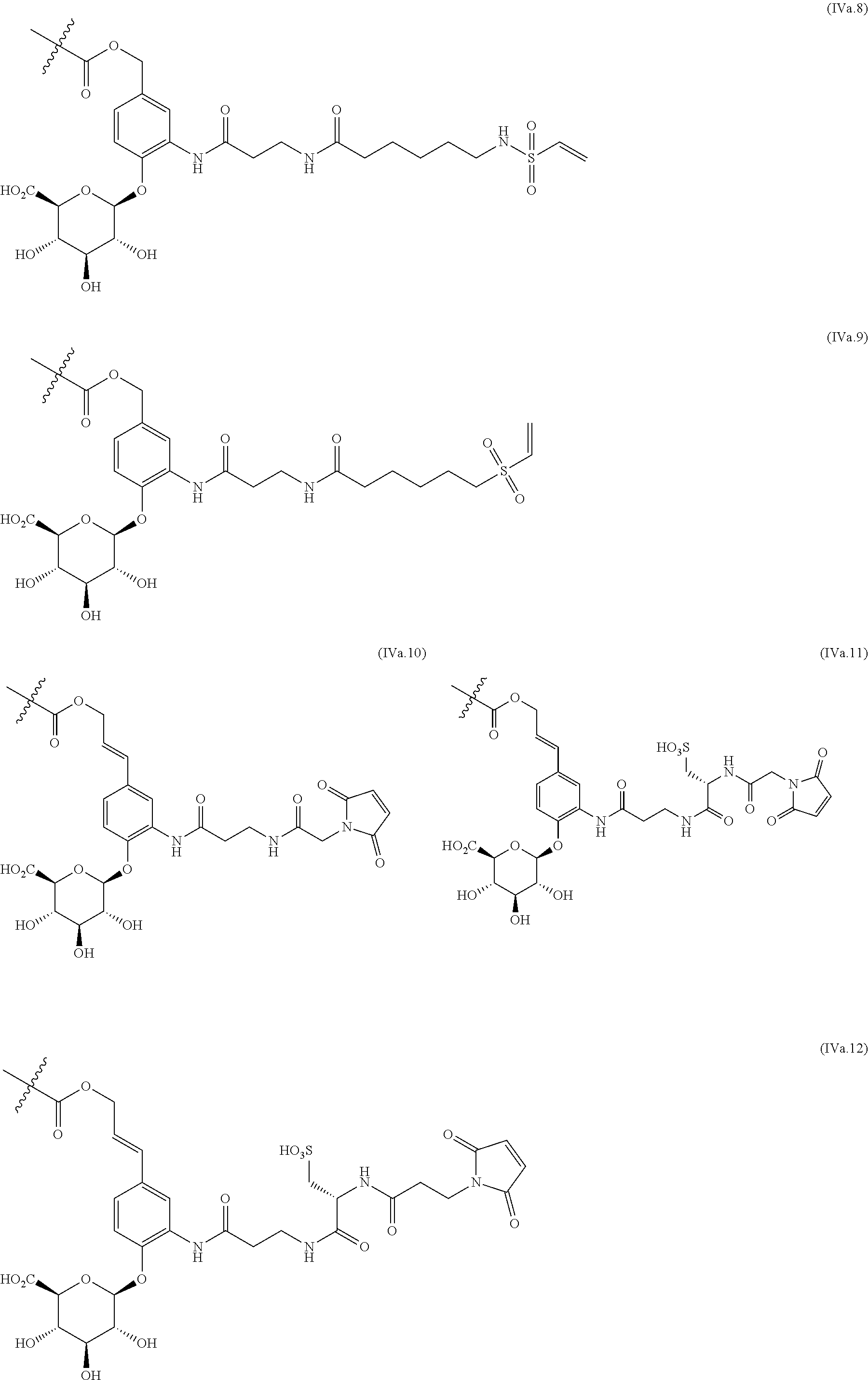

Exemplary embodiments of linkers according to structural formula (IVb) that may be included in the conjugates include the linkers illustrated below (as illustrated, the linkers include a group suitable for covalently linking the linker to an antibody construct):

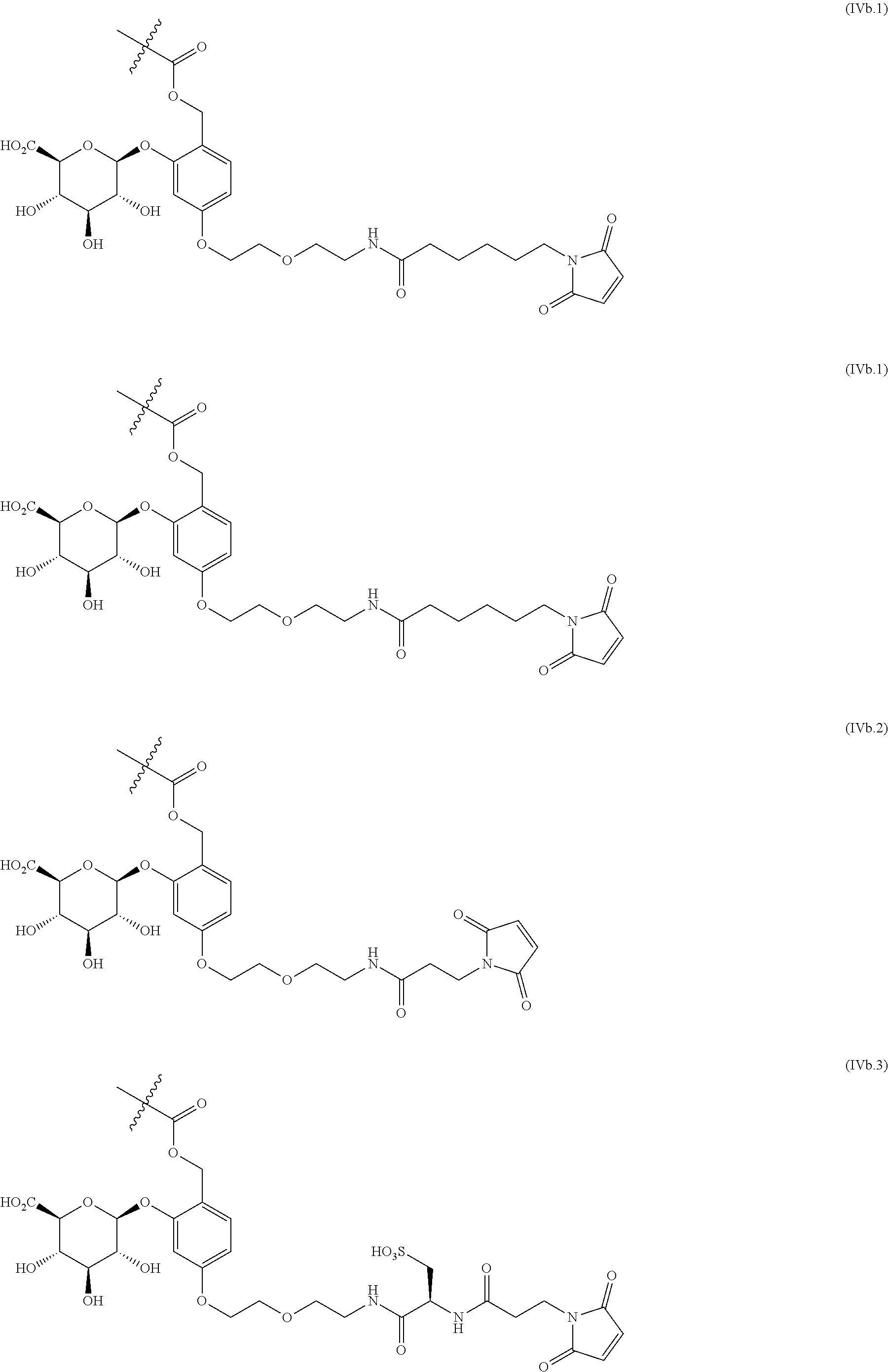 [SMILES: *C(=O)OCc1ccc(OCCOCCNC(=O)CCCCCN2C(=O)C=CC2=O)cc1O[C@@H]1O[C@H](C(=O)O)[C@@H](O)[C@H](O)[C@H]1O ; *C(=O)OCc1ccc(OCCOCCNC(=O)CCCCCN2C(=O)C=CC2=O)cc1O[C@@H]1O[C@H](C(=O)O)[C@@H](O)[C@H](O)[C@H]1O ; *C(=O)OCc1ccc(OCCOCCNC(=O)CCN2C(=O)C=CC2=O)cc1O[C@@H]1O[C@H](C(=O)O)[C@@H](O)[C@H](O)[C@H]1O ; *C(=O)OCc1ccc(OCCOCCNC(=O)[C@@H](CS(=O)(=O)O)NC(=O)CCN2C(=O)C=CC2=O)cc1O[C@@H]1O[C@H](C(=O)O)[C@@H](O)[C@H](O)[C@H]1O]

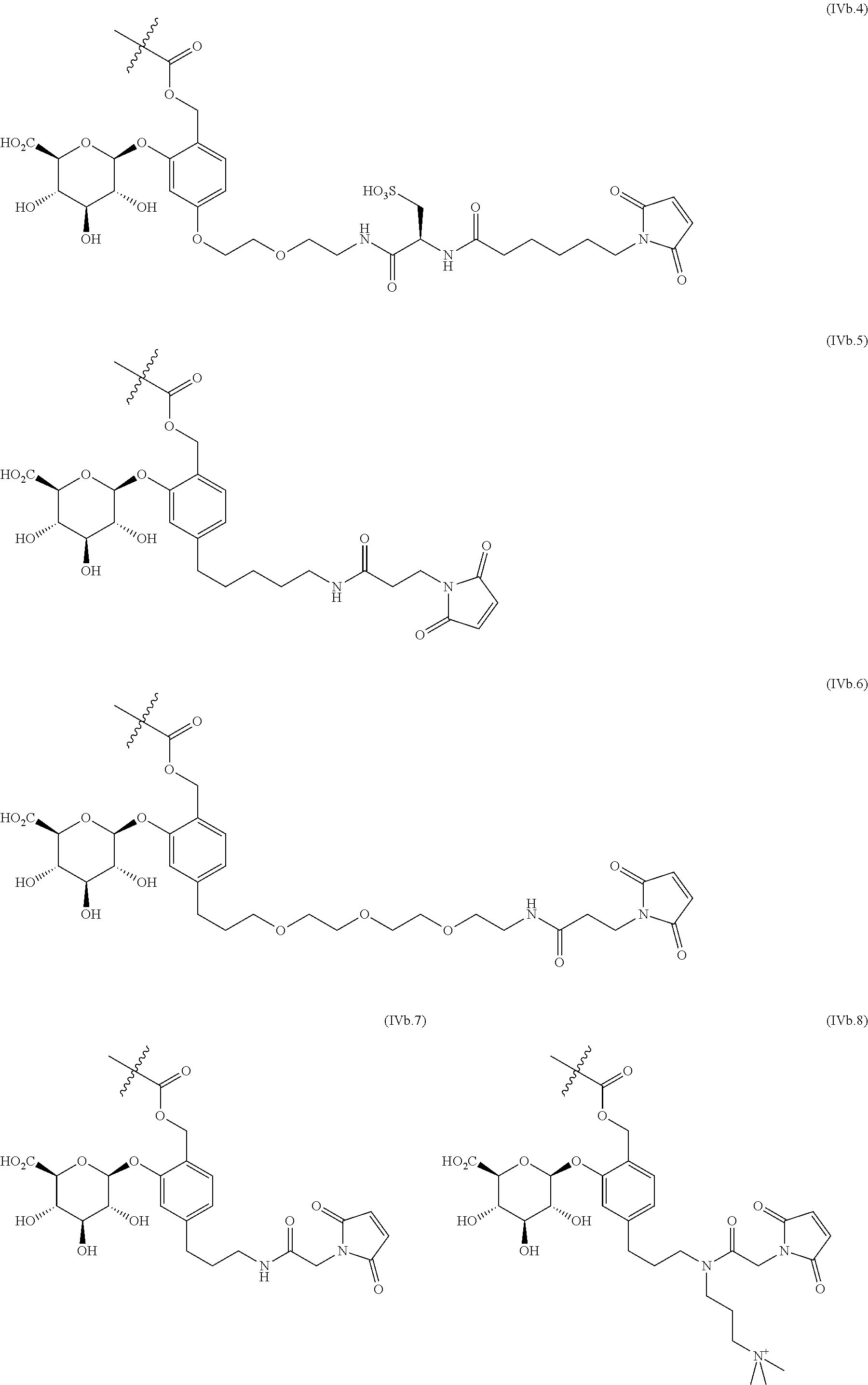

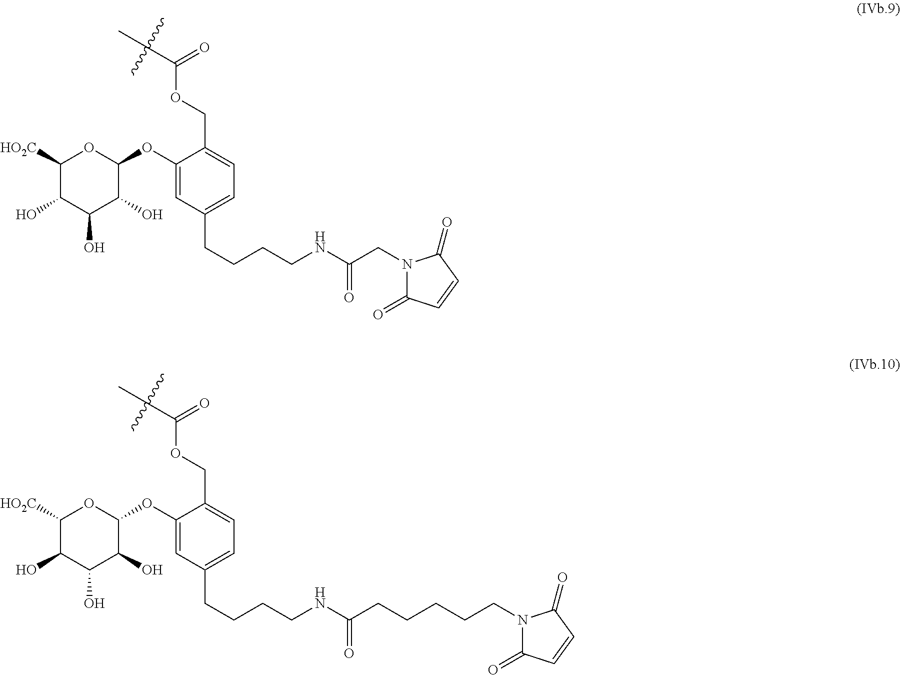 [SMILES: *C(=O)OCc1ccc(CCCCNC(=O)CN2C(=O)C=CC2=O)cc1O[C@@H]1O[C@H](C(=O)O)[C@@H](O)[C@H](O)[C@H]1O ; *C(=O)OCc1ccc(CCCCNC(=O)CCCCCN2C(=O)C=CC2=O)cc1O[C@H]1O[C@@H](C(=O)O)[C@H](O)[C@@H](O)[C@@H]1O]

Exemplary embodiments of linkers according to structural formula (IVc) that may be included in the conjugates include the linkers illustrated below (as illustrated, the linkers include a group suitable for covalently linking the linker to an antibody construct):

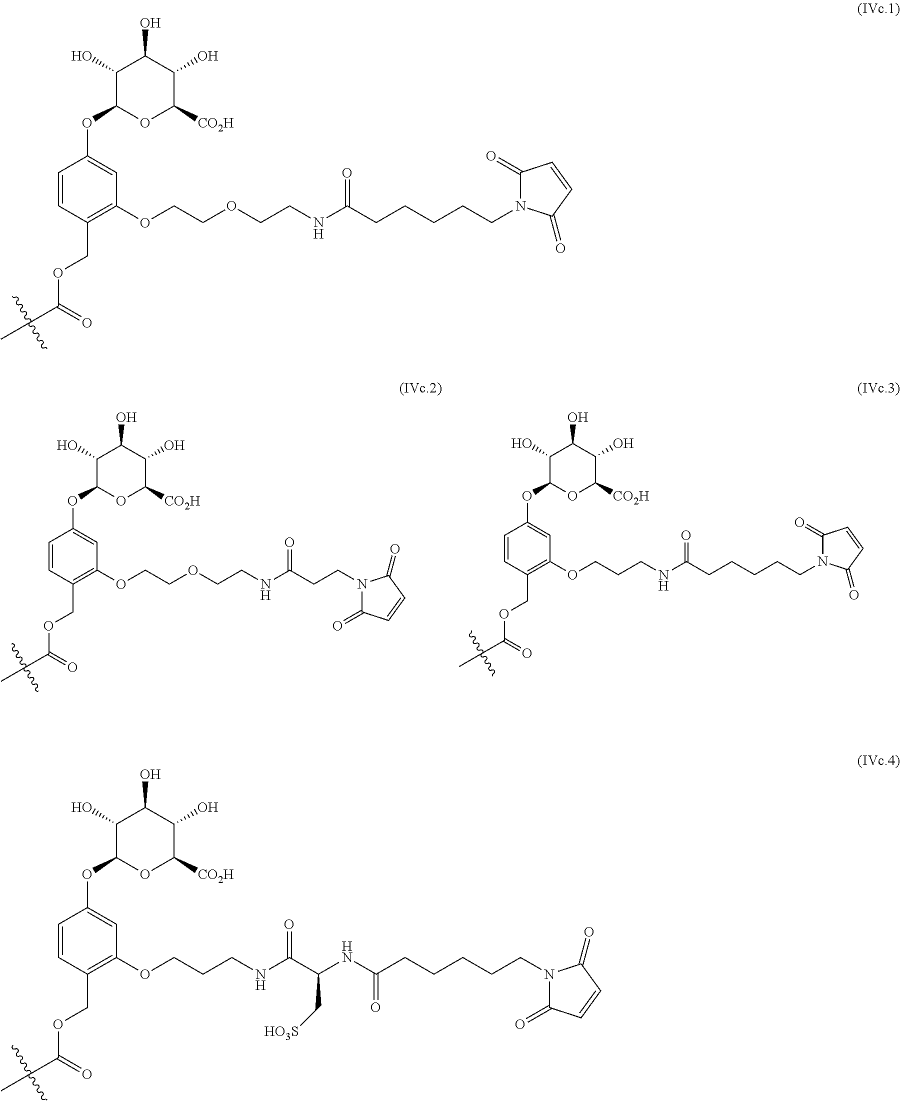 [SMILES: *C(=O)OCc1ccc(O[C@@H]2O[C@H](C(=O)O)[C@@H](O)[C@H](O)[C@H]2O)cc1OCCOCCNC(=O)CCCCCN1C(=O)C=CC1=O ; *C(=O)OCc1ccc(O[C@@H]2O[C@H](C(=O)O)[C@@H](O)[C@H](O)[C@H]2O)cc1OCCOCCNC(=O)CCN1C(=O)C=CC1=O ; *C(=O)OCc1ccc(O[C@@H]2O[C@H](C(=O)O)[C@@H](O)[C@H](O)[C@H]2O)cc1OCCCNC(=O)CCCCCN1C(=O)C=CC1=O ; *C(=O)OCc1ccc(O[C@@H]2O[C@H](C(=O)O)[C@@H](O)[C@H](O)[C@H]2O)cc1OCCCNC(=O)[C@H](CS(=O)(=O)O)NC(=O)CCCCCN1C(=O)C=CC1=O]

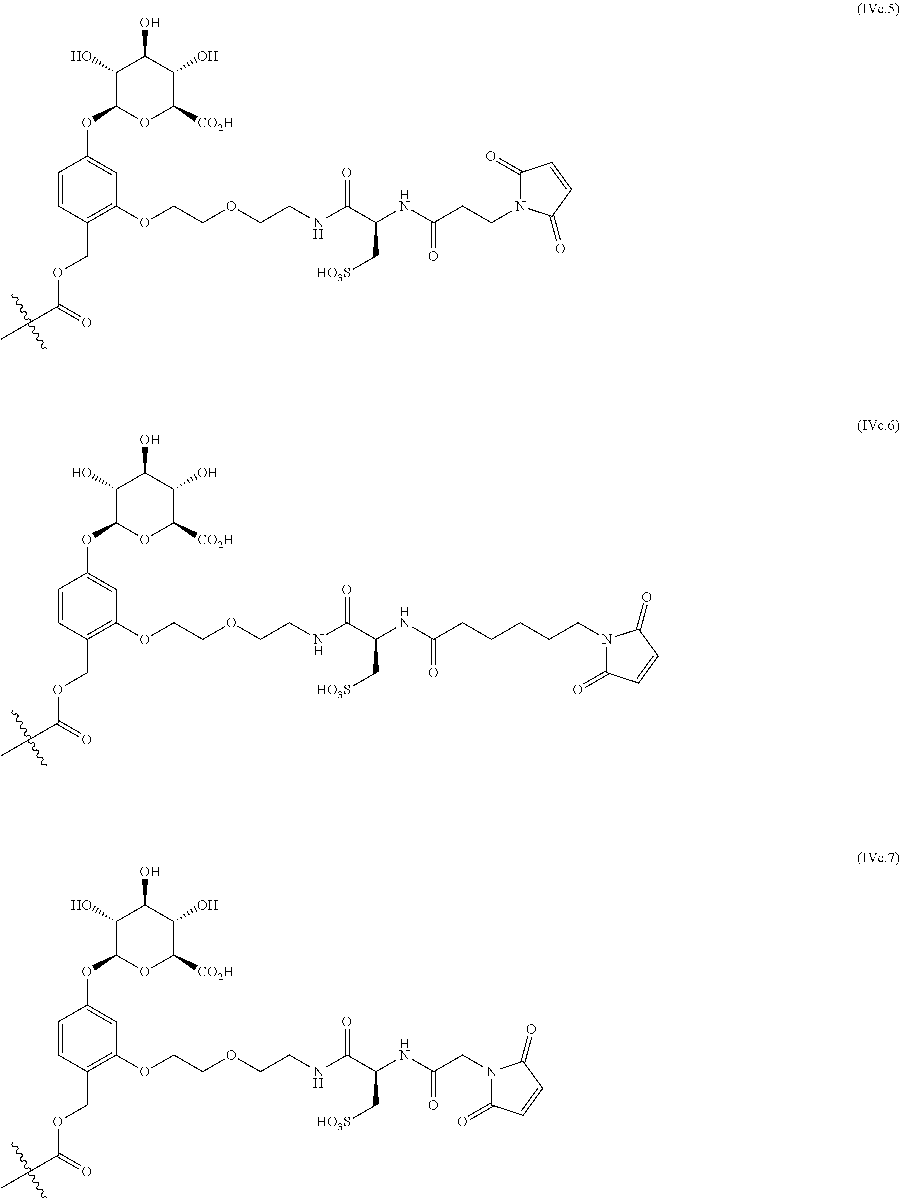 [SMILES: *C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CCN1C(=O)C=CC1=O ; *C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CCCCCN1C(=O)C=CC1=O ; *C(=O)OCc1ccc(O[C@@H]2O[C@H](C(=O)O)[C@@H](O)[C@H](O)[C@H]2O)cc1OCCOCCNC(=O)[C@H](CS(=O)(=O)O)NC(=O)CN1C(=O)C=CC1=O]

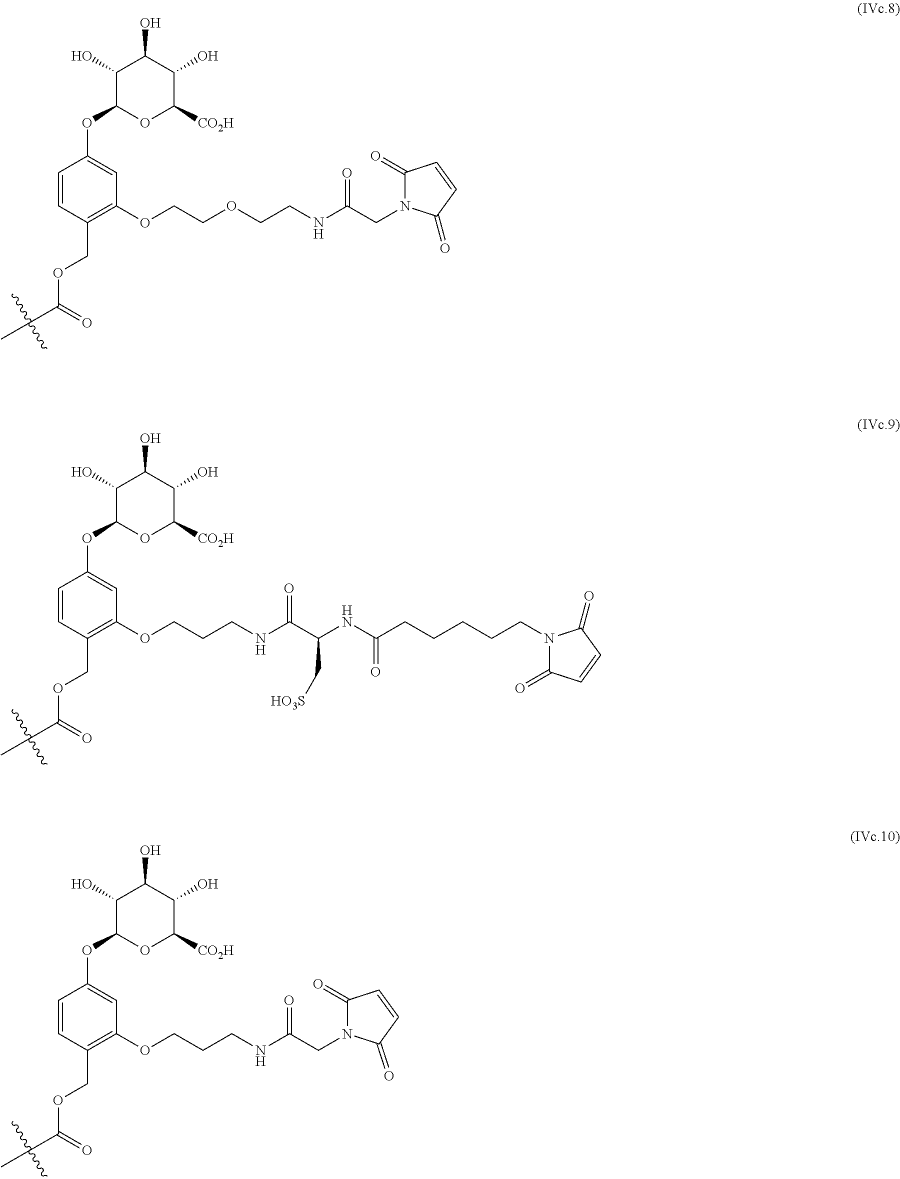 [SMILES: *C(=O)OCc1ccc(O[C@@H]2O[C@H](C(=O)O)[C@@H](O)[C@H](O)[C@H]2O)cc1OCCOCCNC(=O)CN1C(=O)C=CC1=O ; *C(=O)OCc1ccc(O[C@@H]2O[C@H](C(=O)O)[C@@H](O)[C@H](O)[C@H]2O)cc1OCCCNC(=O)[C@H](CS(=O)(=O)O)NC(=O)CCCCCN1C(=O)C=CC1=O ; *C(=O)OCc1ccc(O[C@@H]2O[C@H](C(=O)O)[C@@H](O)[C@H](O)[C@H]2O)cc1OCCCNC(=O)CN1C(=O)C=CC1=O]

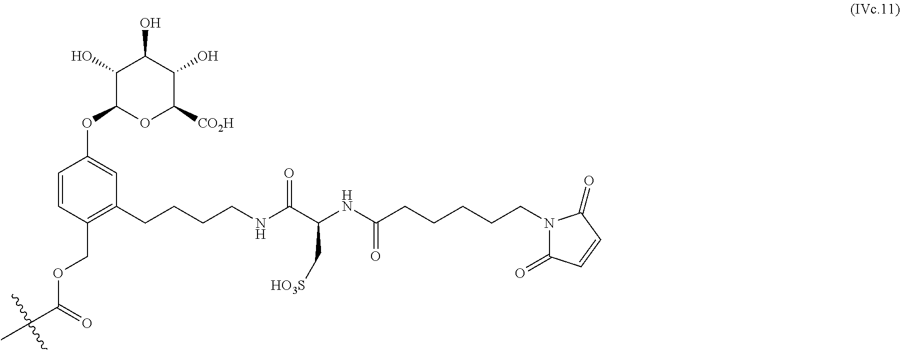 [SMILES: *C(=O)OCc1ccc(O[C@@H]2O[C@H](C(=O)O)[C@@H](O)[C@H](O)[C@H]2O)cc1CCCCNC(=O)[C@H](CS(=O)(=O)O)NC(=O)CCCCCN1C(=O)C=CC1=O]

Exemplary embodiments of linkers according to structural formula (IVd) that may be included in the conjugates include the linkers illustrated below (as illustrated, the linkers include a group suitable for covalently linking the linker to an antibody construct):

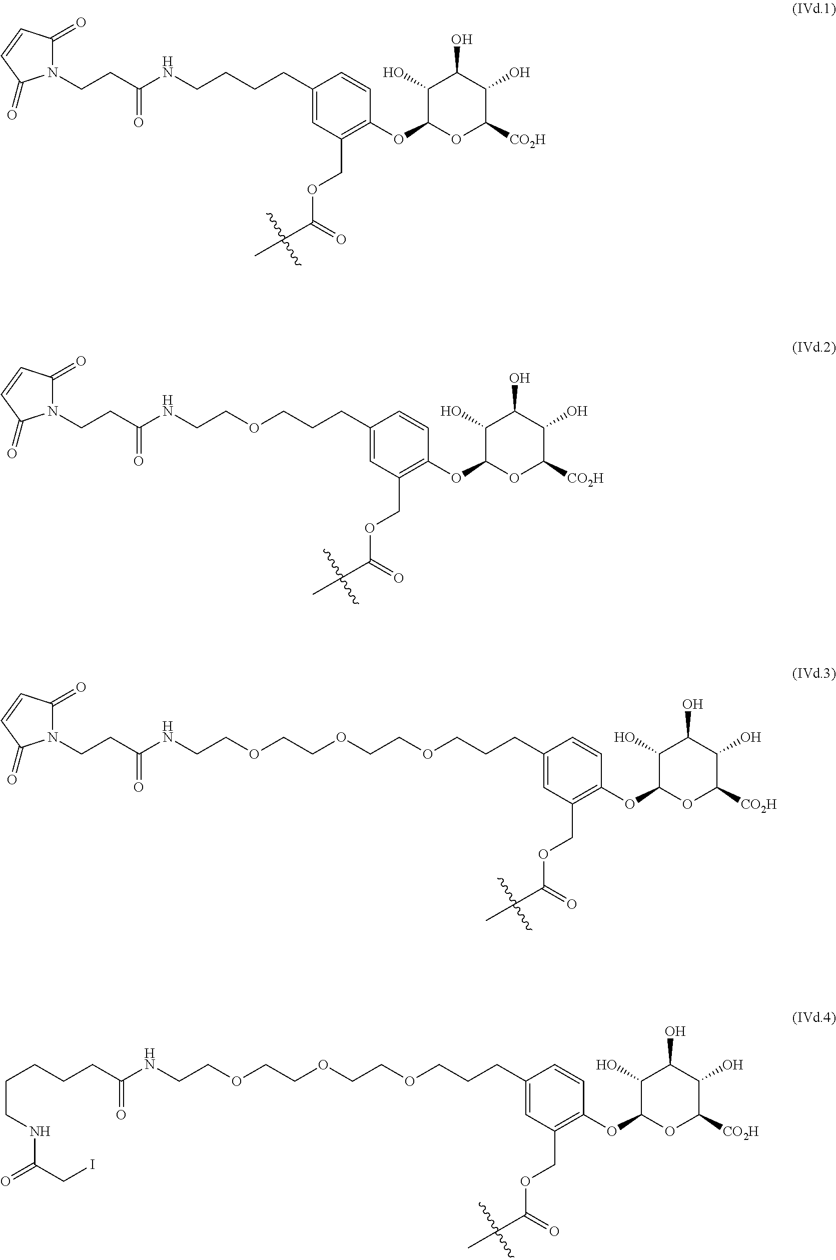 [SMILES: *C(=O)OCc1cc(CCCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCCCCNC(=O)CI)ccc1O[C@@H]1O[C@H](C(=O)O)[C@@H](O)[C@H](O)[C@H]1O]

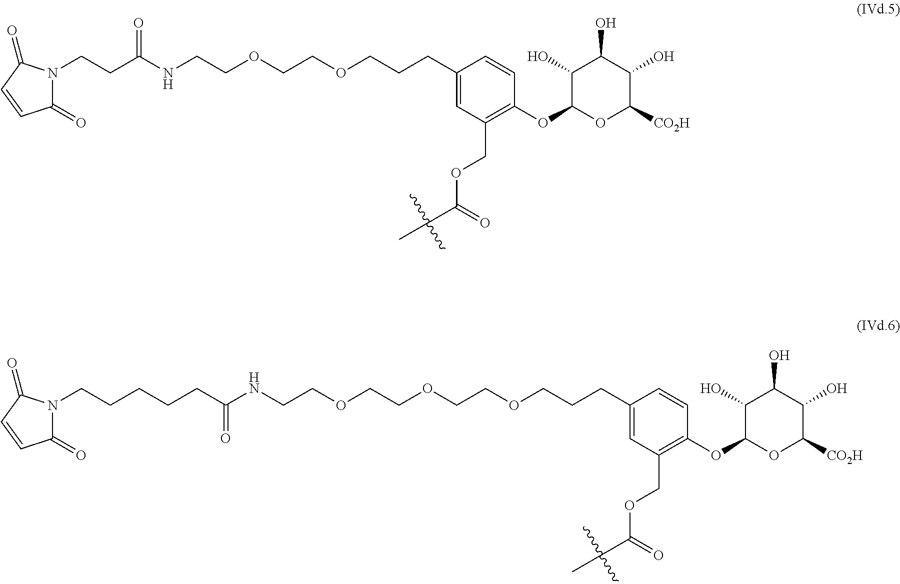 [SMILES: *C(=O)OCc1cc(CCCOCCOCCNC(=O)CCN2C(=O)C=CC2=O)ccc1O[C@@H]1O[C@H](C(=O)O)[C@@H](O)[C@H](O)[C@H]1O ; *C(=O)OCc1cc(CCCOCCOCCOCCNC(=O)CCCCCN2C(=O)C=CC2=O)ccc1O[C@@H]1O[C@H](C(=O)O)[C@@H](O)[C@H](O)[C@H]1O]

Exemplary embodiments of linkers according to structural formula (IVe) that may be included in the conjugates include the linkers illustrated below (as illustrated, the linkers include a group suitable for covalently linking the linker to an antibody construct):

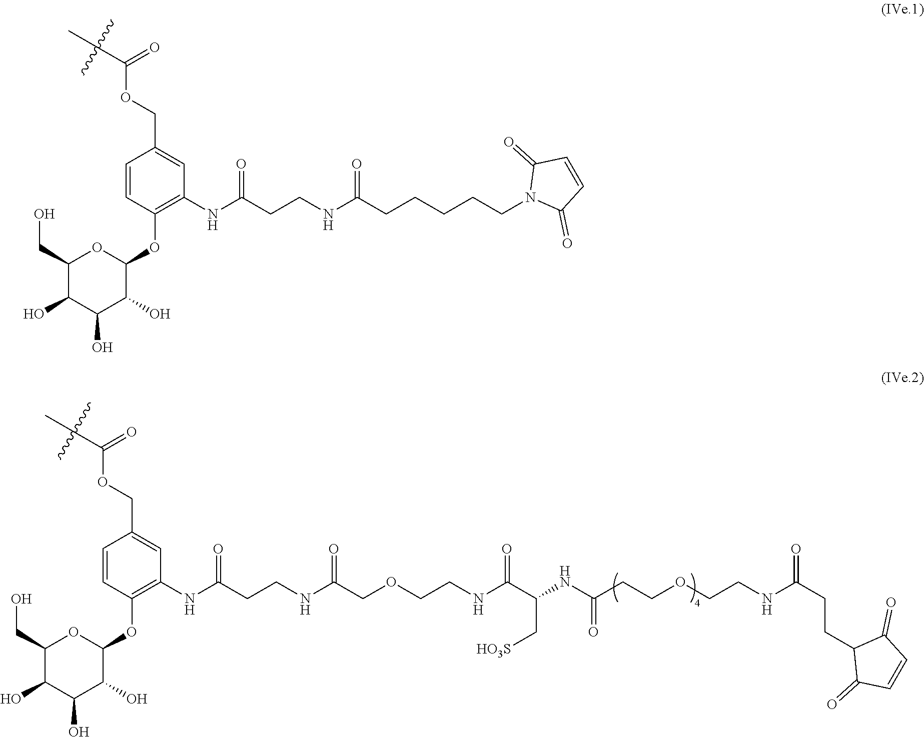

Although cleavable linkers can provide certain advantages, the linkers in the conjugates described herein need not be cleavable. For non-cleavable linkers, benzazepine compound or salt thereof release may not depend on the differential properties between the plasma and some cytoplasmic compartments. The release of the benzazepine compound or salt thereof can occur after internalization of the antibody construct benzazepine compound conjugate via antigen-mediated endocytosis and delivery to lysosomal compartment, where the antibody construct can be degraded to the level of amino acids through intracellular proteolytic degradation. This process can release a benzazepine compound derivative (a metabolite), which is formed by the benzazepine compound or salt thereof, the linker, and the amino acid residue to which the linker was covalently attached. The benzazepine compound derivative from antibody construct benzazepine compound conjugates with non-cleavable linkers can be more hydrophilic and less membrane permeable, which can lead to less bystander effects compared to antibody construct benzazepine compound conjugates with a cleavable linker. Antibody construct benzazepine compound conjugates with non-cleavable linkers can have greater stability in circulation than antibody construct benzazepine compound conjugates with cleavable linkers. Non-cleavable linkers can contain alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units.

The linker can be non-cleavable in vivo, for example, a linker according to the formulations below:

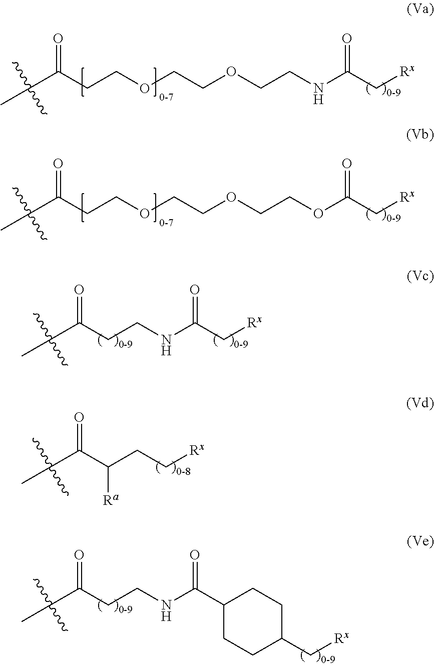

or salts thereof, wherein: R^{a} is selected from hydrogen, alkyl, sulfonate and methyl sulfonate; R^{x} is a moiety including a functional group capable of covalently linking the linker to an antibody construct; and [symbol] represents the point of attachment of the linker to a compound or salt described herein.

Exemplary embodiments of linkers according to structural formula (Va)-(Ve) that may be included in the conjugates include the linkers illustrated below (as illustrated, the linkers include a group suitable for covalently linking the linker to an antibody construct, and [symbol] represents the point of attachment to a compound or salt of any one of Formulas (IA), (IB) and (IC):

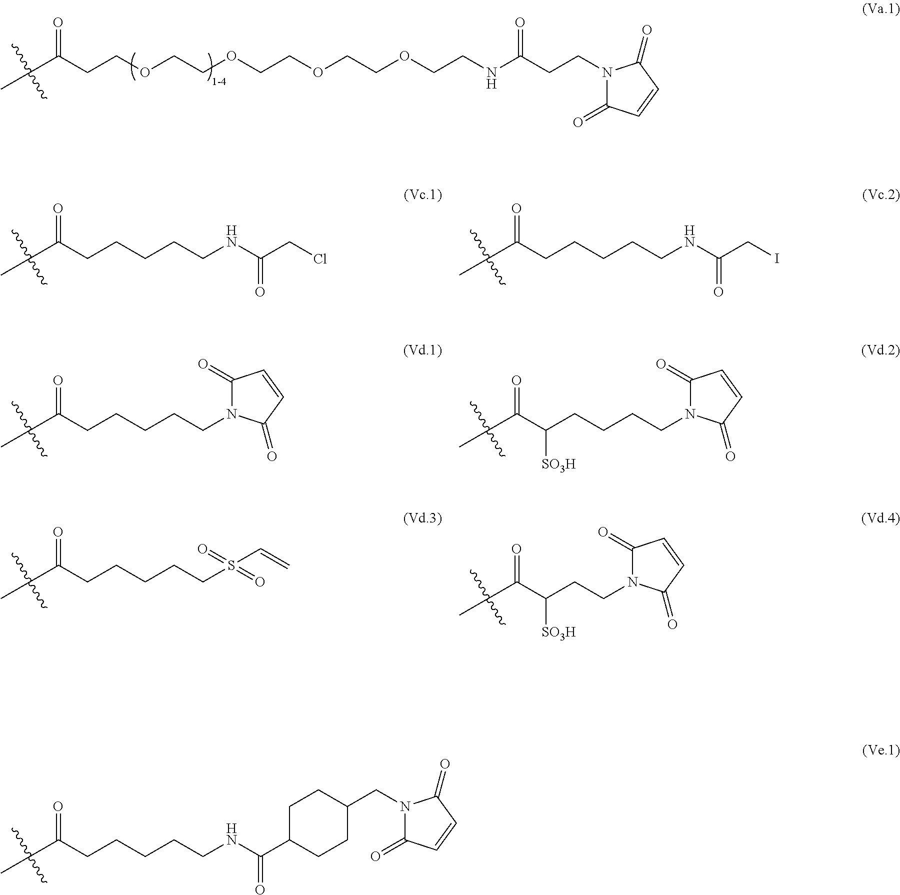

Attachment groups that are used to attach the linkers to an antibody can be electrophilic in nature and include, for example, maleimide groups, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure.

One example of a “self-stabilizing” maleimide group that hydrolyzes spontaneously under antibody conjugation conditions to give a conjugate with improved stability is depicted in the schematic below. Thus, the maleimide attachment group is reacted with a sulfhydryl of an antibody to give an intermediate succinimide ring. The hydrolyzed form of the attachment group is resistant to deconjugation in the presence of plasma proteins.

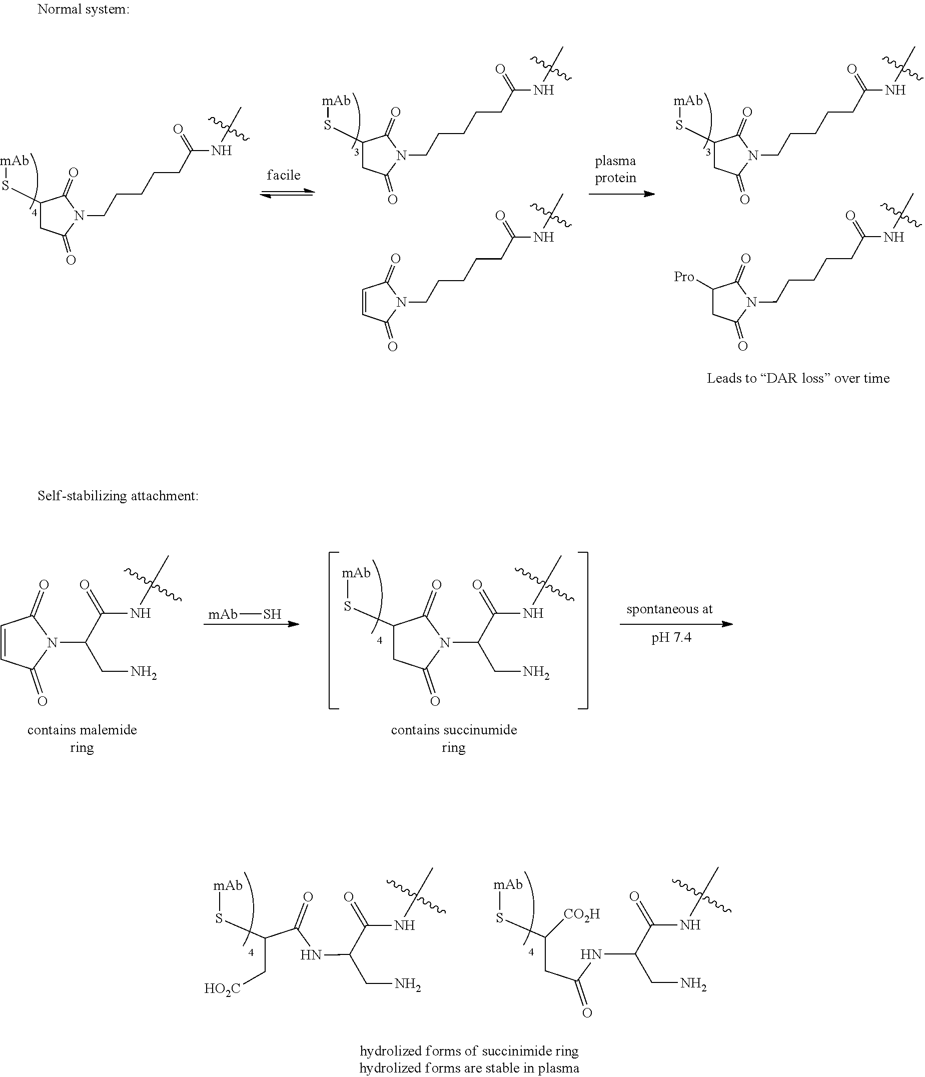

A method for bridging a pair of sulfhydryl groups derived from reduction of a native hinge disulfide bond has been disclosed and is depicted in the schematic below. An advantage of this methodology is the ability to synthesize homogenous DAR4 conjugates by full reduction of IgGs (to give 4 pairs of sulfhydryls) followed by reaction with 4 equivalents of the alkylating agent. Conjugates containing “bridged disulfides” are also claimed to have increased stability.

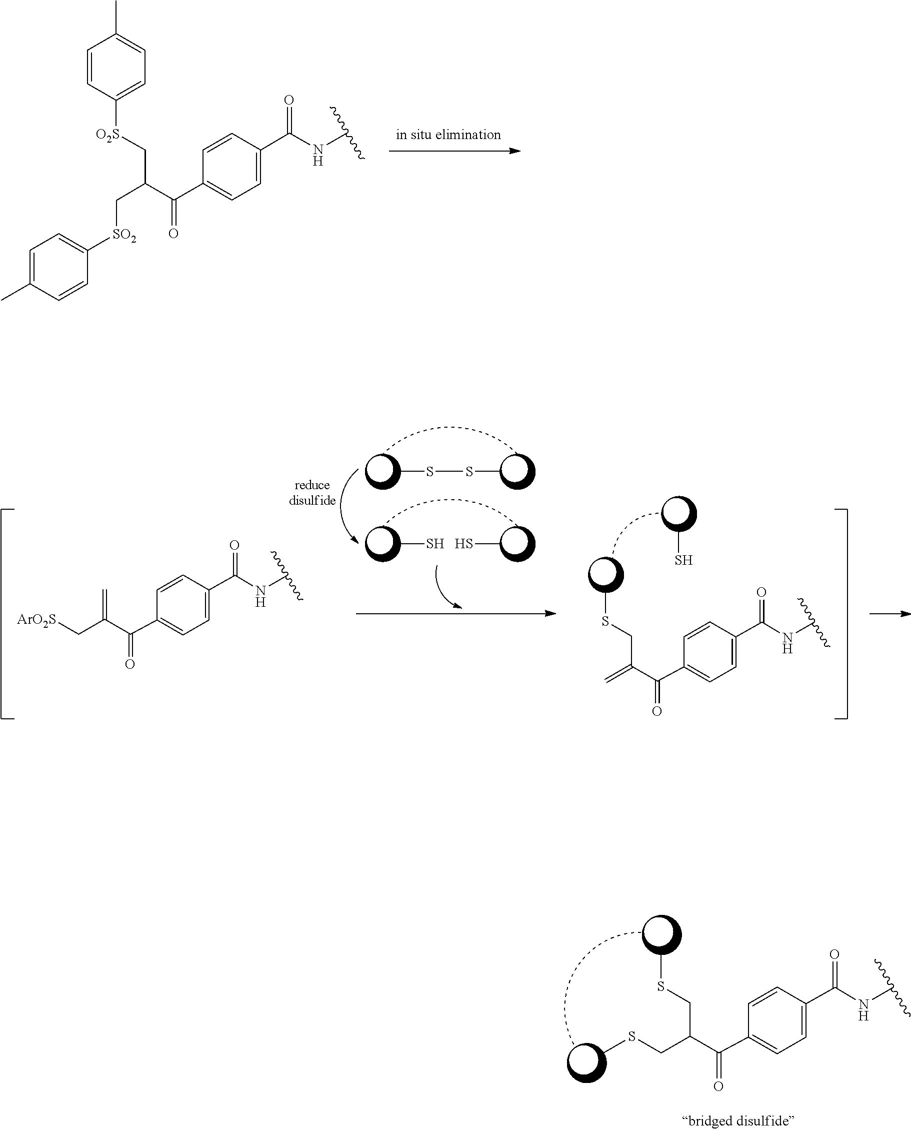

Similarly, as depicted below, a maleimide derivative that is capable of bridging a pair of sulfhydryl groups has been developed.

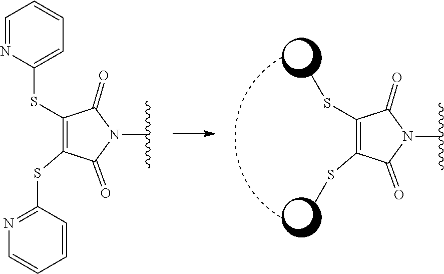 [SMILES: *N1C(=O)C(Sc2ccccn2)=C(Sc2ccccn2)C1=O ; *N1C(=O)C(SC)=C(SC)C1=O]

The attachment moiety can contain the following structural formulas (VIa), (VIb), or (VIc):

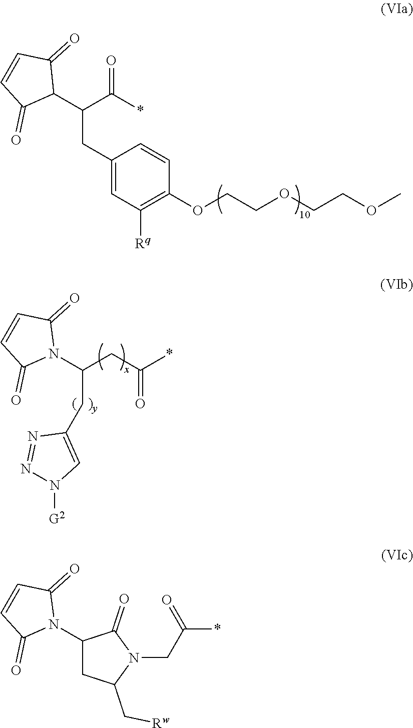

or salts thereof, wherein: R^{q} is H or —O—(CH₂CH₂O)₁₁—CH₃; x is 0 or 1; y is 0 or 1; G² is —CH₂CH₂CH₂SO₃H or —CH₂CH₂O—(CH₂CH₂O)₁₁—CH₃; R^{w} is —O—CH₂CH₂SO₃H or —NH(CO)—CH₂CH₂O—(CH₂CH₂O)₁₂—CH₃; and * represents the point of attachment to the remainder of the linker.

Exemplary embodiments of linkers according to structural formula (VIa) and (VIb) that can be included in the conjugates described herein can include the linkers illustrated below (as illustrated, the linkers can include a group suitable for covalently linking the linker to an antibody construct):

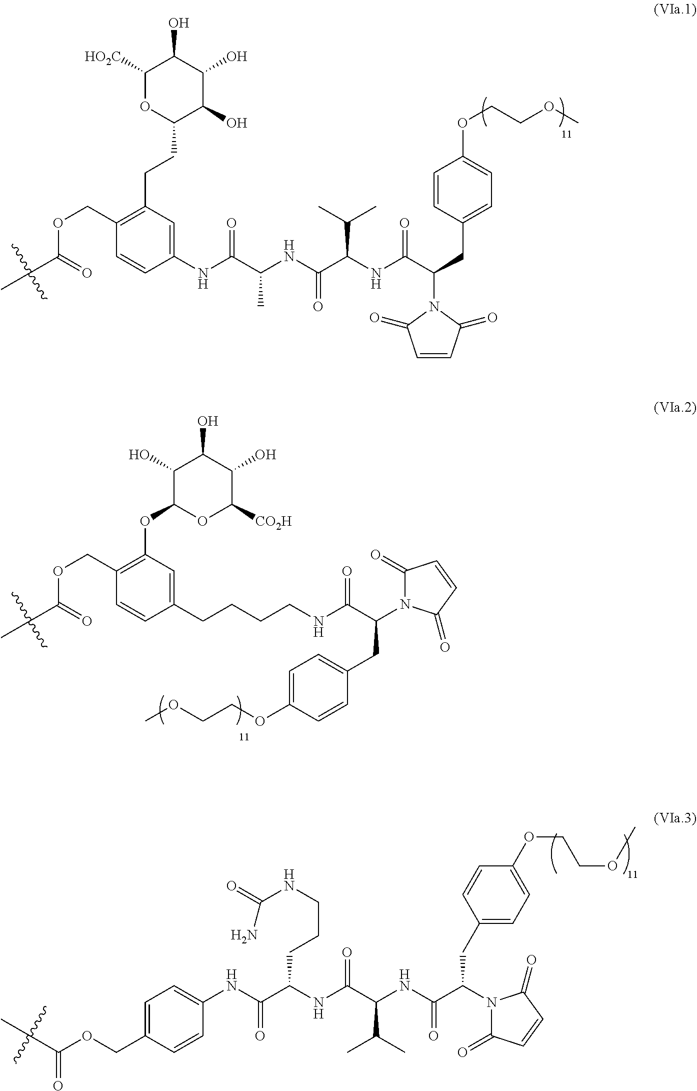

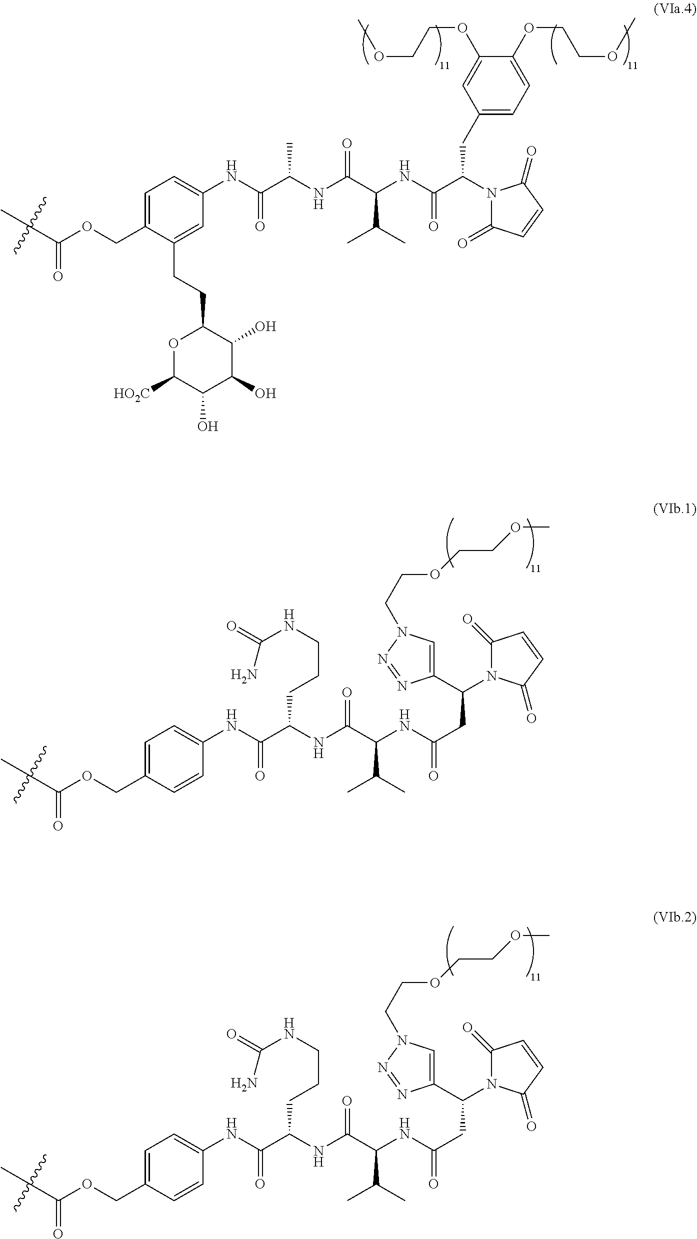

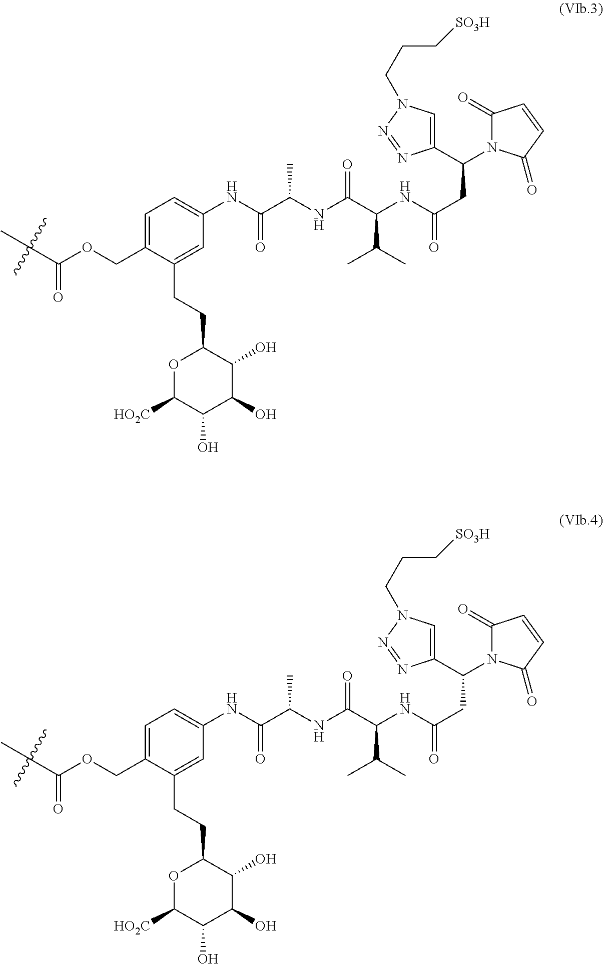 [SMILES: *C(=O)OCc1ccc(NC(=O)[C@H](C)NC(=O)[C@@H](NC(=O)C[C@@H](c2cn(CCCS(=O)(=O)O)nn2)N2C(=O)C=CC2=O)C(C)C)cc1CC[C@@H]1O[C@H](C(=O)O)[C@@H](O)[C@H](O)[C@H]1O ; *C(=O)OCc1ccc(NC(=O)[C@H](C)NC(=O)[C@@H](NC(=O)C[C@H](c2cn(CCCS(=O)(=O)O)nn2)N2C(=O)C=CC2=O)C(C)C)cc1CC[C@@H]1O[C@H](C(=O)O)[C@@H](O)[C@H](O)[C@H]1O]

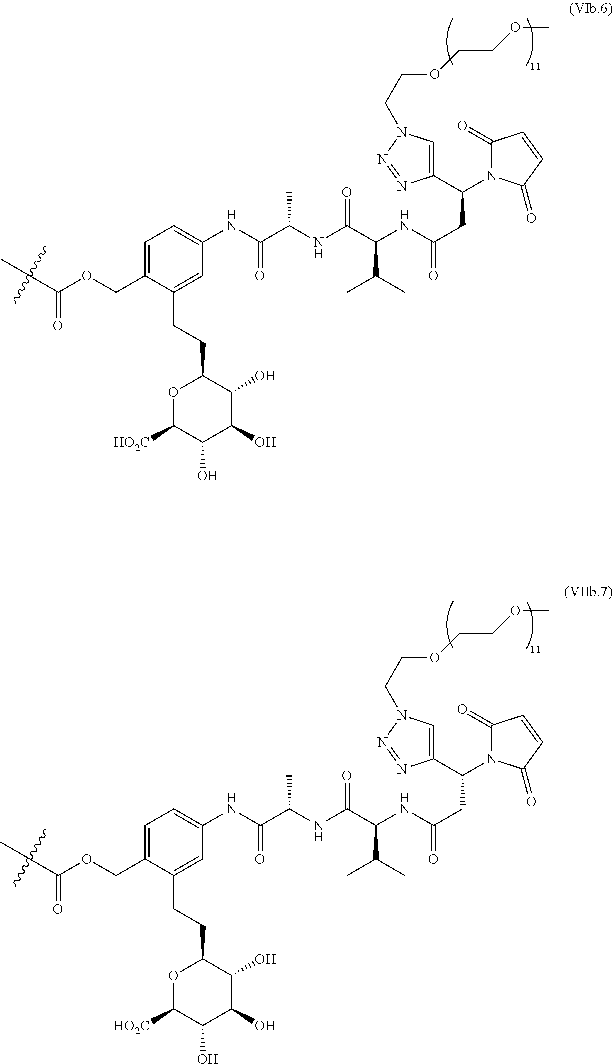

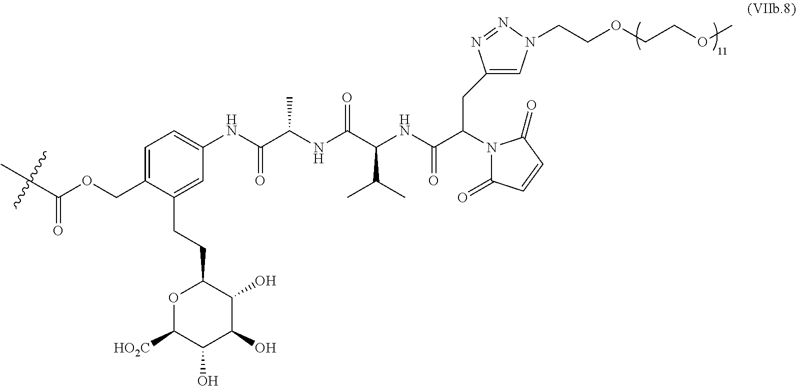

Exemplary embodiments of linkers according to structural formula (VIc) that can be included in the antibody construct benzazepine compound conjugates described herein can include the linkers illustrated below (as illustrated, the linkers can include a group suitable for covalently linking the linker to an antibody construct):

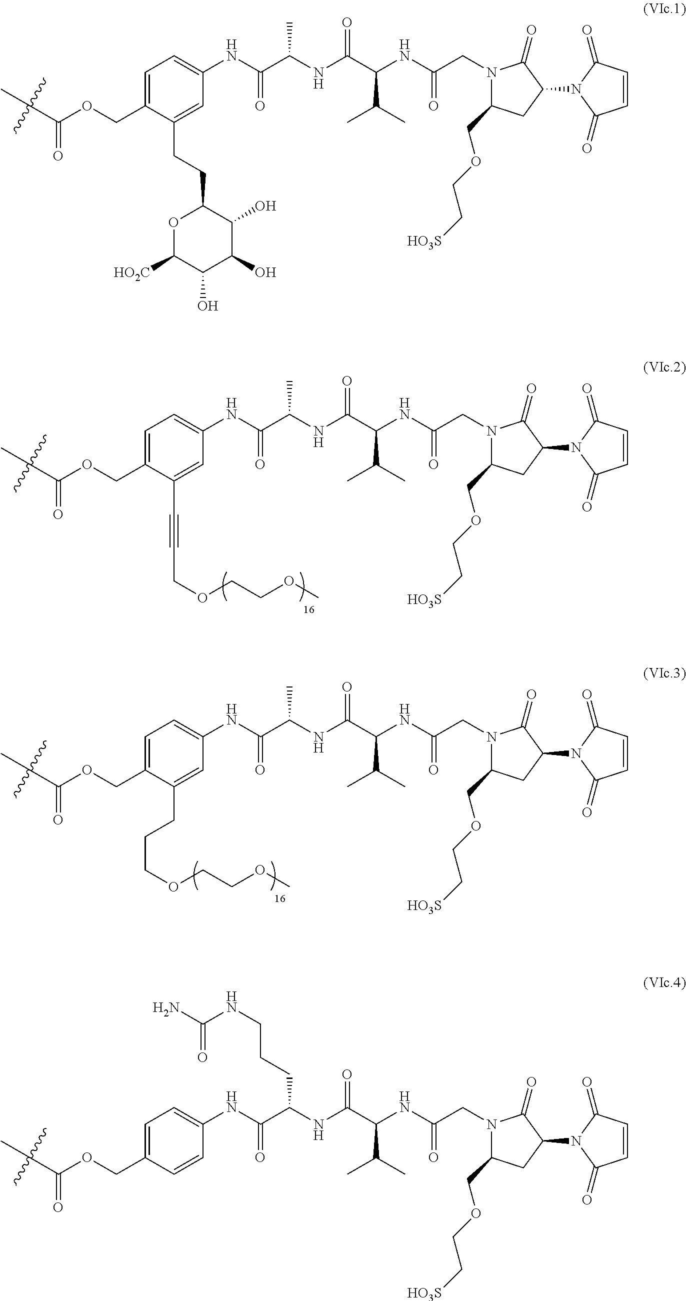

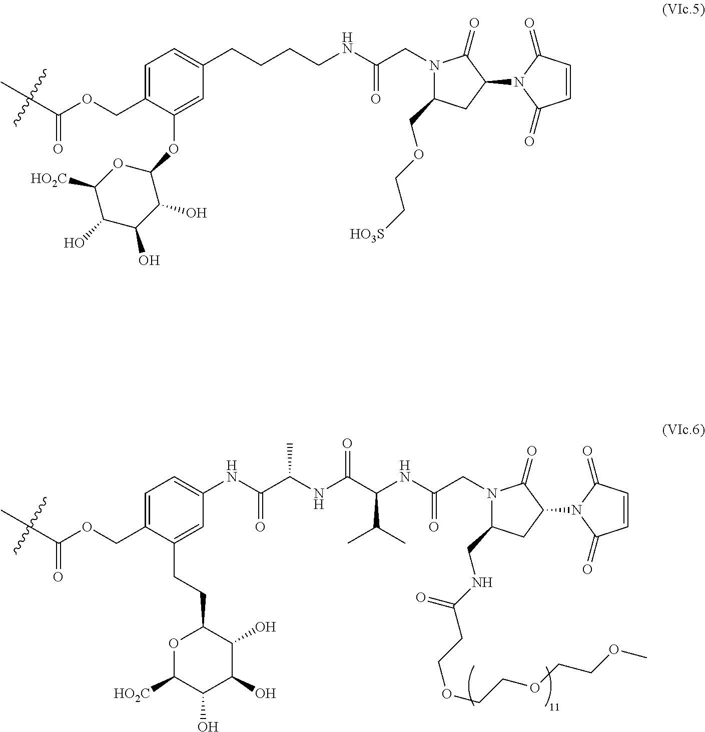

As is known by skilled artisans, the linker selected for a particular conjugate may be influenced by a variety of factors, including but not limited to, the site of attachment to the antibody construct (e.g., lys, cys, gln, or other amino acid residue(s)), structural constraints of the drug pharmacophore and the lipophilicity of the drug. The specific linker selected for a conjugate should seek to balance these different factors for the specific antibody/drug combination.

For example, ADCs have been observed to effect killing of bystander antigen-negative cells present in the vicinity of the antigen-positive tumor cells. The mechanism of bystander cell killing by cytotoxic ADCs has indicated that metabolic products formed during intracellular processing of the conjugates may play a role. Neutral cytotoxic metabolites generated by metabolism of the ADCs in antigen-positive cells appear to play a role in bystander cell killing while charged metabolites may be prevented from diffusing across the membrane into the medium and therefore cannot affect bystander killing. In certain embodiments, the linker is selected to attenuate the bystander effect caused by cellular metabolites of the conjugate. In certain embodiments, the linker is selected to increase the bystander effect.

The properties of the linker may also impact aggregation of the conjugate under conditions of use and/or storage. Typically, ADCs reported in the literature contain no more than 3-4 drug molecules per antibody molecule. Attempts to obtain higher drug-to-antibody ratios (“DAR”) often failed, particularly if both the drug and the linker were hydrophobic, due to aggregation of the ADC. In many instances, DARs higher than 3-4 could be beneficial as a means of increasing potency. In instances where the benzazepine compund is hydrophobic in nature, it may be desirable to select linkers that are relatively hydrophilic as a means of reducing conjugate aggregation, especially in instances where DARs greater than 3-4 are desired. Thus, in certain embodiments, the linker incorporates chemical moieties that reduce aggregation of the conjugate during storage and/or use. A linker may incorporate polar or hydrophilic groups such as charged groups or groups that become charged under physiological pH to reduce the aggregation of the conjugates. For example, a linker may incorporate charged groups such as salts or groups that deprotonate, e.g., carboxylates, or protonate, e.g., amines, at physiological pH.

In particular embodiments, the aggregation of the conjugates during storage or use is less than about 40% as determined by size-exclusion chromatography (SEC). In particular embodiments, the aggregation of the conjugates during storage or use is less than 35%, such as less than about 30%, such as less than about 25%, such as less than about 20%, such as less than about 15%, such as less than about 10%, such as less than about 5%, such as less than about 4%, or even less, as determined by size-exclusion chromatography (SEC).

### Pharmaceutical Formulations

In some aspects, the present disclosure provides a pharmaceutical composition, comprising a conjugate described herein, and a pharmaceutically acceptable excipient. In some embodiments, the average Drug-to-Antibody Ratio (DAR) may be from 1 to 8.

The compounds and conjugates can be considered useful as pharmaceutical compositions for administration to a subject in need thereof. Pharmaceutical compositions can comprise at least a benzazepine compound or salt thereof described herein or a conjugate thereof and one or more pharmaceutically acceptable carriers, diluents, excipients, stabilizers, dispersing agents, suspending agents, and/or thickening agents. A composition can comprise a conjugate having an antibody construct and a benzazepine compound or salt thereof. A composition can comprise a conjugate having an antibody construct, at least one linker and at least one benzazepine compound or salt thereof. A composition can comprise a conjugate having an antibody construct, a target binding domain, at least one linker and at least one benzazepine compound or salt thereof. A composition can comprise any conjugate described herein. In some embodiments, the antibody construct is an anti-HER2, anti-TROP2 MUC16, anti-Liv1 or anti-PD-L1 antibody. In some embodiments, the antibody construct is an anti-HER2, anti-TROP2 or MUC16 antibody. A conjugate can comprise an anti-HER2 antibody and a benzazepine compound or salt thereof. A conjugate can comprise an anti-TROP2 antibody and a benzazepine compound or salt thereof. A conjugate can comprise an anti-MUC16 antibody and a benzazepine compound or salt thereof. A pharmaceutical composition can further comprise buffers, antibiotics, steroids, carbohydrates, drugs (e.g., chemotherapy drugs), radiation, polypeptides, chelators, adjuvants and/or preservatives.

Pharmaceutical compositions may be formulated using one or more physiologically-acceptable carriers comprising excipients and auxiliaries. A formulation may be modified depending upon the route of administration chosen. Pharmaceutical compositions comprising a compound or conjugate may be manufactured, for example, by lyophilizing the conjugate, mixing, dissolving, emulsifying, encapsulating or entrapping the conjugate. The pharmaceutical compositions may also include the benzazepine compounds or salts thereof described herein or conjugates thereof in a free-base form or pharmaceutically-acceptable salt form.

Methods for formulation of the conjugates described herein may include formulating any of the conjugates described herein with one or more inert, pharmaceutically-acceptable excipients or carriers to form a solid, semi-solid, or liquid composition. Solid compositions may include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compositions described herein may be lyophilized or in powder form for re-constitution with a suitable vehicle, e.g., sterile pyrogen-free water, before use

Pharmaceutical compositions of the conjugates described herein may comprise at least an active ingredient. The active ingredients may be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions often further may comprise more than one active compound as necessary for the particular indication being treated. The active compounds may have complementary activities that do not adversely affect each other. For example, the composition may comprise a chemotherapeutic agent, cytotoxic agent, cytokine, growth-inhibitory agent, anti-hormonal agent, anti-angiogenic agent, and/or cardioprotectant. Such molecules may be present in combination in amounts that are effective for the purpose intended.

The compositions, conjugates and formulations may be sterilized. Sterilization may be accomplished by filtration through sterile filtration.

The compositions, compounds and conjugates described herein may be formulated as pharmaceutical compositions for administration as an injection such as an infusion, an intravenous injection or as a subcutaneous injection. Non-limiting examples of formulations for injection may include a sterile suspension, solution or emulsion in oily or aqueous vehicles. Suitable oily vehicles may include, but are not limited to, lipophilic solvents or vehicles such as fatty oils or synthetic fatty acid esters, or liposomes. Aqueous injection suspensions may contain substances which increase the viscosity of the suspension. The suspension may also contain suitable stabilizers. Injections may be formulated for bolus injection or continuous infusion. Alternatively, the compositions, compounds or conjugates described herein may be lyophilized or in powder form for reconstitution with a suitable vehicle, e.g., sterile pyrogen-free water, before use.

For parenteral administration, the conjugates may be formulated in a unit dosage injectable form (e.g., use letter solution, suspension, emulsion) in association with a pharmaceutically acceptable parenteral vehicle. Such vehicles may be inherently non-toxic and non-therapeutic. Vehicles may be water, saline, Ringer's solution, dextrose solution, and 5% human serum albumin. Non-aqueous vehicles such as fixed oils and ethyl oleate may also be used. Liposomes can be used as carriers. The vehicle may contain minor amounts of additives such as substances that enhance isotonicity and chemical stability (e.g., buffers and preservatives).

Sustained-release preparations also may be prepared. Examples of sustained-release preparations can include semipermeable matrices of solid hydrophobic polymers that may contain the conjugate and these matrices can be in the form of shaped articles (e.g., films or microcapsules). Examples of sustained-release matrices may include polyesters, hydrogels (e.g., poly(2-hydroxyethyl-methacrylate), or poly (vinyl alcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations of the compounds or conjugates described herein may be prepared for storage by mixing a conjugate with a pharmaceutically acceptable carrier, excipient, and/or a stabilizer. This formulation may be a lyophilized formulation or an aqueous solution. Acceptable carriers, excipients, and/or stabilizers may be nontoxic to recipients at the dosages and concentrations used. Acceptable carriers, excipients, and/or stabilizers may include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

### Therapeutic Applications

The compositions, compounds, conjugates and methods of the present disclosure may be useful for a plurality of different subjects including, but are not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof. In some embodiments the subject is human.

The compositions, conjugates, compounds and methods described herein may be useful as a therapeutic, for example, a treatment that may be administered to a subject in need thereof, such as a human subject. A therapeutic effect of the present disclosure may be obtained in a subject by reduction, suppression, remission, or eradication of a disease state, including, but not limited to, a symptom thereof. A therapeutic effect in a subject having a disease or condition, or pre-disposed to have or is beginning to have the disease or condition, may be obtained by a reduction, a suppression, a prevention, a remission, or an eradication of the condition or disease, or pre-condition or pre-disease state. A therapeutic effect in a subject can also be obtained by preventing relapse or reoccurance of the disease or condition.

In practicing the methods described herein, therapeutically-effective amounts of the compositions, conjugates or compounds may be administered to a subject in need thereof, often for treating and/or preventing a condition or progression thereof. A pharmaceutical composition may affect the physiology of the subject, such as the immune system, inflammatory response, or other physiologic affect. A therapeutically-effective amount may vary widely depending on the severity of the disease, the age and relative health of the subject, the potency of the compounds or conjugates used, and other factors.

In some aspects, the present disclosure provides a method for the treatment of cancer, comprising administering an effective amount of the compound or salt described herein to a subject in need thereof. In some aspects, the present disclosure provides a method for the treatment of cancer, comprising administering an effective amount of the conjugate described herein or the pharmaceutical composition described herein to a subject in need thereof.

In some aspects, the present disclosure provides a method of killing tumor cells in vivo, comprising contacting a tumor cell population with the conjugate described herein or the pharmaceutical composition described herein.

In some aspects, the present disclosure provides a method for treatment, comprising administering to a subject the conjugate described herein or the pharmaceutical composition described herein. In some aspects, the present disclosure provides a method for the treatment of cancer, comprising administering to a subject in need thereof the conjugate described herein or the pharmaceutical composition described herein.

In some embodiments, the antigen binding domain of the antibody construct specifically binds to HER2, TROP2 or MUC16. In some embodiments, the cancer is breast cancer, gastric cancer or lung cancer.

In some aspects, the present disclosure provides a compound or salt described herein for use in a method of treatment of a subject's body by therapy. In some aspects, the present disclosure provides a conjugate described herein or the pharmaceutical composition described herein for use in a method of treatment of a subject's body by therapy.

In some aspects, the present disclosure provides a compound or salt described herein for use in a method of treating cancer. In some aspects, the present disclosure provides a conjugate described herein or the pharmaceutical composition described herein for use in a method of treating cancer.

Treat and/or treating refer to any indicia of success in the treatment or amelioration of the disease or condition. Treating may include, for example, reducing, delaying or alleviating the severity of one or more symptoms of the disease or condition, or it may include reducing the frequency with which symptoms of a disease, defect, disorder, or adverse condition, and the like, are experienced by a a patient. Treat may be used herein to refer to a method that results in some level of treatment or amelioration of the disease or condition, and may contemplate a range of results directed to that end, including but not restricted to prevention of the condition entirely.

Prevent, preventing and the like may refer to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual. In some embodiments, prevent refers to preventing relapse by a subject, e.g., of a condition (e.g., cancer) for which the subject has already been treated and achieved a remission.

A therapeutically effective amount may be the amount of a composition, conjugate or compound sufficient to provide a beneficial effect or to otherwise reduce a detrimental non-beneficial event to the individual to whom the composition, conjugate or compound is administered. A therapeutically effective dose may be a dose that produces one or more desired or desirable (e.g., beneficial) effects for which it is administered, such administration occurring one or more times over a given period of time. An exact dose may depend on the purpose of the treatment and may be ascertainable by one skilled in the art using known techniques.

The conjugates, compounds and compositions described herein that may be used in therapy may be formulated and dosages established in a fashion consistent with good medical practice taking into account the disorder to be treated, the condition of the individual patient, the site of delivery of the conjugate, compound or composition, the method of administration and other factors known to practitioners. The conjugates and compounds described herein may be prepared according to the description of preparation described herein.

Pharmaceutical compositions may be considered useful with the conjugates and compounds and methods described herein may be administered to a subject in need thereof using a technique known to one of ordinary skill in the art which may be suitable as a therapy for the disease or condition affecting the subject. One of ordinary skill in the art would understand that the amount, duration and frequency of administration of a pharmaceutical composition, conjugate or compound described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

The methods, compositions, conjugates and compounds described herein may be for administration to a subject in need thereof. Often, administration of the compositions, conjugates or compounds may include routes of administration, non-limiting examples of administration routes include intravenous, intraarterial, subcutaneous, subdural, intramuscular, intracranial, intrasternal, intratumoral, or intraperitoneally. Additionally, a pharmaceutical composition, conjugate or compound may be administered to a subject by additional routes of administration, for example, by inhalation, oral, dermal, intranasal, or intrathecal administration.

Compositions, conjugates and compounds of the present disclosure may be administered to a subject in need thereof in a first administration, and in one or more additional administrations. The one or more additional administrations may be administered to the subject in need thereof minutes, hours, days, weeks or months following the first administration. Any one of the additional administrations may be administered to the subject in need thereof less than 21 days, or less than 14 days, less than 10 days, less than 7 days, less than 4 days or less than 1 day after the first administration. Any one of the additional administrations can be administered to the subject in need thereof in intervals of 21 days, or 14 days, 10 days, 7 days, 4 days or 1 day after the first administration. The one or more administrations can occur more than once per day, more than once per week or more than once per month. In some embodiments, a pharmaceutical composition is administered in cycles of weekly, biweekly, once every three weeks, monthly or bi-monthly administrations.

The compositions, conjugates, compounds and methods provided herein may be useful for the treatment of a plurality of diseases, conditions, preventing a disease or a condition in a subject or other therapeutic applications for subjects in need thereof. The compositions, compounds, conjugates and methods provided herein may be useful for treatment of hyperplastic conditions, including but not limited to, neoplasms, cancers, tumors and the like. The compositions, conjugates, compounds and methods provided herein may be useful in specifically activating immune cells in the presence of target cells, such as tumor cells. In one embodiment, the compounds of the present disclosure serve as benzazepine compounds or salts thereof and activate an immune response. In another embodiment, the conjugates serve as target cancer cells and activate an immune response. A condition, such as a cancer, may be associated with expression of an antigen on the cancer cells. The antigen expressed by the cancer cells may comprise an extracellular portion capable of recognition by the antibody construct portion of the conjugate. An antigen expressed by the cancer cells may be a tumor antigen. An antibody portion of the conjugate may recognize a tumor antigen. A tumor antigen may be CD5, CD19, CD20, CD25, CD37, CD30, CD33, CD45, CAMPATH-1, BCMA, CS-1, PD-L1, B7-H3, B7-DC, HLD-DR, carcinoembryonic antigen (CEA), TAG-72, EpCAM, MUC1, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, fibroblast activation protein (FAP), tenascin, a metalloproteinase, endosialin, vascular endothelial growth factor, avB3, WT1, LMP2, HPV E6, HPV E7, EGFRvIII (de2-7 EGFR), Her-2/neu, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin, PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7-H3, Legumain, Tie 3, Page4, VEGFR2, MAD-CT-1, PDGFR-B, MAD-CT-2, ROR2, TRAIL 1, MUC16, MAGE A4, MAGE C2, GAGE, EGFR, CMET, HER3, MUC1, MUC15, CA6, NAPI2B, TROP2, CLDN18.2, RON, LY6E, FRA, DLL3, PTK7, LIV1, ROR1, MAGE-A3 or Fos-related antigen 1.

In certain embodiments, the tumor antigen is selected from CD5, CD25, CD37, CD33, CD45, BCMA, CS-1, PD-L1, B7-H3, B7-DC (PD-L2), HLD-DR, carcinoembryonic antigen (CEA), TAG-72, EpCAM, MUC1, folate-binding protein (FOLR1), A33, G250 (carbonic anhydrase IX), prostate-specific membrane antigen (PSMA), GD2, GD3, GM2, Ley, CA-125, CA19-9 (MUC1 sLe(a)), epidermal growth factor, HER2, IL-2 receptor, EGFRvIII (de2-7 EGFR), fibroblast activation protein (FAP), a tenascin, a metalloproteinase, endosialin, avB3, LMP2, EphA2, PAP, AFP, ALK, polysialic acid, TRP-2, fucosyl GM1, mesothelin (MSLN), PSCA, sLe(a), GM3, BORIS, Tn, TF, GloboH, STn, CSPG4, AKAP-4, SSX2, Legumain, Tie 2, Tim 3, VEGFR2, PDGFR-B, ROR2, TRAIL 1, MUC16, EGFR, CMET, HER3, MUC1, MUC15, CA6, NAPI2B, TROP2, CLDN18.2, RON, LY6E, FRAlpha, DLL3, PTK7, LIV1, ROR1, CLDN6, GPC3, ADAM 12, LRRC15, CDH6, TMEFF2, TMEM238, GPNMB, ALPPL2, UPK1B, UPK2, LAMP-1, LY6K, EphB2, STEAP, ENPP3, CDH3, Nectin4, LYPD3, EFNA4, GPA33, SLITRK6 or HAVCR1.

In certain embodiments, the tumor antigen is a carbohydrate antigen, such as GD2, GD3, GM2, Ley, polysialic acid, fucosyl GM1, GM3, Tn, STn, sLe(animal), or GloboH.

In certain embodiments, the antigen is expressed on an immune cell. In certain embodiments, the antigen is HER2 or TROP2. In certain embodiments, the antigen is HER2. In certain embodiments, the antigen is TROP2. In certain embodiments, the antigen is MUC16. In certain embodiments, the antigen is PD-L1. In certain embodiments, the antigen is LIVE

As described herein, an antigen binding domain of the conjugate may be configured to recognize an antigen expressed by a cancer cell, such as for example, a disease antigen, tumor antigen or a cancer antigen. Often such antigens are known to those of ordinary skill in the art, or newly found to be associated with such a condition, to be commonly associated with, and/or, specific to, such conditions. For example, a disease antigen, tumor antigen or a cancer antigen is, but is not limited to, CD5, CD19, CD20, CD25, CD37, CD30, CD33, CD45, CAMPATH-1, BCMA, CS-1, PD-L1, B7-H3, B7-DC, HLD-DR, carcinoembryonic antigen (CEA), TAG-72, EpCAM, MUC1, folate-binding protein, A33, G250, prostate-specific membrane antigen (PSMA), ferritin, GD2, GD3, GM2, Le^{y}, CA-125, CA19-9, epidermal growth factor, p185HER2, IL-2 receptor, fibroblast activation protein (FAP), tenascin, a metalloproteinase, endosialin, vascular endothelial growth factor, avB3, WT1, LMP2, HPV E6, HPV E7, EGFRvIII (de2-7 EGFR), Her-2/neu, MAGE A3, p53 nonmutant, NY-ESO-1, MelanA/MART1, Ras mutant, gp100, p53 mutant, PR1, bcr-abl, tyrosinase, survivin, PSA, hTERT, a Sarcoma translocation breakpoint fusion protein, EphA2, PAP, ML-IAP, AFP, ERG, NA17, PAX3, ALK, androgen receptor, cyclin B1, polysialic acid, MYCN, RhoC, TRP-2, fucosyl GM1, mesothelin (MSLN), PSCA, MAGE A1, sLe(animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL 1, MUC16, MAGE A4, MAGE C2, GAGE, EGFR, CMET, HER3, MUC1, MUC15, CA6, NAPI2B, TROP2, CLDN18.2, RON, LY6E, FRA, DLL3, PTK7, LIV1, ROR1, MAGE-A3 or Fos-related antigen 1.

In certain embodiments, the disease antigen, tumor antigen or a cancer antigen is selected from CD5, CD25, CD37, CD33, CD45, BCMA, CS-1, PD-L1, B7-H3, B7-DC (PD-L2), HLD-DR, carcinoembryonic antigen (CEA), TAG-72, EpCAM, MUC1, folate-binding protein (FOLR1), A33, G250 (carbonic anhydrase IX), prostate-specific membrane antigen (PSMA), GD2, GD3, GM2, Ley, CA-125, CA19-9 (MUC1 sLe(a)), epidermal growth factor, HER2, IL-2 receptor, EGFRvIII (de2-7 EGFR), fibroblast activation protein (FAP), a tenascin, a metalloproteinase, endosialin, avB3, LMP2, EphA2, PAP, AFP, ALK, polysialic acid, TRP-2, fucosyl GM1, mesothelin (MSLN), PSCA, sLe(a), GM3, BORIS, Tn, TF, GloboH, STn, CSPG4, AKAP-4, SSX2, Legumain, Tie 2, Tim 3, VEGFR2, PDGFR-B, ROR2, TRAIL 1, MUC16, EGFR, CMET, HER3, MUC1, MUC15, CA6, NAPI2B, TROP2, CLDN18.2, RON, LY6E, FRAlpha, DLL3, PTK7, LIV1, ROR1, CLDN6, GPC3, ADAM 12, LRRC15, CDH6, TMEFF2, TMEM238, GPNMB, ALPPL2, UPK1B, UPK2, LAMP-1, LY6K, EphB2, STEAP, ENPP3, CDH3, Nectin4, LYPD3, EFNA4, GPA33, SLITRK6 or HAVCR1.

In certain embodiments, an antigen binding domain specifically binds to a carbohydrate antigen, such as GD2, GD3, GM2, Ley, polysialic acid, fucosyl GM1, GM3, Tn, STn, sLe(animal), or GloboH.

In certain embodiments, the first antigen is expressed on an immune cell. In certain embodiments, the antigen is HER2 or TROP2. In certain embodiments, the antigen is HER2. In certain embodiments, the antigen is TROP2. In certain embodiments, the antigen is MUC16. In certain embodiments, the antigen is LIVE

Additionally, such tumor antigens may be derived from the following specific conditions and/or families of conditions, including but not limited to, cancers such as brain cancers, skin cancers, lymphomas, sarcomas, lung cancer, liver cancer, leukemias, uterine cancer, breast cancer, ovarian cancer, cervical cancer, bladder cancer, kidney cancer, hemangiosarcomas, bone cancers, blood cancers, testicular cancer, prostate cancer, stomach cancer, intestinal cancers, pancreatic cancer, and other types of cancers as well as pre-cancerous conditions such as hyperplasia or the like. In certain embodiments, the cancer is breast cancer, lung cancer or gastric cancer.

Non-limiting examples of cancers can include Acute lymphoblastic leukemia (ALL); Acute myeloid leukemia; Adrenocortical carcinoma; Astrocytoma, childhood cerebellar or cerebral; Basal-cell carcinoma; Bladder cancer; Bone tumor, osteosarcoma/malignant fibrous histiocytoma; Brain cancer; Brain tumors, such as, cerebellar astrocytoma, malignant glioma, ependymoma, medulloblastoma, visual pathway and hypothalamic glioma; Brainstem glioma; Breast cancer; Bronchial adenomas/carcinoids; Burkitt's lymphoma; Cerebellar astrocytoma; Cervical cancer; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Endometrial cancer; Ependymoma; Esophageal cancer; Eye cancers, such as, intraocular melanoma and retinoblastoma; Gallbladder cancer; Glioma; Hairy cell leukemia; Head and neck cancer; Heart cancer; Hepatocellular (liver) cancer; Hodgkin lymphoma; Hypopharyngeal cancer; Islet cell carcinoma (endocrine pancreas); Kaposi sarcoma; Kidney cancer (renal cell cancer); Laryngeal cancer; Leukemia, such as, acute lymphoblastic, acute myeloid, chronic lymphocytic, chronic myelogenous and, hairy cell; Lip and oral cavity cancer; Liposarcoma; Lung cancer, such as, non-small cell and small cell; Lymphoma, such as, AIDS-related, Burkitt; Lymphoma, cutaneous T-Cell, Hodgkin and Non-Hodgkin, Macroglobulinemia, Malignant fibrous histiocytoma of bone/osteosarcoma; Melanoma; Merkel cell cancer; Mesothelioma; Multiple myeloma/plasma cell neoplasm; Mycosis fungoides; Myelodysplastic syndromes; Myelodysplastic/myeloproliferative diseases; Myeloproliferative disorders, chronic; Nasal cavity and paranasal sinus cancer; Nasopharyngeal carcinoma; Neuroblastoma; Oligodendroglioma; Oropharyngeal cancer; Osteosarcoma/malignant fibrous histiocytoma of bone; Ovarian cancer; Pancreatic cancer; Parathyroid cancer; Pharyngeal cancer; Pheochromocytoma; Pituitary adenoma; Plasma cell neoplasia; Pleuropulmonary blastoma; Prostate cancer; Rectal cancer; Renal cell carcinoma (kidney cancer); Renal pelvis and ureter, transitional cell cancer; Rhabdomyosarcoma; Salivary gland cancer; Sarcoma, Ewing family of tumors; Sarcoma, Kaposi; Sarcoma, soft tissue; Sarcoma, uterine; Sézary syndrome; Skin cancer (non-melanoma); Skin carcinoma; Small intestine cancer; Soft tissue sarcoma; Squamous cell carcinoma; Squamous neck cancer with occult primary, metastatic; Stomach cancer; Testicular cancer; Throat cancer; Thymoma and thymic carcinoma; Thymoma; Thyroid cancer; Thyroid cancer, childhood; Uterine cancer; Vaginal cancer; Waldenström macroglobulinemia; Wilms tumor and any combination thereof.

The invention also provides any therapeutic compound or conjugate disclosed herein for use in a method of treatment of the human or animal body by therapy. Therapy may be by any mechanism disclosed herein, such as by stimulation of the immune system. The invention provides any therapeutic compound or conjugate disclosed herein for use in stimulation of the immune system, vaccination or immunotherapy, including for example enhancing an immune response. The invention further provides any therapeutic compound or conjugate disclosed herein for prevention or treatment of any condition disclosed herein, for example cancer, autoimmune disease, inflammation, sepsis, allergy, asthma, graft rejection, graft-versus-host disease, immunodeficiency or infectious disease (typically caused by an infectious pathogen). The invention also provides any therapeutic compound or conjugate disclosed herein for obtaining any clinical outcome disclosed herein for any condition disclosed herein, such as reducing tumour cells in vivo. The invention also provides use of any therapeutic compound or conjugate disclosed herein in the manufacture of a medicament for preventing or treating any condition disclosed herein.

### GENERAL SYNTHETIC SCHEMES AND EXAMPLES

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

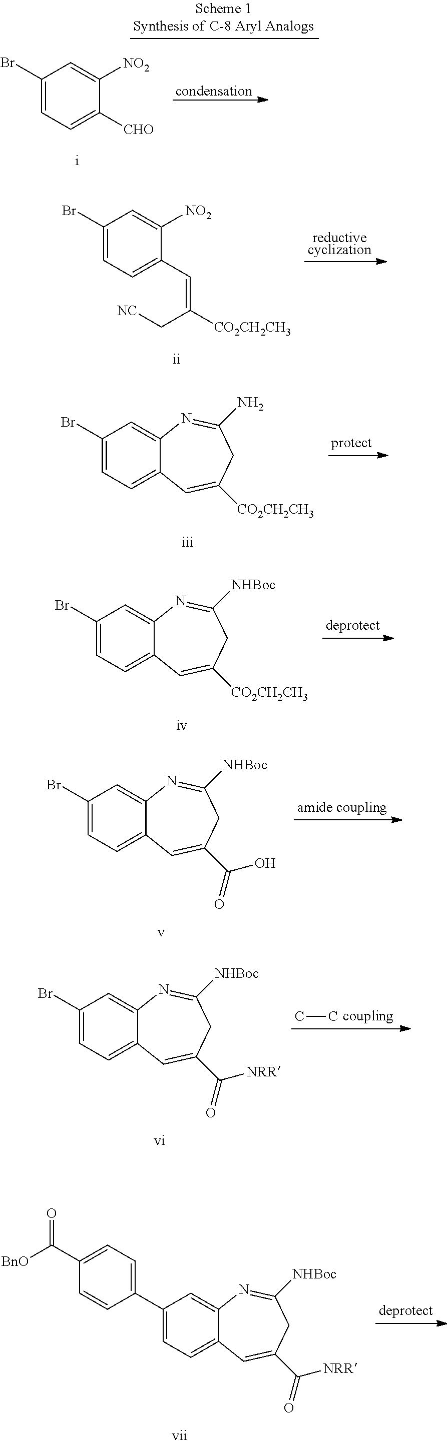

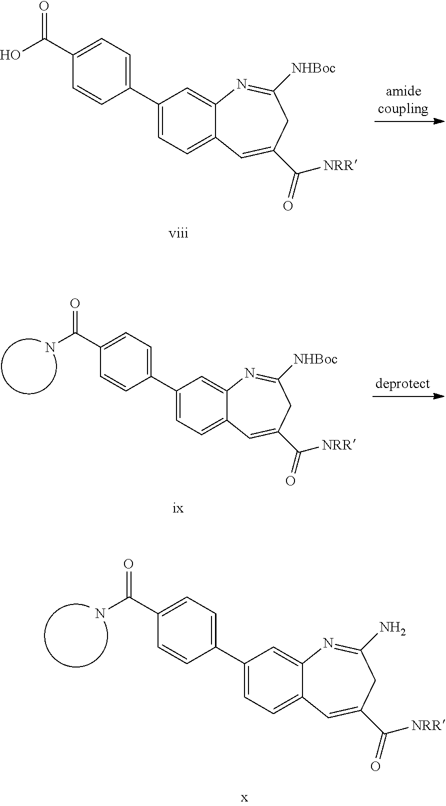

An aldehyde is reacted (i) with an appropriate Wittig reagent, such as tert-butyl 3-cyano-2-(triphenylphosphorylidene)propanoate, at elevated temperatures to afford an olefin (ii), which undergoes reductive cyclization by treating the olefin (ii) with a reducing agent, such as iron powder in hot acetic acid, to afford azepines (iii). The 2-amino substituent of compounds (iii) is protected with a tert-butoxycarbonyl group to give compounds (iv). The C-4 ester group is hydrolyzed by using a strong base such as LiOH in a mixture of THF and methanol to afford to give compounds (v), which is in turn coupled with a substituted amine using a coupling agent, such as BOP reagent to provide compounds (vi). The C-8 bromide of (vi) is converted to the corresponding biphenyl analog (vii) using a palladium catalyst such as tetrakis(triphenylphosphine)palladium(0) and a base such as potassium phosphate in a mixture of acetonitrile and water. Carboxylic esters (vii) can be deprotected by way of catalytic hydrogenation to afford carboxylic acids (viii) which can subsequently be converted to cyclic amide analogs (ix) using known reagents such as HBTU and a tertiary amine base. Acid-mediated deprotection of compounds (ix) using a reagent such as TFA in dichloromethane provides the target compounds (x).

### Example 1

### Synthesis of 2-amino-8-(4-(3-phenylpiperazine-1-carbonyl)phenyl)-N,N-dipropyl-3H-benzo[b]azepine-4-carboxamide (Compound 1.1)

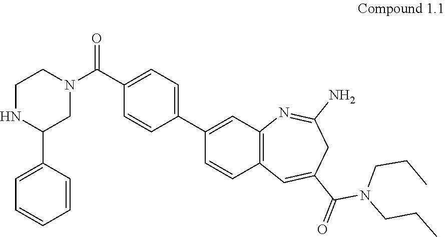 [SMILES: CCCN(CCC)C(=O)C1=Cc2ccc(-c3ccc(C(=O)N4CCNC(c5ccccc5)C4)cc3)cc2N=C(N)C1]

### Step A: Preparation of Int 1.1a

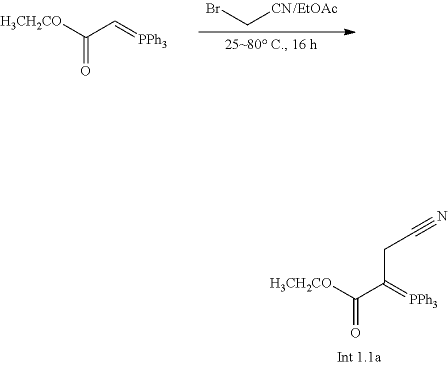

Bromoacetonitrile (8.60 g, 71.7 mmol, 4.78 mL) was added to a solution of ethyl (triphenylphosphorylidine)acetate (45.0 g, 119 mmol, 1.00 eq) in EtOAc (260 mL) at approximately 25° C. The reaction was heated at approximately 80° C. for approximately 16 h, after which time TLC (DCM:MeOH=10:1; R_{f}=0.4) and LCMS showed the reaction was complete. The mixture was cooled, filtered and washed with EtOAc (200 mL) and concentrated to afford crude Int 1.1a as a solid, which was used directly without purification.

### Step B: Preparation of Int 1.1b

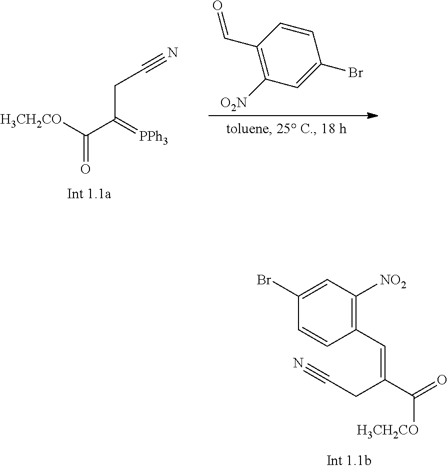

A solution of Int 1.1a (30.0 g, 77.5 mmol, 1.00 eq) and 4-bromo-2-nitrobenzaldehyde (19.6 g, 85.2 mmol, 1.10 eq) in toluene (250 mL) was stirred at approximately 25° C. for approximately 18 h. TLC (hexanes:EtOAc=1:2) showed the reaction was complete and the mixture was concentrated to afford crude product, which was covered in 150 mL of methanol and stored at approximately 4° C. overnight. The resulting precipitate was filtered and provided approximately 16 g of Int 1.1b as a white solid. LCMS (M+H)=339.0.

### Step C: Preparation of Int 1.1c

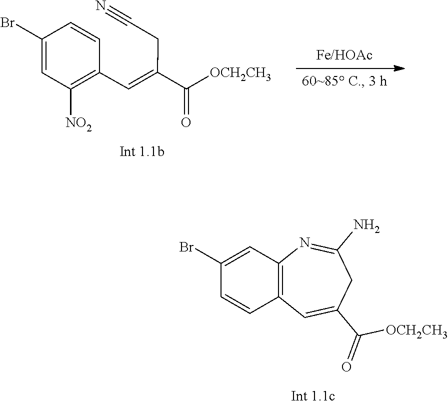 [SMILES: CCOC(=O)/C(=C/c1ccc(Br)cc1[N+](=O)[O-])CC#N ; CCOC(=O)C1=Cc2ccc(Br)cc2N=C(N)C1]

Iron powder (15.5 g, 283.2 mmol, 6.00 eq) was added to a solution of Int 1.1b (16.0 g, 47.2 mmol, 1.00 eq) in glacial acetic acid (250 mL) at approximately 60° C. The mixture was stirred at approximately 80° C. for approximately 3 h. TLC (petroleum ether:EtOAc=1:2; R_{f}=0.43) showed the reaction was completed and the mixture was cooled, filtered, washed with acetic acid (100 mL×2) and concentrated. The crude residue was diluted with EtOAc (100 mL) and washed with aq. NaHCO₃ (50 mL×3) and dried over Na₂SO₄, filtered and concentrated. The residue was purified by silica gel chromatography to afford approximately 15 g of the Int 1.1c as a yellow solid. LCMS (M+H)=309.0.

### Step D: Preparation of Int 1.1d

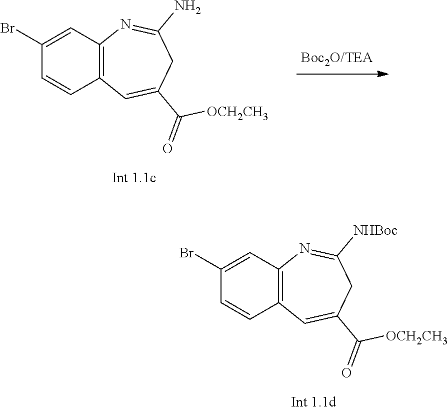 [SMILES: CCOC(=O)C1=Cc2ccc(Br)cc2N=C(N)C1 ; CCOC(=O)C1=Cc2ccc(Br)cc2N=C(NC(=O)OC(C)(C)C)C1]

A solution containing 15 g (48.5 mmol) of Int 1.1c in 500 mL of dichloromethane was cooled to 0° C. and treated with 10.8 mL (77.6 mmol, 1.6 eq) of TEA and then 17 g (77.6 mmol, 1.6 eq) of Boc₂O. The reaction mixture was stirred at room temperature overnight and then quenched with 50 mL of water. The layers were separated and the aqueous was back extracted with dichloromethane (3×30 mL). The combined organic extracts were washed with brine and dried over Na₂SO₄. The solvent was removed and the residue was purified by silica gel chromatography (0% to 100% EtOAc/Hexanes) to afford approximately 12 g of Int 1.1d as a white solid. LCMS (M+H)=409.0.

### Step E: Preparation of Int 1.1e

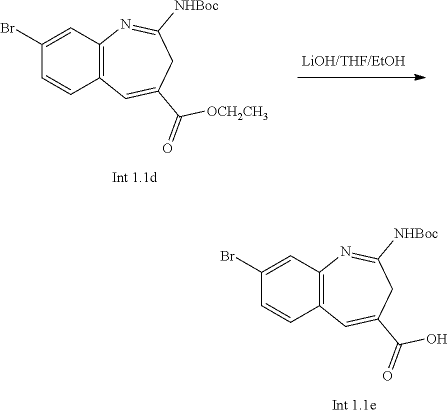 [SMILES: CCOC(=O)C1=Cc2ccc(Br)cc2N=C(NC(=O)OC(C)(C)C)C1 ; CC(C)(C)OC(=O)NC1=Nc2cc(Br)ccc2C=C(C(=O)O)C1]

A solution containing 12.0 g (29.3 mmol) of Int 1.1d in 100 mL of a 1:1 mixture of THF and ethanol was cooled to 0° C. and treated with 44 mL (44 mmol) of 1N LiOH. After stirring for approximately 16 h, ice chips were added, followed by enough 5% citric acid solution to effect a precipitate (at approximately pH 5.5). The resulting mixture was washed three times with EtOAc and the combined organic extracts were washed with brine and dried over Na₂SO₄. The solution was evaporated to afford approximately 9.0 g of Int 1.1e as a pale yellow solid, which was used without purification. LCMS (M+H)=380.

### Step F: Preparation of Int 1.1f

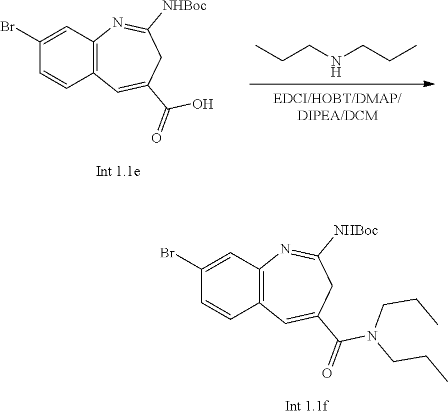

3.59 g (35.5 mmol) of di-n-propylamine, 11.4 g (59.2 mmol) of EDCI, 3.8 g (28.4 mmol) of HOBT, 867 mg (7.11 mmol) of DMAP and 10.4 mL (94.8 mmol) of DIPEA were added to a solution containing 9.0 g (23.7 mmol) of Int 1.1e in 100 mL of dichoromethane. The reaction was stirred for 3 h and then quenched with 20 mL of saturated NH₄Cl and then 20 mL of water. The mixture was extracted with DCM (3×30 mL) and the combined organic extracts were washed with brine (2×) and then dried over Na₂SO₄. After removal of the drying agent and concentration of the DCM solution, the residue was purified on silica gel (80 g column; 0% to 100% hexanes/EtOAc) to afford approximately 7.0 g of Int 1.1f. LCMS (M+H)=464.

### Step G: Preparation of Int 1.1g

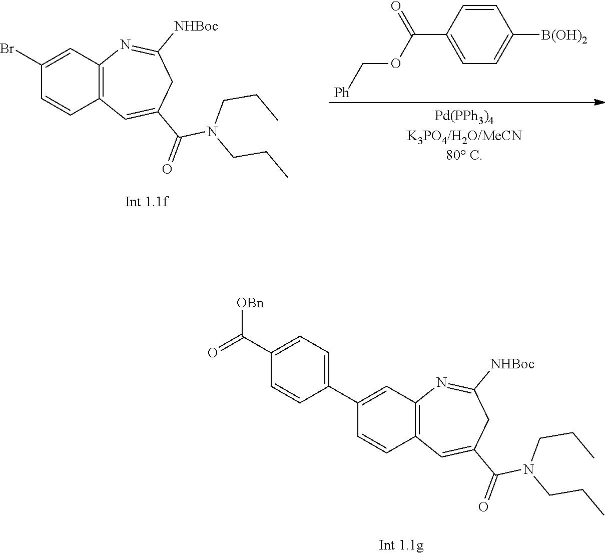 [SMILES: CCCN(CCC)C(=O)C1=Cc2ccc(Br)cc2N=C(NC(=O)OC(C)(C)C)C1 ; O=C(OCc1ccccc1)c1ccc(B(O)O)cc1 ; CCCN(CCC)C(=O)C1=Cc2ccc(-c3ccc(C(=O)OCc4ccccc4)cc3)cc2N=C(NC(=O)OC(C)(C)C)C1]

A solution of Int 1.1f (500 mg, 1.08 mmol), (4-((benzyloxy)carbonyl)phenyl)boronic acid (553 mg, 2.16 mmol), 2.16 mmol of potassium phosphate and Pd(PPh₃)₄ (127 mg, 0.11 mmol) in a 12:1 mixture of acetonitrile/water (10 mL/g) was heated at 80° C. for 16 h. The reaction mixture was cooled to room temperature, evaporated, and then purified by reverse phase chromatography to afford approximately 330 mg of Int 1.1g as a white solid. LCMS (M+H)=596.

### Step H: Preparation of Int 1.1h

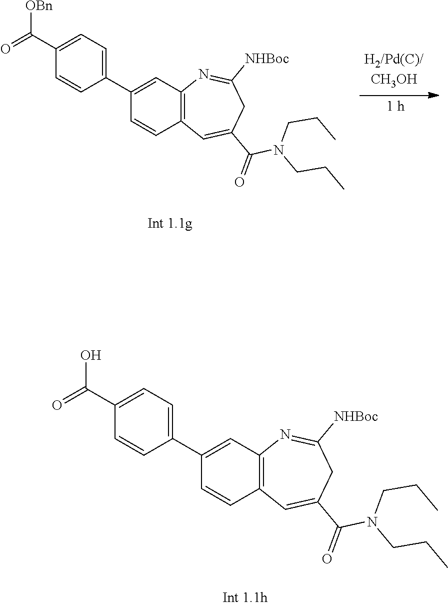 [SMILES: CCCN(CCC)C(=O)C1=Cc2ccc(-c3ccc(C(=O)OCc4ccccc4)cc3)cc2N=C(NC(=O)OC(C)(C)C)C1 ; CCCN(CCC)C(=O)C1=Cc2ccc(-c3ccc(C(=O)O)cc3)cc2N=C(NC(=O)OC(C)(C)C)C1]

A solution of Int 1.1g (330 mg, 0.55 mmol) in 10 mL of methanol and 50 mg of 10% Pd on carbon was stirred under an atmosphere of hydrogen for 1 h and then filtered through Celite and evaporated to provide approximately 290 mg of Int 1.1h as a white solid. LCMS (M+H)=506.

### Step I: Preparation of Int 1.1i

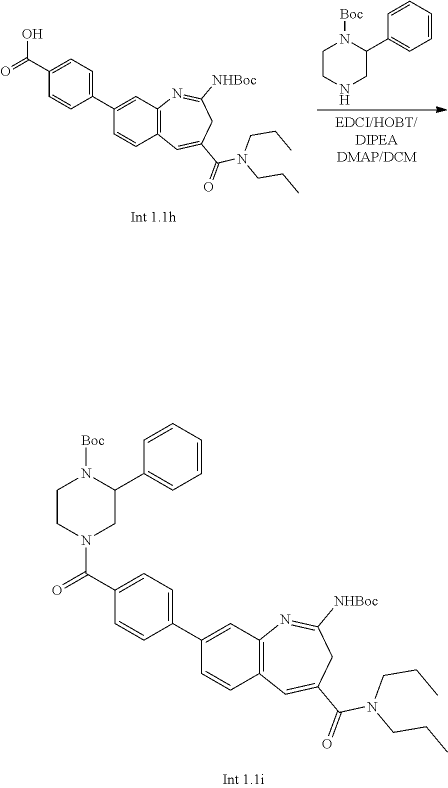 [SMILES: CCCN(CCC)C(=O)C1=Cc2ccc(-c3ccc(C(=O)O)cc3)cc2N=C(NC(=O)OC(C)(C)C)C1 ; CC(C)(C)OC(=O)N1CCNCC1c1ccccc1 ; CCCN(CCC)C(=O)C1=Cc2ccc(-c3ccc(C(=O)N4CCN(C(=O)OC(C)(C)C)C(c5ccccc5)C4)cc3)cc2N=C(NC(=O)OC(C)(C)C)C1]

79 mg (0.30 mmol) of tert-butyl 2-phenylpiperazine-1-carboxylate, 96 mg (0.50 mmol) of EDCI, 32 mg (0.24 mmol) of HOBT, 7 mg (0.06 mmol) of DMAP and 0.11 mL (0.8 mmol) of DIPEA were added to a solution containing 100 mg (0.20 mmol) of Int 1.1h in 2 mL of dichoromethane. The reaction was stirred for 16 h and then quenched with saturated NH₄Cl and then water. The mixture was extracted with DCM (3×5 mL) and the combined organic extracts were washed with brine (2×) and then dried over Na₂SO₄. After removal of the drying agent, the residue was purified by reverse phase chromatography to afford approximately 90 mg of Int 1.1i. LCMS (M+H)=750.

### Step J: Preparation of Compound 1.1

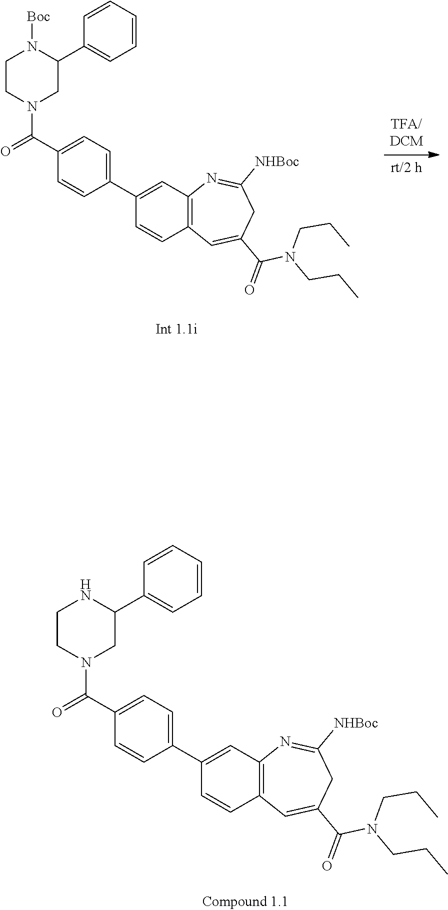 [SMILES: CCCN(CCC)C(=O)C1=Cc2ccc(-c3ccc(C(=O)N4CCN(C(=O)OC(C)(C)C)C(c5ccccc5)C4)cc3)cc2N=C(NC(=O)OC(C)(C)C)C1 ; CCCN(CCC)C(=O)C1=Cc2ccc(-c3ccc(C(=O)N4CCNC(c5ccccc5)C4)cc3)cc2N=C(NC(=O)OC(C)(C)C)C1]

2 mL of TFA was added to a solution containing 90 mg (0.12 mmol) of Int 1.1i in 2 mL of DCM. The solution was stirred for 2 h at room temperature. Evaporation of the solvents afforded a residue, which was purified by reverse phase chromatography to afford approximately 50 mg of Compound 1.1 as a white solid. ¹H NMR (CD₃CN) δ 7.81 (d, J=8.1 Hz, 2H), 7.71-7.58 (m, 5H), 7.48 (bs, 5H), 6.99 (s, 1H), 4.46 (dd, J=3.0, 11.4 Hz, 1H), 3.41 (m, 8H), 1.66 (m, 4H), 0.89 (bs, 6H). LCMS (M+H)=550.4.

The following compounds, as shown in Table 1, could be prepared in a manner similar to that used for the synthesis of Compound 1.1 using Intermediate 1.1h and an appropriately substituted amine.

TABLE 1
Compounds 1.2-1.11

| Cmpd | Structure and IUPAC | ¹H NMR | M + 1 |
|---|---|---|---|
| 1.2 | 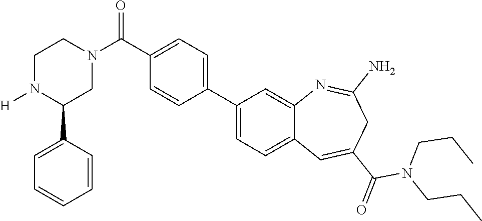 [SMILES: CCCN(CCC)C(=O)C1=Cc2ccc(-c3ccc(C(=O)N4CCN[C@H](c5ccccc5)C4)cc3)cc2N=C(N)C1] | (DMSO-d₆) δ 7.76 (d, J = 8.1 Hz, 2H), 7.51- (d, J = 8.1 Hz, 2H), 7.39- 7.26 (m, 8H), 6.81 (bs, 2H), 6.75 (s, 1H), 3.32-2.55 (m, 12H), 1.57 (m, 4H), 0.84 (bs, 6H). | 550.4 |
| | (R)-2-amino-8-(4-(3-phenylpiperazine-1- | | |
| | carbonyl)phenyl)-N,N-dipropyl-3H- | | |
| | benzo[b]azepine-4-carboxamide | | |
| 1.3 | 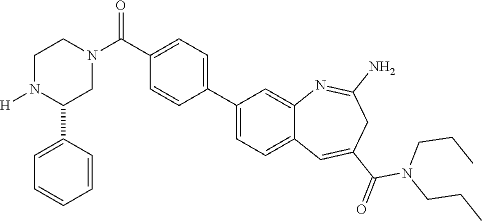 | (DMSO-d₆) δ 7.76 (d, J = 8.1 Hz, 2H), 7.51- (d, J = 8.1 Hz, 2H), 7.39- 7.26 (m, 8H), 6.81 (bs, 2H), 6.75 (s, 1H), 3.32-2.55 (m, 12H), 1.57 (m, 4H), 0.84 (bs, 6H). | 550.4 |
| | (S)-2-amino-8-(4-(3-phenylpiperazine-1- | | |
| | carbonyl)phenyl)-N,N-dipropyl-3H- | | |
| | benzo[b]azepine-4-carboxamide | | |
| 1.4 | 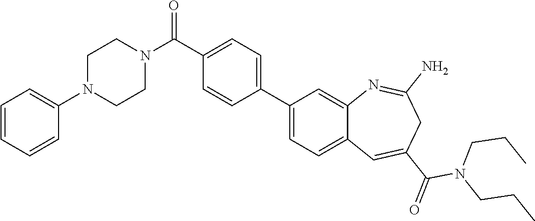 [SMILES: CCCN(CCC)C(=O)C1=Cc2ccc(-c3ccc(C(=O)N4CCN(c5ccccc5)CC4)cc3)cc2N=C(N)C1] | (CD₃CN) δ 7.79 (d, J = 8.4 Hz, 2H), 7.72 (m, 2H), 7.70 (m, 3H), 7.34 (m, 2H), 7.08 (m, 2H), 6.98 (m, 2H), 3.91 (bs, 2H), 3.66 (bs, 2H), 3.42-3.22 (m, 6H), 3.27 (m, 4H), 1.66 (m, 4H), 0.91 (bs, 6H). | 550.3 |
| | 2-amino-8-(4-(4-phenylpiperazine-1- | | |
| | carbonyl)phenyl)-N,N-dipropyl-3H- | | |
| | benzo[b]azepine-4-carboxamide | | |
| 1.5 | 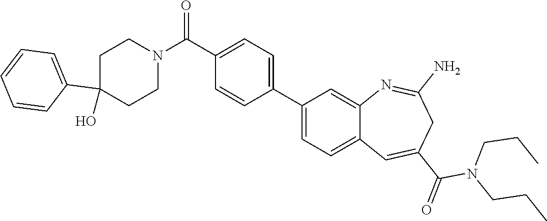 [SMILES: CCCN(CCC)C(=O)C1=Cc2ccc(-c3ccc(C(=O)N4CCC(O)(c5ccccc5)CC4)cc3)cc2N=C(N)C1] | (CD₃CN) δ 7.79 (d, J = 8.4 Hz, 2H), 7.58 (t, J = 8.8 Hz, 4H), 7.4-7.2 (m, 6H), 6.78 (s, 1H), 5.41 (bs, 2H), 3.6-3.2 (m, 8H), 2.21 (m, 4H), 1.52 (m, 4H), 0.87 (bs, 6H). | 565.4 |
| | 2-amino-8-(4-(4-hydroxy-4-phenylpiperidine-1- | | |
| | carbonyl)phenyl)-N,N-dipropyl-3H- | | |
| | benzo[b]azepine-4-carboxamide | | |
| 1.6 | 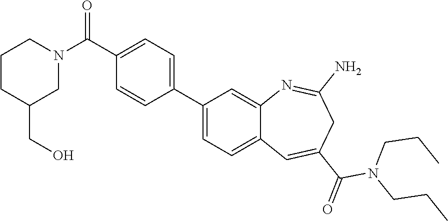 [SMILES: CCCN(CCC)C(=O)C1=Cc2ccc(-c3ccc(C(=O)N4CCCC(CO)C4)cc3)cc2N=C(N)C1] | (CD₃CN) δ 7.74 (d, J = 8.4 Hz, 2H), 7.58- 7.41 (m, 5H), 6.88 (s, 1H), 4.44 (bs, 1H), 3.42 (m, 4H), 3.22- 2.81 (m, 4H), 1.82- 1.25 (m, 9H), 0.91 (bs, 6H). | 503.4 |
| | 2-amino-8-(4-(3-(hydroxymethyl)piperidine-1- | | |
| | carbonyl)phenyl)-N,N-dipropyl-3H- | | |
| | benzo[b]azepine-4-carboxamide | | |
| 1.7 | 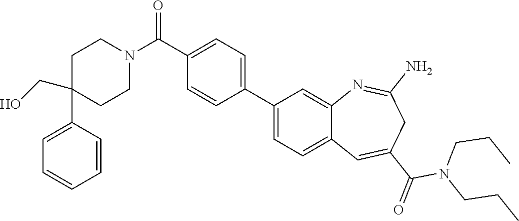 [SMILES: CCCN(CCC)C(=O)C1=Cc2ccc(-c3ccc(C(=O)N4CCC(CO)(c5ccccc5)CC4)cc3)cc2N=C(N)C1] | (DMSO-d₆) δ 7.78 (d, J = 6.8 Hz, 2H), 7.66- 7.22 (m, 9H), 6.88 (bs, 1H), 6.75 (s, 1H), 4.69 (t, J = 8.5 Hz, 1H), 4.22 (m, 1H), 3.55 (m, 1H), 3.40-3.00 (m, 8H), 2.73 (s, 2H), 2.30-1.80 (m, 4H), 1.58 (m, 4H), 0.67 (bs, 6H). | 579.4 |
| | 2-amino-8-(4-(4-(hydroxymethyl)-4- | | |
| | phenylpiperidine-1-carbonyl)phenyl)-N,N-dipropyl- | | |
| | 3H-benzo[b]azepine-4-carboxamide | | |
| 1.8 | 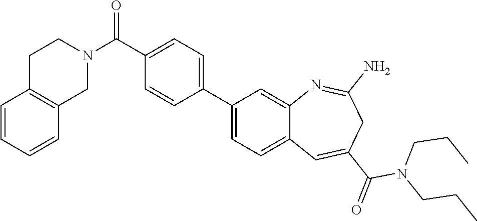 [SMILES: CCCN(CCC)C(=O)C1=Cc2ccc(-c3ccc(C(=O)N4CCc5ccccc5C4)cc3)cc2N=C(N)C1] | (CD₃CN) δ 7.79 (d, J = 8.4 Hz, 2H), 7.71 (m, 2H), 7.68 (m, 3H), 7.22 (m, 4H), 6.97 (s, 1H), 4.82 (bs, 2H), 3.65 (m, 2H), 3.41 (m, 4H), 3.25 (s, 2H), 2.90 (m, 2H), 1.64 (m, 4H), 0.89 (bs, 6H). | 521.4 |
| | 2-amino-N,N-dipropyl-8-(4-(1,2,3,4- | | |
| | tetrahydroisoquinoline-2-carbonyl)phenyl)-3H- | | |
| | benzo[b]azepine-4-carboxamide | | |
| 1.9 | 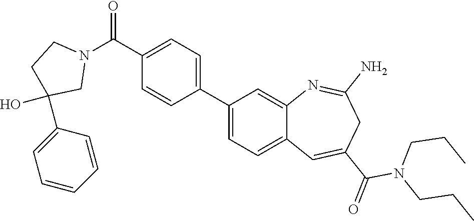 [SMILES: CCCN(CCC)C(=O)C1=Cc2ccc(-c3ccc(C(=O)N4CCC(O)(c5ccccc5)C4)cc3)cc2N=C(N)C1] | (CD₃CN) δ 7.79-7.51 (m, 6H), 7.44-7.22 (m, 6H), 6.79 (s, 1H), 4.82 (bs, 2H), 4.00-3.80 (m, 3H), 3.79-3.65 (m, 2H), 3.41 (m, 4H), 2.75 (s, 2H), 2.30 (m, 2H), 2.11 (m, 1H), 1.66 (m, 4H), 0.88 (bs, 6H). | 551.4 |
| | 2-amino-8-(4-(3-hydroxy-3-phenylpyrrolidine-1- | | |
| | carbonyl)phenyl)-N,N-dipropyl-3H- | | |
| | benzo[b]azepine-4-carboxamide | | |
| 1.10 | 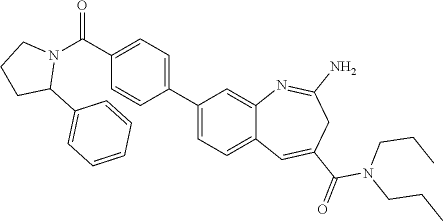 [SMILES: CCCN(CCC)C(=O)C1=Cc2ccc(-c3ccc(C(=O)N4CCCC4c4ccccc4)cc3)cc2N=C(N)C1] | (CD₃OD) δ 7.84 (d, J = 8.4 Hz, 1H), 7.76 (d, J = 8.4 Hz, 1H), 7.68- 7.51 (m, 4H), 7.42- 7.05 (m, 6H), 5.28 (m, 1H), 5.00 (m, 1H), 3.95 (m, 2H), 3.50- 3.33 (m, 6H), 2.53 (m, 1H), 2.20-1.80 (m, 3H), 1.77 (m, 4H), 0.92 (bs, 6H). | 535.3 |
| | 2-amino-8-(4-(2-phenylpyrrolidine-1- | | |
| | carbonyl)phenyl)-N,N-dipropyl-3H- | | |
| | benzo[b]azepine-4-carboxamide | | |
| 1.11 | 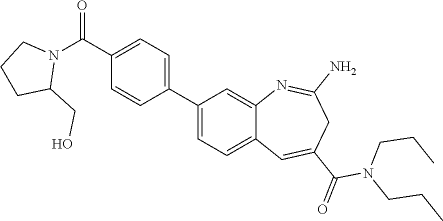 [SMILES: CCCN(CCC)C(=O)C1=Cc2ccc(-c3ccc(C(=O)N4CCCC4CO)cc3)cc2N=C(N)C1] | (CD₃CN) δ 7.72 (d, J = 8.0 Hz, 2H), 7.53 (d, J = 8.1 Hz, 2H), 7.33- 7.23 (m, 3H), 6.73 (s, 1H), 5.5-5.3 (bs, 1H), 4.19 (m, 1H), 3.62- 3.42 (m, 4H), 3.40 (t, J = 8.0 Hz, 4H), 2.75 (s, 2H), 2.30-2.10 (m, 3H), 1.80-1.50 (m, 7H), 0.88 (bs, 6H). | 489.4 |
| | 2-amino-8-(4-(2-(hydroxymethyl)pyrrolidine-1- | | |
| | carbonyl)phenyl)-N,N-dipropyl-3H- | | |
| | benzo[b]azepine-4-carboxamide | | |

### Example 2

### PBMC Screening Assay

Materials and general procedures. Human peripheral blood mononuclear cells (PBMC) were obtained from BenTek, frozen at 25×10⁶ cell/mL in 10% DMSO (Sigma) prepared in fetal bovine serum (Gibco) and stored in liquid nitrogen. For the culture, PBMC were thawed quickly in a 37° C. water bath and diluted into pre-warmed RPMI 1640 (Lonza) supplemented with 10% fetal bovine serum, 2 mM glutamine, 50 μg/mL penicillin, 50 U/mL streptomycin (all from Gibco) and centrifuged for 5 minutes at 500×g. PBMC were suspended into the growth media described above and cultured at a concentration of 1×10⁶ cells per mL at 37° C. in a 5% CO₂ incubator.

General procedure for in vitro small molecule screening. PBMC were thawed, suspended at a concentration of 1×10⁶ cell/mL in growth media and 200 μL was aliquoted into each well of a 96-well plate for a total of 0.2×10⁶ cells per well. PBMC were incubated for approximately 16-18 hours at 37° C. in a 5% CO₂ humidified incubator. PBMC plates were centrifuged at 500×g for 5 minutes and the growth media was removed. 150 μL of twelve concentrations ranging from 1000 to 0.000238 nM of small molecules prepared in growth media were added to PBMC in duplicate and incubated for 24 hours at 37° C. in a 5% CO₂ incubator. Prior to supernatant harvest, cells were spun at 500×g for 5 minutes to remove cell debris. TNF-α activity was assessed in the supernatant by ELISA (eBioscience) or HTRF (CisBio) per the manufacturer's instructions. The optical density at 450 nm and 570 nm (ELISA) or luminescence (HTRF) was analyzed using an Envision (Perkin Elmer) plate reader, as shown in Table 2. In Table 2, compounds of the disclosure with an EC₅₀ value of less than 50 nM have “A” activity, from 50-500 nM have “B” activity and greater than 500 nM have “C” activity.

TABLE 2
In vitro small molecule screening

| Compound | EC₅₀ (nM) |
|---|---|
| 1.1 | A |
| 1.2 | A |
| 1.3 | A |
| 1.4 | B |
| 1.5 | B |
| 1.6 | B |
| 1.7 | C |
| 1.8 | C |
| 1.9 | B |
| 1.10 | B |
| 1.11 | B |

### Example 3

### Protocol for the Preparation of Antibody Benzazepine Conjugates

A monoclonal antibody (mAb) in PBS is exchanged into HEPES (100 mM, pH 7.0, 1 mM DTPA) via molecular weight cut-off centrifugal filtration (Millipore, 30 kDa). The resultant mAb solution is transferred to a 50 mL conical tube. The mAb concentration is determined by A₂₈₀. To the mAb solution is added TCEP (2.0 eq, 1 mM stock) at room temperature and the resultant mixture is incubated at 37° C. for 1 hr, with gentle shaking. Upon being cooled to room temperature, a stir bar is added to the reaction tube. With stirring, DMA (10% v/v, 3.0 mL) is added dropwise to the reaction mixture. A benzazepine compound-linker construct is added dropwise and the resultant reaction mixture is allowed to stir at ambient temperature for 30 minutes, at which point N-ethyl maleimide (3.0 eq, 100 mM DMA) is added. After an additional 15 minutes of stirring, cysteine (6.0 equiv., 50 mM HEPES) is added. The crude conjugate is then exchanged into PBS and purified by preparative SEC (HiLoad 26/600, Superdex 200 pg) using PBS as the mobile phase. The pure fractions are concentrated via molecular weight cut-off centrifugal filtration (Millipore, 30 kDa), sterile filtered and transferred to 15 mL conical tubes. Drug-antibody construct ratios (molar ratios) are determined by methods described in Example 4.

### Example 4

### General Procedure for the Determination of the Drug-Antibody-Ratios

### Hydrophobic Interaction Chromatography

10 μL of a 6 mg/mL solution of the conjugate is injected into an HPLC system set-up with a TOSOH TSKgel Butyl-NPR TM hydrophobic interaction chromatography (HIC) column (2.5 μM particle size, 4.6 mm×35 mm) attached. Then, over the course of 18 minutes, a method is run in which the mobile phase gradient runs from 100% mobile phase A to 100% mobile phase B over the course of 12 minutes, followed by a six-minute re-equilibration at 100% mobile phase A. The flow rate is 0.8 mL/min and the detector is set at 280 nM. Mobile phase A is 1.5 M ammonium sulfate, 25 mM sodium phosphate (pH 7). Mobile phase B is 25% isopropanol in 25 mM sodium phosphate (pH 7). Post-run, the chromatogram is integrated and the molar ratio is determined by summing the weighted peak area.

### Mass Spectrometry

One microgram of conjugate is injected into an LC/MS such as an Agilent 6550 iFunnel Q-TOF equipped with an Agilent Dual Jet Stream ESI source coupled with Agilent 1290 Infinity UHPLC system. Raw data is obtained and is deconvoluted with software such as Agilent MassHunter Qualitative Analysis Software with BioConfirm using the Maximum Entropy deconvolution algorithm. The average mass of intact conjugates is calculated by the software, which used top peak height at 25% for the calculation. This data is then imported into another program to calculate the molar ratio of the conjugate such as Agilent molar ratio calculator.

### Example 5

### TNFα Production by PBMCs was Induced by Benzazepine Antibody Conjugates

This example shows that the conjugates of benzazepine compounds can increase production of a pro-inflammatory cytokine, TNFα, by PBMCs in the presence of tumor cells.

PBMCs are isolated from human blood as described above. Briefly, PBMCs are isolated by Ficoll gradient centrifugation, resuspended in RPMI, and plated in 96-well flat bottom microtiter plates (125,000/well). Antigen-expressing tumor cells are then added (25,000/well) along with titrating concentrations of conjugates or unconjugated parental antibodies as controls. After overnight culture, supernatants are harvested, and TNFα levels are determined by AlphaLISA. TNFα production is measured after 24 hours.

What is claimed is:

1. A compound represented by the structure of Formula (IA): 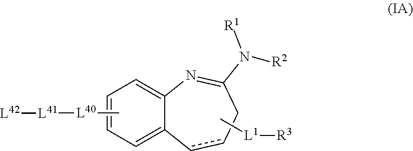
or a pharmaceutically acceptable salt thereof, wherein: [symbol]
represents an optional double bond;
L⁴⁰ is selected from C₃₋₁₂ carbocyclene and 3- to 12-membered heterocyclene, wherein the C₃₋₁₂ carbocyclene and the 3- to 12-membered heterocyclene are optionally substituted with one or more substituents independently selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
L¹ and L⁴¹ are independently selected from a bond, C₁-C₂ alkylene optionally substituted with one or more R³¹, —O—, —S—, —N(R¹⁰)—, —C(O)—, —C(O)O—, —OC(O)—, —C(O)N(R¹⁰)—, —N(R¹⁰)C(O)—, —C(NR¹⁰)—, —P(O)(OR¹⁰)O—, —O(R¹⁰O)(O)P—, —OS(O)—, —S(O)O—, —S(O)—, —OS(O)₂—, —S(O)₂O—, —N(R¹⁰)S(O)₂—, —S(O)₂N(R¹⁰)—, —N(R¹⁰)S(O)—, and —S(O)N(R¹⁰)—;
L⁴² is selected from: 3- to 8-membered saturated heterocycle substituted with a substituent selected from R³⁰, and the 3- to 8-membered saturated heterocycle is optionally substituted with one or more additional substituents selected from R³¹; and optionally substituted C₃₋₁₂ carbocycle, optionally substituted 3- to 12-membered unsaturated heterocycle, optionally substituted heteroaryl, and optionally substituted 8-14 membered bicyclic heterocycle each of which is optionally substituted with one or more substituents independently selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R¹ and R² are independently selected from hydrogen; and C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), and —CN;
R³ is selected from:
—OR¹⁰, —N(R¹⁰)₂, —C(O)N(R¹⁰)₂, —C(O)R¹⁰, —C(O)OR¹⁰, —S(O)R¹⁰, and —S(O)₂R¹⁰;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R¹⁰ is independently selected at each occurrence from:
hydrogen; and
C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R¹¹ is independently selected at each occurrence from C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R³⁰ is selected from:
halogen, —OR¹¹, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R³¹ is selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each is which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl; and
wherein any substitutable carbon on the benzazepine core is optionally substituted by a substituent selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —P(O)(OR¹⁰)₂, —OP(O)(OR¹⁰)₂, —CN, C₁₋₁₀ alkyl, C₂₋₁₀ alkenyl, and C₂₋₁₀ alkynyl, or two substituents on a single carbon atom or two adjacent carbons combine to form a 3- to 7-membered carbocycle.

2. The compound or salt of claim 1, wherein the compound of Formula (IA) is represented by Formula (IB): 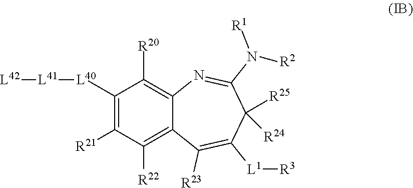
or a pharmaceutically acceptable salt thereof, wherein:
R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and
R²⁴ and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

3. A compound represented by the structure of Formula (IIIA): 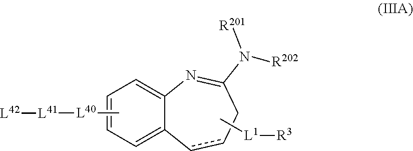
or a pharmaceutically acceptable salt thereof, wherein: [symbol]
represents an optional double bond;
L⁴⁰ is selected from C₃₋₁₂ carbocyclene and 3- to 12-membered heterocyclene, wherein the C₃₋₁₂ carbocyclene and the 3- to 12-membered heterocyclene are optionally substituted with one or more substituents independently selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
L¹ and L⁴¹ are independently selected from a bond, C₁-C₂ alkylene optionally substituted with one or more R³¹, —O—, —S—, —N(R¹⁰)—, —C(O)—, —C(O)O—, —OC(O)—, —C(O)N(R¹⁰)—, —N(R¹⁰)C(O)—, —C(NR¹⁰)—, —P(O)(OR¹⁰)O—, —O(R¹⁰O)(O)P—, —OS(O)—, —S(O)O—, —S(O)—, —OS(O)₂—, —S(O)₂O—, —N(R¹⁰)S(O)₂—, —S(O)₂N(R¹⁰)—, —N(R¹⁰)S(O)—, and —S(O)N(R¹⁰)—;
L⁴² is selected from: 3- to 8-membered saturated heterocycle substituted with a substituent selected from R³⁰, and optionally substituted with one or more additional substituents selected from R³¹; optionally substituted C₃₋₁₂ carbocycle, optionally substituted 3- to 12-membered unsaturated heterocycle, optionally substituted heteroaryl, and optionally substituted 8-14 membered bicyclic heterocycle each of which is optionally substituted with one or more substituents independently selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R²⁰¹ is hydrogen;
R²⁰² is an amine masking group;
R³ is selected from:
—OR¹⁰, —N(R¹⁰)₂, —C(O)N(R¹⁰)₂, —C(O)R¹⁰, —C(O)OR¹⁰, —S(O)R¹⁰, and —S(O)₂R¹⁰;
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R¹⁰ is independently selected at each occurrence from:
hydrogen; and
C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R¹¹ is independently selected at each occurrence from C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R³⁰ is selected from:
halogen, —OR¹¹, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl;
R³¹ is selected from:
halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₃₋₁₂ carbocycle, and 3- to 12-membered heterocycle; and
C₃₋₁₂ carbocycle and 3- to 12-membered heterocycle, each is which is optionally substituted with one or more substituents independently selected from halogen, —OR¹⁰, —SR¹⁰, —C(O)N(R¹⁰)₂, —N(R¹⁰)C(O)R¹⁰, —N(R¹⁰)C(O)N(R¹⁰)₂, —N(R¹⁰)₂, —C(O)R¹⁰, —C(O)OR¹⁰, —OC(O)R¹⁰, —NO₂, ═O, ═S, ═N(R¹⁰), —CN, C₁₋₆ alkyl, C₂₋₆ alkenyl, and C₂₋₆ alkynyl; and
wherein any substitutable carbon on the benzazepine core is optionally substituted by a substituent selected from halogen, —OR¹⁰, —SR¹⁰, —C(O)N(R¹⁰)₂, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —P(O)(OR¹⁰)₂, —OP(O)(OR¹⁰)₂, —CN, C₁₋₁₀ alkyl, C₂₋₁₀ alkenyl, and C₂₋₁₀ alkynyl, or two substituents on a single carbon atom or two adjacent carbons combine to form a 3- to 7-membered carbocycle.

4. The compound or salt of claim 1, wherein the compound of Formula (IIIA) is represented by Formula (IIIB): 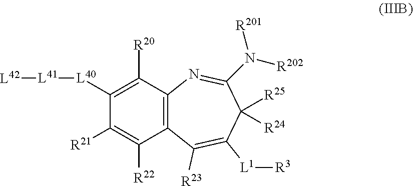
or a pharmaceutically acceptable salt thereof, wherein:
R²⁰, R²¹, R²², and R²³ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; and
R²⁴ and R²⁵ are independently selected from hydrogen, halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

5. The compound or salt of claim 2 or 4, wherein R²⁰, R²¹, R²², and R²³ are independently selected from hydrogen, halogen, —OH, —NO₂, —CN, and C₁₋₁₀ alkyl.

6. The compound or salt of claim 5, wherein R²⁰, R²¹, R²², and R²³ are each hydrogen.

7. The compound or salt of claim 2, 4, 5 or 6, wherein R²⁴ and R²⁵ are independently selected from hydrogen, halogen, —OH, —NO₂, —CN, and C₁₋₁₀ alkyl, or R²⁴ and R²⁵ taken together form an optionally substituted saturated C₃₋₇ carbocycle.

8. The compound or salt of claim 7, wherein R²⁴ and R²⁵ are each hydrogen.

9. The compound or salt of claim 7, wherein R²⁴ and R²⁵ taken together form an optionally substituted saturated C₃₋₅ carbocycle.

10. The compound or salt of claim 1 or 2, wherein R¹ is hydrogen.

11. The compound or salt of claim 1, 2, or 10, wherein R² is hydrogen.

12. The compound or salt of claim 3 or 4, wherein R²⁰² is an enzymatically-cleavable group.

13. The compound or salt of claim 3, 4, or 12, wherein R²⁰² is represented by the formula: 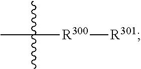
wherein:
R³⁰¹ is selected from an amino acid, a peptide, —O—(C₁-C₆ alkyl) and —C₁-C₆ alkyl, wherein alkyl of —O—(C₁-C₆ alkyl) and —C₁-C₆ alkyl is optionally substituted by one or more substituents independently selected from halogen, —OR¹⁰, —SR¹⁰, —N(R¹⁰)₂, —C(O)R¹⁰, —C(O)N(R¹⁰)₂, —NO₂, —CN, C₃₋₁₃ carbocycle, and 3- to 12-membered heterocycle; and
R³⁰⁰ is C(═O), wherein when R³⁰¹ is selected from an amino acid or peptide R³⁰⁰ is the C-terminus of the amino acid or peptide.

14. The compound or salt of claim 13, wherein R³⁰¹ is a peptide selected from a dipeptide, tripeptide and tetrapeptide.

15. The compound or salt of any one of claims 1 to 14, wherein L¹ is selected from —C(O)—, and —C(O)NR¹⁰—.

16. The compound or salt of claim 15, wherein L¹ is —C(O)—.

17. The compound or salt of claim 15, wherein L¹ is —C(O)NR¹⁰—.

18. The compound or salt of claim 17, wherein R¹⁰ of —C(O)NR¹⁰— is selected from hydrogen and C₁₋₆ alkyl.

19. The compound or salt of claim 18, wherein L¹ is —C(O)NH—.

20. The compound or salt of any one of claims 1 to 19, wherein R³ is selected from: —OR¹⁰, and —N(R¹⁰)₂; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —OR¹⁰, —SR¹⁰, —N(R¹⁰)₂, —S(O)R¹⁰, —S(O)₂R¹⁰, —C(O)R¹⁰, —C(O)OR¹⁰, —OC(O)R¹⁰, —NO₂, ═O, ═S, ═N(R¹⁰), —CN, C₁₋₁₀ alkyl, C₂₋₁₀ alkenyl, and C₂₋₁₀ alkynyl.

21. The compound or salt of claim 20, wherein R³ is —N(R¹⁰)₂.

22. The compound or salt of claim 21, wherein R¹⁰ of —N(R¹⁰)₂ is independently selected at each occurrence from optionally substituted C₁₋₆ alkyl.

23. The compound or salt of claim 22, wherein R¹⁰ of —N(R¹⁰)₂ is independently selected at each occurrence from methyl, ethyl, propyl, and butyl, any one of which is optionally substituted.

24. The compound or salt of claim 23, wherein R³ is

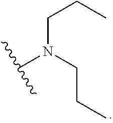 [SMILES: *N(CCC)CCC]

25. The compound or salt of any one of claims 1 to 24, wherein L⁴⁰ is an optionally substituted C₃₋₁₂ carbocyclene.

26. The compound or salt of claim 25, wherein L⁴⁰ is an optionally substituted C₃₋₈ carbocyclene.

27. The compound or salt of claim 26, wherein L⁴⁰ is an optionally substituted C₅₋₆ carbocyclene.

28. The compound or salt of claim 25, wherein L⁴⁰ is an optionally substituted arylene.

29. The compound or salt of claim 28, wherein L⁴⁰ is an optionally substituted arylene wherein substituents are independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl.

30. The compound or salt of claim 29, wherein L⁴⁰ is an optionally substituted phenylene.

31. The compound or salt of any one of claims 1 to 24, wherein L⁴⁰ is an optionally substituted 3- to 12-membered heterocyclene.

32. The compound or salt of claim 31, wherein L⁴⁰ is an optionally substituted 3- to 8-membered heterocyclene.

33. The compound or salt of claim 32, wherein L⁴⁰ is an optionally substituted 5- to 6-membered heterocyclene.

34. The compound or salt of claim 31, wherein L⁴⁰ is an optionally substituted heteroarylene.

35. The compound or salt of claim 34, wherein L⁴⁰ is an optionally substituted heteroaryl ene substituted with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl.

36. The compound or salt of claim 35, wherein L⁴⁰ is an optionally substituted 5- or 6-membered heteroaryl ene.

37. The compound or salt of claim 36, wherein L⁴⁰ is an optionally substituted 6-membered heteroaryl ene.

38. The compound or salt of claim 37, wherein L⁴⁰ is optionally substituted pyridinylene.

39. The compound or salt of any one of claims 1-38, wherein L⁴¹ is selected from —N(R¹⁰)—, —C(O)N(R¹⁰)—, and —C(O)—.

40. The compound or salt of claim 39, wherein L⁴¹ is —C(O)—.

41. The compound or salt of any one of claims 1-40, wherein L⁴² is selected from optionally substituted C₃₋₁₂ carbocycle, optionally substituted 3- to 12-membered unsaturated heterocycle, optionally substituted heteroaryl, and optionally substituted 8-14 membered bicyclic heterocycle.

42. The compound or salt of any one of claims 1-41, wherein L⁴² is an optionally substituted 8- to 14-membered bicyclic heterocycle.

43. The compound or salt of claim 42, wherein L⁴² is an optionally substituted 8- to 12-membered bicyclic heterocycle.

44. The compound or salt of claim 43, wherein L⁴² is an optionally substituted 8- to 12-membered bicyclic heterocycle with one or more substituents independently selected from halogen, —OR¹⁰, —SR¹⁰, —N(R¹⁰)₂, —C(O)R¹⁰, —C(O)OR¹⁰, —OC(O)R¹⁰, —NO₂, ═O, ═S, —CN, C₁₋₆ alkyl, C₂₋₆ alkenyl, and C₂₋₆ alkynyl.

45. The compound or salt of claim 44, wherein L⁴² is an optionally substituted 8- to 12-membered bicyclic heterocycle with one or more substituents independently selected from —OR¹⁰, —N(R¹⁰)₂, and ═O.

46. The compound or salt of any one of claims 1-40, where in L⁴² is a 3- to 8-membered saturated heterocycle substituted with a substituent selected from R³⁰, and optionally substituted with one or more substituents selected from R³¹.

47. The compound or salt of claim 46, wherein L⁴² is a 5- to 6-membered saturated heterocycle substituted with a substituent selected from R³⁰, and optionally substituted with one or more substituents selected from R³¹.

48. The compound or salt of claim 47, wherein R³⁰ is selected from: halogen, —OR¹¹, —SR¹⁰, —C(O)N(R¹⁰)₂, —N(R¹⁰)₂, —C(O)OR¹⁰, —NO₂, and —CN; C₁₋₁₀ alkyl, C₂₋₁₀ alkenyl, and C₂₋₁₀ alkynyl, each of which is independently optionally substituted at each occurrence with one or more substituents; and C₃₋₁₂ carbocycle and 3- to 12-membered heterocycle, each of which is independently optionally substituted with one or more substituents.

49. The compound or salt of claim 48, wherein R³⁰ is selected from —OR¹¹; C₁₋₁₀ alkyl, C₂₋₁₀ alkenyl, and C₂₋₁₀ alkynyl, each of which is independently optionally substituted at each occurrence with one or more substituents; and C₃₋₁₂ carbocycle and 3- to 12-membered heterocycle, each of which is optionally substituted with one or more substituents.

50. The compound or salt of claim 46, wherein R³¹ is selected from halogen, —OR¹⁰, —SR¹⁰, —C(O)N(R¹⁰)₂, —N(R¹⁰)₂, —C(O)OR¹⁰, —NO₂, and —CN; C₁₋₁₀ alkyl, C₂₋₁₀ alkenyl, and C₂₋₁₀ alkynyl, each of which is optionally substituted with one or more independently selected substituents; and C₃₋₁₂ carbocycle and 3- to 12-membered heterocycle, each of which is optionally substituted with one or more independently selected substituents.

51. The compound or salt of claim 50, wherein R³¹ is selected from —OR¹⁰; C₁₋₁₀ alkyl, C₂₋₁₀ alkenyl, and C₂₋₁₀ alkynyl, each of which is optionally substituted with one or more independently selected substituents; and C₃₋₁₂ carbocycle and 3- to 12-membered heterocycle, wherein each of which is optionally substituted with one or more independently selected substituents.

52. The compound or salt of claim 46, wherein L⁴² is pyrrolidine substituted with a substituent selected from R³⁰, and optionally substituted with one or more substituents selected from R³¹.

53. The compound or salt of claim 46, wherein L⁴² is piperidine substituted with a substituent selected from R³⁰, and optionally substituted with one or more substituents selected from R³¹.

54. The compound or salt of claim 1, wherein the compound is selected from: 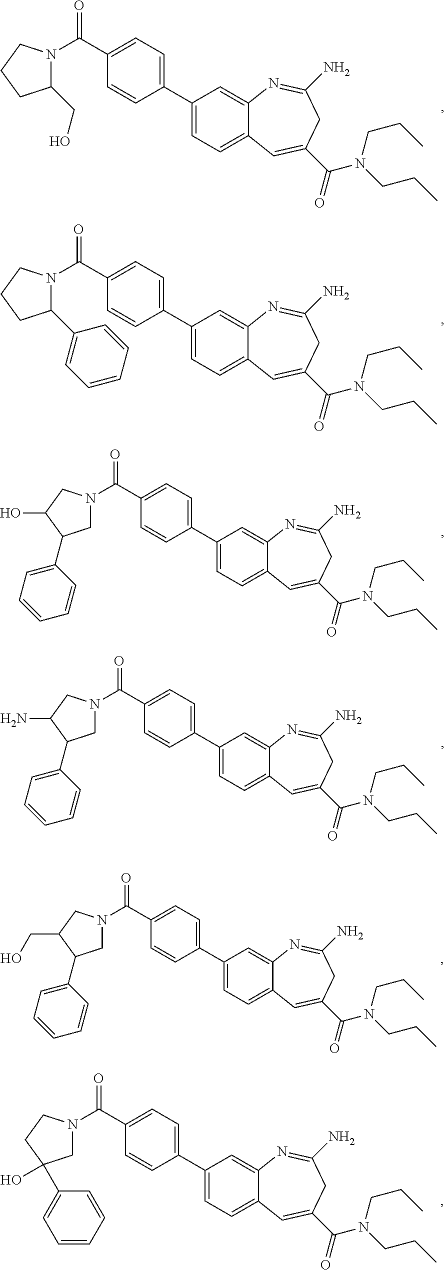 [SMILES: CCCN(CCC)C(=O)C1=Cc2ccc(-c3ccc(C(=O)N4CCCC4CO)cc3)cc2N=C(N)C1 ; CCCN(CCC)C(=O)C1=Cc2ccc(-c3ccc(C(=O)N4CCCC4c4ccccc4)cc3)cc2N=C(N)C1 ; CCCN(CCC)C(=O)C1=Cc2ccc(-c3ccc(C(=O)N4CC(O)C(c5ccccc5)C4)cc3)cc2N=C(N)C1 ; CCCN(CCC)C(=O)C1=Cc2ccc(-c3ccc(C(=O)N4CC(N)C(c5ccccc5)C4)cc3)cc2N=C(N)C1 ; CCCN(CCC)C(=O)C1=Cc2ccc(-c3ccc(C(=O)N4CC(CO)C(c5ccccc5)C4)cc3)cc2N=C(N)C1 ; CCCN(CCC)C(=O)C1=Cc2ccc(-c3ccc(C(=O)N4CCC(O)(c5ccccc5)C4)cc3)cc2N=C(N)C1] 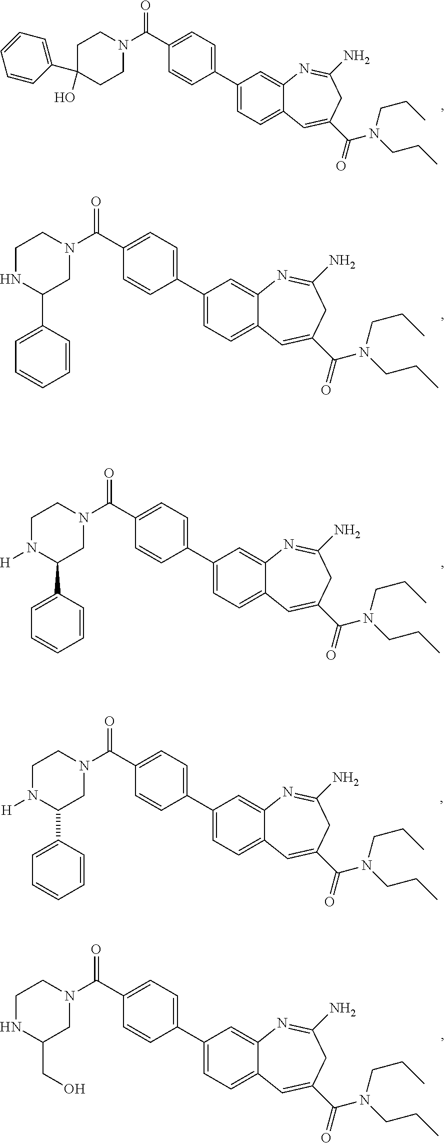 [SMILES: CCCN(CCC)C(=O)C1=Cc2ccc(-c3ccc(C(=O)N4CCC(O)(c5ccccc5)CC4)cc3)cc2N=C(N)C1 ; CCCN(CCC)C(=O)C1=Cc2ccc(-c3ccc(C(=O)N4CCNC(c5ccccc5)C4)cc3)cc2N=C(N)C1 ; CCCN(CCC)C(=O)C1=Cc2ccc(-c3ccc(C(=O)N4CCN[C@H](c5ccccc5)C4)cc3)cc2N=C(N)C1 ; CCCN(CCC)C(=O)C1=Cc2ccc(-c3ccc(C(=O)N4CCN[C@@H](c5ccccc5)C4)cc3)cc2N=C(N)C1 ; CCCN(CCC)C(=O)C1=Cc2ccc(-c3ccc(C(=O)N4CCNC(CO)C4)cc3)cc2N=C(N)C1] 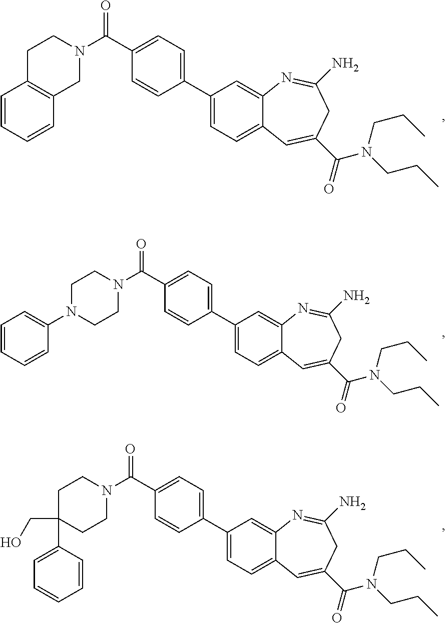 [SMILES: CCCN(CCC)C(=O)C1=Cc2ccc(-c3ccc(C(=O)N4CCc5ccccc5C4)cc3)cc2N=C(N)C1 ; CCCN(CCC)C(=O)C1=Cc2ccc(-c3ccc(C(=O)N4CCN(c5ccccc5)CC4)cc3)cc2N=C(N)C1 ; CCCN(CCC)C(=O)C1=Cc2ccc(-c3ccc(C(=O)N4CCC(CO)(c5ccccc5)CC4)cc3)cc2N=C(N)C1]
and a salt of any one thereof.

55. A compound represented by the structure of Formula (IIA): 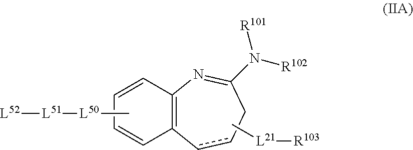
or a pharmaceutically acceptable salt thereof, wherein: [symbol]
represents an optional double bond;
L⁵⁰ is selected from C₃₋₁₂ carbocyclene and 3- to 12-membered heterocyclene, wherein the C₃₋₁₂ carbocyclene and the 3- to 12-membered heterocyclene are optionally substituted with one or more substituents independently selected at each occurrence from:
halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
L²¹ and L⁵¹ are independently selected from a bond, C₁-C₂ alkylene optionally substituted with one or more R³¹⁰, —O—, —S—, —N(R¹⁰⁰)—, —C(O)—, —C(O)O—, —OC(O)—, —C(O)N(R¹⁰⁰)—, —N(R¹⁰⁰)C(O)—, —C(NR¹⁰⁰)—, —P(O)(OR¹⁰⁰)O—, —O(R¹⁰⁰O)(O)P—, —OS(O)—, —S(O)O—, —S(O)—, —OS(O)₂, —S(O)₂O—, —N(R¹⁰⁰)S(O)₂—, —S(O)₂N(R¹⁰⁰)—, —N(R¹⁰⁰)S(O)—, and —S(O)N(R¹⁰⁰)—;
L⁵² is selected from optionally substituted C₃₋₁₂ carbocycle, optionally substituted 3- to 12-membered unsaturated heterocycle, optionally substituted heteroaryl, optionally substituted 8-14 membered bicyclic heterocycle, and optionally substituted 3- to 8-membered saturated heterocycle, each of which is optionally substituted with one or more substituents independently selected from:
halogen, -L², —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
R¹⁰¹ and R¹⁰² are independently selected from L², and hydrogen; and C₁₋₁₀ alkyl, C₂₋₁₀ alkenyl, and C₂₋₁₀ alkynyl, each of which is optionally substituted with one or more substituents independently selected from L², halogen, —OR¹⁰⁰, —SR¹⁰⁰, —C(O)N(R¹⁰⁰)₂, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), and —CN;
R¹⁰³ is selected from:
-L², —OR¹⁰⁰, —N(R¹⁰⁰)₂, —C(O)N(R¹⁰⁰)₂, —C(O)R¹⁰⁰, —C(O)OR¹⁰⁰, —S(O)R¹⁰⁰, and —S(O)₂R¹⁰⁰; and
C₁₋₁₀ alkyl, C₂₋₁₀ alkenyl, and C₂₋₁₀ alkynyl, each of which is optionally substituted with one or more substituents independently selected from L², halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from L², halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
R¹⁰⁰ is independently selected at each occurrence from L² and hydrogen; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R³¹⁰ is selected from:
halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
L² is a linker wherein at least one of R¹⁰¹, R¹⁰², R¹⁰³, and R¹⁰⁰ is L² or at least one substituent on R¹⁰¹, R¹⁰², R¹⁰³, L⁵², L²¹ and L⁵¹ is -L²; and
wherein any substitutable carbon on the benzazepine core is optionally substituted by a substituent selected from halogen, —OR¹⁰⁰, —SR¹⁰⁰, —C(O)N(R¹⁰⁰)₂, —N(R¹⁰⁰)₂, —S(O)R¹⁰⁰, —S(O)₂R¹⁰⁰,
—C(O)R¹⁰⁰, —C(O)OR¹⁰⁰, —OC(O)R¹⁰⁰, —NO₂, ═O, ═S,
═N(R¹⁰⁰), —P(O)(OR¹⁰⁰)₂, —OP(O)(OR¹⁰⁰)₂, —CN, C₁₋₁₀ alkyl, C₂₋₁₀ alkenyl, and C₂₋₁₀ alkynyl, or two substituents on a single carbon atom or two adjacent carbons combine to form a 3- to 7-membered carbocycle.

56. The compound or salt of claim 50, wherein the compound of Formula (IIA) is represented by Formula (IIB): 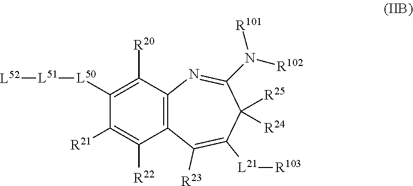
or a pharmaceutically acceptable salt thereof, wherein:
R²⁰, R²¹, R²², and R²³ are independently selected from hydrogen, halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl; and
R²⁴, and R²⁵ are independently selected from hydrogen, halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

57. A compound represented by the structure of Formula (IVA): 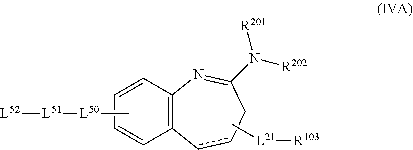
or a pharmaceutically acceptable salt thereof, wherein: [symbol]
represents an optional double bond;
L⁵⁰ is selected from C₃₋₁₂ carbocyclene and 3- to 12-membered heterocyclene, wherein the C₃₋₁₂ carbocyclene and the 3- to 12-membered heterocyclene are optionally substituted with one or more substituents independently selected at each occurrence from:
halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
L²¹ and L⁵¹ are independently selected from a bond, C₁-C₂ alkylene optionally substituted with one or more R³¹⁰, —O—, —S—, —N(R¹⁰⁰)—, —C(O)—, —C(O)O—, —OC(O)—, —C(O)N(R¹⁰⁰)—, —N(R¹⁰⁰)C(O)—, —C(NR¹⁰⁰)—, —P(O)(OR¹⁰⁰)O—, —O(R¹⁰⁰O)(O)P—, —OS(O)—, —S(O)O—, —S(O)—, —OS(O)₂—, —S(O)₂O—, —N(R¹⁰⁰)S(O)₂—, —S(O)₂N(R¹⁰⁰)—, —N(R¹⁰⁰)S(O)—, and —S(O)N(R¹⁰⁰)—;
L⁵² is selected from optionally substituted C₃₋₁₂ carbocycle, optionally substituted 3- to 12-membered unsaturated heterocycle, optionally substituted heteroaryl, optionally substituted 8-14 membered bicyclic heterocycle, and optionally substituted 3- to 8-membered saturated heterocycle, each of which is optionally substituted with one or more substituents independently selected from:
halogen, -L², —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN; and
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
R²⁰¹ is hydrogen;
R²⁰² is an amine masking group;
R¹⁰³ is selected from:
-L², —OR¹⁰⁰, —N(R¹⁰⁰)₂, —C(O)N(R¹⁰⁰)₂, —C(O)R¹⁰⁰, —C(O)OR¹⁰⁰, —S(O)R¹⁰⁰, and —S(O)₂R¹⁰⁰; and
C₁₋₁₀ alkyl, C₂₋₁₀ alkenyl, and C₂₋₁₀ alkynyl, each of which is optionally substituted with one or more substituents independently selected from L², halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from L², halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
R¹⁰⁰ is independently selected at each occurrence from L² and hydrogen; and C₁₋₁₀ alkyl, C₂₋₁₀ alkenyl, C₂₋₁₀ alkynyl, C₃₋₁₂ carbocycle, and 3- to 12-membered heterocycle, each of which is optionally substituted with one or more substituents independently selected from halogen, —CN, —NO₂, —NH₂, ═O, ═S, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and haloalkyl;
R³¹⁰ is selected from:
halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), and —CN;
C₁₋₁₀ alkyl, C₂₋₁₀ alkenyl, and C₂₋₁₀ alkynyl, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰,
—OC(O)R¹⁰⁰, —NO₂, ═O, ═S, ═N(R¹⁰⁰), —CN, C₃₋₁₂ carbocycle, and 3- to 12-membered heterocycle; and
C₃₋₁₂ carbocycle and 3- to 12-membered heterocycle, each of which is optionally substituted with one or more substituents independently selected from halogen, —OR¹⁰⁰, —SR¹⁰⁰, —C(O)N(R¹⁰⁰)₂, —N(R¹⁰⁰)C(O)R¹⁰⁰, —N(R¹⁰⁰)C(O)N(R¹⁰⁰)₂, —N(R¹⁰⁰)₂, —C(O)R¹⁰⁰, —C(O)OR¹⁰⁰, —OC(O)R¹⁰⁰, —NO₂, ═O, ═S, ═N(R¹⁰⁰), —CN, C₁₋₆ alkyl, C₂₋₆ alkenyl, and C₂₋₆ alkynyl;
L² is a linker wherein at least one of R²⁰¹, R²⁰², R¹⁰³, and R¹⁰⁰ is L² or at least one substituent on R²⁰¹, R²⁰², R¹⁰³, L⁵², L²¹ and L⁵¹ is -L²; and
wherein any substitutable carbon on the benzazepine core is optionally substituted by a substituent selected from halogen, —OR¹⁰⁰, —SR¹⁰⁰, —C(O)N(R¹⁰⁰)₂, —N(R¹⁰⁰)₂, —S(O)R¹⁰⁰, —S(O)₂R¹⁰⁰,
—C(O)R¹⁰⁰, —C(O)OR¹⁰⁰, —OC(O)R¹⁰⁰, —NO₂, ═O, ═S,
═N(R¹⁰⁰), —P(O)(OR¹⁰⁰)₂, —OP(O)(OR¹⁰⁰)₂, —CN, C₁₋₁₀ alkyl, C₂₋₁₀ alkenyl, and C₂₋₁₀ alkynyl, or two substituents on a single carbon atom or two adjacent carbons combine to form a 3- to 7-membered carbocycle.

58. The compound or salt of claim 50, wherein the compound of Formula (IVA) is represented by Formula (IVB): 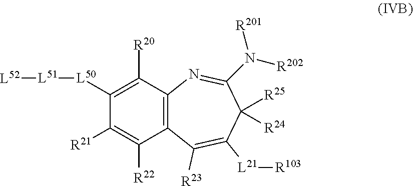
or a pharmaceutically acceptable salt thereof, wherein:
R²⁰, R²¹, R²², and R²³ are independently selected from hydrogen, halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl; and
R²⁴, and R²⁵ are independently selected from hydrogen, halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl; or R²⁴ and R²⁵ taken together form an optionally substituted saturated C₃₋₇ carbocycle.

59. The compound or salt of claim 55 or 56, wherein R¹⁰¹ is -L².

60. The compound or salt of claim 55 or 56, wherein R¹⁰² is -L².

61. The compound or salt of claim 57 or 58, wherein R²⁰² is an enzymatically-cleavable group.

62. The compound or salt of claim 57, 58, or 61, wherein R²⁰² is represented by the formula: 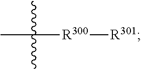
wherein:
R³⁰¹ is selected from an amino acid, a peptide, —O—(C₁-C₆ alkyl) and —C₁-C₆ alkyl, wherein alkyl of —O—(C₁-C₆ alkyl) and —C₁-C₆ alkyl is optionally substituted by one or more substituents independently selected from halogen, —OR¹⁰, —SR¹⁰, —N(R¹⁰)₂, —C(O)R¹⁰, —C(O)N(R¹⁰)₂, —NO₂, —CN, C₃₋₁₃ carbocycle, and 3- to 12-membered heterocycle; and
R³⁰⁰ is C(═O), wherein when R³⁰¹ is selected from an amino acid or peptide R³⁰⁰ is the C-terminus of the amino acid or peptide.

63. The compound or salt of claim 62, wherein R³⁰¹ is a peptide selected from a dipeptide, tripeptide and tetrapeptide.

64. The compound or salt of any one of claims 55-63, wherein L²¹ is —C(O)—.

65. The compound or salt of any one of claims 55-63, wherein L²¹ is —C(O)NR¹⁰⁰—.

66. The compound or salt of claim 65, wherein R¹⁰⁰ of —C(O)NR¹⁰⁰— is selected from hydrogen, C₁₋₆ alkyl, and -L².

67. The compound or salt of claim 66, L²¹ is —C(O)NH—.

68. The compound or salt of any one of claims 55 to 67, wherein R¹⁰³ is selected from -L², —OR¹⁰⁰, and —N(R¹⁰⁰)₂; and C₁₋₁₀ alkyl, C₂₋₁₀ alkenyl, C₂₋₁₀ alkynyl, C₃₋₁₂ carbocycle, 3- to 12-membered heterocycle, aryl, and heteroaryl, each of which is independently optionally substituted at each occurrence with one or more substituents selected from -L², halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, C₁₋₁₀ alkyl, C₂₋₁₀ alkenyl, and C₂₋₁₀ alkynyl.

69. The compound or salt of claim 68, wherein each R¹⁰⁰ of —N(R¹⁰⁰)₂ is selected from -L² and hydrogen, and wherein no more than one R¹⁰⁰ of —N(R¹⁰⁰)₂ is -L².

70. The compound or salt of any one of claims 55 to 69, wherein L⁵⁰ is an optionally substituted arylene wherein substituents are independently selected from halogen, —OR¹⁰⁰, —SR¹⁰⁰, —N(R¹⁰⁰)₂, —C(O)R¹⁰⁰, —C(O)OR¹⁰⁰, —OC(O)R¹⁰⁰, —NO₂, ═O, ═S, —CN, C₁₋₆ alkyl, C₂₋₆ alkenyl, and C₂₋₆ alkynyl.

71. The compound or salt of claim 70, wherein L⁵⁰ is an optionally substituted phenylene.

72. The compound or salt of any one of claims 55 to 71, wherein L⁵¹ is —C(O)N(R¹⁰⁰)—.

73. The compound or salt of claim 72, wherein R¹⁰⁰ of —C(O)N(R¹⁰⁰)— is selected from hydrogen, C₁₋₆ alkyl, and -L².

74. The compound or salt of claim 73, wherein L⁵¹ is —C(O)NH—.

75. The compound or salt of any one of claims 55-74, wherein L⁵² is an optionally substituted 8- to 14-membered bicyclic heterocycle.

76. The compound or salt of claim 75, wherein L⁵² is an optionally substituted 8- to 12-membered bicyclic heterocycle with one or more substituents independently selected from —OR¹⁰⁰, —N(R¹⁰⁰)₂, and ═O.

77. The compound or salt of any one of claims 55-74, wherein L⁵² is a 3- to 8-membered saturated heterocycle optionally substituted with one or more substituents selected from R³¹⁰.

78. The compound or salt of claim 77, wherein R³¹⁰ is selected from L² and —OR¹⁰⁰; C₁₋₁₀ alkyl, C₂₋₁₀ alkenyl, and C₂₋₁₀ alkynyl, each of which is optionally substituted with one or more independently selected substituents; and C₃₋₁₂ carbocycle and 3- to 12-membered heterocycle each of which is optionally substituted with one or more independently selected substituents.

79. The compound or salt of any one of claims 77-78, wherein L⁵² is pyrrolidine optionally substituted with one or more substituents selected from R³¹⁰.

80. The compound or salt of any one of claims 77-78, wherein L⁵² is piperidine optionally substituted with one or more substituents selected from R³¹⁰.

81. The compound or salt of any one of claims 55 to 80, in which L² is a cleavable linker or a noncleavable linker.

82. The compound or salt of claim 81, in which L² is a cleavable linker that is cleavable by a lysosomal enzyme.

83. The compound or salt of any one of claims 55 to 82, wherein L² is represented by the formula: 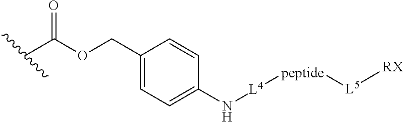
wherein:
L⁴ represents the C-terminus of the peptide and L⁵ is selected from a bond, alkylene and heteroalkylene, wherein L⁵ is optionally substituted with one or more groups independently selected from R³⁰, and RX is a reactive moiety; and
R³⁰ is independently selected at each occurrence from halogen, —OH, —CN, —O-alkyl, —SH, ═O, ═S, —NH₂, and —NO₂; and C₁-C₁₀ alkyl, C₂-C₁₀ alkenyl, and C₂-C₁₀ alkynyl, each of which is independently optionally substituted at each occurrence with one or more substituents selected from halogen, —OH, —CN, —O-alkyl, —SH, ═O, ═S, —NH₂, and —NO₂.

84. The compound or salt of claim 83, wherein RX comprises a leaving group.

85. The compound or salt of claim 83, wherein RX is a maleimide or an alpha-halo carbonyl.

86. The compound or salt of any of claims 83 to 85, wherein the peptide of L² comprises Val-Cit or Val-Ala.

87. The compound or salt of any one of claims 55 to 81, wherein L² is represented by the formula: 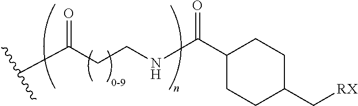
wherein:
RX comprises a reactive moiety; and
n is 0-9.

88. The compound or salt of claim 87, wherein RX comprises a leaving group.

89. The compound or salt of claim 87, wherein RX is a maleimide or an alpha-halo carbonyl.

90. The compound or salt of any one of claims 55 to 89, wherein L² is further covalently bound to a residue of an antibody construct to form a conjugate, the antibody construct comprising an antigen binding domain and an Fc domain.

91. A conjugate represented by the formula: 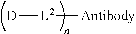
wherein:
Antibody is an antibody construct, the antibody construct comprising an antigen binding domain and an Fc domain;
n is 1 to 20;
D is the compound or salt of any one of claims 1 to 54; and
L² is a linker moiety attached to a residue of the antibody construct and to D.

92. The conjugate of claim 91, wherein n is selected from 1 to 8.

93. The conjugate of claim 92, wherein n is selected from 2 to 5.

94. The conjugate of claim 93, wherein n is 2 or 4.

95. The conjugate of any one of claims 91 to 94, wherein -L² is represented by the formula: 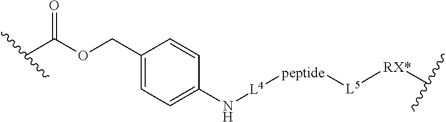
wherein:
L⁴ represents the C-terminus of the peptide and L⁵ is selected from a bond, alkylene and heteroalkylene, wherein L⁵ is optionally substituted with one or more groups independently selected from R³⁰; RX* is a bond, a succinimide moiety, or a hydrolyzed succinimide moiety bound to the residue of the antibody construct, wherein [symbol] on RX* represents the point of attachment to the residue of the antibody construct; and
R³⁰ is independently selected at each occurrence from halogen, —OH, —CN, —O-alkyl, —SH, ═O, ═S, —NH₂, and —NO₂; and C₁-C₁₀alkyl, C₂-C₁₀alkenyl, and C₂-C₁₀alkynyl, each of which is independently optionally substituted at each occurrence with one or more substituents selected from halogen, —OH, —CN, —O-alkyl, —SH, ═O, ═S, —NH₂, and —NO₂.

96. The conjugate of claim 95, wherein RX* is a succinamide moiety, hydrolyzed succinamide moiety or a mixture thereof and is bound to a cysteine residue of an antibody construct.

97. The conjugate of any one of claims 91 to 94, wherein -L² is represented by the formula: 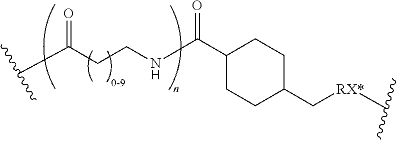
wherein:
RX* is a bond, a succinimide moiety, or a hydrolyzed succinimide moiety bound to the residue of the antibody construct, wherein [symbol] on RX* represents the point of attachment to the residue of the antibody construct; and
n is 0-9.

98. The conjugate of any one of claims 91 to 97, wherein the antigen binding domain specifically binds to an antigen selected from the group consisting of HER2, TROP2 and MUC16.

99. The conjugate of any one of claims 90 to 98, wherein the Fc domain is an Fc null.

100. A pharmaceutical composition, comprising a conjugate of any one of claims 90 to 99, and a pharmaceutically acceptable excipient.

101. The pharmaceutical composition of claim 100, wherein the average Drug-to-Antibody Ratio (DAR) is from 1 to 8.

102. A method for the treatment of cancer, comprising administering an effective amount of the compound or salt of any one of claims 1 to 54 to a subject in need thereof.

103. A method for the treatment of cancer, comprising administering an effective amount of the conjugate of any one of claims 91 to 99 or the pharmaceutical composition of any one of claims 100-101 to a subject in need thereof.

104. A method of killing tumor cells in vivo, comprising contacting a tumor cell population with the conjugate of any one of claims 91 to 99 or the pharmaceutical composition of any one of claims 100-101.

105. A method for treatment, comprising administering to a subject the conjugate of any one of claims 91 to 99 or the pharmaceutical composition of any one of claims 100-101.

106. A method for the treatment of cancer, comprising administering to a subject in need thereof the conjugate of any one of claims 91 to 99 or the pharmaceutical composition of any one of claims 100-101.

107. The method of claim 106, wherein the cancer is breast cancer, gastric cancer or lung cancer.

108. A compound or salt of any one of claims 1 to 54 for use in a method of treatment of a subject's body by therapy.

109. A compound or salt of any one of claims 1 to 54 for use in a method of treating cancer.

110. A conjugate of any one of claims 91 to 99 or the pharmaceutical composition of any one of claims 100-101 for use in a method of treatment of a subject's body by therapy.

111. A conjugate of any one of claims 91 to 99 or the pharmaceutical composition of any one of claims 100-101 for use in a method of treating cancer.

112. A method of preparing an antibody conjugate of the formula: 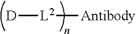
wherein:
Antibody is an antibody construct;
n is selected from 1 to 20; and
D-L² is selected from a compound or salt of any one of claims 55 to 90,
comprising contacting D-L² with an antibody construct to form the antibody conjugate.

113. A method of preparing an antibody conjugate of the formula: 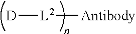
wherein:
Antibody is an antibody construct;
n is selected from 1 to 20;
L² is a linker; and
D is selected from a compound or salt of any one of claims 1-54,
comprising contacting L² with the antibody construct to form L²-antibody and contacting L²-antibody with D to form the antibody conjugate.

114. The method of any one of claim 112 or 113, wherein the antibody construct comprises an antigen binding domain that specifically binds to an antigen selected from the group consisting of HER2, TROP2 and MUC16.

115. The method of any one of claims 112 to 114, further comprising purifying the antibody conjugate.

116. A compound or salt thereof selected from compounds 1.1-1.11.

117. A compound or salt of any one of claims 55 to 56, wherein one of R¹⁰¹, R¹⁰², R¹⁰³, and R¹⁰⁰ is L² or one substituent on R¹⁰¹, R¹⁰², R¹⁰³, L⁵², L²¹ and L⁵¹ is -L².

118. A compound or salt of any one of claims 57 to 58, wherein one of R²⁰¹, R²⁰², R¹⁰³, and R¹⁰⁰ is L² or one substituent on R²⁰¹, R²⁰², R¹⁰³, L⁵², L²¹ and L⁵¹ is -L².

119. A compound or salt of any one of claims 55 to 89, wherein L² is covalently bound to a nitrogen atom or oxygen atom.

120. A compound or salt of claim 119, wherein L² is covalently bound to a nitrogen atom.

121. A compound or salt of any one of claims 55 to 58, wherein L² comprises 15 or more consecutive atoms.


SEQUENCE LISTING (27 sequences)

SEQ ID NO: 1 - protein, 119 aa, Artificial Sequence [sequence omitted]

SEQ ID NO: 2 - protein, 107 aa, Artificial Sequence [sequence omitted]

SEQ ID NO: 3 - protein, 121 aa, Artificial Sequence
  source: /note=“Description of Artificial Sequence Synthetic polypeptide”
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 4 - protein, 107 aa, Artificial Sequence
  source: /note=“Description of Artificial Sequence Synthetic polypeptide”
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 5 - protein, 120 aa, Artificial Sequence
  source: /note=“Description of Artificial Sequence Synthetic polypeptide”
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSS

SEQ ID NO: 6 - protein, 112 aa, Artificial Sequence
  source: /note=“Description of Artificial Sequence Synthetic polypeptide”
DVVMTQSPLSLPVTLGQPASISCRSSQSLLHSSGNTYLEYFQQRPGQSPRPLIYKISTRFSGVPDRFSGSGSGTDFTLKISRVEAEDVGVYYCFQGSHVPYTFGGGTKVEIK

SEQ ID NO: 7 - protein, 120 aa, Artificial Sequence [sequence omitted]

SEQ ID NO: 8 - protein, 107 aa, Artificial Sequence [sequence omitted]

SEQ ID NO: 9 - protein, 123 aa, Artificial Sequence
  source: /note=“Description of Artificial Sequence Synthetic polypeptide”
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 10 - protein, 106 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 11 - protein, 118 aa, Artificial Sequence [sequence omitted]

SEQ ID NO: 12 - protein, 107 aa, Artificial Sequence [sequence omitted]

SEQ ID NO: 13 - protein, 123 aa, Artificial Sequence
  source: /note=“Description of Artificial Sequence Synthetic polypeptide”
EVKLQESGGGFVKPGGSLKVSCAASGFTFSSYAMSWVRLSPEMRLEWVATISSAGGYIFYSDSVQGRFTISRDNAKNTLHLQMGSLRSGDTAMYYCARQGFGNYGDYYAMDYWGQGTTVTVSS

SEQ ID NO: 14 - protein, 112 aa, Artificial Sequence
  source: /note=“Description of Artificial Sequence Synthetic polypeptide”
DIELTQSPSSLAVSAGEKVTMSCKSSQSLLNSRTRKNQLAWYQQKPGQSPELLIYWASTRQSGVPDRFTGSGSGTDFTLTISSVQAEDLAVYYCQQSYNLLTFGPGTKLEVK

SEQ ID NO: 15 - protein, 123 aa, Artificial Sequence
  source: /note=“Description of Artificial Sequence Synthetic polypeptide”
EVKLEESGGGFVKPGGSLKISCAASGFTFRNYAMSWVRLSPEMRLEWVATISSAGGYIFYSDSVQGRFTISRDNAKNTLHLQMGSLRSGDTAMYYCARQGFGNYGDYYAMDYWGQGTTVTVSS

SEQ ID NO: 16 - protein, 112 aa, Artificial Sequence
  source: /note=“Description of Artificial Sequence Synthetic polypeptide”
DIELTQSPSSLAVSAGEKVTMSCKSSQSLLNSRTRKNQLAWYQQKTGQSPELLIYWASTRQSGVPDRFTGSGSGTDFTLTISSVQAEDLAVYYCQQSYNLLTFGPGTKLEIK

SEQ ID NO: 17 - protein, 120 aa, Artificial Sequence
  source: /note=“Description of Artificial Sequence Synthetic polypeptide”
QVQLVQSGAEVKKPGASVKVSCKASGYTIEDYYMHWVRQAPGQGLEWMGWIDPENGDTEYAPTFQGRVTMTRDTSINTAYMELSRLRSDDTAVYYCARHDAHYGTWFAYWGQGTLVTVSS

SEQ ID NO: 18 - protein, 112 aa, Artificial Sequence
  source: /note=“Description of Artificial Sequence Synthetic polypeptide”
DVVMTQSPLSLPVTLGQPASISCRSSQSIIRNDGNTYLEWYQQRPGQSPRRLIYRVSNRFSGVPDRFSGSGSGTDFTLKISRVEAEDVGVYYCFQGSHVPYTFGGGTKVEIK

SEQ ID NO: 19 - protein, 117 aa, Artificial Sequence
  source: /note=“Description of Artificial Sequence Synthetic polypeptide”
EIQLQQSGPELMKPGASVKISCKASTYSFTRYFMHWVKQSHGESLEWIGYIDPFNGGTGYNQKFKGKATLTVDKSSSTAYMHLSSLTSEDSAVYYCVTYGSDYFDYWGQGTTLTVSS

SEQ ID NO: 20 - protein, 108 aa, Artificial Sequence
  source: /note=“Description of Artificial Sequence Synthetic polypeptide”
DIVMTQPQKFMSTSVGDRVSVTCKASQNVETDVVWYQQKPGQPPKALIYSASYRHSGVPDRFTGSGSGTNFTLTISTVQSEDLAEYFCQQYNNYPFTFGSGTKLEIIR

SEQ ID NO: 21 - protein, 120 aa, Artificial Sequence; identical to SEQ ID NO: 5
  source: /note=“Description of Artificial Sequence Synthetic polypeptide”

SEQ ID NO: 22 - protein, 112 aa, Artificial Sequence
  source: /note=“Description of Artificial Sequence Synthetic polypeptide”
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIK

SEQ ID NO: 23 - protein, 116 aa, Artificial Sequence
  source: /note=“Description of Artificial Sequence Synthetic polypeptide”
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSS

SEQ ID NO: 24 - protein, 107 aa, Artificial Sequence
  source: /note=“Description of Artificial Sequence Synthetic polypeptide”
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIK

SEQ ID NO: 25 - protein, 5 aa, Unknown
  source: /note=“Description of Unknown recognition motif sequence”
  MOD_RES (3)..(3): Any amino acid
LPXTG

SEQ ID NO: 26 - protein, 4 aa, Artificial Sequence
  source: /note=“Description of Artificial Sequence Synthetic peptide”
GFLG

SEQ ID NO: 27 - protein, 4 aa, Artificial Sequence
  source: /note=“Description of Artificial Sequence Synthetic peptide”
ALAL